(19) United States
(12) Patent Application Publication
(10) Pub. No.: US 2019/0010139 A1
(43) Pub. Date: Jan. 10, 2019

(54) PYRIDIN-3-YL ACETIC ACID DERIVATIVES AS INHIBITORS OF HUMAN IMMUNODEFICIENCY VIRUS REPLICATION

(71) Applicant: ViiV Healthcare UK (No.5) Limited, Brentford, Middlesex (GB)

(72) Inventors: John F. KADOW, Wallingford, CT (US); B. Narasimhulu NAIDU, Wallington, CT (US); Jeffrey Lee ROMINE, Wallingford, CT (US); Denis R. ST. LAURENT, Wallingford, CT (US); Prasanna SIVAPRAKASAM, Wallington, CT (US)

(21) Appl. No.: 15/748,791

(22) PCT Filed: Aug. 3, 2016
(86) PCT No.: PCT/IB2016/054688

§ 371 Date: Jan. 30, 2018

Related U.S. Application Data
(60) Provisional application No. 62/202,521, filed on Aug. 7, 2015.

(51) Int. Cl. C07D 401/04; A61P 31/18; C07D 401/14; C07D 407/14; C07D 471/10; C07D 493/10

(52) U.S. Cl. CPC C07D 401/04; A61P 31/18; C07D 401/14; A61K 31/5365; C07D 471/10; C07D 493/10; A61K 2300/00; C07D 407/14

(57) ABSTRACT

Disclosed are compounds of Formula I, including pharmaceutically acceptable salts, pharmaceutical compositions comprising the compounds, methods for making the compounds and their use in inhibiting HIV integrase and treating those infected with HIV or AIDS. In the compounds of formula I, R¹ is selected from H, alkyl, haloalkyl, hydroxyalkyl, alkoxyalkyl, (alkoxy)alkoxyalkyl, or (R⁶)alkyl; R² is phenyl substituted with 1 R⁷ substituent and with 0-3 substituents selected from halo, alkyl, haloalkyl, alkoxy, and haloalkoxy; or R² is selected from tetrahydroisoquinolinyl, ((Ar¹)alkyl)tetrahydroisoquinolinyl, or ((N-alkoxycarbonyl) tetrahydroisoquinolinyl; R³ is selected from tetrahydroisoquinolinyl or decahydroisoquinolinyl and is substituted with 0-3 substituents selected from halo, alkyl, and haloalkyl; or R³ is a [5-7.3-7.0-2] fused or bridged bicyclic amine and is substituted with 0-3 alkyl substituents; or R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents; R⁴ is selected from alkyl or haloalkyl; R⁵ is selected from H, alkyl, haloalkyl, hydroxyalkyl, alkoxyalkyl, (alkoxy)alkoxyalkyl, or (R⁶) alkyl; R⁶ is selected from (oxetanyl)oxy, ((oxetanyl)alkoxy)alkyl, (tetrahydropyranyloxy)alkyl, (tetrahydropyranyl)alkoxy)alkyl, or (R⁹)(R⁹)N; R⁷ is selected from (Ar¹)alkoxy or ((Ar¹)alkyl)HNCO; R⁸ is selected from hydrogen, alkyl, (cycloalkyl)alkyl, alkoxyalkyl, (tetrahydropyanyl)alkyl, tetrahydropyanyl, or alkoxyphenyl; R⁹ is selected from hydrogen or alkyl; or (R⁸)(R⁹)N taken together is selected from azetidinyl, pyrrolidinyl, piperidinyl, (spirocyclobutyl) piperidinyl, piperazinyl, or morpholinyl; and Ar¹ is phenyl substituted with 0-3 substituents selected from halo, alkyl, haloalkyl, alkoxy, and haloalkoxy.

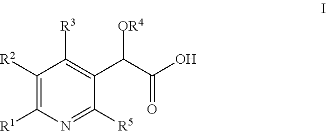

### CROSS REFERENCE TO RELATED INVENTION

This application claims the benefit of U.S. provisional application Ser. No. 62/202,521 filed Aug. 7, 2015.

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics indicate that an estimated 35.3 million people worldwide are infected with the virus (UNAIDS: Report on the Global HIV/AIDS Epidemic, 2013). In addition to the large number of individuals already infected, the virus continues to spread. Estimates from 2013 point to close to 3.4 million new infections in that year alone. In the same year there were approximately 1.6 million deaths associated with HIV and AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

In the US, where combination therapy is widely available, the number of HIV-related deaths has dramatically declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, initial studies suggest that approximately 30-50% of patients ultimately fail at least one drug in the suppressive combination. Treatment failure in most cases is caused by the emergence of viral resistance. Viral resistance in turn is caused by the replication rate of HIV-1 during the course of infection combined with the relatively high viral mutation rate associated with the viral polymerase and the lack of adherence of HIV-infected individuals in taking their prescribed medications. Clearly, there is a need for new antiviral agents, preferably with activity against viruses already resistant to currently approved drugs. Other important factors include improved safety and a more convenient dosing regimen than many of the currently approved drugs.

Compounds which inhibit HIV replication have been disclosed. See, for example, the following patent applications: WO2007131350, WO2009062285, WO2009062288, WO2009062289, WO2009062308, WO2010130034, WO2010130842, WO2011015641, WO2011076765, WO2012033735, WO2013123148, WO2013134113, WO2014164467, WO2014159959, and WO2015126726.

What is now needed in the art are additional compounds which are novel and useful in the treatment of HIV. Additionally, these compounds may desireably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of Formula I, including pharmaceutically acceptable salts thereof, as well as pharmaceutical compositions, and their use in inhibiting HIV and treating those infected with HIV or AIDS.

By virtue of the present invention, it is now possible to provide compounds that are novel and are useful in the treatment of HIV. Additionally, the compounds may provide advantages for pharmaceutical uses, for example, with regard to one or more of their mechanism of action, binding, inhibition efficacy, target selectivity, solubility, safety profiles, or bioavailability.

The invention also provides pharmaceutical compositions comprising the compounds of the invention, including pharmaceutically acceptable salts thereof, and a pharmaceutically acceptable carrier, excipient, and/or diluent.

In addition, the invention provides methods of treating HIV infection comprising administering a therapeutically effective amount of the compounds of the invention to a patient.

In addition, the invention provides methods for inhibiting HIV integrase.

Also provided in accordance with the invention are methods for making the compounds of the invention.

The present invention is directed to these, as well as other important ends, hereinafter described.

### DESCRIPTION OF THE INVENTION

Unless specified otherwise, these terms have the following meanings.

“Alkyl” means a straight or branched saturated hydrocarbon comprised of 1 to 10 carbons, and preferably 1 to 6 carbons.

“Alkenyl” means a straight or branched alkyl group comprised of 2 to 10 carbons with at least one double bond and optionally substituted with 0-3 halo or alkoxy group.

“Alkynyl” means a straight or branched alkyl group comprised of 2 to 10 carbons, preferably 2 to 6 carbons, containing at least one triple bond and optionally substituted with 0-3 halo or alkoxy group.

“Aryl” mean a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic groups include, but are not limited to indanyl, indenyl, naphthyl, phenyl, tetrahydronaphthyl and cyclopropylphenyl. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n=1-5, —CH(CH₃)Ph, —CH(Ph)₂.

“Aryloxy” is an aryl group attached to the parent structure by oxygen.

“Cycloalkyl” means a monocyclic ring system composed of 3 to 7 carbons.

“Halo” includes fluoro, chloro, bromo, and iodo.

“Haloalkyl” and “haloalkoxy” include all halogenated isomers from monohalo to perhalo.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from oxygen, nitrogen and sulfur. The rings could be bridged, fused and/or bonded, through a direct or spiro attachment, with the option to have one or a combination thereof be aromatic. Examples include, but are not limited to, azaindole, azaindoline, azetidine, benzimidazole, bezodioxolyl, benzoisothiazole, benzothiazole, benzothiadiazole, benzothiophene, benzoxazole, carbazole, chroman, dihalobezodioxolyl, dihydrobenzofuran, dihydrobenzo[1,4]oxazine, 1,3-dihydrobenzo[c]thiophene 2,2-dioxide, 2,3-dihydrobenzo[d]isothiazole 1,1-dioxide, 3,4-dihydro-2H-pyrido[3,2-b][1,4]oxazine, 2,3-dihydro-1H-pyrrolo[3,4-c]pyridine and its regioisomeric variants, 6,7-dihydro-5H-pyrrolo[2,3-b]pyrazine and its regioisomeric variants, furanylphenyl, imidazole, imidazo[1,2-a]pyridine, indazole, indole, indoline, isoquinoline, isoquinolinone, isothiazolidine 1,1-dioxide, morpholine, 2-oxa-5-azabicyclo[2.2.1]heptane, oxadiazole-phenyl, oxazole, phenylaztidine, phenylindazole, phenylpiperidine, phenylpiperizine, phenyloxazole, phenylpyrrolidine, piperidine, pyridine, pyridinylphenyl, pyridinylpyrrolidine, pyrimidine, pyrimidinylphenyl, pyrrazole-phenyl, pyrrolidine, pyrrolidin-2-one, 1H-pyrazolo[4,3-c]pyridine and its regioisomeric variants, pyrrole, 5H-pyrrolo[2,3-b]pyrazine, 7H-pyrrolo[2,3-d]pyrimidine and its regioisomeric variants, quinazoline, quinoline, quinoxaline, tetrahydroisoquinoline, 1,2,3,4-tetrahydro-1,8-naphthyridine, tetrahydroquinoline, 4,5,6,7-tetrahydrothieno[3,2-c]pyridine, 1,2,5-thiadiazolidine 1,1-dioxide, thiophene, thiophenylphenyl, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine. In addition the “regioisomer variants” notation as in, for example, “5H-pyrrolo[2,3-b]pyrazine and its regioisomeric variants” would also encompass 7H-pyrrolo[2,3-d]pyrimidine, 7H-pyrrolo[2,3-c]pyridazine, 1H-pyrrolo[2,3-d]pyridazine, 5H-pyrrolo[3,2-c]pyridazine, and 5H-pyrrolo[3,2-d]pyrimidine. Similarly, 6,7-dihydro-5H-pyrrolo[2,3-b]pyrazine and its regioisomeric variants would encompass 6,7-dihydro-5H-pyrrolo[2,3-d]pyrimidine and 6,7-dihydro-5H-pyrrolo[2,3-c]pyridazine. It is also understood that the lack of “regioisomeric variants” notation does not in any way restrict the claim scope to the noted example only.

“Heterocyclylalkyl” is a heterocyclyl moiety attached to the parent structure through C₁-C₅ alkyl group. Examples include, but are not limited to, —(CH₂)ₙ—R^{Z} or —CH(CH₃)—(R^{Z}) where n=1-5 and that R^{Z} is chosen from benzimidazole, imidazole, indazole, isooxazole, phenyl-pyrazole, pyridine, quinoline, thiazole, triazole, triazolone, oxadiazole.

Terms with a hydrocarbon moiety (e.g. alkoxy) include straight and branched isomers for the hydrocarbon portion with the indicated number of carbon atoms.

Bonding and positional bonding relationships are those that are stable as understood by practitioners of organic chemistry.

Parenthetic and multiparenthetic terms are intended to clarify bonding relationships to those skilled in the art. For example, a term such as ((R)alkyl) means an alkyl substituent further substituted with the substituent R.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R.

“Combination,” “coadministration,” “concurrent” and similar terms referring to the administration of a compound of Formula I with at least one anti-HIV agent mean that the components are part of a combination antiretroviral therapy or highly active antiretroviral therapy (“HAART”) as understood by practitioners in the field of AIDS and HIV infection.

“Therapeutically effective” means the amount of agent required to provide a benefit to a patient as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus.

“Treatment,” “therapy,” “regimen,” “HIV infection,” “ARC,” “AIDS” and related terms are used as understood by practitioners in the field of AIDS and HIV infection.

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In an aspect of the invention, there is provided a compound of Formula I:

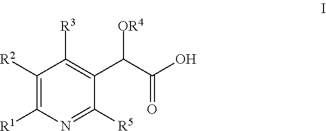

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is selected from tetrahydroisoquinolinyl or decahydroisoquinolinyl and is substituted with 0-3 substituents selected from halo, alkyl, and haloalkyl;
or R³ is a [5-7.3-7.0-2] fused or bridged bicyclic amine and is substituted with 0-3 alkyl substituents;
or R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

In an aspect of the invention, R² is phenyl substituted with 1 R⁷ substituent and with 0-3 substituents selected from halo, alkyl, haloalkyl, alkoxy, and haloalkoxy.

In an aspect of the invention, R² is selected from tetrahydroisoquinolinyl, ((Ar¹)alkyl)tetrahydroisoquinolinyl, or ((N-alkoxycarbonyl)tetrahydroisoquinolinyl.

In an aspect of the invention, R³ is selected from tetrahydroisoquinolinyl or decahydroisoquinolinyl and is substituted with 0-3 substituents selected from halo, alkyl, and haloalkyl.

In an aspect of the invention, R³ is a [5-7.3-7.0-2] fused or bridged bicyclic amine and is substituted with 0-3 alkyl substituents.

In an aspect of the invention, R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents.

In an aspect of the invention, R⁹ is selected from hydrogen or alkyl.

In an aspect of the invention, (R⁸)(R⁹)N taken together is selected from azetidinyl, pyrrolidinyl, piperidinyl, (spirocyclobutyl)piperidinyl, piperazinyl, or morpholinyl.

In an aspect of the invention, there is provided a compound of Formula I:

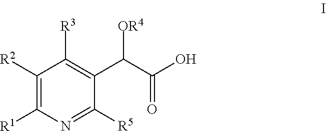

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
R³ is selected from tetrahydroisoquinolinyl or decahydroisoquinolinyl and is substituted with 0-3 substituents selected from halo, alkyl, and haloalkyl;
or R³ is a [5-7.3-7.0-2] fused or bridged bicyclic amine and is substituted with 0-3 alkyl substituents;
or R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

In an aspect of the invention, there is provided a compound of Formula I:

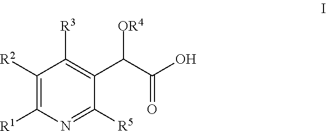

wherein:
R¹ is selected from H, alkyl, haloalkyl, hydroxyalkyl, alkoxyalkyl, (alkoxy)alkoxyalkyl, or (R⁶)alkyl;
R² is selected from tetrahydroisoquinolinyl, ((Ar¹)alkyl)tetrahydroisoquinolinyl, or ((N-alkoxycarbonyl)tetrahydroisoquinolinyl
R³ is is selected from tetrahydroisoquinolinyl or decahydroisoquinolinyl and is substituted with 0-3 substituents selected from halo, alkyl, and haloalkyl;
or R³ is a [5-7.3-7.0-2] fused or bridged bicyclic amine and is substituted with 0-3 alkyl substituents;
or R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

In an aspect of the invention, there is provided a compound of Formula I:

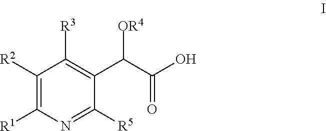

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is is selected from tetrahydroisoquinolinyl or decahydroisoquinolinyl and is substituted with 0-3 substituents selected from halo, alkyl, and haloalkyl;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

In an aspect of the invention, there is provided a compound of Formula I:

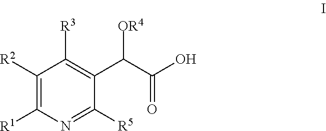

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is a [5-7.3-7.0-2] fused or bridged bicyclic amine and is substituted with 0-3 alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

In an aspect of the invention, there is provided a compound of Formula I:

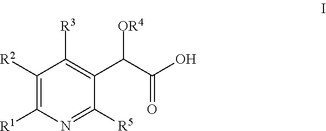

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl,
(tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

In an aspect of the invention, there is provided a compound of Formula I:

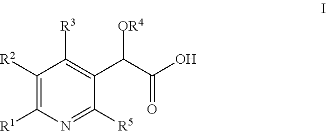

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is is selected from tetrahydroisoquinolinyl or decahydroisoquinolinyl and is substituted with 0-3 substituents selected from halo, alkyl, and haloalkyl;
or R³ is a [5-7.3-7.0-2] fused or bridged bicyclic amine and is substituted with 0-3 alkyl substituents;
or R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or a pharmaceutically acceptable salt thereof.

In an aspect of the invention, there is provided a compound of Formula I:

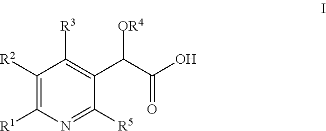

wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is is selected from tetrahydroisoquinolinyl or decahydroisoquinolinyl and is substituted with 0-3 substituents selected from halo, alkyl, and haloalkyl;
or R³ is a [5-7.3-7.0-2] fused or bridged bicyclic amine and is substituted with 0-3 alkyl substituents;
or R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
(R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

For a particular compound of Formula I, the scope of any instance of a variable substituent, including R¹, R², R³, R⁴, R⁵, R⁶, R⁷, R⁸, R⁹, and Ar¹ can be used independently with the scope of any other instance of a variable substituent. As such, the invention includes combinations of the different aspects.

In an aspect of the invention, there is provided a composition useful for treating HIV infection comprising a therapeutic amount of a compound of Formula I and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I.

Preferred compounds in accordance with the present invention include the following:
(S)-2-(5-(4-(benzylcarbamoyl)phenyl)-4-(2-azabicyclo[2.2.1]heptan-2-yl)-2,6-dimethylpyridin-3-yl)-2-(tert-butoxy)acetic acid;
(S)-2-(tert-butoxy)-2-(4-(3,4-dihydroisoquinolin-2(1H)-yl)-5-(4-(4-fluorophenethoxy)phenyl)-2,6-dimethylpyridin-3-yl)acetic acid;
(S)-2-(5-(4-(benzylcarbamoyl)phenyl)-4-(2-azabicyclo[2.2.1]heptan-2-yl)-2,6-dimethylpyridin-3-yl)-2-(tert-butoxy)acetic acid;
(2S)-2-(5-(4-(benzylcarbamoyl)phenyl)-4-(hexahydrocyclopenta[c]pyrrol-2(1H)-yl)-2,6-dimethylpyridin-3-yl)-2-(tert-butoxy)acetic acid;
(S)-2-(5-(4-(benzylcarbamoyl)phenyl)-2,6-dimethyl-4-(1,8,8-trimethyl-3-azabicyclo[3.2.1]octan-3-yl)pyridin-3-yl)-2-(tert-butoxy)acetic acid;
(2 S)-2-(4-(3-azabicyclo[3.1.0]hexan-3-yl)-5-(4-(4-fluorophenethoxy)phenyl)-2,6-dimethylpyridin-3-yl)-2-(tert-butoxy)acetic acid;
(2S)-2-(5-(4-(benzylcarbamoyl)phenyl)-4-(3-azabicyclo[3.1.0]hexan-3-yl)-2,6-dimethylpyridin-3-yl)-2-(tert-butoxy)acetic acid;
(2S)-2-(tert-butoxy)-2-(4-(6,6-dimethyl-3-azabicyclo[3.1.0]hexan-3-yl)-5-(4-(4-fluorophenethoxy)phenyl)-2,6-dimethyl-pyridin-3-yl)acetic acid;
(2S)-2-(5-(4-(benzylcarbamoyl)phenyl)-4-(6,6-dimethyl-3-azabicyclo[3.1.0]hexan-3-yl)-2,6-dimethylpyridin-3-yl)-2-(tert-butoxy)acetic acid;
(S)-2-(tert-butoxy)-2-(4-(3,4-dihydroisoquinolin-2(1H)-yl)-5-(4-(4-fluorophenethoxy)phenyl)-2,6-dimethylpyridin-3-yl)acetic acid;
(S)-2-(5-(4-(benzylcarbamoyl)phenyl)-2,6-dimethyl-4-((4aR,8aR)-octahydroisoquinolin-2(1H)-yl)pyridin-3-yl)-2-(tert-butoxy)acetic acid;
(S)-2-(tert-butoxy)-2-(5-(4-(4-fluorophenethoxy)phenyl)-2,6-dimethyl-4-((4aR,8aS)-octahydroisoquinolin-2(1H)-yl)pyridin-3-yl)acetic acid;
(S)-2-(5-(4-(benzylcarbamoyl) phenyl)-2,6-dimethyl-4-((4aR,8aS)-octahydroisoquinolin-2(1H)-yl)pyridin-3-yl)-2-(tert-butoxy)acetic acid;
(S)-2-(tert-butoxy)-2-(5-(4-(4-fluorophenethoxy)phenyl)-2,6-dimethyl-4-(2-azaspiro[4.4]nonan-2-yl)pyridin-3-yl)acetic acid;
(S)-2-(5-(4-(benzylcarbamoyl)phenyl)-2,6-dimethyl-4-(2-azaspiro[4.4]nonan-2-yl)pyridin-3-yl)-2-(tert-butoxy)acetic acid;
(S)-2-(tert-butoxy)-2-(5-(4-(4-fluorophenethoxy)phenyl)-2,6-dimethyl-4-(6-azaspiro[2.5]octan-6-yl)pyridin-3-yl)acetic acid;
(S)-2-(5-(4-(benzylcarbamoyl)-phenyl)-2,6-dimethyl-4-(6-azaspiro[2.5]octan-6-yl)pyridin-3-yl)-2-(tert-butoxy)acetic acid;
(S)-2-(tert-butoxy)-2-(5-(4-(4-fluorophenethoxy)phenyl)-2,6-dimethyl-4-(7-azaspiro[4.5]decan-7-yl)pyridin-3-yl)acetic acid;
(S)-2-(5-(4-(benzylcarbamoyl)phenyl)-2,6-dimethyl-4-(7-azaspiro[4.5]decan-7-yl)pyridin-3-yl)-2-(tert-butoxy)acetic acid;
(S)-2-(tert-butoxy)-2-(5-(4-(4-fluorophenethoxy)phenyl)-2,6-dimethyl-4-(8-azaspiro[4.5]decan-8-yl)pyridin-3-yl)acetic acid;
(S)-2-(5-(4-(benzylcarbamoyl)phenyl)-2,6-dimethyl-4-(8-azaspiro[4.5]decan-8-yl)pyridin-3-yl)-2-(tert-butoxy)acetic acid;
(S)-2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetic acid;
(S)-2-(5-bromo-2,6-dimethyl-4-(8-azaspiro[4.5]decan-8-yl)pyridin-3-yl)-2-(tert-butoxy)acetic acid;
(S)-2-(5-(4-(benzylcarbamoyl)-3-fluorophenyl)-2,6-dimethyl-4-(8-azaspiro[4.5]decan-8-yl)pyridin-3-yl)-2-(tert-butoxy)acetic acid;
(S)-2-(5-(4-(benzylcarbamoyl)-3-methoxyphenyl)-2,6-dimethyl-4-(8-azaspiro[4.5]decan-8-yl)pyridin-3-yl)-2-(tert-butoxy)acetic acid;
(S)-2-(tert-butoxy)-2-(5-(2-(2-fluorobenzyl)-1,2,3,4-tetrahydroisoquinolin-6-yl)-2,6-dimethyl-4-(8-azaspiro[4.5]decan-8-yl)pyridin-3-yl)acetic acid;
(S)-2-(tert-butoxy)-2-(5-(4-(4-fluorophenethoxy)phenyl)-2,6-dimethyl-4-(7-azaspiro[3.5]nonan-7-yl)pyridin-3-yl)acetic acid;
(S)-2-(tert-butoxy)-2-(5-(4-(2-(4-fluorophenyl)-2-methylpropoxy)phenyl)-2,6-dimethyl-4-(7-azaspiro[3.5]nonan-7-yl)pyridin-3-yl)acetic acid;
(S)-2-(5-(4-(benzylcarbamoyl)phenyl)-2,6-dimethyl-4-(7-azaspiro[3.5]nonan-7-yl)pyridin-3-yl)-2-(tert-butoxy)acetic acid;
(S)-2-(5-bromo-2,6-dimethyl-4-(7-azaspiro[3.5]nonan-7-yl)pyridin-3-yl)-2-(tert-butoxy)acetic acid;
(S)-2-(5-(4-(benzylcarbamoyl)-3-fluorophenyl)-2,6-dimethyl-4-(7-azaspiro[3.5]nonan-7-yl)pyridin-3-yl)-2-(tert-butoxy)acetic acid;
(S)-2-(5-(4-(benzylcarbamoyl)-3-methoxyphenyl)-2,6-dimethyl-4-(7-azaspiro[3.5]nonan-7-yl)pyridin-3-yl)-2-(tert-butoxy)acetic acid;
(S)-2-(tert-butoxy)-2-(5-(2-(2-fluorobenzyl)-1,2,3,4-tetrahydroisoquinolin-6-yl)-2,6-dimethyl-4-(7-azaspiro[3.5]nonan-7-yl)pyridin-3-yl)acetic acid;
(S)-2-(tert-butoxy)-2-(5-(2-(2-fluoro-6-methylbenzyl)-1,2,3,4-tetrahydroisoquinolin-6-yl)-2,6-dimethyl-4-(7-azaspiro[3.5]nonan-7-yl)pyridin-3-yl)acetic acid;
(S)-2-(tert-butoxy)-2-(5-(2-(4-fluoro-2-methylbenzyl)-1,2,3,4-tetrahydroisoquinolin-6-yl)-2,6-dimethyl-4-(7-azaspiro[3.5]nonan-7-yl)pyridin-3-yl)acetic acid;
(S)-2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetic acid;
(S)-2-(tert-butoxy)-2-(5-(2-(2-fluoro-4-methylbenzyl)-1,2,3,4-tetrahydroisoquinolin-6-yl)-2,6-dimethyl-4-(7-azaspiro[3.5]nonan-7-yl)pyridin-3-yl)acetic acid;
(S)-2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-2-(hydroxymethyl)-6-methyl-4-(7-azaspiro[3.5]nonan-7-yl)pyridin-3-yl)acetic acid;
(S)-2-(tert-butoxy)-2-(6-(fluoromethyl)-5-(4-(4-fluorophenethoxy)phenyl)-2-methyl-4-(7-azaspiro[3.5]nonan-7-yl)pyridin-3-yl)acetic acid;
(S)-2-(tert-butoxy)-2-(6-(ethoxymethyl)-5-(4-(4-fluorophenethoxy)phenyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-6-(((2-methoxyethyl)amino)methyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-6-(((2-methoxyethyl)(methyl)amino)methyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-6-(pyrrolidin-1-ylmethyl)-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-4-(7-azaspiro[3.5]nonan-7-yl)-6-((((tetrahydro-2H-pyran-4-yl)methyl)amino)methyl)pyridin-3-yl)acetic acid;
(S)-2-(tert-butoxy)-2-(5-(4-(4-fluorophenethoxy)phenyl)-2-methyl-6-((methyl((tetrahydro-2H-pyran-4-yl)methyl)amino)methyl)-4-(7-azaspiro[3.5]nonan-7-yl)pyridin-3-yl)acetic acid;
(S)-2-(tert-butoxy)-2-(5-(4-(4-fluorophenethoxy)phenyl)-6-(((4-methoxyphenyl)amino)methyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-6-((oxetan-3-ylmethoxy)methyl)-4-(7-azaspiro[3.5]nonan-7-yl)pyridin-3-yl)acetic acid;
(S)-2-(tert-butoxy)-2-(6-((2-ethoxyethoxy)methyl)-5-(4-(4-fluoro-phenethoxy)phenyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-6-((methyl(tetrahydro-2H-pyran-4-yl)amino)methyl)-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-4-(7-azaspiro[3.5]nonan-7-yl)-6-(((tetrahydro-2H-pyran-4-yl)methoxy)methyl)pyridin-3-yl)acetic acid;
(S)-2-(tert-butoxy)-2-(5-(4-(4-fluorophenethoxy)phenyl)-2-methyl-4-(7-azaspiro[3.5]nonan-7-yl)-6-(((tetrahydro-2H-pyran-4-yl)oxy)methyl)pyridin-3-yl)acetic acid;
(S)-2-(tert-butoxy)-2-(5-(4-(4-fluorophenethoxy)phenyl)-2-methyl-6-(piperi din-1-ylmethyl)-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-6-(morpholinomethyl)-4-(7-azaspiro[3.5]nonan-7-yl)pyridin-3-yl)acetic acid;
(S)-2-(tert-butoxy)-2-(5-(4-(4-fluorophenethoxy)phenyl)-2-methyl-4-(7-azaspiro[3.5]nonan-7-yl)-6-(((tetrahydro-2H-pyran-4-yl)amino)methyl)pyridin-3-yl)acetic acid;
(S)-2-(tert-butoxy)-2-(6-(((cyclohexylmethyl)amino)methyl)-5-(4-(4-fluorophenethoxy)phenyl)-2-methyl-4-(7-azaspiro[3.5]nonan-7-yl)pyridin-3-yl)acetic acid;
(S)-2-(6-(7-azaspiro[3.5]nonan-7-ylmethyl)-5-(4-(4-fluorophenethoxy)phenyl)-2-methyl-4-(7-azaspiro[3.5]nonan-7-yl)pyridin-3-yl)-2-(tert-butoxy)acetic acid;
(S)-2-(tert-butoxy)-2-(5-(4-(4-fluorophenethoxy)phenyl)-2-methyl-6-((oxetan-3-yloxy)methyl)-4-(7-azaspiro[3.5]nonan-7-yl)pyridin-3-yl)acetic acid;
(S)-2-(tert-butoxy)-2-(6-(((cyclohexylmethyl)(methyl)amino) methyl)-5-(4-(4-fluorophenethoxy)phenyl)-2-methyl-4-(7-azaspiro[3.5]nonan-7-yl)pyridin-3-yl)acetic acid;
(S)-2-(tert-butoxy)-2-(5-(4-(4-fluorophenethoxy)phenyl)-2,6-dimethyl-4-(3-azaspiro[5.5]undecan-3-yl)pyridin-3-yl)acetic acid;
(S)-2-(5-(4-(benzylcarbamoyl)phenyl)-2,6-dimethyl-4-(3-azaspiro[5.5]undecan-3-yl)pyridin-3-yl)-2-(tert-butoxy)acetic acid;
(S)-2-(tert-butoxy)-2-(5-(4-(4-fluorophenethoxy)phenyl)-2,6-dimethyl-4-(2-azaspiro[4.5]decan-2-yl)pyridin-3-yl)acetic acid;
(S)-2-(5-(4-(benzylcarbamoyl)phenyl)-2,6-dimethyl-4-(2-azaspiro[4.5]decan-2-yl)pyridin-3-yl)-2-(tert-butoxy)acetic acid;
(S)-2-(tert-Butoxy)-2-(5-(4-(4-fluorophenethoxy)phenyl)-2,6-dimethyl-4-(2-azaspiro[4.6]undecan-2-yl)pyridin-3-yl)acetic acid;
(S)-2-(5-(4-(benzylcarbamoyl)phenyl)-2,6-dimethyl-4-(2-azaspiro[4.6]undecan-2-yl)pyridin-3-yl)-2-(tert-butoxy) acetic acid;
(S)-2-(tert-butoxy)-2-(5-(4-(4-fluorophenethoxy)phenyl)-2,6-dimethyl-4-(1-oxa-8-azaspiro[4.5]decan-8-yl)pyridin-3-yl)acetic acid;
(S)-2-(tert-butoxy)-2-(5-(4-(4-fluorophenethoxy)phenyl)-2,6-dimethyl-4-(2-oxa-8-azaspiro[4.5]decan-8-yl)pyridin-3-yl)acetic acid;
(S)-2-(tert-butoxy)-2-(5-(4-(4-fluorophen ethoxy)phenyl)-2,6-dimethyl-4-(2,8-diazaspiro[4.5]decan-8-yl)pyridin-3-yl)acetic acid;
(S)-2-(5-(4-(benzylcarbamoyl) phenyl)-2,6-dimethyl-4-(2,8-diazaspiro[4.5]decan-8-yl)pyridin-3-yl)-2-(tert-butoxy)acetic acid;
(S)-2-(tert-butoxy)-2-(4-(1,1-difluoro-6-azaspiro[2.5]octan-6-yl)-5-(4-(4-fluorophenethoxy)phenyl)-2,6-dimethylpyridin-3-yl)acetic acid; and
(S)-2-(5-(4-(benzylcarbamoyl)phenyl)-4-(1,1-difluoro-6-azaspiro[2.5]octan-6-yl)-2,6-dimethylpyridin-3-yl)-2-(tert-butoxy)acetic acid; and
pharmaceutically acceptable salts thereof.

The compounds of the invention herein described may typically be administered as pharmaceutical compositions. These compositions are comprised of a therapeutically effective amount of a compound of Formula I or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 milligram (“mg”) of the active ingredient per dose are typical. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 milligram per milliliter (“mg/mL”). Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 milligram per kilogram (“mg/kg”) body weight daily. Generally, more compound is required orally and less parenterally. The specific dosing regimen, however, will be determined by a physician using sound medical judgment.

The compounds of this invention desireably have activity against HIV. Accordingly, another aspect of the invention is a method for treating HIV infection in a human patient comprising administering a therapeutically effective amount of a compound of Formula I, or a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, HIV capsid inhibitors, anti-infectives, and immunomodulators, such as, for example, PD-1 inhibitors, PD-L1 inhinitors, antibodies, and the like. In these combination methods, the compound of Formula I will generally be given in a daily dose of about 1-100 mg/kg body weight daily in conjunction with other agents. The other agents generally will be given in the amounts used therapeutically. The specific dosing regimen, however, will be determined by a physician using sound medical judgment.

Examples of nucleoside HIV reverse transcriptase inhibitors include abacavir, didanosine, emtricitabine, lamivudine, stavudine, tenofovir, zalcitabine, and zidovudine.

Examples of non-nucleoside HIV reverse transcriptase inhibitors include delavirdine, efavirenz, etrivirine, nevirapine, and rilpivirine.

Examples of HIV protease inhibitors include amprenavir, atazanavir, darunavir, fosamprenavir, indinavir, lopinavir, nelfinavir, ritonavir, saquinavir and, tipranavir.

An example of an HIV fusion inhibitor is enfuvirtide or T-1249.

An example of an HIV entry inhibitor is maraviroc.

Examples of HIV integrase inhibitors include dolutegravir, elvitegravir, or raltegravir.

An example of an HIV attachment inhibitor is fostemsavir.

An example of an HIV maturation inhibitor is BMS-955176, having the following structure:

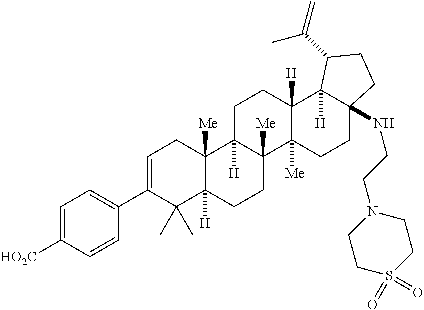

Thus, as set forth above, contemplated herein are combinations of the compounds of Formula I, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

| | | |
|---|---|---|
| Drug Name | Manufacturer | Indication |

ANTIVIRALS

| | | |
|---|---|---|
| Rilpivirine | Tibotec | HIV infection, AIDS, ARC |
| | | (non-nucleoside |
| | | reverse transcriptase |
| | | inhibitor) |
| COMPLERA ® | Gilead | HIV infection, AIDS, |
| | | ARC; combination |
| | | with emtricitabine, rilpivirine, |
| | | and tenofovir disoproxil |
| | | fumarate |
| 097 | Hoechst/Bayer | HIV infection, |
| | | AIDS, ARC |
| | | (non-nucleoside |
| | | reverse transcriptase |
| | | (RT) |
| | | inhibitor) |
| Amprenavir | Glaxo Wellcome | HIV infection, |
| 141 W94 | | AIDS, ARC |
| GW 141 | | (protease inhibitor) |
| Abacavir (1592U89) | Glaxo Wellcome | HIV infection, |
| GW 1592 | | AIDS, ARC |
| | | (RT inhibitor) |
| Acemannan | Carrington Labs | ARC |
| | (Irving, TX) | |
| Acyclovir | Burroughs Wellcome | HIV infection, AIDS, |
| | | ARC |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, |
| | | ARC |
| Beta-fluoro-ddA | Nat'l Cancer Institute | AIDS-associated |
| | | diseases |
| CI-1012 | Warner-Lambert | HIV-1 infection |
| Cidofovir | Gilead Science | CMV retinitis, |
| | | herpes, papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV |
| | | peripheral CMV |
| | | retinitis |
| Darunavir | Tibotec-J & J | HIV infection, AIDS, ARC |
| | | (protease inhibitor) |
| Delaviridine | Pharmacia-Upjohn | HIV infection, |
| | | AIDS, ARC |
| | | (RT inhibitor) |
| Dextran Sulfate | Ueno Fine Chem. | AIDS, ARC, HIV |
| | Ind. Ltd. (Osaka, | positive |
| | Japan) | asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination |
| | | with AZT/d4T |
| DMP-450 | AVID | HIV infection, |
| | (Camden, NJ) | AIDS, ARC |
| | | (protease inhibitor) |
| Efavirenz | Bristol Myers Squibb | HIV infection, |
| (DMP 266, SUSTIVA ®) | | AIDS, ARC |
| (−)6-Chloro-4-(S)- | | (non-nucleoside RT |
| cyclopropylethynyl- | | inhibitor) |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside |
| | | reverse transcriptase |
| | | inhibitor) |
| Famciclovir | Smith Kline | herpes zoster, |
| | | herpes simplex |
| GS 840 | Gilead | HIV infection, |
| | | AIDS, ARC |
| | | (reverse transcriptase |
| | | inhibitor) |
| HBY097 | Hoechst Marion | HIV infection, |
| | Roussel | AIDS, ARC |
| | | (non-nucleoside |
| | | reverse transcriptase |
| | | inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic |
| | | HIV positive, also in |
| | | combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, |
| | | AIDS, ARC |
| | | (reverse |
| | | transcriptase |
| | | inhibitor); also |
| | | with AZT |
| Lobucavir | Bristol-Myers Squibb | CMV infection |
| Nelfinavir | Agouron | HIV infection, |
| | Pharmaceuticals | AIDS, ARC |
| | | (protease inhibitor) |
| Nevirapine | Boeheringer | HIV infection, |
| | Ingleheim | AIDS, ARC |
| | | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, |
| | | AIDS, ARC |
| | | (protease inhibitor) |
| Probucol | Vyrex | HIV infection, AIDS |
| RBC-CD4 | Sheffield Med. | HIV infection, |
| | Tech (Houston, TX) | AIDS, ARC |
| Ritonavir | Abbott | HIV infection, |
| | | AIDS, ARC |
| | | (protease inhibitor) |
| Saquinavir | Hoffmann- | HIV infection, |
| | LaRoche | AIDS, ARC |
| | | (protease inhibitor) |
| Stavudine; d4T | Bristol-Myers Squibb | HIV infection, AIDS, |
| Didehydrodeoxy- | | ARC |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV |
| Ribavirin | (Costa Mesa, CA) | positive, LAS, ARC |
| VX-478 | Vertex | HIV infection, AIDS, |
| | | ARC |
| Zalcitabine | Hoffmann-LaRoche | HIV infection, AIDS, |
| | | ARC, with AZT |
| Zidovudine; AZT | Glaxo Wellcome | HIV infection, AIDS, |
| | | ARC, Kaposi's |
| | | sarcoma, in combination with |
| | | other therapies |
| Tenofovir disoproxil, | Gilead | HIV infection, |
| fumarate salt (VIREAD ®) | | AIDS, |
| | | (reverse transcriptase |
| | | inhibitor) |
| EMTRIVA ® | Gilead | HIV infection, |
| (Emtricitabine) (FTC) | | AIDS, |
| | | (reverse transcriptase |
| | | inhibitor) |
| COMBIVIR ® | GSK | HIV infection, |
| | | AIDS, |
| | | (reverse transcriptase |
| | | inhibitor) |
| Abacavir succinate | GSK | HIV infection, |
| (or ZIAGEN ®) | | AIDS, |
| | | (reverse transcriptase |
| | | inhibitor) |
| REYATAZ ® | Bristol-Myers Squibb | HIV infection |
| (or atazanavir) | | AIDs, protease |
| | | inhibitor |
| FUZEON ® | Roche/Trimeris | HIV infection |
| (Enfuvirtide or T-20) | | AIDs, viral Fusion |
| | | inhibitor |
| LEXIVA ® | GSK/Vertex | HIV infection |
| (or Fosamprenavir calcium) | | AIDs, viral protease |
| | | inhibitor |
| SELZENTRY ™ | Pfizer | HIV infection |
| Maraviroc; (UK 427857) | | AIDs, (CCR5 antagonist, in |
| | | development) |
| TRIZIVIR ® | GSK | HIV infection |
| | | AIDs, (three drug combination) |
| Sch-417690 (vicriviroc) | Schering-Plough | HIV infection |
| | | AIDs, (CCR5 antagonist, in |
| | | development) |
| TAK-652 | Takeda | HIV infection |
| | | AIDs, (CCR5 antagonist, in |
| | | development) |
| GSK 873140 | GSK/ONO | HIV infection |
| (ONO-4128) | | AIDs, (CCR5 antagonist, |
| | | in development) |
| Integrase Inhibitor | Merck | HIV infection |
| MK-0518 | | AIDs |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection |
| GS917/JTK-303 | | AIDs |
| Elvitegravir | | in development |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection |
| | | AIDs |
| | | in development |
| CMX-157 | Chimerix | HIV infection |
| Lipid conjugate of | | AIDs |
| nucleotide tenofovir | | |
| GSK1349572 | GSK | HIV infection |
| Integrase inhibitor | | AIDs |
| TIVICAY ® | | |
| dolutegravir | | |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, |
| Macrophage Colony | | combination |
| Stimulating Factor | | w/AZT |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

### Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention. The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

Abbreviations used in the schemes and examples generally follow conventions used in the art. Chemical abbreviations used in the specification and examples are defined as follows: “KHMDS” for potasium bis(trimethylsilyl)amide; “DMF” for N,N-dimethylformamide; “HATU” for O-(t-Azabenzotriazol-1-yl)-N,N,N′,N′-tetramethyluronium hexafluorophosphate, “MeOH” for methanol; “Ar” for aryl; “TFA” for trifluoroacetic acid, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; and “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Some compounds can be synthesized from an appropriately substituted heterocycle I-1 according to Scheme I. Compounds I-1 and I-6 are commercially available or synthesized by reactions well known in the art. Treatment of compound I-1 with bromine provided the dibromo intermediates I-2 which was converted to the chloropyridine I-3 by reacting with POCl₃. Intermediate I-3 conveniently transformed to ketoester I-5 using conditions well-known to those skilled in the art, including reacting I-3 with Grignard reagent in the presence of catalytic copper(I) bromide dimethylsulfide complex followed by alkyl 2-chloro-2-oxoacetate. Coupling of amines 1-5 with intermediate I-6 in the presence of an organic base such as Hunig's base provided intermediate I-7. Chiral Lewis acid such as I-8 mediated reduction of ketoester I-7 with catecholborane furnished chiral alcohol I-9. Tertiary butylation of alcohol I-9 by well-known conditions, including but not limited to tertiary-butyl acetate and perchloric acid, gave intermediate I-10. Intermediates I-10 are conveniently transformed to intermediates I-11 using conditions well-known in the art, including but not limited to the Suzuki coupling between intermediates I-10 and R⁶B(OR)₂. The boronate or boronic acid coupling reagents, well-known in the art, are commercially available or are prepared by reactions well-known to those skilled in the art. Hydrolysis of intermediate I-11 by using conditions well-known to those skilled in the art furnished carboxylic acid I-12.

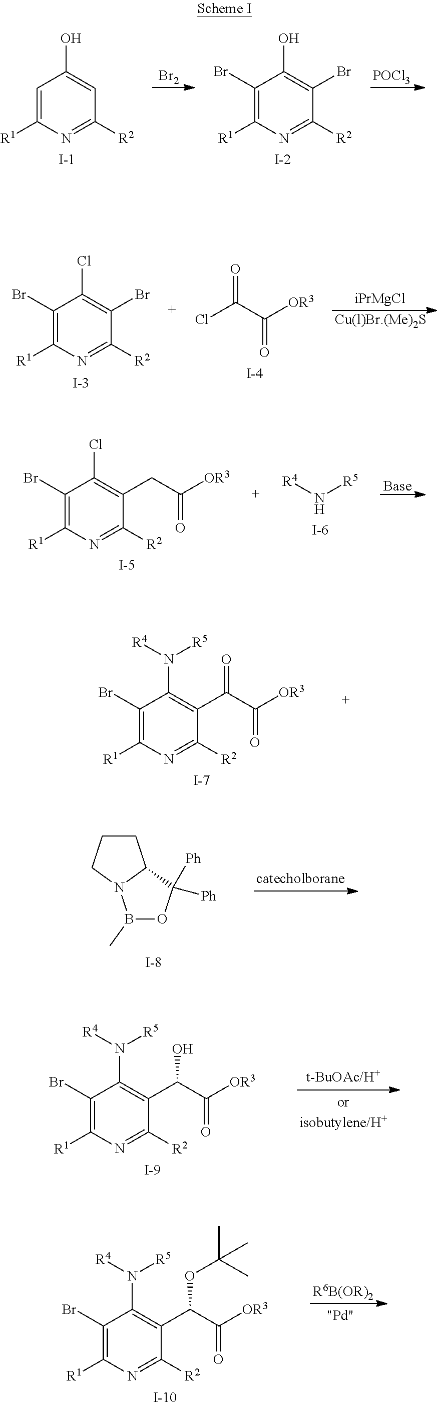

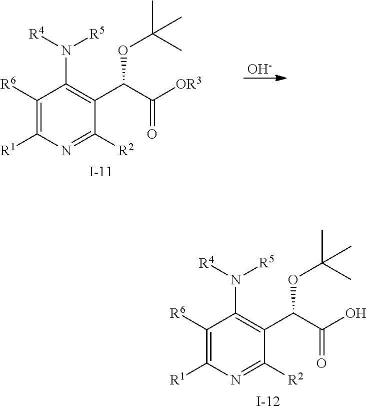

Intermediates I-10 are conveniently transformed to intermediates II-2 using conditions well-known in the art, including but not limited to the Suzuki coupling between intermediates I-10 and II-1. Cleavage of protecting group in II-2 provided phenol II-3. Alkylation of the phenol II-3 was achieved by using conditions well known to those skilled in the art, including but not limited to Mitshunobu reaction to provide the intermediate I-4. Hydrolysis of intermediate II-4 by using conditions well-known in the literature furnished carboxylic acid II-5.

Some compounds of this invention can be synthesized according to Scheme II.

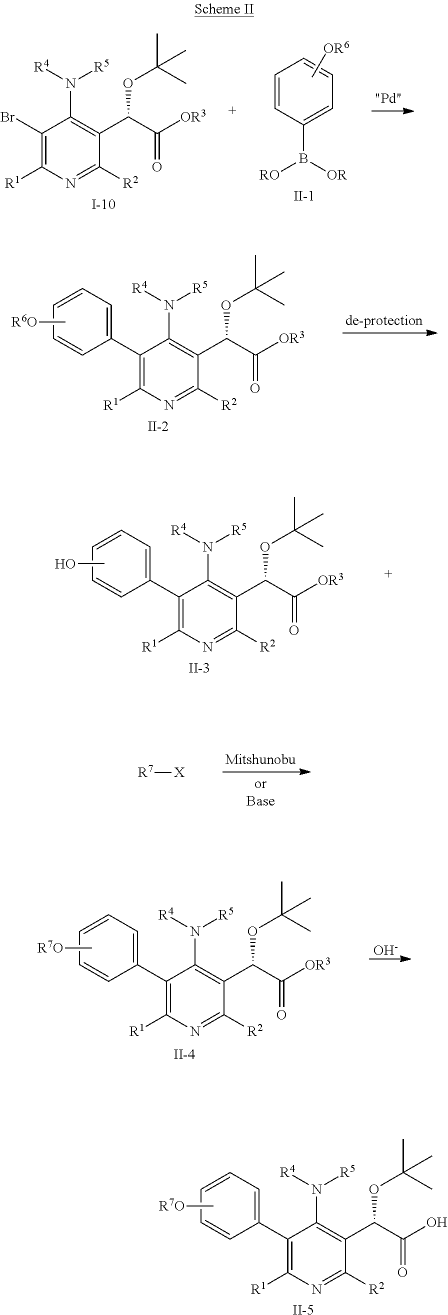

Some compounds of this invention can be synthesized according to Scheme III.

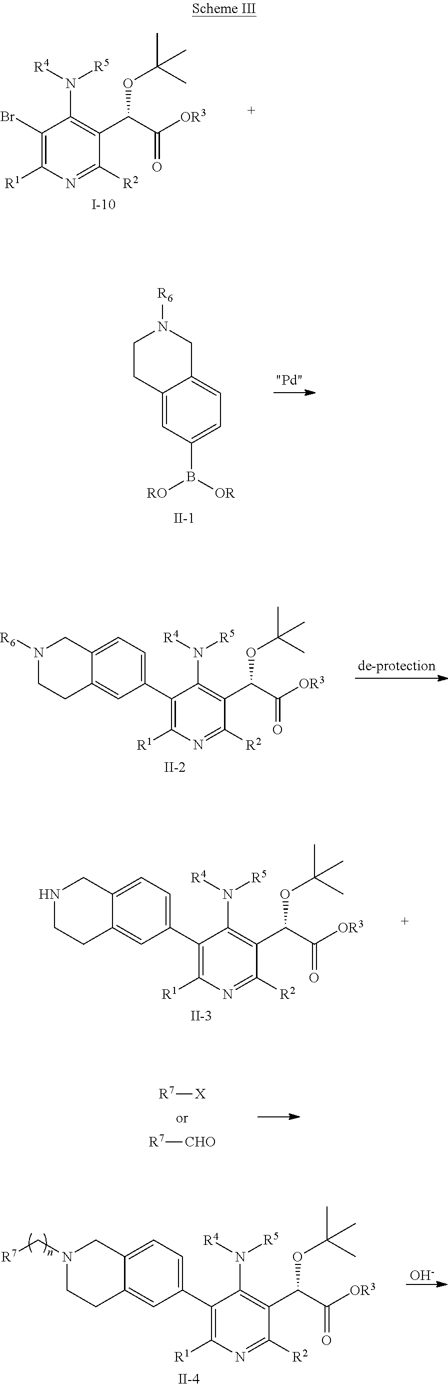

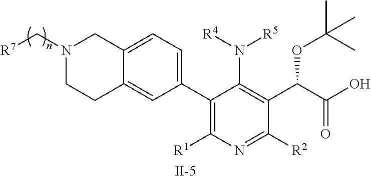

Some compounds of this invention can be synthesized according to Scheme IV. In Scheme IV, pyridine IV-1, can be produced using methods similar to those described in the previous schemes. This intermediate can be carried on to the final product according to a variety of paths. In one, the C2 and C6 alkyl groups can be oxidized to furnish intermediates IV-3 and/or IV-4 which can be further transformed to final compounds IV-9 or IV-10 by several paths.

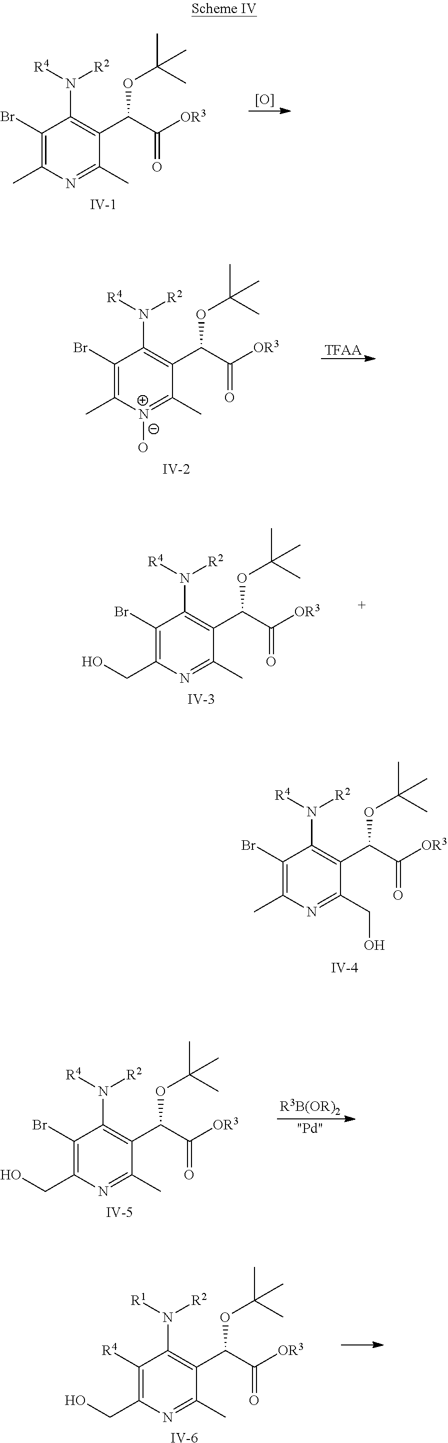

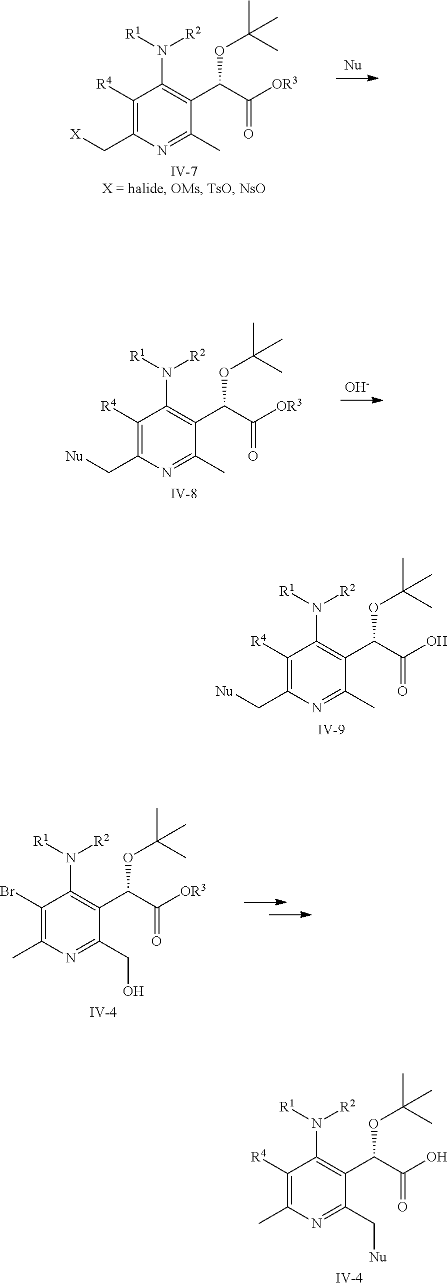

The compounds described herein were purified by the methods well known to those skilled in art by normal phase column chromatography on silica gel column using appropriate solvent system described. Preparative HPLC purifications mentioned in this experimentation section were carried out gradient elution either on Sunfire Prep C18 ODB column (5 μm; 19 or 30×100 mm) or Waters Xbridge C18 column (5 μM; 19×200 or 30×100 mm) or Water Atlantis (5 μm; 19 or 30×100 mm) using the following mobile phases. Mobile phase A: 9:1 H₂O/acetonitrile with 10 mM NH₄OAc and mobile phase B: A: 9:1 acetonitrile/H₂O with 10 mM NH₄OAc; or mobile phase A: 9:1 H₂O/acetonitrile with 0.1% TFA and mobile phase B: A: 9:1 acetonitrile/H₂O with 0.1% TFA; or mobile phase A: water/MeOH (9:1) with 20 mM NH₄OAc and mobile phase B: 95:5 MeOH/H₂O with 20 mM NH₄OAc or mobile phase A: water/MeOH (9:1) with 0.1% TFA and mobile phase B: 95:5 MeOH/H₂O with 0.1% TFA or mobile Phase A: 5:95 acetonitrile:water with 10-mM ammonium acetate; Mobile Phase B: 95:5 acetonitrile:water with 10-mM ammonium acetate.

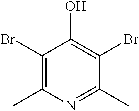

### 3,5-Dibromo-2,6-dimethylpyridin-4-ol

A 3-neck R.B-flask equipped with mechanical stirrer, addition funnel and condenser is charged with 2,6-dimethylpyridin-4-ol (100 g, 812 mmol), CH₂Cl₂ (1000 mL) and MeOH (120 mL). To the resulting light brown or tan solution was added tert-BuNH₂ (176 ml, 1665 mmol), cooled in water bath maintained between 5-10° C. (ice-water) and added drop wise Br2 (84 ml, 1624 mmol) over 70 min. After the addition was complete cold bath was removed and stirred for 1.5 h at rt. Then, the light orange slurry was filtered and the filter cake was washed with ether (250 mL) and dried to afford 3,5-dibromo-2,6-dimethylpyridin-4-ol, hydrobromide (280.75 g, 776 mmol, 96% yield) as white solid which was used in the next step without further purification. ¹H NMR (500 MHz, DMSO-d₆) δ 12.08 (br. s., 1H), 2.41 (s, 6H). LCMS (M+H)=281.9.

Alternative Procedure:

Bromine (72.8 mL, 1.4 mol) was added via addition funnel over 60 min to a mechanically stirred cold (ice-water bath) solution of 2,6-dimethylpyridin-4-ol (87 g, 706 mmol) and 4-methylmorpholine (156 mL, 1.4 mol) in dichloromethane (1 L) and methanol (100 mL) and then stirred for 2 h at rt. Additional bromine (˜15 mL) was added based on monitoring by LCMS. The product was filtered, washed with ether, and dried under vacuum to give 3,5-dibromo-2,6-dimethylpyridin-4-ol 176.8 g (88%).

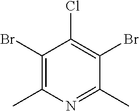

### 3,5-Dibromo-4-chloro-2,6-dimethyl-pyridine

Triethylamine (28.8 mL, 206 mmol) was added to a nitrogen purged solution of 3,5-dibromo-2,6-dimethylpyridin-4-ol (58 g, 206 mmol) and phosphorous oxychloride (57.7 mL, 619 mmol) in chloroform (450 mL) and stirred for 1 h at rt, then 3 h at 80° C. The reaction was removed from heating and immediately concentrated under house vaccum; then under high vacuum. The appearance was a cream colored solid, which was azeotroped with toluene (2×100 mL); treated with ice (200 g) for 10 min and carefully neutralized with NaHCO₃ (powder), and 1N NaOH solution, and extracted with DCM (2×400 mL). The combined organic layers were dried (MgSO₄), concentrated, and a beige solid was obtained that was washed with hexanes and dried under high vacuum to give 3,5-dibromo-4-chloro-2,6-dimethyl-pyridine 52.74 g (85.1%). Concentration of the hexanes gave 3.5 g of less pure product. ¹H NMR (500 MHz, CDCl₃) δ 2.59 (s, 6H). LCMS (M+H)=300.0.

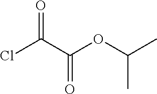

### 2-Chloro-2-oxoacetate

The propan-2-ol (38.2 mL, 499 mmol) was added drop wise over 15 min to a cold (0° C.), nitrogen purged solution of oxalyl chloride (101 g, 799 mmol) and the reaction was stirred at room temperature for 2.5 h. Then a reflux condenser was fitted and a slight vacuum was applied for about 1 h until HCl gas was removed (the HCl was trapped in by a sat'd solution of NaHCO₃). The reflux condenser was removed and the flask was fitted with a short path distillation head. Excess reagent was removed by distillation under house vacuum (oil bath heated to 65° C.), and then the temperature was raised to between 85-95° C. and the product was distilled (NOTE: The 1^{st} fraction of −5 mL was discarded) to provide isopropyl 2-chloro-2-oxoacetate 52.62 g (70%).

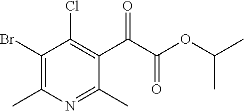 [SMILES: Cc1nc(C)c(C(=O)C(=O)OC(C)C)c(Cl)c1Br]

### 2-(5-Bromo-4-chloro-2,6-dimethylpyridin-3-yl)-2-oxoacetate

A solution of 2M isopropyl magnesium chloride (84 mL, 168 mmol) was added drop wise over 20 min to a cold (−70° C.), nitrogen purged solution of 3,5-dibromo-4-chloro-2,6-dimethylpyridine (48 g, 160 mmol) and copper(I)bromide-dimethyl sulfide complex (1.65 g, 8.02 mmol) in THF (240 mL), which was then allowed to warm to −10° C. over 60 min. The reaction mixture was transferred via cannula into a 1 L RB-flask containing isopropyl 2-chloro-2-oxoacetate (26.6 g, 176 mmol) in THF (160 mL) maintained at −60° C., and the reaction stirred an additional 2.5 h while being allowed to warm to −10° C. The reaction was quenched upon diluted with a mixture of 10% NH₄Cl solution (80 mL) in ether (320 mL). The organic layer was washed with 160 mL of sat'd NaHCO₃/10% NH₄Cl solution (1:1), brine, and dried (Na₂SO₄). The crude product was charged (DCM solution) to a 330 g ISCO silica gel cartridge and gradient eluted (5-20% EtOAc/hexanes) using an Isolera chromatography station gave isopropyl 2-(5-bromo-4-chloro-2,6-dimethylpyridin-3-yl)-2-oxoacetate 40.38 g (76%). ¹H NMR (500 MHz, CDCl₃) δ 5.28-5.21 (m, 1H), 2.77 (s, 3H), 2.47 (s, 3H), 1.40 (d, J=6.3 Hz, 6H). LCMS (M+H)=336.04.

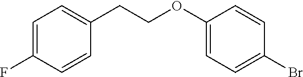 [SMILES: Fc1ccc(CCOc2ccc(Br)cc2)cc1]

### 1-Bromo-4-(4-fluorophenethoxy)benzene

To a stirred solution of 4-bromophenol (81.7 g, 472 mmol), 2-(4-fluorophenyl)ethanol (79 g, 567 mmol) and Ph₃P (149 g, 567 mmol) in THF (100 mL) cooled in an ice-water bath was added drop wise DEAD (93 ml, 590 mmol) over 20 min. Note: The reaction is exothermic and efficient cooling is highly recommended before initiating large scale reaction. After 1 h, cold bath was removed and stirred overnight (17 h) at rt. Then, the reaction mixture was concentrated, the resulting residue triturated with hexanes, filtered and the filter cake washed with 10% ether/hexanes (2-lit). The filtrate was concentrated and purified by flash chromatography (silica gel column 3″×11″) using 4-lit hexanes and 2-lit 2% EtOAc/Hex to afford 1-bromo-4-(4-fluorophenethoxy)benzene (142 g, 469 mmol, 99% yield) as colorless liquid (contaminated with ˜2.5% Ph₃P by ¹HNMR). ¹H NMR (500 MHz, CDCl₃) δ 7.41-7.36 (m, 2H), 7.28-7.22 (m, 2H), 7.05-6.99 (m, 2H), 6.82-6.76 (m, 2H), 4.14 (t, J=6.9 Hz, 2H), 3.08 (t, J=6.9 Hz, 2H).

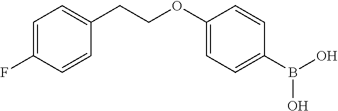 [SMILES: OB(O)c1ccc(OCCc2ccc(F)cc2)cc1]

### (4-(4-Fluorophenethoxy)phenyl)boronic acid

To a stirred solution of 1-bromo-4-(4-fluorophenethoxy)benzene (142 g, 469 mmol) in THF (1000 mL) was added 2M n-BuLi/cyclohexane (293 ml, 586 mmol) over 15 min at −78° C. After 1.5 h, triisopropyl borate (131 ml, 563 mmol) was added to the light pink reaction mixture over 5 min and stirred for 2 h at −78° C. Then, the reaction was quenched by careful addition of 3M HCl (375 mL), cold bath was replaced with water bath, stirred for 1 h, diluted with ether (500 mL), aq. layer separated and organic layer washed with water (2×200 mL). The combined aq. layers extracted with ether (200 mL) and combined ether layers washed with brine (100 mL), dried (MgSO₄), filtered and concentrated to 200 mL. To this was added 250 mL hexanes and concentrated to about 300 mL and allowed to stand at rt. The precipitated solid was triturated with hexanes and filtered to give white solid which was used in next step without purification. ¹H NMR (500 MHz, CDCl₃) δ 8.18-8.15 (m, 2H), 7.32-7.28 (m, 2H), 7.07-7.00 (m, 4H), 4.26 (t, J=6.9 Hz, 2H), 3.14 (t, J=6.9 Hz, 2H).

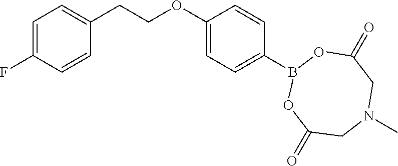 [SMILES: CN1CC(=O)OB(c2ccc(OCCc3ccc(F)cc3)cc2)OC(=O)C1]

### 2-(4-(4-Fluorophenethoxy)phenyl)-6-methyl-1,3,6,2-dioxazaborocane-4,8-dione

A slurry of (4-(4-fluorophenethoxy)phenyl)boronic acid (122 g, 469 mmol) and 2,2′-(methylazanediyl)diacetic acid (76 g, 516 mmol) in anhydrous toluene (500 mL) and DMSO (200 mL) was refluxed for 4 h. Then, cooled, diluted with EtOAc (500 mL), washed with water (5×200 mL), brine (2×100 mL), dried (MgSO₄), filtered and concentrated to give light orange foam which was purified by flash chromatography using 5-40% acetone/CH₂Cl₂ (5% increment per 2-lit) to afford 2-(4-(4-fluorophenethoxy)phenyl)-6-methyl-1,3,6,2-dioxazaborocane-4,8-dione (131.38 g, 354 mmol, 75% yield) as white solid. ¹H NMR (500 MHz, CDCl₃) δ 7.43 (d, J=8.4 Hz, 2H), 7.28-7.24 (m, 2H), 7.04-6.99 (m, 2H), 6.92 (d, J=8.5 Hz, 2H), 4.17 (t, J=6.9 Hz, 2H), 4.00 (d, J=16.6 Hz, 2H), 3.76 (d, J=16.6 Hz, 2H), 3.08 (t, J=6.8 Hz, 2H), 2.54 (s, 3H). LCMS (M+H)=372.3.

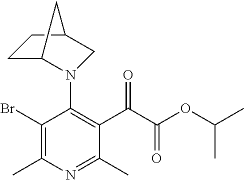 [SMILES: Cc1nc(C)c(C(=O)C(=O)OC(C)C)c(N2CC3CCC2C3)c1Br]

### Isopropyl 2-(4-(2-azabicyclo[2.2.1]heptan-2-yl)-5-bromo-2,6-dimethylpyridin-3-yl)-2-oxoacetate

To a solution of 2-azabicyclo[2.2.1]heptane (290 mg, 2.99 mmol) and DIEA (1.57 mL, 8.97 mmol) in anhydrous CH₃CN (15 mL) was added isopropyl 2-(5-bromo-4-chloro-2,6-dimethylpyridin-3-yl)-2-oxoacetate (1 g, 2.99 mmol) at rt. The resulting mixture was placed in a pre-heated oil bath (80° C.) and stirred for 18 h before being cooled, concentrated, and charged (DCM) to a 80 g ISCO silica gel cartridge and gradient elution (5-20% EtOAc/hexanes) using an Isolera chromatography station to give isopropyl 2-(4-(2-azabicyclo[2.2.1]heptan-2-yl)-5-bromo-2,6-dimethylpyridin-3-yl)-2-oxoacetate 400 mg (34%). ¹H NMR (500 MHz, CDCl3) δ 5.15-5.10 (m, 1H), 3.89 (s, 1H), 3.84 (d, J=9.3 Hz, 1H), 2.85 (t, J=2.8 Hz, 1H), 2.93 (t, J=2.6 Hz, 1H), 2.70 (s, 3H), 2.59 (br. s, 1H), 2.43 (s, 3H), 1.88-1.85 (m, 1H), 1.76-1.73 (m, 1H), 1.70-1.64 (m, 2H), 1.57-1.54 (m, 1H), 1.41 (d, J=9.5 Hz, 1H), 1.35 (d, J=6.3 Hz, 3H), 1.32 (d, J=6.3 Hz, 3H). UPLC (M+H)=397.2.

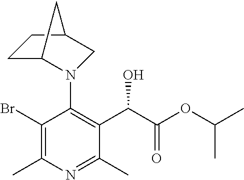 [SMILES: Cc1nc(C)c([C@H](O)C(=O)OC(C)C)c(N2CC3CCC2C3)c1Br]

### (S)-Isopropyl 2-(4-(2-azabicyclo[2.2.2.1]heptan-2-yl)-5-bromo-2,6-dimethylpyridin-3-yl)-2-hydroxyacetate

The benzo[d][1,3,2]dioxaborole (0.42 mL, 1.78 mmol; 50% soln in toluene) was added to a nitrogen purged solution of isopropyl 2-(4-(2-azabicyclo[2.2.1]heptan-2-yl)-5-bromo-2,6-dimethylpyridin-3-yl)-2-oxoacetate (370 mg, 0.94 mmol) and 0.28 mL of 1M (R)-1-methyl-3,3-diphenylhexahydropyrrolo[1,2-c][1,3,2]oxazaborole (78 mg, 0.28 mmol) in toluene (12 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 40 g ISCO silica gel cartridge and gradient elution (5-50% EtOAc/hexanes) using an Isolera chromatography station gave (S)-isopropyl 2-(4-(2-azabicyclo[2.2.1]heptan-2-yl)-5-bromo-2,6-dimethylpyridin-3-yl)-2-hydroxyacetate 124 mg (33%) and a second diasteromer (69 mg); major isomer: ¹H NMR (500 MHz, DMSO) δ 6.02 (s, 1H), 5.45 (d, J=4.4 Hz, 1H), 5.01-4.96 (m, 1H), 3.96 (s, 1H), 3.01 (d, J=7.0 Hz, 1H), 2.62 (s, 1H), 2.52 (s, 3H), 2.31 (s, 3H), 1.96 (d, J=8.4 Hz, 1H), 1.70-1.53 (m, 4H), 1.44 (d, J=8.4 Hz, 1H), 1.17 (d, J=6.2 Hz, 3H), 1.13 (d, J=6.2 Hz, 3H). UPLC (M+H)=399.3.

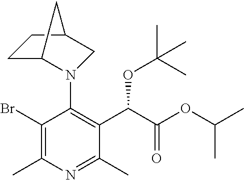 [SMILES: Cc1nc(C)c([C@H](OC(C)(C)C)C(=O)OC(C)C)c(N2CC3CCC2C3)c1Br]

### (S)-Isopropyl 2-(4-(2-azabicyclo[2.2.2.1]heptan-2-yl)-5-bromo-2,6-dimethylpyridin-3-yl)-2-(tert-butoxy)acetate

The isobutylene gas was bubbled into a nitrogen purged, cooled (0° C.) solution of (S)-isopropyl 2-(4-(2-azabicyclo[2.2.1]heptan-2-yl)-5-bromo-2,6-dimethylpyridin-3-yl)-2-hydroxyacetate (120 mg, 0.30 mmol) and 0.07 mL of 70% HClO₄ in DCM (5 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 24 g ISCO silica gel cartridge and gradient elution (5-35% EtOAc/hexanes) using an Isolera chromatography station gave (S)-isopropyl 2-(4-(2-azabicyclo[2.2.1]heptan-2-yl)-5-bromo-2,6-dimethylpyridin-3-yl)-2-(tert-butoxy)acetate 96 mg (70%): ¹H NMR (500 MHz, DMSO) δ 5.85 (s, 1H), 4.94-4.89 (m, 1H), 4.00 (br. s, 1H), 3.55 (s, 1H), 2.83 (d, J=7.7 Hz, 1H), 2.71 (s, 1H), 2.52 (s, 3H), 2.39 (s, 3H), 2.26 (d, J=8.8 Hz, 1H), 1.91-1.87 (m, 1H), 1.76-1.71 (m, 1H), 1.65-1.59 (m, 1H), 1.50-1.43 (m, 2H), 1.16 (d, J=6.2 Hz, 3H), 1.14 (s, 9H), 1.07 (d, J=6.2 Hz, 3H). UPLC (M+H)=455.1.

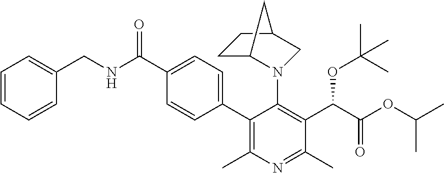 [SMILES: Cc1nc(C)c([C@H](OC(C)(C)C)C(=O)OC(C)C)c(N2CC3CCC2C3)c1-c1ccc(C(=O)NCc2ccccc2)cc1]

### (S)-Isopropyl 2-(5-(4-(benzylcarbamoyl)phenyl)-4-(2-azabicyclo[2.2.1]heptan-2-yl)-2,6-dimethylpyridin-3-yl)-2-(tert-butoxy)acetate

The Pd(Ph₃P)₄ (43 mg, 0.037 mmol) was added to a nitrogen purged and degassed solution of (S)-isopropyl 2-(4-(2-azabicyclo[2.2.1]heptan-2-yl)-5-bromo-2,6-dimethylpyridin-3-yl)-2-(tert-butoxy)acetate (85 mg, 0.19 mmol), (4-(benzylcarbamoyl)phenyl)boronic acid (53 mg, 0.21 mmol), and potassium phosphate tribasic (278 mg, 1.3 mmol) in 1,4-dioxane (2.5 mL) and water (0.5 mL). The reaction mixture was stirred in a screw-capped pressure vessel for 4 h at 90° C., cooled, diluted with EtOAc, and the organic layer was washed with brine and dried (Na₂CO₃). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-4-(2-azabicyclo[2.2.1]heptan-2-yl)-2,6-dimethylpyridin-3-yl)-2-(tert-butoxy)acetate 38 mg (35%) as a mixture of diasteromers: ¹H NMR (500 MHz, DMSO) δ 9.17-9.15 (m, 1H), 7.98 (t, J 8.4 Hz, 2H), 7.36-7.33 (m, 5H), 7.29-7.25 (m, 2H), 5.81 (s, 1H), 4.96-4.91 (m, 1H), 4.51 (d, J=5.9 Hz, 2H) 3.53 (s, 1H), 2.97 (br. s, 1H), 2.50 (s, 1H), 2.44 (s, 3H), 2.18 (s, 1H), 1.94 (s, 3H), 1.82-1.78 (m, 1H), 1.54-1.46 (m, 2H), 1.23-1.20 (m, 1H), 1.19 (d, J=6.2 Hz, 3H), 1.15 (s, 9H), 1.13 (d, J=6.2 Hz, 3H), 1.03 (d, J=8.8 Hz, 1H), 0.90 (d, J=8.8 Hz, 1H). UPLC (M+H)=584.6.

### Example 1

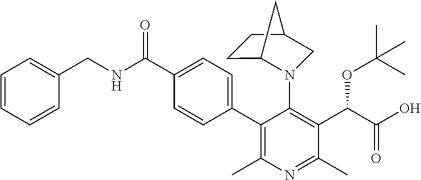 [SMILES: Cc1nc(C)c([C@H](OC(C)(C)C)C(=O)O)c(N2CC3CCC2C3)c1-c1ccc(C(=O)NCc2ccccc2)cc1]

### (S)-2-(5-(4-(Benzylcarbamoyl)phenyl)-4-(2-azabicyclo[2.2.1]heptan-2-yl)-2,6-dimethylpyridin-3-yl)-2-(tert-butoxy)acetic acid

The 0.13 mL of 1M sodium hydroxide (5.14 mg, 0.13 mmol) was added to a solution of (S)-isopropyl 2-(5-(4-(benzylcarbamoyl)phenyl)-4-(2-azabicyclo[2.2.1]heptan-2-yl)-2,6-dimethylpyridin-3-yl)-2-(tert-butoxy)acetate (30 mg, 0.05 mmol) in ethanol (2.5 mL) and stirred for 18 h at 90° C. The reaction mixture was neutralized with 1N HCl soln, extracted with EtOAc, and the organic layer was washed with brine, and dried (MgSO₄). The crude material was purified by prep HPLC to afford (S)-2-(5-(4-(benzylcarbamoyl)phenyl)-4-(2-azabicyclo[2.2.1]heptan-2-yl)-2,6-dimethylpyridin-3-yl)-2-(tert-butoxy)acetic acid 12.4 mg (45%). ¹H NMR (500 MHz, DMSO) δ 9.17-9.14 (m, 1H), 7.97 (t, J 8.4 Hz, 2H), 7.36-7.25 (m, 7H), 5.62 (s, 1H), 4.51 (t, J=5.9 Hz, 2H) 3.73 (s, 1H), 3.00-2.98 (m, 1H), 2.53 (s, 1H), 2.45 (s, 3H), 2.18 (s, 1H), 1.95 (s, 3H), 1.91 (s, 1H), 1.83-1.79 (m, 1H), 1.51-1.45 (m, 2H), 1.21-1.18 (m, 1H), 1.14 (s, 9H), 1.05-0.98 (m, 2H). UPLC (M+H)=542.5.

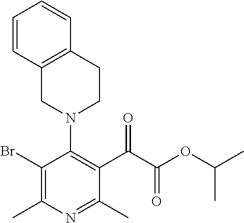 [SMILES: Cc1nc(C)c(C(=O)C(=O)OC(C)C)c(N2CCc3ccccc3C2)c1Br]

### Isopropyl 2-(5-bromo-4-(3,4-dihydroisoquinolin-2(1H)-yl)-2,6-dimethylpyridin-3-yl)-2-oxoacetate

To a solution of 1,2,3,4-tetrahydroisoquinoline, HCl (1.39 g, 8.22 mmol) and DIEA (2.61 mL, 14.94 mmol) in anhydrous CH₃CN (40 mL) was added isopropyl 2-(5-bromo-4-chloro-2,6-dimethylpyridin-3-yl)-2-oxoacetate (2.5 g, 7.47 mmol) at rt. The resulting mixture was placed in a pre-heated oil bath (80° C.) and stirred for 18 h. Additional 1,2,3,4-tetrahydroisoquinoline, HCl (700 mg, 4.11 mmol) was added and the reaction continued for 18 h before being cooled, concentrated, and charged (DCM) to a 80 g ISCO silica gel cartridge and gradient elution (5-35% EtOAc/hexanes) using an Isolera chromatography station to give isopropyl 2-(5-bromo-4-(3,4-dihydroisoquinolin-2(1H)-yl)-2,6-dimethylpyridin-3-yl)-2-oxoacetate 918 mg (28.5%). ¹H NMR (500 MHz, CDCl3) δ 7.18-7.12 (m, 3H), 7.04-7.02 (m, 1H), 4.72-4.67 (m, 1H), 3.51 (s, 1H), 3.45 (br. s, 1H), 2.87 (br. s, 2H), 2.74 (s, 3H), 2.49 (s, 3H), 1.59 (s, 2H), 1.04 (s, 6H). UPLC (M+H)=433.2.

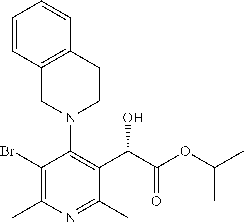 [SMILES: Cc1nc(C)c([C@H](O)C(=O)OC(C)C)c(N2CCc3ccccc3C2)c1Br]

### (S)-Isopropyl 2-(5-bromo-4-(3,4-dihydroisoquinolin-2(1H)-yl)-2,6-dimethylpyridin-3-yl)-2-hydroxyacetate

The benzo[d][1,3,2]dioxaborole (0.61 mL, 2.10 mmol; 50% soln in toluene) was added to a nitrogen purged solution isopropyl 2-(5-bromo-4-(3,4-dihydroisoquinolin-2(1H)-yl)-2,6-dimethylpyridin-3-yl)-2-oxoacetate (626 mg, 1.45 mmol) and 0.44 mL of 1M (R)-1-methyl-3,3-diphenylhexahydropyrrolo[1,2-c][1,3,2]oxazaborole (121 mg, 0.44 mmol) in toluene (15 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 40 g ISCO silica gel cartridge and gradient elution (5-50% EtOAc/hexanes) using an Isolera chromatography station gave (S)-isopropyl 2-(5-bromo-4-(3,4-dihydroisoquinolin-2(1H)-yl)-2,6-dimethylpyridin-3-yl)-2-hydroxyacetate 290 mg (46%) as a mixture of diasteromers: ¹H NMR (500 MHz, CDCl3) δ 7.21-7.17 (m, 3H), 7.05-7.01 (m, 1H), 5.74/5.71 (d, J=5.2 Hz, 1H), 5.10-5.01/4.90-4.86 (m, 1H), 4.92-4.82 (m, 1H), 4.06-3.93 (m, 2H), 3.51 (s, 1H), 3.27-3.13 (m, 2H), 2.83/2.80 (s, 1H), 2.70 (s, 3H), 2.54/2.53 (s, 3H), 1.23/1.20 (d, J=6.2 Hz, 3H), 1.17/1.15 (d, J=6.2 Hz, 3H). UPLC (M+H)=435.3.

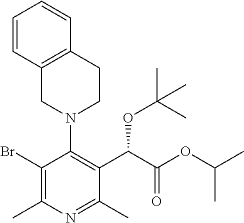 [SMILES: Cc1nc(C)c([C@H](OC(C)(C)C)C(=O)OC(C)C)c(N2CCc3ccccc3C2)c1Br]

### (S)-Isopropyl 2-(5-bromo-4-(3,4-dihydroisoquinolin-2(1H)-yl)-2,6-dimethylpyridin-3-yl)-2-(tert-butoxy)acetate

The isobutylene gas was bubbled into a nitrogen purged, cooled (0° C.) solution of (S)-isopropyl 2-(5-bromo-4-(3,4-dihydroisoquinolin-2(1H)-yl)-2,6-dimethylpyridin-3-yl)-2-hydroxyacetate (270 mg, 0.62 mmol) and 0.06 mL of 70% HClO₄ in DCM (15 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 40 g ISCO silica gel cartridge and gradient elution (5-35% EtOAc/hexanes) using an Isolera chromatography station gave (S)-isopropyl 2-(5-bromo-4-(3,4-dihydroisoquinolin-2(1H)-yl)-2,6-dimethylpyridin-3-yl)-2-(tert-butoxy)acetate 111 mg (34%) as a mixture of diastereomers: ¹H NMR (500 MHz, CDCl3) δ 7.21-7.16 (m, 3H), 7.02-6.99 (m, 1H), 6.06 (s, 1H), 5.10/4.61 (d, J=15.4 Hz, 1H), 5.03-4.97 (m, 1H), 4.27-4.19 (m, 1H), 3.85-3.78 (m, 1H), 3.34-3.16 (m, 2H), 2.89-2.76 (m, 1H), 2.69 (s, 3H), 2.59/2.55 (s, 3H), 1.21 (d, J=6.2 Hz, 3H), 1.14 (s, 9H), 1.07 (d, J=6.2 Hz, 3H). UPLC (M+H)=491.4.

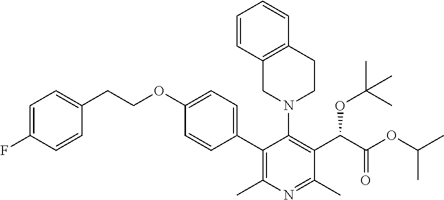 [SMILES: Cc1nc(C)c([C@H](OC(C)(C)C)C(=O)OC(C)C)c(N2CCc3ccccc3C2)c1-c1ccc(OCCc2ccc(F)cc2)cc1]

### (S)-Isopropyl 2-(tert-butoxy)-2-(4-(3,4-dihydroisoquinolin-2(1H)-yl)-5-(4-(4-fluorophenethoxy)phenyl)-2,6-dimethylpyridin-3-yl)acetate

The palladium acetate (4.6 mg, 0.02 mmol) was added to a nitrogen purged and degassed solution of (S)-isopropyl 2-(5-bromo-4-(3,4-dihydroisoquinolin-2(1H)-yl)-2,6-dimethylpyridin-3-yl)-2-(tert-butoxy)acetate (100 mg, 0.20 mmol), 2-(4-(4-fluorophenethoxy)phenyl)-6-methyl-1,3,6,2-dioxazaborocane-4,8-dione (83 mg, 0.23 mmol), and potassium phosphate tribasic (325 mg, 1.53 mmol) in 1,4-dioxane (3 mL) and water (0.6 mL). The reaction mixture was stirred in a screw-capped pressure vessel for 4 h at 90° C., cooled, diluted with EtOAc, and the organic layer was washed with brine and dried (Na2CO3). The crude product was charged (DCM) to a 24 g ISCO silica gel cartridge and gradient elution (5-70% EtOAc/hexanes) using an Isolera chromatography station gave (S)-isopropyl 2-(tert-butoxy)-2-(4-(3,4-dihydroisoquinolin-2(1H)-yl)-5-(4-(4-fluorophenethoxy)phenyl)-2,6-dimethylpyridin-3-yl)acetate 29 mg (23%) as a mixture of diasteromers: ¹H NMR (500 MHz, DMSO) δ 7.37-7.29 (m, 3H), 7.12-6.96 (m, 8H), 6.92-6.91/6.74-6.73 (m, 1H), 5.99/5.77 (s, 1H), 4.96-4.91 (m, 1H), 4.38/4.02 (d, J=16.0 Hz, 1H) 4.19 (t, J=7.0 Hz, 2H), 3.53-3.49/3.30-3.27 (m, 1H), 3.18-3.10/2.94-2.87 (m, 1H), 3.02 (t, J=7.0 Hz, 2H), 2.85-2.81 (m, 1H), 2.64-2.61/2.55-2.50 (m, 1H), 2.47/2.43 (s, 3H), 2.09 (s, 3H), 1.20 (d, J=6.2 Hz, 3H), 1.13 (d, J=6.2 Hz, 3H), 0.99/0.92 (s, 9H). UPLC (M+H)=625.7.

### Example 2

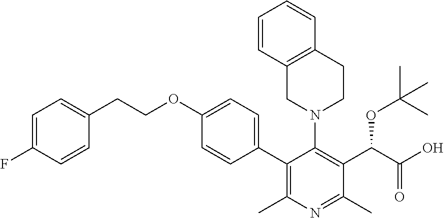 [SMILES: Cc1nc(C)c([C@H](OC(C)(C)C)C(=O)O)c(N2CCc3ccccc3C2)c1-c1ccc(OCCc2ccc(F)cc2)cc1]

### (S)-2-(tert-Butoxy)-2-(4-(3,4-dihydroisoquinolin-2(1H)-yl)-5-(4-(4-fluorophenethoxy)phenyl)-2,6-dimethylpyridin-3-yl)acetic acid

The 0.10 mL of 1M sodium hydroxide (4.0 mg, 0.10 mmol) was added to a solution of (S)-isopropyl 2-(tert-butoxy)-2-(4-(3,4-dihydroisoquinolin-2(1H)-yl)-5-(4-(4-fluorophenethoxy)phenyl)-2,6-dimethylpyridin-3-yl)acetate (25 mg, 0.04 mmol) in ethanol (1.5 mL) and stirred for 18 h at 90° C. An addition 0.1 mL of sodium hydroxide soln was added and the reaction continued 18 h. The reaction mixture was neutralized with 1N HCl soln, extracted with EtOAc, and the organic layer was washed with brine, and dried (MgSO₄). The crude material was purified by prep to afford (S)-2-(tert-butoxy)-2-(4-(3,4-dihydroisoquinolin-2(1H)-yl)-5-(4-(4-fluorophenethoxy)phenyl)-2,6-dimethylpyridin-3-yl)acetic acid 8.5 mg (37%). ¹H NMR (500 MHz, DMSO) δ 7.37-7.28 (m, 3H), 7.12-6.95 (m, 8H), 6.91/6.72 (d, J=7.0 Hz, 1H), 5.89/5.66 (s, 1H), 4.62/3.98 (d, J=16 Hz, 1H), 4.19 (t, J=7.0 Hz, 2H) 3.67-3.63/3.44-3.42 (m, 1H), 3.39 (br. s, 2H), 3.24-3.10 (m, 1H), 3.02 (t, J=6.6 Hz, 2H), 2.94-2.81 (m, 1H), 2.47/2.45 (s, 3H), 2.08 (s, 3H), 0.97/0.90 (s, 9H). UPLC (M+H)=583.6.

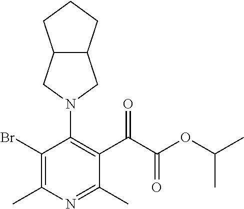 [SMILES: Cc1nc(C)c(C(=O)C(=O)OC(C)C)c(N2CC3CCCC3C2)c1Br]

### Isopropyl 2-(5-bromo-4-(hexahydrocyclopenta[c]pyrrol-2(1H)-yl)-2,6-dimethylpyridin-3-yl)-2-oxoacetate

To a solution of octahydrocyclopenta[c]pyrrole, HCl (1.1 g, 7.47 mmol) and DIEA (2.61 mL, 14.94 mmol) in anhydrous CH₃CN (40 mL) was added isopropyl 2-(5-bromo-4-chloro-2,6-dimethylpyridin-3-yl)-2-oxoacetate (2.5 g, 7.47 mmol) at rt. The resulting mixture was placed in a pre-heated oil bath (80° C.) and stirred for 18 h before being cooled, concentrated, and charged (DCM) to a 50 g Biotage SNAP silica gel cartridge and gradient elution (5-35% EtOAc/hexanes) using an Isolera chromatography station to give isopropyl 2-(5-bromo-4-(hexahydrocyclopenta[c]pyrrol-2(1H)-yl)-2,6-dimethylpyridin-3-yl)-2-oxoacetate 1.64 g (54%). ¹H NMR (500 MHz, CDCl3) δ 5.16-5.11 (m, 1H), 3.36 (t, J=9.6 Hz, 2H), 3.10-3.07 (m, 2H), 2.73-2.70 (m, 2H), 2.70 (s, 3H), 2.44 (s, 3H), 1.76-1.60 (m, 4H), 1.51-1.47 (m, 2H), 1.37 (d, J=6.2 Hz, 6H). UPLC (M+H)=411.3.

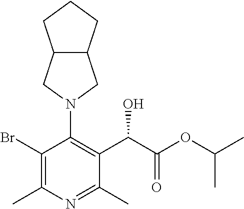 [SMILES: Cc1nc(C)c([C@H](O)C(=O)OC(C)C)c(N2CC3CCCC3C2)c1Br]

### (2S)-Isopropyl 2-(5-bromo-4-(hexahydrocyclopenta[c]pyrrol-2(1H)-yl)-2,6-dimethylpyridin-3-yl)-2-hydroxyacetate

The benzo[d][1,3,2]dioxaborole (1.2 mL, 5.13 mmol; 50% soln in toluene) was added to a nitrogen purged solution of isopropyl 2-(5-bromo-4-(hexahydrocyclopenta[c]pyrrol-2(1H)-yl)-2,6-dimethylpyridin-3-yl)-2-oxoacetate (1.4 g, 3.42 mmol) and 1.0 mL of 1M (R)-1-methyl-3,3-diphenylhexahydropyrrolo[1,2-c][1,3,2]oxazaborole (248 mg, 1.0 mmol) in toluene (40 mL) cooled to −50° C. The reaction was allowed to slowly warm to −15° C. and placed in the freezer for 18 h before being quenched with 1M Na2CO3 (3 mL) and stirred for 20 min. The organic layer was diluted with EtOAc and washed with brine and dried (MgSO4). The crude product was charged (DCM) to a 80 g ISCO silica gel cartridge and gradient elution (5-30% EtOAc/hexanes) using an Isolera chromatography station gave (2S)-isopropyl 2-(5-bromo-4-(hexahydrocyclopenta[c]pyrrol-2(1H)-yl)-2,6-dimethylpyridin-3-yl)-2-hydroxyacetate 1.07 g (71%) as a mixture of diastereomers. ¹H NMR (500 MHz, DMSO) δ 5.79 (d, J=4.4 Hz, 1H), 4.95-4.90 (m, 1H), 3.95 (t, J=7.7 Hz, 1H), 3.44-3.42 (m, 1H), 2.99-2.97 (m, 1H), 2.86-2.84 (m, 1H), 2.75-2.66 (m, 2H), 2.52 (s, 3H), 2.36 (s, 3H), 1.78-1.71 (m, 3H), 1.55-1.45 (m, 3), 1.15 (d, J=6.2 Hz, 3H), 1.08 (d, J=6.2 Hz, 3H). UPLC (M+H)=413.3.

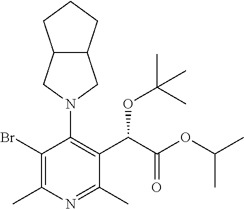 [SMILES: Cc1nc(C)c([C@H](OC(C)(C)C)C(=O)OC(C)C)c(N2CC3CCCC3C2)c1Br]

### (2S)-Isopropyl 2-(5-bromo-4-(hexahydrocyclopenta[c]pyrrol-2(1H)-yl)-2,6-dimethylpyridin-3-yl)-2-(tert-butoxy)acetate

The isobutylene gas was bubbled into a nitrogen purged, cooled (0° C.) solution of (2S)-isopropyl 2-(5-bromo-4-(hexahydrocyclopenta[c]pyrrol-2(1H)-yl)-2,6-dimethylpyridin-3-yl)-2-hydroxyacetate (900 mg, 2.18 mmol) and 0.22 mL of 70% HClO₄ in DCM (35 mL) for 20 min. The reaction mixture was allowed to warm to rt and stirred for 18 h in a pressure sealed vessel, diluted with DCM, washed with 1M Na₂CO₃ soln, and dried over MgSO4. The crude product was charged (DCM) to a 80 g ISCO silica gel cartridge and gradient elution (5-35% EtOAc/hexanes) using an Isolera chromatography station gave (2S)-isopropyl 2-(5-bromo-4-(hexahydrocyclopenta[c]pyrrol-2(1H)-yl)-2,6-dimethylpyridin-3-yl)-2-(tert-butoxy)acetate 690 mg (67.5%): ¹H NMR (500 MHz, DMSO) δ 5.99 (s, 1H), 4.93-4.88 (m, 1H), 3.79 (br. s, 1H), 3.35-3.33 (m, 1H), 3.16 (br. s, 1H), 2.82-7.78 (m, 2H), 2.73 (br. s, 1H), 2.52 (s, 3H), 2.42 (s, 3H), 1.80-1.73 (m, 3H), 1.61-1.57 (m, 1H), 1.54-1.51 (m, 2H), 1.17 (d, J=6.2 Hz, 3H), 1.14 (s, 9H), 1.08 (d, J=6.2 Hz, 3H). UPLC (M+H)=469.4.

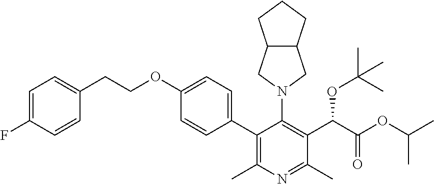 [SMILES: Cc1nc(C)c([C@H](OC(C)(C)C)C(=O)OC(C)C)c(N2CC3CCCC3C2)c1-c1ccc(OCCc2ccc(F)cc2)cc1]

### (2S)-Isopropyl 2-(tert-butoxy)-2-(5-(4-(4-fluorophenethoxy) phenyl)-4-(hexahydrocyclopenta[c]pyrrol-2(1H)-yl)-2,6-dimethylpyridin-3-yl)acetate

The Pd(Ph₃P)₄ (49 mg, 0.043 mmol) was added to a nitrogen purged and degassed solution of (2S)-isopropyl 2-(5-bromo-4-(hexahydrocyclopenta[c]pyrrol-2(1H)-yl)-2,6-dimethylpyridin-3-yl)-2-hydroxyacetate (100 mg, 0.214 mmol), (4-(4-fluorophenethoxy)phenyl)boronic acid (61 mg, 0.24 mmol), and potassium phosphate tribasic (317 mg, 1.50 mmol) in 1,4-dioxane (3 mL) and water (0.6 mL). The reaction mixture was stirred in a screw-capped pressure vessel for 4 h at 90° C., cooled, diluted with EtOAc, and the organic layer was washed with brine and dried (Na₂CO₃). The crude product was charged (DCM) to a 24 g ISCO silica gel cartridge and gradient elution (5-70% EtOAc/hexanes) using an Isolera chromatography station gave (2S)-isopropyl 2-(tert-butoxy)-2-(5-(4-(4-fluorophenethoxy) phenyl)-4-(hexahydrocyclopenta[c]pyrrol-2(1H)-yl)-2,6-dimethylpyridin-3-yl)acetate 60 mg (46.5%) as a mixture of diasteromers: ¹H NMR (500 MHz, DMSO) δ 7.39-7.36 (m, 2H), 7.18-7.12 (m, 3H), 7.02-6.96 (m, 3H), 5.81 (s, 1H), 4.98-4.93 (m, 1H), 4.23 (t, J=6.2 Hz, 2H), 3.38-3.34 (m, 2H), 3.17 (br, s, 1H), 3.05 (t, J=6.6 Hz, 2H), 2.72 (t, J=8.1 Hz, 1H), 2.40 (s, 3H), 2.37-2.34 (m, 2H), 2.06 (br. s, 1H), 2.04 (s, 3H), 1.52 (br. s, 2H), 1.39-1.24 (m, 3H), 1.20 (d, J=6.2 Hz, 3H), 1.15 (d, J=6.2 Hz, 3H) 1.10 (s, 9H). UPLC (M+H)=603.5.

### Example 3

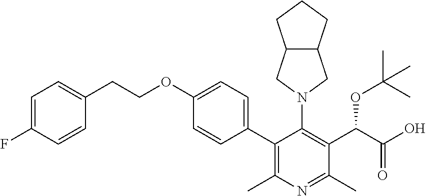 [SMILES: Cc1nc(C)c([C@H](OC(C)(C)C)C(=O)O)c(N2CC3CCCC3C2)c1-c1ccc(OCCc2ccc(F)cc2)cc1]

### (S)-2-(5-(4-(Benzylcarbamoyl)phenyl)-4-(2-azabicyclo[2.2.1]heptan-2-yl)-2,6-dimethylpyridin-3-yl)-2-(tert-butoxy)acetic acid

The 0.40 mL of 1M sodium hydroxide (15.9 mg, 0.40 mmol) was added to a solution of (2S)-isopropyl 2-(tert-butoxy)-2-(5-(4-(4-fluorophenethoxy)phenyl)-4-(hexahydrocyclopenta[c]pyrrol-2(1H)-yl)-2,6-dimethylpyridin-3-yl)acetate (60 mg, 0.10 mmol) in ethanol (2 mL) and stirred for 18 h at 90° C. The reaction mixture was neutralized with 1N HCl soln, extracted with EtOAc, and the organic layer was washed with brine, and dried (MgSO₄). The crude material was purified by prep-HPLC to obtain (S)-2-(5-(4-(benzylcarbamoyl)phenyl)-4-(2-azabicyclo[2.2.1]heptan-2-yl)-2,6-dimethylpyridin-3-yl)-2-(tert-butoxy)acetic acid 21.5 mg (38%). ¹H NMR (500 MHz, DMSO) δ 7.38-7.36 (m, 2H), 7.17-7.12 (m, 3H), 7.02-6.96 (m, 3H), 5.68 (s, 1H), 4.22 (t, J=7.3 Hz, 2H), 3.38 (br. s, 1H), 3.06-3.03 (m, 2H), 2.88-2.84 (m, 1H), 2.69 (t, J=8.8 Hz, 1H), 2.49-2.44 (m, 1H), 2.42 (s, 3H), 2.35-2.32 (m, 1H), 2.03 (s, 3H), 2.01 (br. s, 1H), 1.52-1.44 (m, 2H), 1.38-1.34 (m, 1H), 1.28-1.24 (m, 2H), 1.09 (s, 9H), 1.05 (br. s, 1H). UPLC (M+H)=561.5.

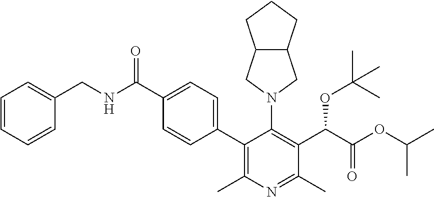 [SMILES: Cc1nc(C)c([C@H](OC(C)(C)C)C(=O)OC(C)C)c(N2CC3CCCC3C2)c1-c1ccc(C(=O)NCc2ccccc2)cc1]

### (2S)-Isopropyl 2-(5-(4-(benzylcarbamoyl)phenyl)-4-(hexahydrocyclopenta[c]pyrrol-2(1H)-yl)-2,6-dimethylpyridin-3-yl)-2-(tert-butoxy)acetate

The Pd(Ph₃P)₄ (54 mg, 0.047 mmol) was added to a nitrogen purged and degassed solution of (2S)-isopropyl 2-(5-bromo-4-(hexahydrocyclopenta[c]pyrrol-2(1H)-yl)-2,6-dimethylpyridin-3-yl)-2-hydroxyacetate (110 mg, 0.24 mmol), (4-(benzylcarbamoyl)phenyl)boronic acid (60 mg, 0.24 mmol), and potassium phosphate tribasic (349 mg, 1.64 mmol) in 1,4-dioxane (3 mL) and water (0.6 mL). The reaction mixture was stirred in a screw-capped pressure vessel for 4 h at 90° C., cooled, diluted with EtOAc, and the organic layer was washed with brine and dried (Na₂CO₃). The crude product was charged (DCM) to a 24 g ISCO silica gel cartridge and gradient elution (5-70% EtOAc/hexanes) using an Isolera chromatography station gave (2S)-isopropyl 2-(5-(4-(benzylcarbamoyl)phenyl)-4-(hexahydrocyclopenta[c]pyrrol-2(1H)-yl)-2,6-dimethylpyridin-3-yl)-2-(tert-butoxy)acetate 83 mg (59%) as a mixture of diasteromers: UPLC (M+H)=561.45.

### Example 4

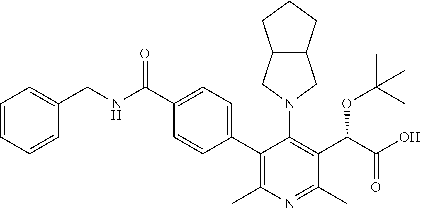 [SMILES: Cc1nc(C)c([C@H](OC(C)(C)C)C(=O)O)c(N2CC3CCCC3C2)c1-c1ccc(C(=O)NCc2ccccc2)cc1]

### (2S)-2-(5-(4-(Benzylcarbamoyl)phenyl)-4-(hexahydrocyclopenta[c]pyrrol-2(1H)-yl)-2,6-dimethylpyridin-3-yl)-2-(tert-butoxy)acetic acid

The 0.21 mL of 1M sodium hydroxide (8.4 mg, 0.21 mmol) was added to a solution of (2S)-isopropyl 2-(5-(4-(benzylcarbamoyl)phenyl)-4-(hexahydrocyclopenta[c]pyrrol-2(1H)-yl)-2,6-dimethylpyridin-3-yl)-2-(tert-butoxy)acetate (63 mg, 0.11 mmol) in ethanol (2 mL) and stirred for 18 h at 90° C. An additional 0.2 mL of sodium hydroxide soln was added and the reaction continued for 18 h. The reaction mixture was neutralized with 1N HCl soln, extracted with EtOAc, and the organic layer was washed with brine, and dried (MgSO4). The crude material was purified by prep HPLC to obtain (2S)-2-(5-(4-(benzylcarbamoyl)phenyl)-4-(hexahydrocyclopenta[c]pyrrol-2(1H)-yl)-2,6-dimethylpyridin-3-yl)-2-(tert-butoxy)acetic acid 21.2 mg (36%). ¹H NMR (500 MHz, DMSO) δ 9.18-9.16 (m, 1H), 7.99-7.95 (m, 2H), 7.42 (d, J=7.7 Hz, 1H), 7.34-7.31 (m, 4H), 7.26-7.23 (m, 1H), 7.18 (d, J=7.0 Hz, 1H), 5.56 (s, 1H), 4.51 (d, J=5.9 Hz, 2H), 3.42 (br. S, 1H), 2.73-2.69 (m, 1H), 2.43 (s, 3H), 2.41 (br. S, 2H), 2.35-2.32 (m, 1H), 2.06 (br. S, 1H), 2.02 (s, 3H), 1.52-1.44 (m, 2H), 1.35-1.32 (m, 1H), 1.27-1.24 (m, 2H), 1.09 (s, 9H), 1.02 (br. S, 1H). UPLC (M+H)=556.7.

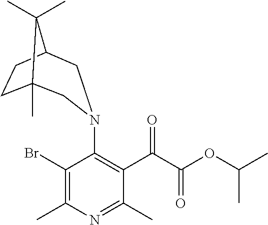 [SMILES: Cc1nc(C)c(C(=O)C(=O)OC(C)C)c(N2CC3CCC(C)(C2)C3(C)C)c1Br]

### Isopropyl 2-(5-bromo-2,6-dimethyl-4-(1,8,8-trimethyl-3-azabicyclo[3.2.1]octan-3-yl)pyridin-3-yl)-2-oxoacetate

To a solution of 1,8,8-trimethyl-3-azabicyclo[3.2.1]octane (1.0 g, 6.52 mmol) and DIEA (3.29 mL, 18.8 mmol) in anhydrous CH₃CN (40 mL) was added isopropyl 2-(5-bromo-4-chloro-2,6-dimethylpyridin-3-yl)-2-oxoacetate (2.1 g, 6.28 mmol) at rt. The resulting mixture was placed in a pre-heated oil bath (80° C.) and stirred for 18 h before being cooled, concentrated, and charged (DCM) to a 50 g Biotage SNAP silica gel cartridge and gradient eluted (5-55% EtOAc/hexanes) using an Isolera chromatography station to give isopropyl 2-(5-bromo-2,6-dimethyl-4-(1,8,8-trimethyl-3-azabicyclo[3.2.1]octan-3-yl)pyridin-3-yl)-2-oxoacetate 2.2 g (78%). ¹H NMR (500 MHz, DMSO) δ 5.12-5.07 (m, 1H), 3.69 (br. s, 1H), 3.42-4.41 (m, 1H), 2.61 (s, 3H), 2.40 (d, J=10.3 Hz, 1H), 2.21 (s, 3H), 2.44 (s, 3H), 2.14 (d, J 10.3 Hz, 1H), 1.72-1.65 (m, 2H), 1.19-1.42 (m, 2H), 1.34-1.30 (m, 1H), 1.27 (d, J=6.2 Hz, 6H), 1.09 (s, 3H), 0.83 (s, 3H), 0.73 (s, 3H). UPLC (M+H)=453.2.

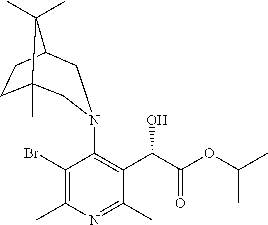 [SMILES: Cc1nc(C)c([C@H](O)C(=O)OC(C)C)c(N2CC3CCC(C)(C2)C3(C)C)c1Br]

### (S)-Isopropyl 2-(5-bromo-2,6-dimethyl-4-(1,8,8-trimethyl-3-azabicyclo[3.2.1]octan-3-yl)pyridin-3-yl)-2-hydroxyacetate

The benzo[d][1,3,2]dioxaborole (1.8 mL, 7.31 mmol; 50% soln in toluene) was added to a nitrogen purged solution of isopropyl 2-(5-bromo-2,6-dimethyl-4-(1,8,8-trimethyl-3-azabicyclo[3.2.1]octan-3-yl)pyridin-3-yl)-2-oxoacetate (2.1 g, 4.85 mmol) and 1.5 mL of 1M (R)-1-methyl-3,3-diphenylhexa-hydropyrrolo[1,2-c][1,3,2]oxazaborole (405 mg, 1.46 mmol) in toluene (50 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 80 g ISCO silica gel cartridge and gradient elution (5-65% EtOAc/hexanes) using an Isolera chromatography station gave (S)-isopropyl 2-(5-bromo-2,6-dimethyl-4-(1,8,8-trimethyl-3-azabicyclo[3.2.1]octan-3-yl)pyridin-3-yl)-2-hydroxyacetate 1.8 g (82%) as a mixture of diastereomers (5:1). Maj or isomer: ¹H NMR (500 MHz, DMSO) δ 6.30 (s, 1H), 4.99-4.94 (m, 1H), 3.90 (d, J=8.8 Hz, 1H), 3.50 (d, J=9.9 Hz, 1H), 2.54 (s, 3H), 2.49 (br. s, 1H), 2.35 (s, 3H), 2.25 (d, J=10.3 Hz, 1H), 1.91-1.85 (m, 1H), 1.76-1.60 (m, 4H), 1.18 (s, 3H), 1.14 (d, J=6.2 Hz, 3H), 1.05 (d, J=6.2 Hz, 3H), 0.90 (s, 3H), 0.78 (s, 3H). UPLC (M+H)=454.75.

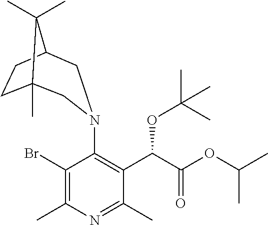 [SMILES: Cc1nc(C)c([C@H](OC(C)(C)C)C(=O)OC(C)C)c(N2CC3CCC(C)(C2)C3(C)C)c1Br]

### (S)-Isopropyl 2-(5-bromo-2,6-dimethyl-4-(1,8,8-trimethyl-3-azabicyclo[3.2.1]octan-3-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(1,8,8-trimethyl-3-azabicyclo[3.2.1]octan-3-yl)pyridin-3-yl)-2-hydroxyacetate (1.3 g, 2.87 mmol) and 0.7 mL of 70% HClO₄ in DCM (20 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 80 g ISCO silica gel cartridge and gradient elution (5-35% EtOAc/hexanes) using an Isolera chromatography station gave recovered starting material 1.4 g (78%) and (S)-isopropyl 2-(5-bromo-2,6-dimethyl-4-(1,8,8-trimethyl-3-azabicyclo[3.2.1]octan-3-yl)pyridin-3-yl)-2-(tert-butoxy)acetate 507 mg (35%) as a mixture of diastereomers: UPLC (M+H)=511.25.

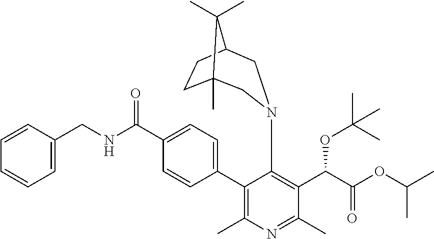 [SMILES: Cc1nc(C)c([C@H](OC(C)(C)C)C(=O)OC(C)C)c(N2CC3CCC(C)(C2)C3(C)C)c1-c1ccc(C(=O)NCc2ccccc2)cc1]

### (S)-Isopropyl 2-(5-(4-(benzylcarbamoyl)phenyl)-2,6-dimethyl-4-(1,8,8-trimethyl-3-azabicyclo[3.2.1]octan-3-yl)pyridin-3-yl)-2-(tert-butoxy)acetate

The Pd(Ph₃P)₄ (113 mg, 0.098 mmol) was added to a nitrogen purged and degassed solution of (S)-isopropyl 2-(5-bromo-2,6-dimethyl-4-(1,8,8-trimethyl-3-azabicyclo[3.2.1]octan-3-yl)pyridin-3-yl)-2-(tert-butoxy)acetate (250 mg, 0.491 mmol), (4-(benzylcarbamoyl)phenyl)boronic acid (138 mg, 0.54 mmol), and potassium phosphate tribasic (728 mg, 3.43 mmol) in 1,4-dioxane (7.5 mL) and water (1.5 mL). The reaction mixture was stirred in a screw-capped pressure vessel for 4 h at 90° C., cooled, diluted with EtOAc, and the organic layer was washed with brine and dried (Na₂CO₃). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-2,6-dimethyl-4-(1,8,8-trimethyl-3-azabicyclo[3.2.1]octan-3-yl)pyridin-3-yl)-2-(tert-butoxy)acetate 41 mg (13%) as a mixture of diasteromers: ¹H NMR (500 MHz, DMSO) δ 9.15-9.14 (m, 1H), 8.01-8.00 (m, 2H), 7.44 (d, J=8.1 Hz, 1H), 7.38 (d, J=7.7 Hz, 1H), 7.35-7.34 (m, 4H), 7.27-7.24 (m, 1H), 6.02 (s, 1H), 4.99-4.94 (m, 1H), 4.52 (d, J=5.9 Hz, 2H), 3.42-3.38 (m, 1H), 3.20-3.18 (m, 1H), 2.80-2.78 (m, 1H), 2.72 (br. s, 1H), 2.50 (s, 3H), 2.06 (d, J 9.9 Hz, 1H), 1.95 (s, 3H), 1.86 (br. s, 1H), 1.78-1.75 (m, 2H), 1.56 (br. s, 1H), 1.20-1.18 (m, 12H), 1.15 (d, J=6.2 Hz, 3H), 0.69 (s, 3H), 0.56 (s, 3H), 0.13 (s, 3H). UPLC (M+H)=640.6.

### Example 5

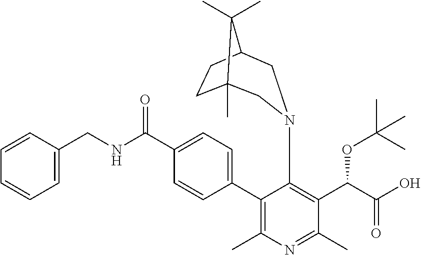 [SMILES: Cc1nc(C)c([C@H](OC(C)(C)C)C(=O)O)c(N2CC3CCC(C)(C2)C3(C)C)c1-c1ccc(C(=O)NCc2ccccc2)cc1]

### (S)-2-(5-(4-(Benzylcarbamoyl)phenyl)-2,6-dimethyl-4-(1,8,8-trimethyl-3-azabicyclo[3.2.1]octan-3-yl)pyridin-3-yl)-2-(tert-butoxy)acetic acid

The 0.26 mL of 1M sodium hydroxide (10.25 mg, 0.26 mmol) was added to a solution of (S)-isopropyl 2-(5-(4-(benzylcarbamoyl)phenyl)-2,6-dimethyl-4-(1,8,8-trimethyl-3-azabicyclo[3.2.1]octan-3-yl)pyridin-3-yl)-2-(tert-butoxy)acetate (41 mg, 0.064 mmol) in ethanol (2.5 mL) and stirred for 18 h at 90° C. An additional 0.26 mL of sodium hydroxide soln was added and the reaction continued for 72 h. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(1,8,8-trimethyl-3-azabicyclo[3.2.1]octan-3-yl)pyridin-3-yl)-2-(tert-butoxy)acetic acid 3.5 mg (9%). ¹H NMR (500 MHz, DMSO) δ 9.14-9.12 (m, 1H), 8.00-7.96 (m, 2H), 7.42 (d, J=7.3 Hz, 1H), 7.36-7.34 (m, 5H), 7.27-7.25 (m, 1H), 6.02 (s, 1H), 4.52 (d, J=4.4 Hz, 2H), 3.11 (br. s, 1H), 3.04 (br. s, 1H), 2.94 (br. s, 1H), 2.84 (br. s, 1H), 2.50 (s, 3H), 2.28-2.26 (m, 1H), 2.40-1.93 (s, 3H), 1.75 (br. s, 2H), 1.55-1.42 (m, 2H), 1.16/1.15 (s, 9H), 0.69 (s, 3H), 0.62 (s, 3H), 0.54 (s, 3H). UPLC (M+H)=598.6.

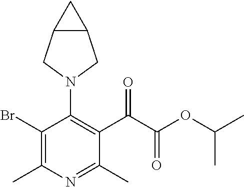 [SMILES: Cc1nc(C)c(C(=O)C(=O)OC(C)C)c(N2CC3CC3C2)c1Br]

### Isopropyl 2-(4-(3-azabicyclo[3.1.0]hexan-3-yl)-5-bromo-2,6-dimethylpyridin-3-yl)-2-oxoacetate

To a solution of 3-azabicyclo[3.1.0]hexane, HCl (250 mg, 2.1 mmol) and DIEA (1.46 mL, 8.36 mmol) in anhydrous CH₃CN (5 mL) was added isopropyl 2-(5-bromo-4-chloro-2,6-dimethylpyridin-3-yl)-2-oxoacetate (699 mg, 2.1 mmol) at rt. After heating at 80° C. for 20 h, the reaction mixture was cooled, diluted with ether, washed with water, brine, dried (MgSO₄), filtered. The crude product was charged (DCM) to a 80 g ISCO silica gel cartridge and gradient elution (0-10% EtOAc/hexanes) using an Isolera chromatography station to give isopropyl 2-(4-(3-azabicyclo[3.1.0]hexan-3-yl)-5-bromo-2,6-dimethylpyridin-3-yl)-2-oxoacetate 365 mg (46%). ¹H NMR (500 MHz, CDCl3) δ 5.07-5.02 (m, 1H), 3.52 (d, J=8.4 Hz, 2H), 3.01 (d, J 9.2 Hz, 2H), 2.60 (s, 3H), 2.29 (s, 3H), 1.58-1.56 (m, 2H), 1.30 (d, J=6.2 Hz, 6H), 0.58-0.54 (m, 1H), 0.49-0.46 (m, 1H). UPLC (M+H)=383.2.

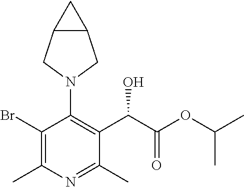 [SMILES: Cc1nc(C)c([C@H](O)C(=O)OC(C)C)c(N2CC3CC3C2)c1Br]

### (2S)-Isopropyl 2-(4-(3-azabicyclo[3.1.0]hexan-3-yl)-5-bromo-2,6-dimethylpyridin-3-yl)-2-hydroxyacetate

The benzo[d][1,3,2]dioxaborole (0.4 mL, 1.89 mmol; 50% soln in toluene) was added to a nitrogen purged solution of isopropyl 2-(4-(3-azabicyclo[3.1.0]hexan-3-yl)-5-bromo-2,6-dimethylpyridin-3-yl)-2-oxoacetate (360 mg, 0.94 mmol) and 0.38 mL of 1M (R)-1-methyl-3,3-diphenylhexahydropyrrolo-[1,2-c][1,3,2]oxazaborole (105 mg, 0.38 mmol) in toluene (7 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 80 g ISCO silica gel cartridge and gradient elution (0-50% EtOAc/hexanes) using an Isolera chromatography station gave (2S)-isopropyl 2-(4-(3-azabicyclo[3.1.0]hexan-3-yl)-5-bromo-2,6-dimethylpyridin-3-yl)-2-hydroxyacetate 360 mg (100%) as a mixture of diastereomers. ¹H NMR (500 MHz, DMSO) δ 5.54 (s, 1H), 4.94-4.90 (m, 1H), 3.69 (d, J=7.3 Hz, 1H), 3.61 (d, J=7.3 Hz, 1H), 3.04 (d, J=7.7 Hz, 1H), 2.98 (d, J=8.1 Hz, 1H), 2.52 (s, 3H), 2.35 (s, 3H), 1.59-1.58 (m, 2H), 1.15 (d, J=6.2 Hz, 3H), 1.08 (d, J=6.2 Hz, 3H), 0.68-0.65 (m, 1H), 0.61-0.57 (m, 1H). UPLC (M+H)=385.9.

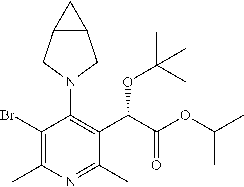 [SMILES: Cc1nc(C)c([C@H](OC(C)(C)C)C(=O)OC(C)C)c(N2CC3CC3C2)c1Br]

### (2S)-Isopropyl 2-(4-(3-azabicyclo[3.1.0]hexan-3-yl)-5-bromo-2,6-dimethylpyridin-3-yl)-2-(tert-butoxy)acetate

The isobutylene gas was bubbled into a nitrogen purged, cooled (0° C.) solution of (2S)-isopropyl 2-(4-(3-azabicyclo[3.1.0]hexan-3-yl)-5-bromo-2,6-dimethylpyridin-3-yl)-2-hydroxyacetate (350 mg, 2.18 mmol) and 0.11 mL of 70% HClO₄ in DCM (5 mL) for 20 min. The reaction mixture was allowed to warm to rt and stirred for 18 h in a pressure sealed vessel, diluted with DCM, washed with 1M Na2CO3 soln, and dried over MgSO₄. The crude product was charged (DCM) to a 40 g ISCO silica gel cartridge and gradient elution (0-10% EtOAc/hexanes) using an Isolera chromatography station gave (2S)-isopropyl 2-(4-(3-azabicyclo[3.1.0]hexan-3-yl)-5-bromo-2,6-dimethylpyridin-3-yl)-2-(tert-butoxy)acetate 290 mg (57.5%): ¹H NMR (500 MHz, DMSO) δ 5.73 (s, 1H), 4.92-4.87 (m, 1H), 3.93 (d, J=7.7 Hz, 1H), 3.54 (d, J=7.3 Hz, 1H), 3.14 (d, J=8.1 Hz, 1H), 2.91 (d, J=7.7 Hz, 1H), 2.53 (s, 3H), 2.44 (s, 3H), 1.63-1.61 (m, 2H), 1.18 (d, J=6.2 Hz, 3H), 1.14 (s, 9H), 1.09 (d, J=6.2 Hz, 3H), 0.74 (br. s, 1H), 0.64 (br. s, 1H). UPLC (M+H)=441.3

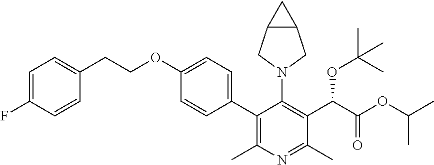 [SMILES: Cc1nc(C)c([C@H](OC(C)(C)C)C(=O)OC(C)C)c(N2CC3CC3C2)c1-c1ccc(OCCc2ccc(F)cc2)cc1]

### (2S)-Isopropyl 2-(4-(3-azabicyclo[3.1.0]hexan-3-yl)-5-(4-(4-(4-fluorophenethoxy) phenyl)-2,6-dimethylpyridin-3-yl)-2-(tert-butoxy)acetate

The Pd(Ph3P)4 (79 mg, 0.068 mmol) was added to a nitrogen purged and degassed solution of (2S)-isopropyl 2-(4-(3-azabicyclo[3.1.0]hexan-3-yl)-5-bromo-2,6-dimethylpyridin-3-yl)-2-(tert-butoxy)acetate (150 mg, 0.34 mmol), (4-(4-fluorophenethoxy)phenyl)boronic acid (98 mg, 0.38 mmol), and potassium phosphate tribasic (543 mg, 2.56 mmol) in 1,4-dioxane (2 mL) and water (0.5 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (0-20% EtOAc/hexanes) using an Isolera chromatography station gave (2S)-isopropyl 2-(4-(3-azabicyclo[3.1.0]hexan-3-yl)-5-(4-(4-fluorophenethoxy)phenyl)-2,6-dimethylpyridin-3-yl)-2-(tert-butoxy)acetate 109 mg (56%) as a mixture of diasteromers: ¹H NMR (500 MHz, DMSO) δ 7.40-7.37 (m, 2H), 7.16-7.12 (m, 3H), 7.02-6.98 (m, 3H), 5.58 (s, 1H), 4.96-4.92 (m, 1H), 4.22 (t, J=6.6 Hz, 2H), 3.23 (d, J=8.8 Hz, 1H), 3.06 (t, J=6.6 Hz, 2H), 2.98-2.96 (m, 1H), 2.60-2.54 (m, 2H), 2.43 (s, 3H), 2.00 (s, 3H), 1.32-1.29 (m, 1H), 1.20 (d, J=6.2 Hz, 3H), 1.19-1.18 (m, 1H), 1.15 (d, J=6.2 Hz, 3H) 1.10 (s, 9H), 0.63-0.62 (m, 1H), 0.46-0.43 (m, 1H). UPLC (M+H)=575.5.

### Example 6

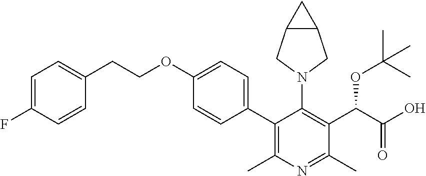 [SMILES: Cc1nc(C)c([C@H](OC(C)(C)C)C(=O)O)c(N2CC3CC3C2)c1-c1ccc(OCCc2ccc(F)cc2)cc1]

### (2S)-2-(4-(3-Azabicyclo[3.1.0]hexan-3-yl)-5-(4-(4-fluorophenethoxy)phenyl)-2,6-dimethylpyridin-3-yl)-2-(tert-butoxy)acetic acid

The 1M sodium hydroxide (0.63 mL, 0.63 mmol) was added to a solution of (2S)-isopropyl 2-(4-(3-azabicyclo[3.1.0]hexan-3-yl)-5-(4-(4-fluorophenethoxy)phenyl)-2,6-dimethylpyridin-3-yl)-2-(tert-butoxy)acetate (60 mg, 0.10 mmol) in ethanol (1 mL) and stirred for 18 h at 85° C. An additional 0.31 mL sodium hydroxide solution was added and stirring was continued for 18 h. The reaction mixture was neutralized with 1N HCl soln, extracted with EtOAc, and the organic layer was washed with brine, and dried (MgSO₄). The crude material was purified by prep HPLC to obtain (2S)-2-(4-(3-azabicyclo[3.1.0]hexan-3-yl)-5-(4-(4-fluorophenethoxy)phenyl)-2,6-dimethylpyridin-3-yl)-2-(tert-butoxy)acetic acid 36 mg (66%). ¹H NMR (500 MHz, DMSO) δ 7.41-7.38 (m, 2H), 7.16-7.13 (m, 3H), 7.02-7.00 (m, 3H), 5.54 (s, 1H), 4.23 (t, J=6.6 Hz, 2H), 3.36-3.35 (m, 2H), 3.06 (t, J=6.2 Hz, 2H), 3.00-2.97 (m, 1H), 2.56 (d, J=8.8 Hz, 1H), 2.43 (s, 3H), 2.01 (s, 3H), 1.31 (br. s, 1H), 1.19 (br. s, 1H), 1.10 (s, 9H), 0.65 (br. s, 1H), 0.47-0.43 (m, 1H). UPLC (M+H)=533.5.

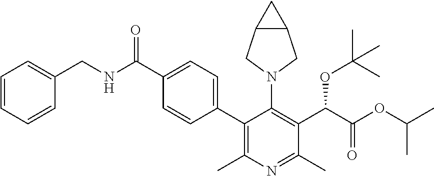 [SMILES: Cc1nc(C)c([C@H](OC(C)(C)C)C(=O)OC(C)C)c(N2CC3CC3C2)c1-c1ccc(C(=O)NCc2ccccc2)cc1]

### (2S)-Isopropyl 2-(5-(4-(benzylcarbamoyl)phenyl)-4-(3-azabicyclo[3.1. O]hexan-3-yl)-2,6-dimethylpyridin-3-yl)-2-(tert-butoxy)acetate

The Pd(Ph₃P)₄ (53 mg, 0.046 mmol) was added to a nitrogen purged and degassed solution of (2S)-isopropyl 2-(4-(3-azabicyclo[3.1.0]hexan-3-yl)-5-bromo-2,6-dimethylpyridin-3-yl)-2-(tert-butoxy)acetate (100 mg, 0.23 mmol), (4-(benzylcarbamoyl)phenyl)boronic acid (64 mg, 0.25 mmol), and potassium phosphate tribasic (362 mg, 1.71 mmol) in 1,4-dioxane (2 mL) and water (0.5 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (0-20% EtOAc/hexanes) using an Isolera chromatography station gave (2S)-isopropyl 2-(5-(4-(benzylcarbamoyl)phenyl)-4-(3-azabicyclo[3.1.0]hexan-3-yl)-2,6-dimethylpyridin-3-yl)-2-(tert-butoxy)acetate 104 mg (77%) as a mixture of diasteromers: δ 9.19-9.17 (m, 1H), 8.00-7.95 (m, 2H), 7.41 (d, J=7.7 Hz, 1H), 7.37-7.33 (m, 4H), 7.27-7.23 (m, 2H), 5.56 (s, 1H), 4.98-4.93 (m, 1H), 4.51 (d, J=5.9 Hz, 2H), 3.25 (d, J=8.8 Hz, 1H), 3.02-2.99 (m, 1H), 2.63 (d, J=8.7 Hz, 1H), 2.45 (s, 3H), 2.01 (s, 3H), 1.31 (br. s, 1H), 1.24-1.18 (m, 1H), 1.21 (d, J=6.2 Hz, 3H), 1.17 (d, J=6.2 Hz, 3H), 1.11 (s, 9H), 0.61-0.60 (m, 1H), 0.47-0.43 (m, 1H). UPLC (M+H)=570.5.

### Example 7

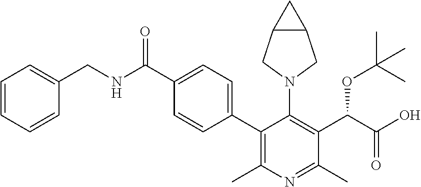 [SMILES: Cc1nc(C)c([C@H](OC(C)(C)C)C(=O)O)c(N2CC3CC3C2)c1-c1ccc(C(=O)NCc2ccccc2)cc1]

### (2S)-2-(5-(4-(Benzylcarbamoyl)phenyl)-4-(3-azabicyclo[3.1.0]hexan-3-yl)-2,6-dimethylpyridin-3-yl)-2-(tert-butoxy)acetic acid

The 1M sodium hydroxide (0.63 mL, 0.63 mmol) was added to a solution of (2S)-isopropyl 2-(5-(4-(benzylcarbamoyl)phenyl)-4-(3-azabicyclo[3.1.0]hexan-3-yl)-2,6-dimethylpyridin-3-yl)-2-(tert-butoxy)acetate (60 mg, 0.11 mmol) in ethanol (1 mL) and stirred for 18 h at 90° C. An additional 0.3 mL of sodium hydroxide soln was added and the reaction continued for 18 h. The reaction mixture was neutralized with 1N HCl soln, extracted with EtOAc, and the organic layer was washed with brine, and dried (MgSO₄). The crude material was purified by prep HPLC to obtain (2S)-2-(5-(4-(benzylcarbamoyl)phenyl)-4-(3-azabicyclo[3.1.0]hexan-3-yl)-2,6-dimethylpyridin-3-yl)-2-(tert-butoxy)acetic acid 32 mg (58%). ¹H NMR (500 MHz, DMSO) δ 9.19-9.16 (m, 1H), 8.00-7.96 (m, 2H), 7.42 (d, J=7.3 Hz, 1H), 7.37-7.33 (m, 4H), 7.27-7.23 (m, 2H), 5.52 (s, 1H), 4.51 (d, J=5.9 Hz, 2H), 3.39 (br. s, 2H), 3.03-3.01 (m, 1H), 2.59 (d, J=8.8 Hz, 1H), 2.45 (s, 3H), 2.01 (s, 3H), 1.31 (br. s, 1H), 1.18 (br. s, 1H), 1.10 (s, 9H), 0.63-0.62 (m, 1H), 0.47-0.44 (m, 1H). UPLC (M+H)=528.5.

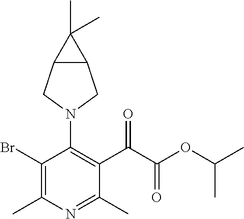 [SMILES: Cc1nc(C)c(C(=O)C(=O)OC(C)C)c(N2CC3C(C2)C3(C)C)c1Br]

### Isopropyl 2-(5-bromo-4-(6,6-dimethyl-3-azabicyclo[3.1.0]hexan-3-yl)-2,6-dimethylpyridin-3-yl)-2-oxoacetate

To a solution of 6,6-dimethyl-3-azabicyclo[3.1.0]hexane, HCl (1.0 g, 6.77 mmol) and DIEA (4.73 mL, 27.1 mmol) in anhydrous CH₃CN (20 mL) was added isopropyl 2-(5-bromo-4-chloro-2,6-dimethylpyridin-3-yl)-2-oxoacetate (2.27 g, 6.77 mmol) at rt. The reaction was heated at 80° C. for 20 h, after which an additional 2.4 mL of DIEA was added, and heating was continued for 18 h. The reaction mixture was cooled, diluted with ether, washed with water, brine, dried (MgSO₄). The crude product was charged (DCM) to a 80 g ISCO silica gel cartridge and gradient eluted (0-35% EtOAc/hexanes) using an Isolera chromatography station and gave isopropyl 2-(5-bromo-4-(6,6-dimethyl-3-azabicyclo[3.1.0]hexan-3-yl)-2,6-dimethylpyridin-3-yl)-2-oxoacetate 1.33 g (48). UPLC (M+H)=411.3.

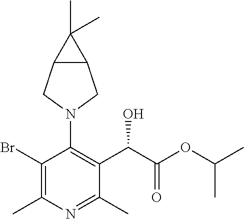 [SMILES: Cc1nc(C)c([C@H](O)C(=O)OC(C)C)c(N2CC3C(C2)C3(C)C)c1Br]

### (2S)-Isopropyl 2-(5-bromo-4-(6,6-dimethyl-3-azabicyclo[3.1.0]hexan-3-yl)-2,6-dimethylpyridin-3-yl)-2-hydroxyacetate

The benzo[d][1,3,2]dioxaborole (1.36 mL, 6.35 mmol; 50% soln in toluene) was added to a nitrogen purged solution of isopropyl 2-(5-bromo-4-(6,6-dimethyl-3-azabicyclo[3.1.0]hexan-3-yl)-2,6-dimethylpyridin-3-yl)-2-oxoacetate (1.3 g, 3.18 mmol) and 1.27 mL of 1M (R)-1-methyl-3,3-diphenylhexahydropyrrolo[1,2-c][1,3,2]oxazaborole (352 mg, 1.27 mmol) in toluene (7 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 80 g ISCO silica gel cartridge and gradient elution (0-50% EtOAc/hexanes) using an Isolera chromatography station gave (2S)-isopropyl 2-(5-bromo-4-(6,6-dimethyl-3-azabicyclo[3.1.0]hexan-3-yl)-2,6-dimethylpyridin-3-yl)-2-hydroxyacetate 1.31 g (100%) as a mixture of diastereomers. ¹H NMR (500 MHz, DMSO) δ 5.69 (s, 1H), 4.94-4.89 (m, 1H), 3.52-3.50 (m, 2H), 3.03-3.00 (m, 2H), 2.53 (s, 3H), 2.33 (s, 3H), 1.55 (br. s, 2H), 1.77 (s, 3H), 1.13 (d, J=6.2 Hz, 3H), 1.05 (d, J=6.2 Hz, 3H), 1.04 (s, 3H). UPLC (M+H)=413.2.

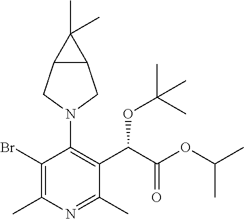 [SMILES: Cc1nc(C)c([C@H](OC(C)(C)C)C(=O)OC(C)C)c(N2CC3C(C2)C3(C)C)c1Br]

### (2S)-Isopropyl 2-(5-bromo-4-(6,6-dimethyl-3-azabicyclo[3.1.0]hexan-3-yl)-2,6-dimethylpyridin-3-yl)-2-(tert-butoxy)acetate

The isobutylene gas was bubbled into a nitrogen purged, cooled (0° C.) solution of (2S)-isopropyl 2-(5-bromo-4-(6,6-dimethyl-3-azabicyclo[3.1.0]hexan-3-yl)-2,6-dimethylpyridin-3-yl)-2-hydroxyacetate (1.30 g, 3.16 mmol) and 0.30 mL of 70% HClO₄ in DCM (20 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 80 g ISCO silica gel cartridge and gradient elution (0-12% EtOAc/hexanes) using an Isolera chromatography station gave (2S)-isopropyl 2-(5-bromo-4-(6,6-dimethyl-3-azabicyclo[3.1.0]hexan-3-yl)-2,6-dimethylpyridin-3-yl)-2-(tert-butoxy)acetate 1.0 g (68%): ¹H NMR (500 MHz, DMSO) δ 5.94 (br. s, 1H), 4.89-4.87 (m, 1H), 3.67 (br. s, 1H), 3.38 (br. s, 1H), 2.15 (d, J=9.1 Hz, 1H), 3.02 (d, J=8.4 Hz, 1H), 2.54 (s, 3H), 2.43 (s, 3H), 1.59 (s, 2H), 1.26 (s, 3H), 1.15-1.13 (m, 12H), 1.06 (s, 3H), 1.03 (d, J=5.9 Hz, 3H). UPLC (M+H)=469.4.

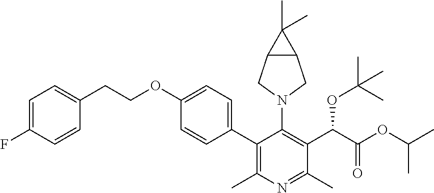 [SMILES: Cc1nc(C)c([C@H](OC(C)(C)C)C(=O)OC(C)C)c(N2CC3C(C2)C3(C)C)c1-c1ccc(OCCc2ccc(F)cc2)cc1]

### (2S)-Isopropyl 2-(tert-butoxy)-2-(4-(6,6-dimethyl-3-azabicyclo[3.1.0]hexan-3-yl)-5-(4-(4-fluorophenethoxy)phenyl)-2,6-dimethylpyridin-3-yl)acetate

The Pd(Ph₃P)₄ (79 mg, 0.068 mmol) was added to a nitrogen purged and degassed solution of (2S)-isopropyl 2-(5-bromo-4-(6,6-dimethyl-3-azabicyclo[3.1.0]hexan-3-yl)-2,6-dimethylpyridin-3-yl)-2-(tert-butoxy)acetate (150 mg, 0.32 mmol), (4-(4-fluorophenethoxy)phenyl)boronic acid (92 mg, 0.35 mmol), and potassium phosphate tribasic (511 mg, 2.41 mmol) in 1,4-dioxane (2 mL) and water (0.5 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (0-20% EtOAc/hexanes) using an Isolera chromatography station gave (2S)-isopropyl 2-(tert-butoxy)-2-(4-(6,6-dimethyl-3-azabicyclo[3.1.0]hexan-3-yl)-5-(4-(4-fluorophenethoxy)phenyl)-2,6-dimethylpyridin-3-yl)acetate 173 mg (90%) as a mixture of diasteromers: ¹H NMR (500 MHz, DMSO) δ 7.40-7.38 (m, 2H), 7.21 (d, J=7.7 Hz, 1H), 7.14 (t, J=8.8 Hz, 2H), 7.04-7.01 (m, 3H) 5.77 (s, 1H), 4.94-4.90 (m, 1H), 4.23 (t, J=6.6 Hz, 2H), 3.36-3.32 (m, 1H), 3.18-3.15 (m, 2H), 3.06 (t, J=6.6 Hz, 2H), 2.69 (d, J=9.1 Hz, 1H), 2.44 (s, 3H), 2.05 (s, 3H), 1.26 (t, J=6.2 Hz, 1H), 1.18 (d, J=6.6 Hz, 3H), 1.12-1.11 (m, 12H), 0.99 (s, 3H), 0.94 (t, J=6.2 Hz, 1H), 0.89 (s, 3H). UPLC (M+H)=603.6.

### Example 8

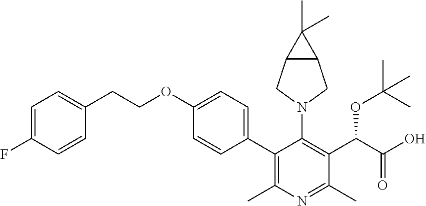 [SMILES: Cc1nc(C)c([C@H](OC(C)(C)C)C(=O)O)c(N2CC3C(C2)C3(C)C)c1-c1ccc(OCCc2ccc(F)cc2)cc1]

### (2S)-2-(tert-Butoxy)-2-(4-(6,6-dimethyl-3-azabicyclo[3.1.0]hexan-3-yl)-5-(4-(4-fluorophenethoxy)phenyl)-2,6-dimethyl-pyridin-3-yl)acetic acid

The 1M sodium hydroxide (1.36 mL, 1.36 mmol) was added to a solution of (2S)-isopropyl 2-(tert-butoxy)-2-(4-(6,6-dimethyl-3-azabicyclo[3.1.0]hexan-3-yl)-5-(4-(4-fluorophenethoxy)phenyl)-2,6-dimethylpyridin-3-yl)acetate (136 mg, 0.23 mmol) in ethanol (1 mL) and stirred for 18 h at 85° C. An additional 0.31 mL sodium hydroxide solution was added and stirring was continued for 18 h. The reaction mixture was neutralized with 1N HCl soln, extracted with EtOAc, and the organic layer was washed with brine, and dried (MgSO₄). The crude material was purified by prep HPLC to obtain (2S)-2-(tert-butoxy)-2-(4-(6,6-dimethyl-3-azabicyclo[3.1.0]hexan-3-yl)-5-(4-(4-fluorophenethoxy)phenyl)-2,6-dimethyl-pyridin-3-yl)acetic acid 75 mg (59%). ¹H NMR (500 MHz, DMSO) δ 7.38-7.35 (m, 2H), 7.18 (d, J=8.1 Hz, 1H), 7.12 (t, J=8.4 Hz, 2H), 7.05-6.99 (m, 3H) 5.69 (s, 1H), 4.21 (t, J=6.2 Hz, 2H), 3.18-3.14 (m, 2H), 3.07-3.03 (m, 3H), 2.66 (d, J=9.2 Hz, 1H), 2.43 (s, 3H), 2.03 (s, 3H), 1.26 (t, J=6.9 Hz, 1H), 1.10 (s, 9H), 0.95 (s, 3H), 0.91 (t, J=7.3 Hz, 1H), 0.86 (s, 3H). UPLC (M+H)=561.5.

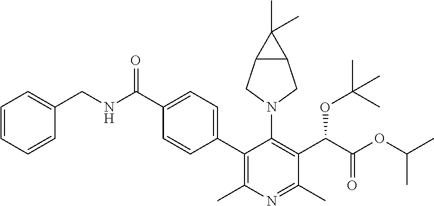 [SMILES: Cc1nc(C)c([C@H](OC(C)(C)C)C(=O)OC(C)C)c(N2CC3C(C2)C3(C)C)c1-c1ccc(C(=O)NCc2ccccc2)cc1]

### (2S)-Isopropyl 2-(5-(4-(benzylcarbamoyl)phenyl)-4-(6,6-dimethyl-3-azabicyclo[3.1.0]hexan-3-yl)-2,6-dimethylpyridin-3-yl)-2-(tert-butoxy)acetate

The Pd(Ph₃P)₄ (74 mg, 0.064 mmol) was added to a nitrogen purged and degassed solution of (2S)-isopropyl 2-(5-bromo-4-(6,6-dimethyl-3-azabicyclo[3.1.0]hexan-3-yl)-2,6-dimethylpyridin-3-yl)-2-(tert-butoxy)acetate (150 mg, 0.32 mmol), (4-(benzylcarbamoyl)phenyl)boronic acid (90 mg, 0.35 mmol), and potassium phosphate tribasic (511 mg, 2.41 mmol) in 1,4-dioxane (2 mL) and water (0.5 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (0-20% EtOAc/hexanes) using an Isolera chromatography station gave (2S)-isopropyl 2-(5-(4-(benzylcarbamoyl)phenyl)-4-(6,6-dimethyl-3-azabicyclo[3.1.0]hexan-3-yl)-2,6-dimethylpyridin-3-yl)-2-(tert-butoxy)acetate 152 mg (80%) as a mixture of diasteromers: δ 9.16-9.13 (m, 1H), 8.02-8.00 (m, 2H), 7.46 (d, J=7.3 Hz, 1H), 7.36-7.33 (m, 4H), 7.30-7.25 (m, 2H), 5.75 (s, 1H), 4.96-4.91 (m, 1H), 4.52 (d, J=5.9 Hz, 2H), 3.22-3.19 (m, 1H), 3.14-3.11 (m, 1H), 3.05 (d, J=9.5 Hz, 1H), 2.71 (d, J=9.5 Hz, 1H), 2.47 (s, 3H), 2.06 (s, 3H), 1.27 (t, J=6.9 Hz, 1H), 1.19 (d, J=6.2 Hz, 3H), 1.13 (br. s, 12H), 0.97 (s, 3H), 0.92 (t, J=6.9 Hz, 1H), 0.88 (m, 3H). UPLC (M+H)=598.5.

### Example 9

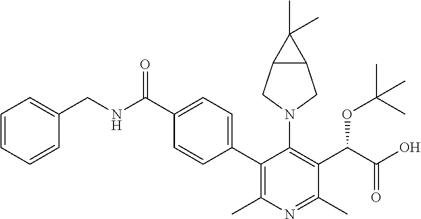 [SMILES: Cc1nc(C)c([C@H](OC(C)(C)C)C(=O)O)c(N2CC3C(C2)C3(C)C)c1-c1ccc(C(=O)NCc2ccccc2)cc1]

### (2S)-2-(5-(4-(Benzylcarbamoyl)phenyl)-4-(6,6-dimethyl-3-azabicyclo[3.1.0]hexan-3-yl)-2,6-dimethylpyridin-3-yl)-2-(tert-butoxy)acetic acid

The 1M sodium hydroxide (1.35 mL, 1.35 mmol) was added to a solution of (2S)-isopropyl 2-(5-(4-(benzylcarbamoyl)phenyl)-4-(6,6-dimethyl-3-azabicyclo[3.1.0]hexan-3-yl)-2,6-dimethylpyridin-3-yl)-2-(tert-butoxy)acetate (134 mg, 0.22 mmol) in ethanol (1 mL) and stirred for 18 h at 90° C. An additional 0.7 mL of sodium hydroxide soln was added and the reaction continued for 18 h. The reaction mixture was neutralized with 1N HCl soln, extracted with EtOAc, and the organic layer was washed with brine, and dried (MgSO₄). The crude material was purified by prep HPLC to obtain (2S)-2-(5-(4-(benzylcarbamoyl)phenyl)-4-(6,6-dimethyl-3-azabicyclo[3.1.0]hexan-3-yl)-2,6-dimethylpyridin-3-yl)-2-(tert-butoxy)acetic acid 108 mg (87%). ¹H NMR (500 MHz, DMSO) δ 9.16-9.13 (m, 1H), 8.00-7.98 (m, 2H), 7.45 (d, J=7.3 Hz, 1H), 7.35-7.32 (m, 4H), 7.29-7.24 (m, 2H), 5.69 (s, 1H), 4.51 (d, J=5.9 Hz, 2H), 3.21-3.18 (m, 1H), 3.15-3.12 (m, 1H), 3.07 (d, J=9.9 Hz, 1H), 2.69 (d, J=10.3 Hz, 1H), 2.46 (s, 3H), 2.05 (s, 3H), 1.28 (t, J=7.7 Hz, 1H), 1.12 (s, 9H), 0.93 (s, 3H), 0.93-0.90 (m, 1H), 0.87 (s, 3H). UPLC (M+H)=556.7.

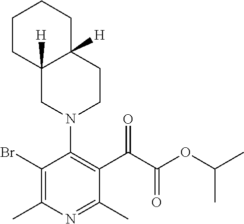

### Isopropyl 2-(5-bromo-2,6-dimethyl-4-((4aR,8aR)-octahydroisoquinolin-2(1H)-yl)pyridin-3-yl)-2-oxoacetate

To a solution of (4aR,8aR)-decahydroisoquinoline (1.05 mL, 7.17 mmol) and DIEA (2.51 mL, 14.35 mmol) in anhydrous CH₃CN (15 mL) was added isopropyl 2-(5-bromo-4-chloro-2,6-dimethylpyridin-3-yl)-2-oxoacetate (2.4 g, 7.17 mmol) at rt. The resulting mixture was placed in a pre-heated oil bath (80° C.) and stirred for 18 h. The reaction mixture was cooled, diluted with ether, washed with water, brine, dried (MgSO₄). The crude product was charged (DCM) to a 80 g ISCO silica gel cartridge and gradient eluted (0-10% EtOAc/hexanes) using an Isolera chromatography station to give isopropyl 2-(5-bromo-2,6-dimethyl-4-((4aR,8aR)-octahydroisoquinolin-2(1H)-yl)pyridin-3-yl)-2-oxoacetate 2.77 g (88%). UPLC (M+H)=439.4.

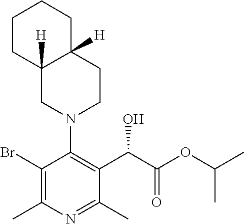

### (S)-Isopropyl 2-(5-bromo-2,6-dimethyl-4-((4aR,8aR)-octahydroisoquinolin-2 (1H)-yl)pyridin-3-yl)-2-hydroxyacetate

The benzo[d][1,3,2]dioxaborole (2.45 mL, 11.43 mmol; 50% soln in toluene) was added to a nitrogen purged solution of isopropyl 2-(5-bromo-2,6-dimethyl-4-((4aR,8aR)-octahydroisoquinolin-2(1H)-yl)pyridin-3-yl)-2-oxoacetate (2.5 g, 5.72 mmol) and 2.29 mL of 1M (R)-1-methyl-3,3-diphenylhexahydropyrrolo[1,2-c][1,3,2]oxazaborole (2.29 mmol) in toluene (40 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 40 g ISCO silica gel cartridge and gradient elution (0-30% EtOAc/hexanes) using an Isolera chromatography station gave (S)-isopropyl 2-(5-bromo-2,6-dimethyl-4-((4aR,8aR)-octahydroisoquinolin-2(1H)-yl)pyridin-3-yl)-2-hydroxyacetate 2.5 g (100%): ¹H NMR (500 MHz, DMSO) δ 6.16/5.97 (s, 1H), 4.98-4.93 (m, 1H), 3.64 (br. s, 1H), 3.29/2.93 (br. s, 1H), 2.67-2.57 (m, 2H), 2.50 (s, 3H), 2.35/2.30 (s, 3H), 1.97-1.23 (series of m, 12H), 1.12 (d, J=6.2 Hz, 3H), 1.06 (d, J=6.2 Hz, 3H). UPLC (M+H)=441.3.

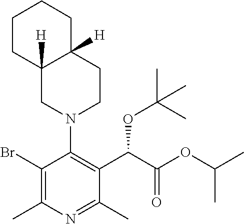

### (S)-Isopropyl 2-(5-bromo-2,6-dimethyl-4-((4aR,8aR)-octahydroisoquinolin-2 (1H)-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-((4aR,8aR)-octahydroisoquinolin-2(1H)-yl)pyridin-3-yl)-2-hydroxyacetate (2.0 g, 4.55 mmol) and 0.43 mL of 70% HClO₄ in DCM (25 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 80 g ISCO silica gel cartridge and gradient elution (0-12% EtOAc/hexanes) using an Isolera chromatography station gave (S)-isopropyl 2-(5-bromo-2,6-dimethyl-4-((4aR,8aR)-octahydroisoquinolin-2(1H)-yl)pyridin-3-yl)-2-(tert-butoxy)acetate 1.56 g (69%): ¹H NMR (500 MHz, DMSO) δ 6.35/6.01 (s, 1H), 4.92-4.87 (m, 1H), 3.93 (t, J=9.1 Hz, 1H), 3.59-3.51 (m, 1H), 2.74-2.72 (m, 1H), 2.64-2.61 (m, 1H), 2.52 (s, 3H), 2.40/2.34 (s, 3H), 2.05-1.25 (series of m, 12H), 1.17-1.11 (m, 12H), 1.05 (d, J=5.9 Hz, 3H). UPLC (M+H)=497.5.

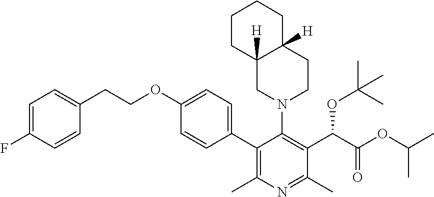

### (S)-Isopropyl 2-(tert-butoxy)-2-(4-(3,4-dihydroisoquinolin-2(1H)-yl)-5-(4-(4-fluorophenethoxy)phenyl)-2,6-dimethylpyridin-3-yl)acetate

The palladium acetate (4.6 mg, 0.02 mmol) was added to a nitrogen purged and degassed solution of (S)-isopropyl 2-(5-bromo-4-(3,4-dihydroisoquinolin-2(1H)-yl)-2,6-dimethylpyridin-3-yl)-2-(tert-butoxy)acetate (100 mg, 0.20 mmol), 2-(4-(4-fluorophenethoxy)phenyl)-6-methyl-1,3,6,2-dioxazaborocane-4,8-dione (83 mg, 0.23 mmol), and potassium phosphate tribasic (325 mg, 1.53 mmol) in 1,4-dioxane (3 mL) and water (0.6 mL). The reaction mixture was stirred in a screw-capped pressure vessel for 4 h at 90° C., cooled, diluted with EtOAc, and the organic layer was washed with brine and dried (Na₂CO₃). The crude product was charged (DCM) to a 24 g ISCO silica gel cartridge and gradient elution (5-70% EtOAc/hexanes) using an Isolera chromatography station gave (S)-isopropyl 2-(tert-butoxy)-2-(4-(3,4-dihydroisoquinolin-2(1H)-yl)-5-(4-(4-fluorophenethoxy)phenyl)-2,6-dimethylpyridin-3-yl)acetate 29 mg (23%): ¹H NMR (500 MHz, DMSO) δ 7.37-7.29 (m, 3H), 7.12-6.96 (m, 8H), 6.92-6.91/6.74-6.73 (m, 1H), 5.99/5.77 (s, 1H), 4.96-4.91 (m, 1H), 4.38/4.02 (d, J=16.0 Hz, 1H) 4.19 (t, J=7.0 Hz, 2H), 3.53-3.49/3.30-3.27 (m, 1H), 3.18-3.10/2.94-2.87 (m, 1H), 3.02 (t, J=7.0 Hz, 2H), 2.85-2.81 (m, 1H), 2.64-2.61/2.55-2.50 (m, 1H), 2.47/2.43 (s, 3H), 2.09 (s, 3H), 1.20 (d, J=6.2 Hz, 3H), 1.13 (d, J=6.2 Hz, 3H), 0.99/0.92 (s, 9H). UPLC (M+H)=625.7.

### Example 10

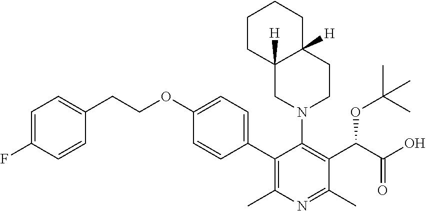

### (S)-2-(tert-Butoxy)-2-(4-(3,4-dihydroisoquinolin-2(1H)-yl)-5-(4-(4-fluorophenethoxy)phenyl)-2,6-dimethylpyridin-3-yl)acetic acid

The 0.10 mL of 1M sodium hydroxide (4.0 mg, 0.10 mmol) was added to a solution of (S)-isopropyl 2-(tert-butoxy)-2-(4-(3,4-dihydroisoquinolin-2(1H)-yl)-5-(4-(4-fluorophenethoxy)phenyl)-2,6-dimethylpyridin-3-yl)acetate (25 mg, 0.04 mmol) in ethanol (1.5 mL) and stirred for 18 h at 90° C. An addition 0.1 mL of sodium hydroxide soln was added and the reaction continued 18 h. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(4-(3,4-dihydroisoquinolin-2(1H)-yl)-5-(4-(4-fluorophenethoxy)phenyl)-2,6-dimethylpyridin-3-yl)acetic acid 8.5 mg (37%). ¹H NMR (500 MHz, DMSO) δ 7.37-7.28 (m, 3H), 7.12-6.95 (m, 8H), 6.91/6.72 (d, J=7.0 Hz, 1H), 5.89/5.66 (s, 1H), 4.62/3.98 (d, J=16 Hz, 1H), 4.19 (t, J=7.0 Hz, 2H) 3.67-3.63/3.44-3.42 (m, 1H), 3.39 (br. s, 2H), 3.24-3.10 (m, 1H), 3.02 (t, J=6.6 Hz, 2H), 2.94-2.81 (m, 1H), 2.47/2.45 (s, 3H), 2.08 (s, 3H), 0.97/0.90 (s, 9H). UPLC (M+H)=583.6.

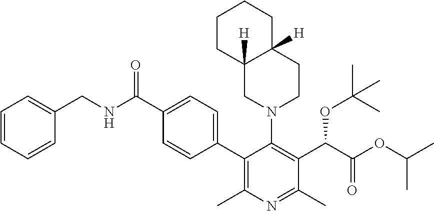

### (S)-Isopropyl 2-(5-(4-(benzylcarbamoyl)phenyl)-2,6-dimethyl-4-((4aR,8aR)-octahydroisoquinolin-2(1H)-yl)pyridin-3-yl)-2-(tert-butoxy)acetate

The Pd(Ph₃P)₄ (35 mg, 0.03 mmol) was added to a nitrogen purged and degassed solution of (S)-isopropyl 2-(5-bromo-4-(3,4-dihydroisoquinolin-2(1H)-yl)-2,6-dimethylpyridin-3-yl)-2-(tert-butoxy)acetate (100 mg, 0.20 mmol), (4-(benzylcarbamoyl)phenyl)boronic acid (85 mg, 0.33 mmol), and potassium phosphate tribasic (482 mg, 2.27 mmol) in 1,4-dioxane (2 mL) and water (0.4 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (0-70% EtOAc/hexanes) using an Isolera chromatography station gave (S)-isopropyl 2-(5-(4-(benzylcarbamoyl)phenyl)-2,6-dimethyl-4-((4aR,8aR)-octahydroisoquinolin-2(1H)-yl)pyridin-3-yl)-2-(tert-butoxy)acetate 146 mg (77%): ¹H NMR (500 MHz, DMSO) δ 9.17-9.15 (m, 1H), 8.07-8.03 (m, 1H), 8.00-7.95 (m, 1H), 7.49-7.42 (m, 2H), 7.36-7.31 (m, 4H), 7.27-7.25 (m, 1H), 6.22/6.00 (s, 1H), 5.00-4.94 (m, 1H), 4.54-4.50 (m, 2H) 3.42-3.39 (m, 2H), 3.05-2.98 (m, 1H), 2.89-2.87 (m, 1H), 2.45/2.40 (s, 3H), 2.10/2.05 (s, 3H), 1.91-1.25 (series of m, 12H), 1.20 (d, J=6.2 Hz, 3H), 1.17-1.12 (m, 9H). UPLC (M+H)=626.6.

### Example 11

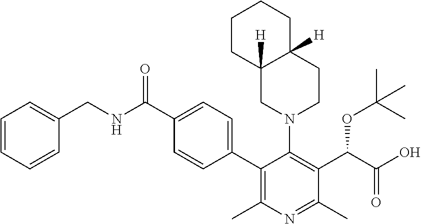

### (S)-2-(5-(4-(Benzylcarbamoyl)phenyl)-2,6-dimethyl-4-((4aR,8aR)-octahydroisoquinolin-2(1H)-yl)pyridin-3-yl)-2-(tert-butoxy)acetic acid

The 0.24 mL of 1M sodium hydroxide (9.6 mg, 0.24 mmol) was added to a solution of (S)-isopropyl 2-(5-(4-(benzylcarbamoyl)phenyl)-2,6-dimethyl-4-((4aR,8aR)-octahydroisoquinolin-2(1H)-yl)pyridin-3-yl)-2-(tert-butoxy)acetate (50 mg, 0.08 mmol) in ethanol (1.0 mL) and stirred for 18 h at 90° C. An addition 0.2 mL of sodium hydroxide soln was added and the reaction continued 18 h. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-((4aR,8aR)-octahydroisoquinolin-2(1H)-yl)pyridin-3-yl)-2-(tert-butoxy)acetic acid 13 mg (28%). ¹H NMR (500 MHz, DMSO) δ 9.17-9.15 (m, 1H), 8.07-8.03 (m, 1H), 7.99-7.96 (m, 1H), 7.49-7.44 (m, 1H), 7.35-7.24 (m, 6H), 6.13/5.80 (s, 1H), 4.52 (d, J=5.9 Hz, 2H), 3.42 (br. s, 1H), 3.28 (br. s, 1H), 3.23-3.18 (m, 1H), 3.00-2.95/2.88-2.83 (m, 1H), 2.46/2.43 (s, 3H), 2.10/2.04 (s, 3H), 1.97-1.26 (series of m, 12H), 1.15/1.12 (s, 9H). UPLC (M+H)=584.5.

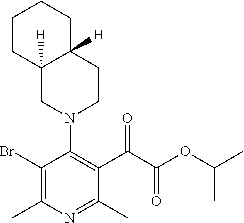

### Isopropyl 2-(5-bromo-2,6-dimethyl-4-((4aR,8aS)-octahydroisoquinolin-2(1H)-yl)pyridin-3-yl)-2-oxoacetate

To a solution of (4aR,8aS)-decahydroisoquinoline (0.89 mL, 5.98 mmol) and DIEA (2.1 mL, 11.95 mmol) in anhydrous CH₃CN (15 mL) was added isopropyl 2-(5-bromo-4-chloro-2,6-dimethylpyridin-3-yl)-2-oxoacetate (2.0 g, 5.98 mmol) at rt. The resulting mixture was placed in a pre-heated oil bath (75° C.) and stirred for 16 h. The reaction mixture was cooled, diluted with ether, washed with water, brine, dried (MgSO₄). The crude product was charged (DCM) to a 80 g ISCO silica gel cartridge and gradient eluted (0-10% EtOAc/hexanes) using an Isolera chromatography station to give isopropyl 2-(5-bromo-2,6-dimethyl-4-((4aR,8aS)-octahydroisoquinolin-2(1H)-yl)pyridin-3-yl)-2-oxoacetate 1.8 g (70%). ¹H NMR (500 MHz, DMSO) δ 5.08-5.02 (m, 1H), 3.34 (br. s, 1H), 3.02 (br. s, 1H), 2.80 (br. s, 1H), 2.67 (br. s, 1H), 2.60 (s, 3H), 2.29 (s, 3H), 1.68 (d, J=10.6 Hz, 2H), 1.63 (d, J=11.0 Hz, 1H), 1.50 (d, J=10.6 Hz, 1H), 1.43 (d, J=12.5 Hz, 1H), 1.30 (d, J=6.2 Hz, 6H) 1.26-1.08 (m, 4H), 1.02-0.89 (m, 3H). UPLC (M+H)=439.2.

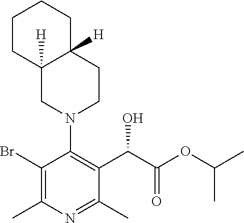

### (S)-Isopropyl 2-(5-bromo-2,6-dimethyl-4-((4aR,8aS)-octahydroisoquinolin-2(1H)-yl)pyridin-3-yl)-2-hydroxyacetate

The benzo[d][1,3,2]dioxaborole (1.4 mL, 6.63 mmol; 50% soln in toluene) was added to a nitrogen purged solution of isopropyl 2-(5-bromo-2,6-dimethyl-4-((4aR,8aR)-octahydroisoquinolin-2(1H)-yl)pyridin-3-yl)-2-oxoacetate (1.45 g, 3.32 mmol) and 1.33 mL of 1M (R)-1-methyl-3,3-diphenylhexahydropyrrolo[1,2-c][1,3,2]oxazaborole (1.33 mmol) in toluene (30 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 80 g ISCO silica gel cartridge and gradient elution (0-50% EtOAc/hexanes) using an Isolera chromatography station gave (S)-isopropyl 2-(5-bromo-2,6-dimethyl-4-((4aR,8aS)-octahydroisoquinolin-2(1H)-yl)pyridin-3-yl)-2-hydroxyacetate 1.45 g (100%): ¹H NMR (500 MHz, DMSO) δ 5.99/5.94 (s, 1H), 4.97-4.91 (m, 1H), 3.61/3.26 (t, J=11.4 Hz, 1H), 3.37/3.03 (t, J=11.0 Hz, 1H), 2.91/2.79 (d, J=11.0 Hz, 1H), 2.75/2.650 (d, J=7.3 Hz, 1H), 2.51 (s, 3H), 2.37/2.29 (s, 3H), 1.71-1.83 (m, 3H), 1.54-1.25 (m, 6H), 1.14 (d, J=6.2 Hz, 3H), 1.07-0.93 (m, 6H). UPLC (M+H)=441.3.

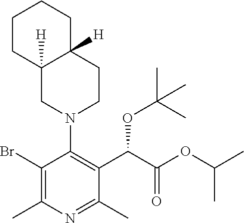

### (S)-Isopropyl 2-(5-bromo-2,6-dimethyl-4-((4aR,8aS)-octahydroisoquinolin-2(1H)-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-((4aR,8aS)-octahydroisoquinolin-2(1H)-yl)pyridin-3-yl)-2-hydroxyacetate (1.45 g, 3.30 mmol) and 0.31 mL of 70% HClO₄ in DCM (15 mL) for 20 min. The reaction mixture was allowed to warm to rt and stirred for 16 h in a pressure sealed vessel, diluted with DCM, washed with 1M Na₂CO₃ soln, and dried over MgSO₄. The crude product was charged (DCM) to a 80 g ISCO silica gel cartridge and gradient elution (0-12% EtOAc/hexanes) using an Isolera chromatography station gave (S)-isopropyl 2-(5-bromo-2,6-dimethyl-4-((4aR,8aS)-octahydroisoquinolin-2(1H)-yl)pyridin-3-yl)-2-(tert-butoxy)acetate 1.29 g (79%): ¹H NMR (500 MHz, DMSO) δ 6.15 (s, 1H), 4.93-4.87 (m, 1H), 3.85-3.78/3.47-3.43 (m, 1H), 3.29-3.25/2.99-2.94 (m, 1H), 2.92-2.89/2.82-2.79 (m, 1H), 2.73/2.61 (br. s, 1H), 2.51 (s, 3H), 2.40 (s, 3H), 1.71-1.65 (m, 4H), 1.56-1.21 (series of m, 6H), 1.15-1.09 (m, 12H), 1.06-1.00 (m, 5H). UPLC (M+H)=497.5.

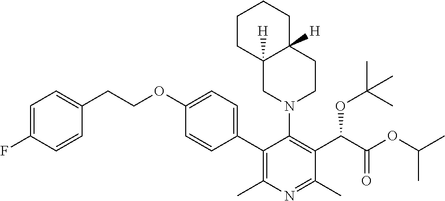

### (S)-Isopropyl 2-(tert-butoxy)-2-(5-(4-(4-fluorophenethoxy)phenyl)-2,6-dimethyl-4-((4aR,8aS)-octahydroisoquinolin-2(1H)-yl)pyridin-3-yl)acetate

The palladium acetate (6.8 mg, 0.03 mmol) was added to a nitrogen purged and degassed solution of (S)-isopropyl 2-(5-bromo-2,6-dimethyl-4-((4aR,8aS)-octahydroisoquinolin-2(1H)-yl)pyridin-3-yl)-2-(tert-butoxy)acetate (150 mg, 0.30 mmol), 2-(4-(4-fluorophenethoxy)phenyl)-6-methyl-1,3,6,2-dioxazaborocane-4,8-dione (124 mg, 0.33 mmol), and potassium phosphate tribasic (482 mg, 2.3 mmol) in 1,4-dioxane (2 mL) and water (0.5 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (5-70% EtOAc/hexanes) using an Isolera chromatography station gave (S)-isopropyl 2-(tert-butoxy)-2-(5-(4-(4-fluorophenethoxy)phenyl)-2,6-dimethyl-4-((4aR,8aS)-octahydroisoquinolin-2(1H)-yl)pyridin-3-yl)acetate 61 mg (32%). UPLC (M+H)=625.7.

### Example 12

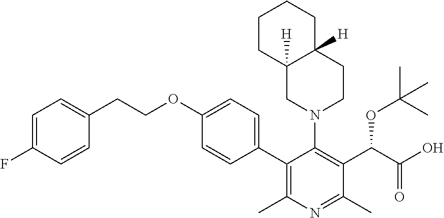

### (S)-2-(tert-Butoxy)-2-(5-(4-(4-fluorophenethoxy)phenyl)-2,6-dimethyl-4-((4aR,8aS)-octahydroisoquinolin-2(H)-yl)pyridin-3-yl)acetic acid

The 0.21 mL of 1M sodium hydroxide (8.7 mg, 0.21 mmol) was added to a solution of (S)-isopropyl 2-(tert-butoxy)-2-(5-(4-(4-fluorophenethoxy)phenyl)-2,6-dimethyl-4-((4aR,8aS)-octahydroisoquinolin-2(1H)-yl)pyridin-3-yl)acetate (45 mg, 0.07 mmol) in ethanol (1.0 mL) and stirred for 16 h at 95° C. An addition 0.21 mL of sodium hydroxide soln was added and the reaction continued 24 h. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)phenyl)-2,6-dimethyl-4-((4aR,8aS)-octahydroisoquinolin-2(1H)-yl)pyridin-3-yl)acetic acid 17.5 mg (42%). ¹H NMR (500 MHz, DMSO) δ 7.41-7.37 (m, 2H), 7.20-7.12 (m, 3H), 7.01-6.93 (m, 3H), 5.83/5.81 (s, 1H), 4.25-4.21 (m, 2H), 3.38-3.33 (m, 3H), 3.06 (d, J=5.9 Hz, 2H), 2.61/2.24 (t, J=12 Hz, 1H) 2.43 (s, 3H), 2.03 (s, 3H), 1.77-1.06 (series of m, 10H), 1.12 (s, 9H), 0.93-0.80 (m, 2H). UPLC (M+H)=589.6.

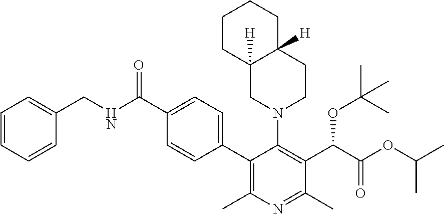

### (S)-Isopropyl 2-(5-(4-(benzylcarbamoyl)phenyl)-2,6-dimethyl-4-((4aR,8aS)-octahydroisoquinolin-2(1H)-yl)pyridin-3-yl)-2-(tert-butoxy)acetate

The Pd(Ph₃P)₄ (35 mg, 0.03 mmol) was added to a nitrogen purged and degassed solution of (S)-isopropyl 2-(5-bromo-2,6-dimethyl-4-((4aR,8aS)-octahydroisoquinolin-2(1H)-yl)pyridin-3-yl)-2-(tert-butoxy)acetate (150 mg, 0.30 mmol), (4-(benzylcarbamoyl)phenyl)boronic acid (85 mg, 0.33 mmol), and potassium phosphate tribasic (482 mg, 2.27 mmol) in 1,4-dioxane (2 mL) and water (0.4 mL). The reaction mixture was stirred in a screw-capped pressure vessel for 16 h at 80° C., cooled, diluted with EtOAc, and the organic layer was washed with brine and dried (Na₂CO₃). The crude product was charged (DCM) to a 40 g ISCO silica gel cartridge and gradient elution (0-70% EtOAc/hexanes) using an Isolera chromatography station gave (S)-isopropyl 2-(5-(4-(benzylcarbamoyl)phenyl)-2,6-dimethyl-4-((4aR,8aS)-octahydroisoquinolin-2(1H)-yl)pyridin-3-yl)-2-(tert-butoxy)acetate 58 mg (30%): ¹H NMR (500 MHz, DMSO) δ 9.19-9.16 (m, 1H), 7.98-7.96 (m, 2H), 7.44-7.41 (m, 1H), 7.34-7.30 (m, 4H), 7.23 (t, J=7.0 Hz, 1H), 7.16 (t, J=8.4 Hz, 1H), 5.92 (s, 1H), 4.96-4.91 (m, 1H), 4.51 (d, J=5.5 Hz 2H) 3.29 (d, J=11 Hz, 1H), 3.17-3.15 (m, 1H), 2.62 (t, J=10.6H, 1H), 2.54 (s, 3H), 2.35-2.26 (m, 1H), 2.02 (s, 3H), 1.72-1.24 (series of m, 7H), 1.18-1.16 (m, 3H), 1.13-1.11 (m, 14H), 0.87-0.82 (m, 2H), 0.59 (br. s, 1H). UPLC (M+H)=626.5.

### Example 13

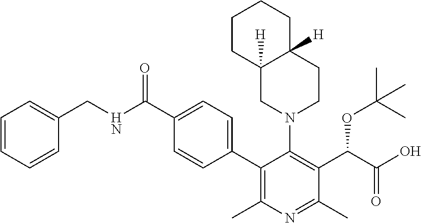

### (S)-2-(5-(4-(Benzylcarbamoyl) phenyl)-2,6-dimethyl-4-((4aR,8aS)-octahydroisoquinolin-2(1H)-yl)pyridin-3-yl)-2-(tert-butoxy)acetic acid

The 0.24 mL of 1M sodium hydroxide (9.6 mg, 0.24 mmol) was added to a solution of (S)-isopropyl 2-(5-(4-(benzylcarbamoyl)phenyl)-2,6-dimethyl-4-((4aR,8aS)-octahydroisoquinolin-2(1H)-yl)pyridin-3-yl)-2-(tert-butoxy)acetate (50 mg, 0.08 mmol) in ethanol (1.0 mL) and stirred for 18 h at 90° C. An addition 0.2 mL of sodium hydroxide soln was added and the reaction continued 18 h. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl) phenyl)-2,6-dimethyl-4-((4aR,8aS)-octahydroisoquinolin-2(1H)-yl)pyridin-3-yl)-2-(tert-butoxy)acetic acid 21 mg (46%). ¹H NMR (500 MHz, DMSO) δ 9.17-9.15 (m, 1H), 7.99-7.96 (m, 2H), 7.46 (t, J=7.7 Hz, 1H), 7.36-7.34 (m, 4H), 7.27-7.25 (m, 1H), 7.18 (t, J=5.1 Hz, 1H), 5.81 (s, 1H), 4.52-4.51 (m 2H), 3.42 (br. s, 1H), 2.65-2.60 (m, 1H), 2.48 (s, 3H), 2.34-2.24 (m, 2H), 2.04 (s, 3H), 1.69-1.17 (series of m, 9H), 1.13 (s, 9H), 0.89-0.82 (m, 2H), 0.61 (br. s, 1H). UPLC (M+H)=584.6.

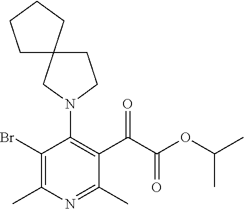 [SMILES: Cc1nc(C)c(C(=O)C(=O)OC(C)C)c(N2CCC3(CCCC3)C2)c1Br]

### 2-(5-Bromo-2,6-dimethyl-4-(2-azaspiro[4.4]nonan-2-yl)pyridin-3-yl)-2-oxoacetate

To a solution 2-azaspiro[4.4]nonane, HCl (676 mg, 4.18 mmol) and DIEA (2.2 mL, 12.6 mmol) in anhydrous CH₃CN (25 mL) was added isopropyl 2-(5-bromo-4-chloro-2,6-dimethylpyridin-3-yl)-2-oxoacetate (1.4 g, 4.18 mmol) at rt. The resulting mixture was placed in a pre-heated oil bath (80° C.) and stirred for 18 h; cooled, and concentrated. The crude product was charged (DCM) to a 80 g ISCO silica gel cartridge and gradient elution (5-35% EtOAc/hexanes) using an Isolera chromatography station to give isopropyl 2-(5-bromo-2,6-dimethyl-4-(2-azaspiro[4.4]nonan-2-yl)pyridin-3-yl)-2-oxoacetate 1.35 g (76%). ¹HNMR (500 MHz, CDCl3) δ 5.05-5.01 (m, 1H), 3.29 (t, J=6.6 Hz, 2H), 2.97 (s, 2H), 2.61 (s, 3H), 2.31 (s, 3H), 1.83 (t, J=6.6 Hz, 2H), 1.63-1.51 (m, 8H), 1.29 (d, J=6.2 Hz, 6H). UPLC (M+H)=425.4.

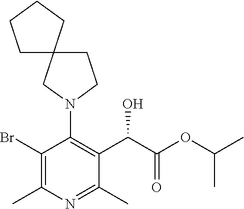 [SMILES: Cc1nc(C)c([C@H](O)C(=O)OC(C)C)c(N2CCC3(CCCC3)C2)c1Br]

### (S)-Isopropyl 2-(5-bromo-2,6-dimethyl-4-(2-azaspiro[4.4]nonan-2-yl)pyridin-3-yl)-2-hydroxyacetate

The 1.2 mL of benzo[d][1,3,2]dioxaborole (574 mg, 4.78 mmol) was added to a nitrogen purged solution of isopropyl 2-(5-bromo-2,6-dimethyl-4-(2-azaspiro[4.4]nonan-2-yl)pyridin-3-yl)-2-oxoacetate (1.35 g, 4.87 mmol) and 1.0 mL of (R)-1-methyl-3,3-diphenylhexahydropyrrolo[1,2-c][1,3,2]oxazaborole (265 mg, 1.0 mmol) in toluene (40 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine and dried (MgSO₄). The crude product was charged (DCM) to a 40 g ISCO silica gel cartridge and gradient elution (5-50% EtOAc/hexanes) using an Isolera chromatography station gave (S)-isopropyl 2-(5-bromo-2,6-dimethyl-4-(2-azaspiro[4.4]nonan-2-yl)pyridin-3-yl)-2-hydroxyacetate 1.2 g (85%) as a mixture of diastereomers. ¹H NMR (500 MHz, DMSO) δ 5.70 (s, 1H), 4.96-4.91 (m, 1H), 3.39-3.36 (m, 1H), 3.27-3.22 (m, 1H), 3.12 (d, J=7.7 Hz, 1H), 3.07 (d, J=7.3 Hz, 1H), 2.53 (s, 3H), 2.36 (s, 3H), 1.94-1.89 (m, 1H), 1.87-1.82 (m, 1H), 1.69-1.59 (m, 8H), 1.15 (d, J=6.3 Hz, 3H), 1.07 (d, J=6.2 Hz, 3H). UPLC (M+H)=427.3.

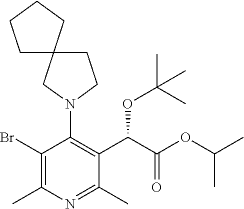 [SMILES: Cc1nc(C)c([C@H](OC(C)(C)C)C(=O)OC(C)C)c(N2CCC3(CCCC3)C2)c1Br]

### (S)-Isopropyl 2-(5-bromo-2,6-dimethyl-4-(2-azaspiro[4.4]nonan-2-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(2-azaspiro[4.4]nonan-2-yl)pyridin-3-yl)-2-hydroxyacetate (1.2 g, 2.8 mmol) and 0.7 mL of 70% HClO₄ in DCM (50 mL) for 20 min. The reaction mixture was allowed to warm to rt and stirred for 18 h in a pressure sealed vessel. The reaction mixture was diluted with DCM, washed with 1M Na₂CO₃ soln, and dried over MgSO₄. The crude product was charged (DCM) to a 80 g ISCO silica gel cartridge and gradient elution (5-35% EtOAc/hexanes) using an Isolera chromatography station gave (S)-isopropyl 2-(5-bromo-2,6-dimethyl-4-(2-azaspiro[4.4]nonan-2-yl)pyridin-3-yl)-2-(tert-butoxy)acetate 840 mg (62%) as a mixture of diastereomers. ¹H NMR (500 MHz, DMSO) δ 5.99 (br. s, 1H), 4.92-4.87 (m, 1H), 3.57 (br. s, 1H), 3.21-3.16 (m, 2H), 3.02 (d, J=7.3 Hz, 1H), 2.53 (s, 3H), 2.42 (s, 3H), 2.04-1.98 (m, 1H), 1.87-1.83 (m, 1H), 1.75-1.57 (m, 8H), 1.15-1.14 (m, 12H), 1.05 (d, J=5.8 Hz, 3H). UPLC (M+H)=482.8.

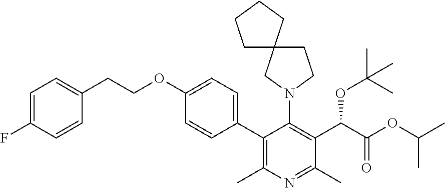 [SMILES: Cc1nc(C)c([C@H](OC(C)(C)C)C(=O)OC(C)C)c(N2CCC3(CCCC3)C2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(2-azaspiro[4.4]nonan-2-yl)pyridin-3-yl)acetate

The diacetoxypalladium (4.66 mg, 0.021 mmol) was added to an argon purged and degassed solution of (S)-isopropyl 2-(5-bromo-2,6-dimethyl-4-(2-azaspiro[4.4]nonan-2-yl)pyridin-3-yl)-2-(tert-butoxy)acetate (100 mg, 0.21 mmol), 2-(4-(4-fluorophenethoxy)phenyl)-6-methyl-1,3,6,2-dioxazaborocane-4,8-dione (85 mg, 0.23 mmol), and potassium phosphate tribasic (330 mg, 1.6 mmol) in dioxane (3 mL) and water (0.6 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(2-azaspiro[4.4]nonan-2-yl)pyridin-3-yl)acetate 61 mg (48%). ¹H NMR (500 MHz, DMSO) δ 7.39-7.36 (m, 2H), 7.19 (d, J=8.4 Hz, 1H), 7.13 (t, J=8.8 Hz, 2H), 7.03-6.97 (m, 3H), 5.8 (s, 1H), 4.96-4.91 (m, 1H), 4.23-4.21 (m, 2H), 3.05 (t, J=6.6 Hz, 2H), 2.90-2.80 (m, 3H), 2.66 (d, J=8.4 Hz, 1H), 2.41 (s, 3H), 2.05 (s, 3H), 1.53-1.23 (m, 8H), 1.18 (d, J=6.2 Hz, 3H), 1.13-1.33 (m, 12H). UPLC (M+H)=617.6.

### Example 14

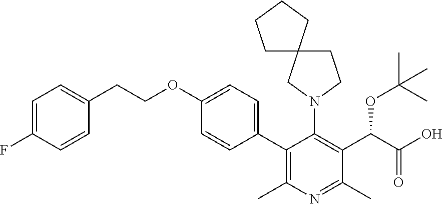 [SMILES: Cc1nc(C)c([C@H](OC(C)(C)C)C(=O)O)c(N2CCC3(CCCC3)C2)c1-c1ccc(OCCc2ccc(F)cc2)cc1]

### (S)-2-(tert-Butoxy)-2-(5-(4-(4-fluorophenethoxy)phenyl)-2,6-dimethyl-4-(2-azaspiro[4.4]nonan-2-yl)pyridin-3-yl)acetic acid

The 0.2 ml of 1M sodium hydroxide (8.1 mg, 0.2 mmol) was added to a solution of (S)-isopropyl 2-(tert-butoxy)-2-(5-(4-(4-fluorophenethoxy)phenyl)-2,6-dimethyl-4-(2-azaspiro[4.4]nonan-2-yl)pyridin-3-yl)acetate (50 mg, 0.08 mmol) in ethanol (2 mL) and stirred for 18 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)phenyl)-2,6-dimethyl-4-(2-azaspiro[4.4]nonan-2-yl)pyridin-3-yl)acetic acid 5.8 mg (13%) as a mixture of diastereomers (NOTE: About equal amount of recovered starting material was also obtained). ¹H NMR (500 MHz, DMSO) δ 7.39-7.36 (m, 2H), 7.19 (d, J=8.4 Hz, 1H), 7.13 (t, J=8.8 Hz, 2H), 7.03-6.99 (m, 3H), 5.71 (s, 1H), 4.22 (t, J=6.2 Hz, 2H), 3.05 (t, J=6.6 Hz, 2H), 2.97-2.93 (m, 1H), 2.89-2.87 (m, 2H), 2.85-2.81 (m, 1H), 2.65 (d, J=8.4 Hz, 1H), 2.42 (s, 3H), 2.05 (s, 3H), 1.53-1.30 (m, 10H), 1.11 (s, 9H). UPLC (M+H)=575.4.

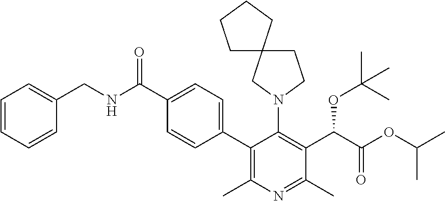 [SMILES: Cc1nc(C)c([C@H](OC(C)(C)C)C(=O)OC(C)C)c(N2CCC3(CCCC3)C2)c1-c1ccc(C(=O)NCc2ccccc2)cc1]

### (S)-Isopropyl 2-(5-(4-(benzylcarbamoyl)phenyl)-2,6-dimethyl-4-(2-azaspiro[4.4]nonan-2-yl)pyridin-3-yl)-2-(tert-butoxy)acetate

The Pd(Ph₃P)₄ (48 mg, 0.042 mmol) was added to a nitrogen purged and degassed solution of (S)-isopropyl 2-(5-bromo-2,6-dimethyl-4-(2-azaspiro[4.4]nonan-2-yl)pyridin-3-yl)-2-(tert-butoxy)acetate (100 mg, 0.21 mmol), (4-(benzylcarbamoyl)phenyl)boronic acid (58 mg, 0.23 mmol), and potassium phosphate tribasic (308 mg, 1.45 mmol) in dioxane (3 mL) and water (0.6 mL) and stirred in a screw-capped pressure vessel for 4 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-75% EtOAc/hexanes) using an Isolera chromatography station gave (S)-isopropyl 2-(5-(4-(benzylcarbamoyl)phenyl)-2,6-dimethyl-4-(2-azaspiro[4.4]nonan-2-yl)pyridin-3-yl)-2-(tert-butoxy)acetate 75 mg (59%). ¹H NMR (500 MHz, DMSO) δ 9.16-9.14 (m, 1H), 8.00-7.98 (m, 2H), 7.46 (d, J=7.7 Hz, 1H), 7.35-7.32 (m, 4H), 7.27-7.25 (m, 1H), 7.22 (d, J=7.7 Hz, 1H), 5.77 (s, 1H), 4.98-4.93 (m, 1H), 4.52 (d, J=5.9 Hz, 2H), 2.87-2.84 (m, 3H), 2.69 (d, J 8.4 Hz, 1H), 2.43 (s, 3H), 2.06 (s, 3H), 1.54-1.25 (m, 10H), 1.19 (d, J=6.2 Hz, 3H), 1.15 (d, J=5.9 Hz, 3H), 1.12 (s, 9H). UPLC (M+H)=612.5.

### Example 15

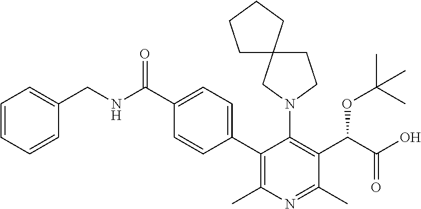 [SMILES: Cc1nc(C)c([C@H](OC(C)(C)C)C(=O)O)c(N2CCC3(CCCC3)C2)c1-c1ccc(C(=O)NCc2ccccc2)cc1]

### (S)-2-(5-(4-(Benzylcarbamoyl)phenyl)-2,6-dimethyl-4-(2-azaspiro[4.4]nonan-2-yl)pyridin-3-yl)-2-(tert-butoxy)acetic acid

The 0.2 ml of 1M sodium hydroxide (7.8 mg, 0.2 mmol) was added to a solution of (S)-isopropyl 2-(5-(4-(benzylcarbamoyl)phenyl)-2,6-dimethyl-4-(2-azaspiro[4.4]nonan-2-yl)pyridin-3-yl)-2-(tert-butoxy)acetate (60 mg, 0.098 mmol) in ethanol (2 mL) and stirred for 18 h at 95° C. An additional 0.2 mL of sodium hydroxide was added, and the reaction was continued for 6 h, cooled, and neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(2-azaspiro[4.4]nonan-2-yl)pyridin-3-yl)-2-(tert-butoxy)acetic acid 16.4 mg (29%) as a mixture of diastereomers. (NOTE: Starting material was also recovered)¹H NMR (500 MHz, DMSO) δ 9.16-9.13 (m, 1H), 8.00-7.98 (m, 2H), 7.46 (d, J=7.3 Hz, 1H), 7.35-7.32 (m, 4H), 7.27-7.25 (m, 1H), 7.22 (d, J=8.8 Hz, 1H), 5.66 (s, 1H), 4.52 (d, J=5.9 Hz, 2H), 2.96-2.92 (m, 1H), 2.88-2.83 (m, 2H), 2.69 (d, J=8.4 Hz, 1H), 2.45 (s, 3H), 2.06 (s, 3H), 1.53-1.26 (m, 10H), 1.12 (s, 9H). UPLC (M+H)=570.5.

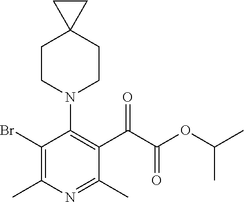 [SMILES: Cc1nc(C)c(C(=O)C(=O)OC(C)C)c(N2CCC3(CC2)CC3)c1Br]

### Isopropyl 2-(5-bromo-2,6-dimethyl-4-(6-azaspiro[2.5]octan-6-yl)pyridin-3-yl)-2-oxoacetate

To a solution of 6-azaspiro[2.5]octane (1.0 g, 8.99 mmol) and DIEA (3.14 mL, 17.99 mmol) in anhydrous CH₃CN (15 mL) was added isopropyl 2-(5-bromo-4-chloro-2,6-dimethylpyridin-3-yl)-2-oxoacetate (3.01 g, 8.99 mmol) at rt. The resulting mixture was placed in a pre-heated oil bath (80° C.) and stirred for 24 h; cooled, diluted with ether, washed with water, brine, and dried (MgSO₄). The crude product was charged (DCM) to a 120 g ISCO silica gel cartridge and gradient elution (0-20% EtOAc/hexanes) using an Isolera chromatography station to give isopropyl 2-(5-bromo-2,6-dimethyl-4-(6-azaspiro[2.5]octan-6-yl)pyridin-3-yl)-2-oxoacetate 2.94 g (79%). ¹H NMR (500 MHz, CDCl3) δ 5.24-5.19 (m, 1H), 3.20 (br. s, 4H), 2.70 (s, 3H), 2.42 (s, 3H), 1.44-1.34 (m 10H), 0.33 (s, 4H). UPLC (M+H)=411.3.

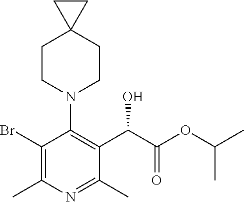 [SMILES: Cc1nc(C)c([C@H](O)C(=O)OC(C)C)c(N2CCC3(CC2)CC3)c1Br]

### Isopropyl 2-(5-bromo-2,6-dimethyl-4-(6-azaspiro[2.5]octan-6-yl)pyridin-3-yl)-2-hydroxyacetate

The 1.7 mL of benzo[d][1,3,2]dioxaborole (967 mg, 8.06 mmol) was added to a nitrogen purged solution of isopropyl 2-(5-bromo-2,6-dimethyl-4-(6-azaspiro[2.5]octan-6-yl)pyridin-3-yl)-2-oxoacetate (2.9 g, 7.1 mmol) and 1.07 mL of (R)-1-methyl-3,3-diphenylhexahydropyrrolo[1,2-c][1,3,2]oxazaborole (298 mg, 1.07 mmol) in toluene (40 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ soln, brine and dried (MgSO₄). The crude product was charged (DCM) to a 40 g ISCO silica gel cartridge and gradient elution (0-100% EtOAc/hexanes) using an Isolera chromatography station gave isopropyl 2-(5-bromo-2,6-dimethyl-4-(6-azaspiro[2.5]octan-6-yl)pyridin-3-yl)-2-hydroxyacetate 2.87 g (98%). ¹H NMR (500 MHz, DMSO) δ 5.93 (s, 1H), 4.98-4.93 (m, 1H), 3.60 (t, J=10.3 Hz, 1H), 3.42-3.38 (m, 2H), 2.90-2.79 (m, 1H), 2.53 (s, 3H), 2.37 (s, 3H), 1.95-1.88 (m, 2H), 1.14 (d, J=6.3 Hz, 3H), 1.07 (d, J=6.2 Hz, 3H), 0.98-092 (m, 2H), 0.33 (s, 4H). UPLC (M+H)=413.3.

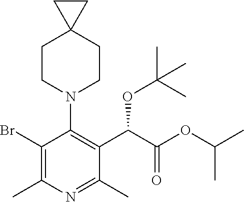 [SMILES: Cc1nc(C)c([C@H](OC(C)(C)C)C(=O)OC(C)C)c(N2CCC3(CC2)CC3)c1Br]

### (S)-Isopropyl 2-(5-bromo-2,6-dimethyl-4-(6-azaspiro[2.5]octan-6-yl)pyridin-3-yl)-2-(tert-butoxy)acetate

The isobutylene gas was bubbled into a nitrogen purged, cooled (0° C.) solution of isopropyl 2-(5-bromo-2,6-dimethyl-4-(6-azaspiro[2.5]octan-6-yl)pyridin-3-yl)-2-hydroxyacetate (2.75 g, 4.19 mmol) and 0.63 mL of 70% HClO₄ in DCM (30 mL) for 20 min. The reaction mixture was allowed to warm to rt and stirred for 18 h in a pressure sealed vessel, after which it was recooled, and an additional 0.63 mL of 70% HClO4 was added at 0° C., and the reaction was stirred for 24 h at rt. The reaction mixture was diluted with DCM, washed with 1M Na₂CO₃ soln, and dried over MgSO₄. The crude product was charged (DCM) to a 80 g ISCO silica gel cartridge and gradient elution (5-12% EtOAc/hexanes) using an Isolera chromatography station gave (S)-isopropyl 2-(5-bromo-2,6-dimethyl-4-(6-azaspiro[2.5]octan-6-yl)pyridin-3-yl)-2-(tert-butoxy)acetate 1.6 g (51.4%). ¹H NMR (500 MHz, DMSO) δ 6.23 (br. s, 1H), 4.94-4.89 (m, 1H), 3.90 (br. s, 1H), 3.33 (t, J=7.7 Hz, 1H), 2.97-2.95 (m, 1H), 2.75 (s, 1H), 2.53 (s, 3H), 2.42 (s, 3H), 2.07-2.02 (m, 2H), 1.15-1.14 (m, 12H), 1.07 (d, J=6.2 Hz, 3H), 0.97 (br. s, 1H), 0.88-0.84 (m, 1H) 0.36 (s, 4H). UPLC (M+H)=469.5.

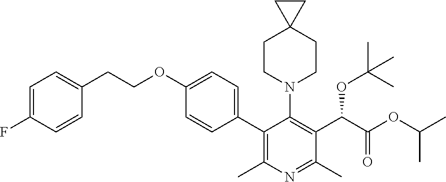 [SMILES: Cc1nc(C)c([C@H](OC(C)(C)C)C(=O)OC(C)C)c(N2CCC3(CC2)CC3)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(6-azaspiro[2.5]octan-6-yl)pyridin-3-yl)acetate

The Pd(Ph₃P)₄ (247 mg, 0.214 mmol) was added to an argon purged and degassed solution of (S)-isopropyl 2-(5-bromo-2,6-dimethyl-4-(6-azaspiro[2.5]octan-6-yl)pyridin-3-yl)-2-(tert-butoxy)acetate (500 mg, 1.07 mmol), (4-(4-fluorophenethoxy)phenyl)boronic acid (306 mg, 1.17 mmol), and potassium phosphate tribasic (1.7 g, 8.02 mmol) in dioxane (6 mL) and water (1.2 mL) and stirred in a screw-capped pressure vessel for 24 h at 80° C. The reaction was allowed to cool, diluted with EtOAc, and the organic layer was washed with brine and dried (MgSO₄). The crude product was charged (DCM) to a 80 g ISCO silica gel cartridge and gradient elution (0-20% EtOAc/hexanes) using an Isolera chromatography station gave (S)-isopropyl 2-(tert-butoxy)-2-(5-(4-(4-fluorophenethoxy)phenyl)-2,6-dimethyl-4-(6-azaspiro[2.5]octan-6-yl)pyridin-3-yl)acetate 524 mg (81%). ¹H NMR (500 MHz, DMSO) δ 7.40-7.37 (m, 2H), 7.20 (d, J=8.4 Hz, 1H), 7.14 (t, J=8.8 Hz, 2H), 7.04-7.00 (m, 3H), 6.05 (br. s, 1H), 4.99-4.93 (m, 1H), 4.26-4.20 (m, 2H), 3.40-3.36 (m, 2H), 3.26-3.24 (m, 1H), 3.06 (t, J=6.6 Hz, 2H), 2.74 (t, J=10.3 Hz, 1H), 2.44 (s, 3H), 2.04 (s, 3H), 1.91 (t, J=10.6 Hz, 1H), 1.83-1.77 (m, 1H), 1.18 (d, J=6.2 Hz, 3H), 1.15 (s, 9H), 1.14 (d, J=6.2 Hz, 3H), 0.73-0.70 (m, 1H), 0.58-0.56 (m, 1H), 0.25-0.19 (m 2H), 0.08 (br. s, 1H), 0.06 (br. s, 1H). UPLC (M+H)=603.7.

### Example 16

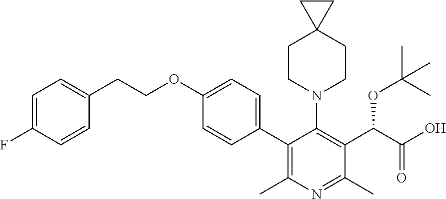 [SMILES: Cc1nc(C)c([C@H](OC(C)(C)C)C(=O)O)c(N2CCC3(CC2)CC3)c1-c1ccc(OCCc2ccc(F)cc2)cc1]

### (S)-2-(tert-Butoxy)-2-(5-(4-(4-fluorophenethoxy)phenyl)-2,6-dimethyl-4-(6-azaspiro[2.5]octan-6-yl)pyridin-3-yl)acetic acid

The 5.2 mL of 1M sodium hydroxide (207 mg, 5.18 mmol) was added to a solution of (S)-isopropyl 2-(tert-butoxy)-2-(5-(4-(4-fluorophenethoxy)phenyl)-2,6-dimethyl-4-(6-azaspiro[2.5]octan-6-yl)pyridin-3-yl)acetate (520 mg, 0.86 mmol) in ethanol (8 mL) and stirred for 24 h at 90° C. An additional 5.2 mL sodium hydroxide was added and the reaction was continued for 24 h. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)phenyl)-2,6-dimethyl-4-(6-azaspiro[2.5]octan-6-yl)pyridin-3-yl)acetic acid 481 mg (99%). ¹H NMR (500 MHz, DMSO) δ 7.40-7.37 (m, 2H), 7.20 (d, J=8.0 Hz, 1H), 7.15 (t, J=8.8 Hz, 2H), 7.04-6.98 (m, 3H), 5.88 (s, 1H), 4.26-4.22 (m, 2H), 3.40 (br. s, 4H), 3.06 (t, J=6.6 Hz, 2H), 2.44 (s, 3H), 2.03 (s, 3H), 1.91-1.86 (m, 1H), 1.83-1.79 (m, 1H), 1.15 (s, 9H), 0.72-0.69 (m, 1H), 0.55-0.53 (m, 1H), 0.23-0.18 (m, 2H), 0.07 (br. s, 1H), −0.07 (br. s, 1H). UPLC (M+H)=561.5.

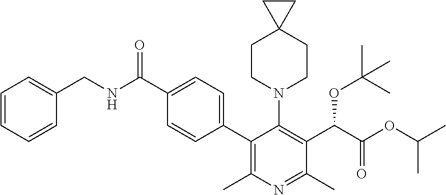 [SMILES: Cc1nc(C)c([C@H](OC(C)(C)C)C(=O)OC(C)C)c(N2CCC3(CC2)CC3)c1-c1ccc(C(=O)NCc2ccccc2)cc1]

### (S)-Isopropyl 2-(5-(4-(benzylcarbamoyl)phenyl)-2,6-dimethyl-4-(6-azaspiro[2.5]octan-6-yl)pyridin-3-yl)-2-(tert-butoxy)acetate

The Pd(Ph₃P)₄ (37 mg, 0.032 mmol) was added to an argon purged and degassed solution of (S)-isopropyl 2-(5-bromo-2,6-dimethyl-4-(6-azaspiro[2.5]octan-6-yl)pyridin-3-yl)-2-(tert-butoxy)acetate (150 mg, 0.32 mmol), (4-(benzylcarbamoyl)phenyl)boronic acid (90 mg, 0.35 mmol), and potassium phosphate tribasic (511 mg, 2.47 mmol) in dioxane (2 mL) and water (1 mL) and stirred in a screw-capped pressure vessel for 24 h at 80° C. The reaction was allowed to cool, diluted with EtOAc, and the organic layer was washed with brine and dried (MgSO₄). The crude product was charged (DCM) to a 40 g ISCO silica gel cartridge and gradient elution (0-70% EtOAc/hexanes) using an Isolera chromatography station gave (S)-isopropyl 2-(5-(4-(benzylcarbamoyl)phenyl)-2,6-dimethyl-4-(6-azaspiro[2.5]octan-6-yl)pyridin-3-yl)-2-(tert-butoxy)acetate 153 mg (80%). ¹H NMR (500 MHz, DMSO) δ 9.19-9.17 (m, 1H), 8.02 (d, J=8.1 Hz, 1H), 7.99 (d, J=8.1 Hz, 1H), 7.48 (d, J=7.7 Hz, 1H), 7.37-7.33 (m, 4H), 7.26-7.26 (m, 1H), 7.23 (d, J=8.4 Hz, 1H), 6.03 (s, 1H), 4.99-4.94 (m, 1H), 4.53 (d, J=5.9 Hz, 2H), 3.41-3.39 (m, 2H), 3.31-3.29 (m, 1H), 2.77-2.74 (m, 1H), 2.47 (s, 3H), 2.04 (s, 3H), 1.87-1.79 (m, 2H), 1.18 (d, J=6.2 Hz, 3H), 1.16-1.14 (m, 12H), 0.75-0.72 (m, 1H), 0.58-0.56 (m, 1H), 0.25-0.24 (m 1H), 0.20-0.18 (m, 1H), 0.08-0.07 (m, 1H), −0.06-0.08 (m, 1H). UPLC (M+H)=598.6.

### Example 17

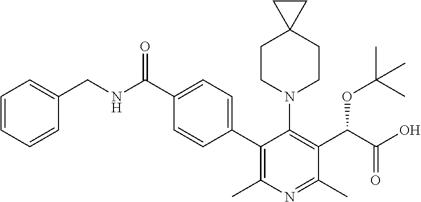 [SMILES: Cc1nc(C)c([C@H](OC(C)(C)C)C(=O)O)c(N2CCC3(CC2)CC3)c1-c1ccc(C(=O)NCc2ccccc2)cc1]

### (S)-2-(5-(4-(Benzylcarbamoyl)-phenyl)-2,6-dimethyl-4-(6-azaspiro[2.5]octan-6-yl)pyridin-3-yl)-2-(tert-butoxy)acetic acid

The 0.25 mL of 1M sodium hydroxide (10.4 mg, 0.25 mmol) was added to a solution of (S)-isopropyl 2-(5-(4-(benzylcarbamoyl)phenyl)-2,6-dimethyl-4-(6-azaspiro[2.5]octan-6-yl)pyridin-3-yl)-2-(tert-butoxy)acetate (50 mg, 0.084 mmol) in ethanol (1 mL) and stirred for 24 h at 90° C. An additional 0.25 mL sodium hydroxide solution was added and the reaction was continued for 24 h at 90° C. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)-phenyl)-2,6-dimethyl-4-(6-azaspiro[2.5]octan-6-yl)pyridin-3-yl)-2-(tert-butoxy)acetic acid 24 mg (52%). ¹H NMR (500 MHz, DMSO) δ 9.19-9.16 (m, 1H), 8.02 (d, J=8.1 Hz, 1H), 7.99-7.96 (m, 1H), 7.47 (d, J=7.7 Hz, 1H), 7.37-7.33 (m, 4H), 7.27-7.25 (m, 1H), 7.21 (d, J=7.7 Hz, 1H), 5.84 (s, 1H), 4.52 (d, J=5.9 Hz, 2H), 3.55-3.53 (m, 2H), 2.88-2.84 (m, 1H), 2.74-2.70 (m, 1H), 2.46 (s, 3H), 2.03 (s, 3H), 1.82-1.79 (m, 2H), 1.15 (s, 9H), 0.73-0.70 (m, 1H), 0.55-0.53 (m, 1H), 0.24-0.23 (m, 1H), 0.19-0.17 (m, 1H), 0.07-0.05 (m, 1H), −0.06-0.09 (m, 1H). UPLC (M+H)=556.5.

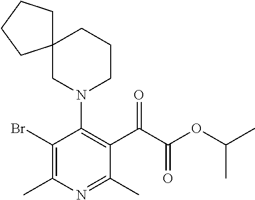 [SMILES: Cc1nc(C)c(C(=O)C(=O)OC(C)C)c(N2CCCC3(CCCC3)C2)c1Br]

### Isopropyl 2-(5-bromo-2,6-dimethyl-4-(7-azaspiro[4.5]decan-7-yl)pyridin-3-yl)-2-oxoacetate

To a solution of 7-azaspiro[4.5]decane (500 mg, 1.79 mmol) and DIEA (0.88 mL, 5.39 mmol) in anhydrous CH₃CN (12 mL) was added isopropyl 2-(5-bromo-4-chloro-2,6-dimethylpyridin-3-yl)-2-oxoacetate (601 mg, 1.79 mmol) at rt. The resulting mixture was placed in a pre-heated oil bath (80° C.) and stirred for 18 h; cooled, and concentrated. The reaction was repeated, and crude product was charged (DCM) to a 80 g ISCO silica gel cartridge and gradient elution (5-35% EtOAc/hexanes) using an Isolera chromatography station to give isopropyl 2-(5-bromo-2,6-dimethyl-4-(7-azaspiro[4.5]decan-7-yl)pyridin-3-yl)-2-oxoacetate 1.50 g. ¹H NMR (500 MHz, DMSO) δ 5.11-5.08 (m, 1H), 3.35-3.51 (m, 4H), 2.59 (s, 3H), 2.25 (s, 3H), 1.58 (br. s, 2H), 1.53-1.48 (m, 3H), 1.40-1.34 (m, 7H), 1.29 (d, J=6.2 Hz, 6H). UPLC (M+H)=439.3.

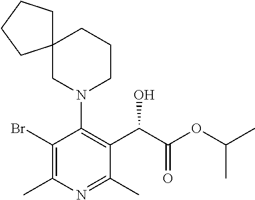 [SMILES: Cc1nc(C)c([C@H](O)C(=O)OC(C)C)c(N2CCCC3(CCCC3)C2)c1Br]

### (S)-Isopropyl 2-(5-bromo-2,6-dimethyl-4-(7-azaspiro[4.5]decan-7-yl)pyridin-3-yl)-2-hydroxyacetate

The 1.3 mL of benzo[d][1,3,2]dioxaborole (617 mg, 5.14 mmol) was added to a nitrogen purged solution of isopropyl 2-(5-bromo-2,6-dimethyl-4-(7-azaspiro[4.5]decan-7-yl)pyridin-3-yl)-2-oxoacetate (1.5 g, 3.43 mmol) and 1.0 mL of 1M (R)-1-methyl-3,3-diphenylhexahydropyrrolo[1,2-c][1,3,2]oxazaborole (285 mg, 1.0 mmol) in toluene (40 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine, and dried (MgSO₄). The crude product was charged (DCM) to a 40 g ISCO silica gel cartridge and gradient elution (5-45% EtOAc/hexanes) using an Isolera chromatography station gave (S)-isopropyl 2-(5-bromo-2,6-dimethyl-4-(7-azaspiro[4.5]decan-7-yl)pyridin-3-yl)-2-hydroxyacetate 1.3 g (86%) as a mixture of diastereomers. ¹H NMR (500 MHz, DMSO) δ 6.07/6.03 (s, 1H), 4.96-4.93 (m, 1H), 3.39-3.36 (m, 1H), 3.27/3.17 (t, J=11.0 Hz, 1H), 2.91 (d, J=11.3 Hz, 1H), 2.60 (d, J=11.7 Hz, 1H), 2.52 (s, 3H), 2.34/2.30 (s, 3H), 1.71-1.32 (m, 12H), 1.13 (d, J=6.2 Hz, 3H), 1.08 (d, J=6.2 Hz, 3H). UPLC (M+H)=441.3.

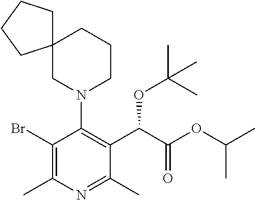 [SMILES: Cc1nc(C)c([C@H](OC(C)(C)C)C(=O)OC(C)C)c(N2CCCC3(CCCC3)C2)c1Br]

### (S)-Isopropyl 2-(5-bromo-2,6-dimethyl-4-(7-azaspiro[4.5]decan-7-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(7-azaspiro[4.5]decan-7-yl)pyridin-3-yl)-2-hydroxyacetate (1.3 g, 2.96 mmol) and 0.6 mL of 70% HClO₄ in DCM (20 mL) for 20 min. The reaction mixture was allowed to warm to rt and stirred for 18 h in a pressure sealed vessel. The reaction mixture was diluted with DCM, washed with 1M Na₂CO₃ soln, and dried over MgSO₄. The crude product was charged (DCM) to a 40 g ISCO silica gel cartridge and gradient elution (5-35% EtOAc/hexanes) using an Isolera chromatography station gave (S)-isopropyl 2-(5-bromo-2,6-dimethyl-4-(7-azaspiro[4.5]decan-7-yl)pyridin-3-yl)-2-(tert-butoxy)acetate 516 mg (35%) as a mixture of diastereomers. ¹H NMR (500 MHz, DMSO) δ 6.30 (s, 1H), 4.93-4.88 (m, 1H), 3.97 (br. s, 1H), 3.18-3.17 (m, 1H), 2.73-2.71 (m, 1H), 2.64 (d, J=11.0 Hz, 1H), 2.53 (s, 3H), 2.35 (s, 3H), 1.72-1.45 (m, 8H), 1.35-1.28 (m, 3H), 1.16 (s, 9H), 1.15 (d, J=6.6 Hz, 3H), 1.12 (br. s, 1H), 1.06 (d, J=6.3 Hz, 3H). UPLC (M+H)=497.4.

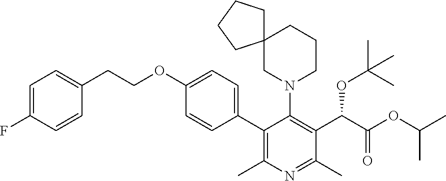 [SMILES: Cc1nc(C)c([C@H](OC(C)(C)C)C(=O)OC(C)C)c(N2CCCC3(CCCC3)C2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(7-azaspiro[4.5]decan-7-yl)pyridin-3-yl)acetate

The tretrakis (46 mg, 0.04 mmol) was added to a nitrogen purged and degassed solution (S)-isopropyl 2-(5-bromo-2,6-dimethyl-4-(7-azaspiro[4.5]decan-7-yl)pyridin-3-yl)-2-(tert-butoxy)acetate (100 mg, 0.20 mmol), (4-(4-fluorophenethoxy)phenyl)boronic acid (53 mg, 0.20 mmol), and sodium carbonate (150 mg, 1.4 mmol) in dioxane (3 mL) and water (0.6 mL) and stirred in a screw-capped pressure vessel for 4 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(7-azaspiro[4.5]decan-7-yl)pyridin-3-yl)acetate 34 mg (27%) as a mixture of diastereomers. ¹H NMR (500 MHz, DMSO) δ 7.40-7.38 (m, 2H), 7.19-7.12 (m, 3H), 7.01-6.99 (m, 3H), 6.32/5.91 (s, 1H), 4.96-4.91 (m, 1H), 4.23 (t, J=6.6 Hz, 2H), 3.40-3.33 (m, 2H), 3.06 (t, J=6.6 Hz, 2H), 2.88 (d, J=10.6 Hz, 1H), 2.56 (d, J=11.4 Hz, 1H), 2.39 (s, 3H), 2.02-1.99 (m, 1H), 1.97 (s, 3H), 1.73-1.67 (m, 1H), 1.63-1.48 (m, 4H), 1.47-1.40 (m, 3H), 1.28-1.24 (m, 1H), 1.19 (d, J=6.6 Hz, 3H), 1.16 (s, 9H), 1.13-1.11 (m, 5H). UPLC (M+H)=631.6.

### Example 18

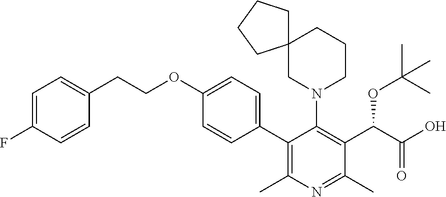 [SMILES: Cc1nc(C)c([C@H](OC(C)(C)C)C(=O)O)c(N2CCCC3(CCCC3)C2)c1-c1ccc(OCCc2ccc(F)cc2)cc1]

### (S)-2-(tert-Butoxy)-2-(5-(4-(4-fluorophenethoxy)phenyl)-2,6-dimethyl-4-(7-azaspiro[4.5]decan-7-yl)pyridin-3-yl)acetic acid

The 0.21 mL of 1M sodium hydroxide (8.4 mg, 0.21 mmol) was added to a solution of (S)-isopropyl 2-(tert-butoxy)-2-(5-(4-(4-fluorophenethoxy)phenyl)-2,6-dimethyl-4-(7-azaspiro[4.5]decan-7-yl)pyridin-3-yl)acetate (33 mg, 0.052 mmol) in ethanol (1 mL) and stirred for 48 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)phenyl)-2,6-dimethyl-4-(7-azaspiro[4.5]decan-7-yl)pyridin-3-yl)acetic acid 18.6 mg (60%) as a mixture of diastereomers. ¹H NMR (500 MHz, DMSO) δ 7.41-7.38 (m, 2H), 7.16-7.13 (m, 3H), 7.04-6.98 (m, 3H), 6.28 (s, 1H), 4.23 (t, J=6.2 Hz, 2H), 3.38-3.25 (m, 2H), 3.06 (t, J=5.8 Hz, 2H), 3.02 (d, J=11.3 Hz, 1H), 2.51-2.48 (m, 1H), 2.41 (s, 3H), 2.01-1.95 (m, 1H), 1.96/1.91 (s, 3H), 1.78 (br. s, 1H), 1.60-1.54 (m, 3H), 1.49-1.35 (m, 4H), 1.27-1.24 (m, 1H), 1.14 (s, 9H), 1.05-0.99 (m, 1H), 0.79-0.74 (m, 1H). UPLC (M+H)=590.5.

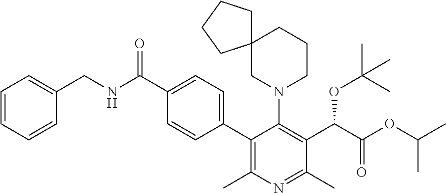 [SMILES: Cc1nc(C)c([C@H](OC(C)(C)C)C(=O)OC(C)C)c(N2CCCC3(CCCC3)C2)c1-c1ccc(C(=O)NCc2ccccc2)cc1]

### Isopropyl 2-(5-(4-(benzylcarbamoyl)phenyl)-2,6-dimethyl-4-(7-azaspiro[4.5]decan-7-yl)pyridin-3-yl)-2-(tert-butoxy)acetate

The Pd(Ph₃P)₄ (46 mg, 0.04 mmol) was added to a nitrogen purged and degassed solution (S)-isopropyl 2-(5-bromo-2,6-dimethyl-4-(7-azaspiro[4.5]decan-7-yl)pyridin-3-yl)-2-(tert-butoxy)acetate (100 mg, 0.20 mmol), (4-(benzylcarbamoyl)phenyl)boronic acid (57 mg, 0.22 mmol), and sodium carbonate (150 mg, 1.4 mmol) in dioxane (3 mL) and water (0.6 mL) and stirred in a screw-capped pressure vessel for 4 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-2,6-dimethyl-4-(7-azaspiro[4.5]decan-7-yl)pyridin-3-yl)-2-(tert-butoxy)acetate 21 mg (17%) as a mixture of diastereomers. ¹H NMR (500 MHz, DMSO) δ 9.19-9.16 (m, 1H), 8.01-7.97 (m, 2H), 7.47/7.44 (d, J=7.7 Hz, 1H), 7.38-7.34 (m, 4H), 7.29-7.25 (m, 2H), 6.31/5.90 (s, 1H), 4.99-4.94 (m, 1H), 4.52 (d, J=6.2 Hz, 2H), 3.36-3.34 (m, 2H), 3.01/2.94 (d, J=11.7 Hz, 1H), 2.59-2.54 (m, 1H), 2.41 (s, 3H), 1.97 (s, 3H), 1.93-1.87 (m, 1H), 1.72-1.67 (m, 1H), 1.62-1.34 (m, 8H), 1.27-1.23 (m, 1H), 1.21 (d, J=6.2 Hz, 3H), 1.17 (s, 9H), 1.15-1.13 (m, 4H). UPLC (M+H)=626.6.

### Example 19

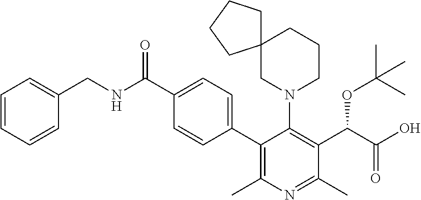 [SMILES: Cc1nc(C)c([C@H](OC(C)(C)C)C(=O)O)c(N2CCCC3(CCCC3)C2)c1-c1ccc(C(=O)NCc2ccccc2)cc1]

### (S)-2-(5-(4-(Benzylcarbamoyl)phenyl)-2,6-dimethyl-4-(7-azaspiro[4.5]decan-7-yl)pyridin-3-yl)-2-(tert-butoxy)acetic acid

The 0.31 mL of 1M sodium hydroxide (13 mg, 0.31 mmol) was added to a solution of (S)-isopropyl 2-(5-(4-(benzylcarbamoyl)phenyl)-2,6-dimethyl-4-(7-azaspiro[4.5]decan-7-yl)pyridin-3-yl)-2-(tert-butoxy)acetate (50 mg, 0.08 mmol) in ethanol (1 mL) and stirred for 24 h at 90° C. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(7-azaspiro[4.5]decan-7-yl)pyridin-3-yl)-2-(tert-butoxy)acetic acid 1.4 mg (3%) as a mixture of diastereomers, and recovered starting material. UPLC (M+H)=584.6.

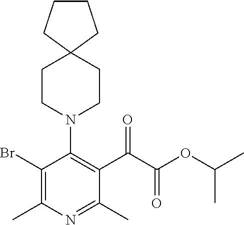 [SMILES: Cc1nc(C)c(C(=O)C(=O)OC(C)C)c(N2CCC3(CCCC3)CC2)c1Br]

### Isopropyl 2-(5-bromo-2,6-dimethyl-4-(8-azaspiro[4.5]decan-8-yl)pyridin-3-yl)-2-oxoacetate

To a solution of 8-azaspiro[4.5]decane (1.0 g, 7.18 mmol) and DIEA (3.76 mL, 21.6 mmol) in anhydrous CH₃CN (30 mL) was added isopropyl 2-(5-bromo-4-chloro-2,6-dimethylpyridin-3-yl)-2-oxoacetate (2.4 g, 7.18 mmol) at rt. The resulting mixture was placed in a pre-heated oil bath (80° C.) and stirred for 72 h; cooled, diluted with ether, washed with water, brine, and dried (MgSO₄). The crude product was charged (DCM) to a 80 g ISCO silica gel cartridge and gradient elution (0-15% EtOAc/hexanes) using an Isolera chromatography station to give isopropyl 2-(5-bromo-2,6-dimethyl-4-(8-azaspiro[4.5]decan-8-yl)pyridin-3-yl)-2-oxoacetate 2.35 g (75%). ¹H NMR (500 MHz, CDCl3) δ 5.07-5.02 (m, 1H), 4.2-4.19 (m, 2H), 3.52-3.51 (m, 2H), 2.60 (s, 3H), 2.29 (s, 3H), 1.57 (br. s, 4H), 1.40-1.37 (m, 8H), 1.27 (d, J=5.9 Hz, 6H). UPLC (M+H)=439.4.

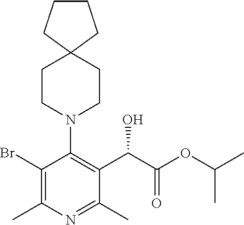 [SMILES: Cc1nc(C)c([C@H](O)C(=O)OC(C)C)c(N2CCC3(CCCC3)CC2)c1Br]

### Isopropyl (S)-isopropyl 2-(5-bromo-2,6-dimethyl-4-(8-azaspiro[4.5]decan-8-yl)pyridin-3-yl)-2-hydroxyacetate

The 2.1 mL of benzo[d][1,3,2]dioxaborole (1.16 g, 9.74 mmol) was added to a nitrogen purged solution of isopropyl 2-(5-bromo-2,6-dimethyl-4-(8-azaspiro[4.5]decan-8-yl)pyridin-3-yl)-2-oxoacetate (2.1 g, 4.87 mmol) and 1.95 mL of (R)-1-methyl-3,3-diphenylhexahydropyrrolo[1,2-c][1,3,2]oxazaborole (540 mg, 1.95 mmol) in toluene (50 mL) at −60° C. and allowed to warm to −15° C. before being placed in the freezer 72 h. The reaction was quenched with 1M Na₂CO₃, diluted with EtOAc, and stirred for 30 min. The organic layer was washed with sat'd Na₂CO₃ soln, brine and dried (MgSO₄). The crude product was charged (DCM) to a 80 g ISCO silica gel cartridge and gradient elution (0-50% EtOAc/hexanes) using an Isolera chromatography station gave isopropyl (S)-isopropyl 2-(5-bromo-2,6-dimethyl-4-(8-azaspiro[4.5]decan-8-yl)pyridin-3-yl)-2-hydroxyacetate 1.84 g (72%). ¹H NMR (500 MHz, DMSO) δ 5.93-5.90 (m, 1H), 4.97-4.92 (m, 1H), 3.61 (t, J=11 Hz, 1H), 3.41 (t, J=11 Hz, 1H), 2.78 (d, J=11.4 Hz, 1H), 2.69 (d, J=11 Hz, 1H), 2.53 (s, 3H), 2.37 (s, 3H), 1.63-1.57 (m, 6H), 1.54-1.51 (m, 2H), 1.44-1.39 (m, 4H), 1.14 (d, J=6.3 Hz, 3H), 1.07 (d, J=6.2 Hz, 3H). UPLC (M+H)=441.1.

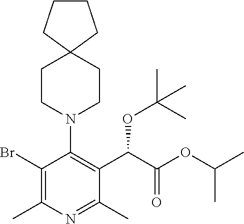 [SMILES: Cc1nc(C)c([C@H](OC(C)(C)C)C(=O)OC(C)C)c(N2CCC3(CCCC3)CC2)c1Br]

### (S)-Isopropyl 2-(5-bromo-2,6-dimethyl-4-(8-azaspiro[4.5]decan-8-yl)pyridin-3-yl)-2-(tert-butoxy)acetate

The isobutylene gas was bubbled into a nitrogen purged, cooled (0° C.) solution isopropyl (S)-isopropyl 2-(5-bromo-2,6-dimethyl-4-(8-azaspiro[4.5]decan-8-yl)pyridin-3-yl)-2-hydroxyacetate (1.84 g, 4.19 mmol) and 0.54 mL of 70% HClO₄ in DCM (30 mL) for 20 min. The reaction mixture was allowed to warm to rt and stirred for 72 h in a pressure sealed vessel. The reaction mixture was diluted with DCM, washed with 1M Na₂CO₃ soln, and dried over MgSO₄. The crude product was charged (DCM) to a 80 g ISCO silica gel cartridge and gradient elution (0-20% EtOAc/hexanes) using an Isolera chromatography station gave (S)-isopropyl 2-(5-bromo-2,6-dimethyl-4-(8-azaspiro[4.5]decan-8-yl)pyridin-3-yl)-2-(tert-butoxy)acetate 1.96 g (94%). ¹H NMR (500 MHz, DMSO) δ 6.20 (br. s, 1H), 4.94-4.89 (m, 1H), 3.90 (br. s, 1H), 3.36-3.32 (m, 1H), 2.88-2.86 (m, 1H), 2.63-2.61 (m, 1H), 2.52 (s, 3H), 2.42 (s, 3H), 1.65-1.54 (m, 9H), 1.43-1.40 (m, 3H), 1.15-1.14 (m, 12H), 1.07 (d, J=5.8 Hz, 3H). UPLC (M+H)=497.4.

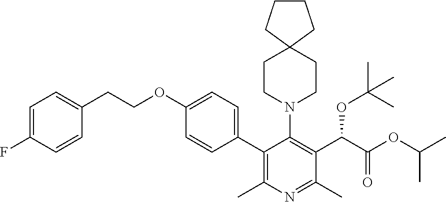 [SMILES: Cc1nc(C)c([C@H](OC(C)(C)C)C(=O)OC(C)C)c(N2CCC3(CCCC3)CC2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(8-azaspiro[4.5]decan-8-yl)pyridin-3-yl)acetate

The Pd(Ph₃P)₄ (140 mg, 0.121 mmol) was added to an argon purged and degassed solution of (S)-isopropyl 2-(5-bromo-2,6-dimethyl-4-(8-azaspiro[4.5]decan-8-yl)pyridin-3-yl)-2-(tert-butoxy)acetate (300 mg, 0.61 mmol), (4-(4-fluorophenethoxy)phenyl)boronic acid (173 mg, 0.66 mmol), and potassium phosphate tribasic (964 mg, 4.54 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-isopropyl 2-(tert-butoxy)-2-(5-(4-(4-fluorophenethoxy)phenyl)-2,6-dimethyl-4-(8-azaspiro[4.5]decan-8-yl)pyridin-3-yl)acetate 207 mg (54%). ¹H NMR (500 MHz, DMSO) δ 7.37-7.34 (m, 2H), 7.18 (d, J 8.8 Hz, 1H), 7.11 (t, J 8.8 Hz, 2H), 7.02-7.00 (m, 3H), 6.01 (br. s, 1H), 4.96-4.91 (m, 1H), 4.25-4.20 (m, 2H), 3.54-3.51 (m, 2H), 3.17-3.13 (m, 1H), 3.03 (t, J=6.2 Hz, 2H), 2.73 (t, J=10.3 Hz, 1H), 2.42 (s, 3H), 2.26-2.24 (m, 1H), 2.04 (s, 3H), 1.90-1.85 (m, 1H), 1.52-1.36 (m, 6H), 1.25 (br. s, 3H), 1.16 (d, J=5.9 Hz, 3H), 1.11 (br. s, 12H), 0.92 (br. s, 1H). UPLC (M+H)=631.7.

### Example 20

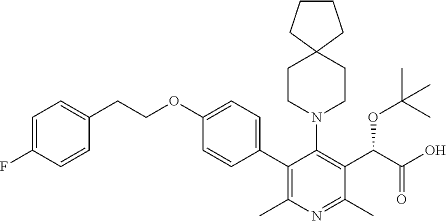 [SMILES: Cc1nc(C)c([C@H](OC(C)(C)C)C(=O)O)c(N2CCC3(CCCC3)CC2)c1-c1ccc(OCCc2ccc(F)cc2)cc1]

### (S)-2-(tert-Butoxy)-2-(5-(4-(4-fluorophenethoxy)phenyl)-2,6-dimethyl-4-(8-azaspiro[4.5]decan-8-yl)pyridin-3-yl)acetic acid

The potassium hydroxide (160 mg, 2.85 mmol) was added to a solution of (S)-isopropyl 2-(tert-butoxy)-2-(5-(4-(4-fluorophenethoxy)phenyl)-2,6-dimethyl-4-(8-azaspiro[4.5]decan-8-yl)pyridin-3-yl)acetate (180 mg, 0.285 mmol) in ethanol (3 mL) and stirred for 4 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)phenyl)-2,6-dimethyl-4-(8-azaspiro[4.5]decan-8-yl)pyridin-3-yl)acetic acid 41 mg (100%). ¹H NMR (500 MHz, DMSO) δ 7.38-7.35 (m, 2H), 7.19 (d, J=8.4 Hz, 1H), 7.13 (t, J=8.8 Hz, 2H), 7.04-7.00 (m, 3H), 5.89 (s, 1H), 4.28-4.21 (m, 2H), 3.54 (br. s, 2H), 3.31-3.29 (m, 1H), 3.05 (t, J=6.2 Hz, 2H), 2.74-2.72 (m, 1H), 2.43 (s, 3H), 2.26-2.24 (m, 1H), 2.05 (s, 3H), 1.88-1.83 (m, 1H), 1.58-1.54 (m, 1H), 1.49-1.36 (m, 5H), 1.28-1.26 (m, 3H), 1.12 (s, 9H), 0.93 (br. s, 1H). UPLC (M+H)=589.6.

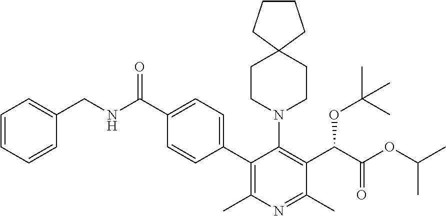 [SMILES: Cc1nc(C)c([C@H](OC(C)(C)C)C(=O)OC(C)C)c(N2CCC3(CCCC3)CC2)c1-c1ccc(C(=O)NCc2ccccc2)cc1]

### (S)-Isopropyl 2-(5-(4-(benzylcarbamoyl)phenyl)-2,6-dimethyl-4-(8-azaspiro[4.5]decan-8-yl)pyridin-3-yl)-2-(tert-butoxy)acetate

The Pd(Ph₃P)₄ (140 mg, 0.121 mmol) was added to an argon purged and degassed solution of (S)-isopropyl 2-(5-bromo-2,6-dimethyl-4-(8-azaspiro[4.5]decan-8-yl)pyridin-3-yl)-2-(tert-butoxy)acetate (300 mg, 0.61 mmol), (4-(benzylcarbamoyl)phenyl)boronic acid (170 mg, 0.66 mmol), and potassium phosphate tribasic (964 mg, 4.54 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-isopropyl 2-(5-(4-(benzylcarbamoyl)phenyl)-2,6-dimethyl-4-(8-azaspiro[4.5]decan-8-yl)pyridin-3-yl)-2-(tert-butoxy)acetate 355 mg (94%). ¹H NMR (500 MHz, DMSO) δ 9.19-9.16 (m, 1H), 8.02-7.98 (m, 2H), 7.45 (d, J=7.7 Hz, 1H), 7.34-7.31 (m, 4H), 7.21-7.23 (m, 2H), 6.00 (br. s, 1H), 4.97-4.92 (m, 1H), 4.52 (d, J=5.5 Hz, 2H), 3.52 (d, J=10.3 Hz, 1H), 3.20-3.17 (m, 1H), 2.76-2.71 (m, 1H), 2.45 (s, 3H), 2.30 (d, J=10.6 Hz, 1H), 2.04 (s, 3H), 1.80 (t, J=12 Hz, 1H), 1.56-1.25 (m, 10H), 1.18 (d, J=6.2 Hz, 3H), 1.14-1.12 (m, 12H), 0.89 (br. s, 1H). UPLC (M+H)=626.7.

### Example 21

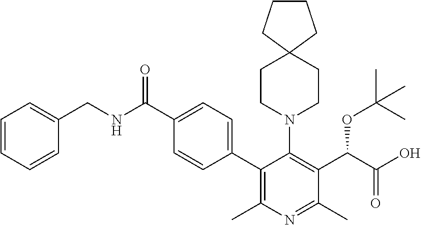 [SMILES: Cc1nc(C)c([C@H](OC(C)(C)C)C(=O)O)c(N2CCC3(CCCC3)CC2)c1-c1ccc(C(=O)NCc2ccccc2)cc1]

### (S)-2-(5-(4-(Benzylcarbamoyl)phenyl)-2,6-dimethyl-4-(8-azaspiro[4.5]decan-8-yl)pyridin-3-yl)-2-(tert-butoxy)acetic acid

The potassium hydroxide (291 mg, 5.19 mmol) was added to a solution of (S)-isopropyl 2-(5-(4-(benzylcarbamoyl)phenyl)-2,6-dimethyl-4-(8-azaspiro[4.5]decan-8-yl)pyridin-3-yl)-2-(tert-butoxy)acetate (325 mg, 0.519 mmol) in ethanol (5 mL) and stirred for 4 h at 90° C. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(8-azaspiro[4.5]decan-8-yl)pyridin-3-yl)-2-(tert-butoxy)acetic acid 44 mg (14.4%). ¹H NMR (500 MHz, DMSO) δ 9.18-9.15 (m, 1H), 8.03-7.98 (m, 2H), 7.47 (d, J=8.1 Hz, 1H), 7.35-7.32 (m, 4H), 7.25-7.23 (m, 2H), 5.89 (s, 1H), 4.52 (d, J=5.9 Hz, 2H), 3.34-3.32 (m, 2H), 2.76-2.71 (m, 1H), 2.46 (s, 3H), 2.30-2.28 (m, 1H), 2.05 (s, 3H), 1.78 (t, J=11 Hz, 1H), 1.59-1.54 (m, 1H), 1.48-1.37 (m, 5H), 1.30-1.26 (m, 3H), 1.14-1.08 (s, 10H), 0.89 (br. s, 1H). UPLC (M+H)=584.6.

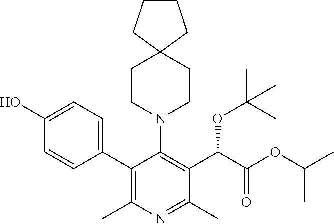 [SMILES: Cc1nc(C)c([C@H](OC(C)(C)C)C(=O)OC(C)C)c(N2CCC3(CCCC3)CC2)c1-c1ccc(O)cc1]

### Isopropyl 2-(tert-butoxy)-2-(5-(4-hydroxyphenyl)-2,6-dimethyl-4-(8-azaspiro[4.5]decan-8-yl)pyridin-3-yl)acetate

The Pd(Ph₃P)₄ (140 mg, 0.121 mmol) was added to an argon purged and degassed solution of (S)-isopropyl 2-(5-bromo-2,6-dimethyl-4-(8-azaspiro[4.5]decan-8-yl)pyridin-3-yl)-2-(tert-butoxy)acetate (300 mg, 0.61 mmol), (4-((tert-butyldimethylsilyl)oxy)phenyl)boronic acid (168 mg, 0.666 mmol), and potassium phosphate tribasic (964 mg, 4.54 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-20% EtOAc/hexanes) using an Isolera chromatography station gave (S)-isopropyl 2-(tert-butoxy)-2-(5-(4-((tert-butyldimethylsilyl)oxy) phenyl)-2,6-dimethyl-4-(8-azaspiro[4.5]decan-8-yl)pyridin-3-yl)acetate 353 mg (94% yield). This material was taken up in dry THF (4 mL) and treated to TBAF (0.61 ml, 0.61 mmol) at 0° C. The mixture was stirred 2 h at rt, diluted with EtOAc and water, and the organic layer was washed with brine and dried (MgSO4). The crude product was charged (DCM) to a 40 g ISCO silica gel cartridge and gradient elution (0-50% EtOAc/hexanes) using an Isolera chromatography station and gave (S)-isopropyl 2-(tert-butoxy)-2-(5-(4-hydroxyphenyl)-2,6-dimethyl-4-(8-azaspiro[4.5]decan-8-yl)pyridin-3-yl)acetate 230 mg (75%). ¹H NMR (500 MHz, DMSO) δ 7.07-7.06 (m, 1H), 6.91-6.98 (m, 1H), 6.85-6.83 (m, 2H), 6.03 (br. s, 1H), 4.97-4.92 (m, 1H), 3.14 (d, J=9.9 Hz, 1H), 2.74 (t, J=13.9 Hz, 1H), 2.42 (s, 3H), 2.26 (d, J=12.8 Hz, 1H), 2.06 (s, 3H), 1.94 (t, J=12.1 Hz, 1H), 1.54-1.48 (m, 5H), 1.40-1.35 (m, 1H), 1.32-1.25 (m, 3H), 1.18 (d, J=6.2 Hz, 3H), 1.16-1.12 (m, 14H), 1.01-0.97 (m, 1H). UPLC (M+H)=509.4.

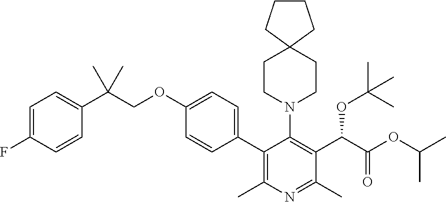 [SMILES: Cc1nc(C)c([C@H](OC(C)(C)C)C(=O)OC(C)C)c(N2CCC3(CCCC3)CC2)c1-c1ccc(OCC(C)(C)c2ccc(F)cc2)cc1]

### (S)-Isopropyl 2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetate

To a stirred solution of (S)-isopropyl 2-(tert-butoxy)-2-(5-(4-hydroxyphenyl)-2,6-dimethyl-4-(8-azaspiro[4.5]decan-8-yl)pyridin-3-yl)acetate (100 mg, 0.197 mmol), 2-(4-fluorophenyl)-2-methylpropan-1-ol (166 mg, 0.985 mmol) and triphenylphosphine (258 mg, 0.985 mmol) in dry THF (3 mL) was added DIAD (0.19 mL, 0.98 mmol) at rt, and the mixture was heated at 70° C. for 16 h in a screw top vial. Additional 2-(4-fluorophenyl)-2-methylpropan-1-ol (125 mg), triphenylphosphine (190 mg) and DIAD (0.14 mL) were added at rt, and the heating was continued another 16 h. The reaction mixture was diluted with EtOAc, washed with brine, and dried. The crude product was charged (DCM) to a 80 g ISCO silica gel cartridge and gradient elution (0-75% EtOAc/hexanes) using an Isolera chromatography station and gave (S)-isopropyl 2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetate 450 mg (contained impurities) which was forward for hydrolysis without further purification. A 70 mg sample was purified for characterization was purified by prep HPLC to obtain (S)-isopropyl 2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetate 8 mg: ¹H NMR (500 MHz, DMSO) δ 7.53-7.50 (m, 2H), 7.19-7.12 (m, 3H), 7.02 (s, 3H), 6.02 (s, 1H), 4.97-4.92 (m, 1H), 4.10 (d, J=9.5 Hz, 1H), 4.04 (d, J=9.5 Hz, 1H), 3.18-3.13 (m, 1H), 2.73 (t, J=11.4 Hz, 1H), 2.43 (s, 3H), 2.25 (d, J=10.3 Hz, 1H), 2.05 (s, 3H), 1.88 (t, J=14.3 Hz, 1H), 1.54-1.44 (m, 5H), 1.41 (s, 6H), 1.38-1.26 (m, 4H), 1.18 (d J=6.2 Hz, 3H), 1.14-1.13 (m, 14H), 0.95 (br. s, 1H). UPLC (M+H)=659.4.

To a stirred solution of 2-((S)-2-(tert-butoxy)-2-(5-(4-hydroxyphenyl)-2,6-dimethyl-4-(8-azaspiro [4.5]decan-8-yl)pyridin-3-yl)acetoxy)propan-1-ylium (100 mg, 0.197 mmol), 2-(4-fluorophenyl)-2-methylpropan-1-ol (166 mg, 0.985 mmol) and triphenylphosphine (258 mg, 0.985 mmol) in dry THF (3 mL) was added DIAD (0.191 mL, 0.985 mmol) at rt and the solution was heated at 70° C. for 16 h in a screw capped vessel. An additional equivalent of reagents was added at rt and the reaction mixture was heated at 70° C. for 16 h, cooled, diluted with EtOAc, washed with brine, and dried (MgSO4). The crude product was charged (DCM) to a 40 g ISCO silica gel cartridge and gradient elution (0-75% EtOAc/hexanes) using an Isolera chromatography station gave (S)-isopropyl 2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetate 65 mg (49%). UPLC (M+H)=659.4.

### Example 22

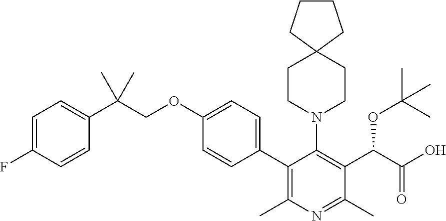 [SMILES: Cc1nc(C)c([C@H](OC(C)(C)C)C(=O)O)c(N2CCC3(CCCC3)CC2)c1-c1ccc(OCC(C)(C)c2ccc(F)cc2)cc1]

### (S)-2-(tert-Butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetic acid

The potassium hydroxide (111 mg, 1.97 mmol) was added to the solution of crude (S)-isopropyl 2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetate (180 mg, 0.285 mmol) in ethanol (3 mL) and stirred for 6 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetic acid 45.1 mg (36.3%). ¹H NMR (500 MHz, MeOD) δ 7.52-7.49 (m, 2H), 7.26-7.24 (m, 1H), 7.06-7.03 (m, 5H), 5.58 (s, 1H), 4.09 (d, J=9.0 Hz, 1H), 4.07 (d, J=9.1 Hz, 1H), (protons adjacent to nitrogen not evident), 2.26 (s, 3H), 1.95 (s, 3H), 1.59-1.56 (m, 3H), 1.50 (s, 6H), 1.47-1.42 (m, 3H), 1.37-1.32 (m, 3H), 1.27-1.21 (m, 3H), 1.19 (s, 9H). UPLC (M+H)=617.5.

Potassium hydroxide (111 mg, 1.97 mmol) was added to a solution of (S)-isopropyl 2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetate (65 mg, 0.099 mmol) in absolute EtOH (3 mL) and stirred for 6 h at 90° C. The mixture was diluted with EtOAc (15 mL) and neutralized with 1N HCl (4 mL) to pH 4. Buffer (5 mL, pH=5) was added and the mixture extracted with EtOAc. The organic layer was washed with brine and dried (MgSO₄). The crude product was subjected to prep HPLC (gradient: 10-100% B over 25 min @ 40 ml/min) Phenomenex Gemini column (30×100 mm, 10 u) solvent B=90% AcCN—10% H2O— 0.1% TFA and solvent A=10% AcCN—90% H2O— 0.1% TFA, and there was obtained (S)-2-(tert-butoxy)-2-(5-(4-(2-(4-fluorophenyl)-2-methylpropoxy)phenyl)-2,6-dimethyl-4-(8-azaspiro[4.5]decan-8-yl)pyridin-3-yl)acetic acid 45 mg (73%). ¹HNMR (500 MHz, MeOD) δ 7.52-7.49 (m, 2H), 7.25 (d, J 10.2 Hz, 1H), 7.06-7.03 (m, 5H), 5.58 (s, 1H), 4.09, 4.07 (AB, J_{AB}=9 Hz, 2H), 2.68 (s, 3H), 2.26 (s, 3H), 1.59-156 (m, 4H), 1.50 (s, 6H), 1.47-1.24 (series m, 12H), 1.19 (s, 9H). UPLC (M+H)=656.5.

### Example 23

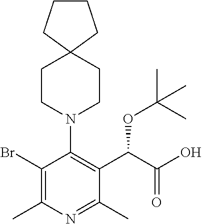 [SMILES: Cc1nc(C)c([C@H](OC(C)(C)C)C(=O)O)c(N2CCC3(CCCC3)CC2)c1Br]

### (S)-2-(5-Bromo-2,6-dimethyl-4-(8-azaspiro[4.5]decan-8-yl)pyridin-3-yl)-2-(tert-butoxy)acetic acid

Potassium hydroxide (226 mg, 4.04 mmol) was added to a solution of (S)-isopropyl 2-(5-bromo-2,6-dimethyl-4-(8-azaspiro[4.5]decan-8-yl)pyridin-3-yl)-2-(tert-butoxy)acetate (200 mg, 0.404 mmol) in absolute EtOH (4 mL) and stirred for 6 h at 90° C. and then at rt for 10 h. The mixture was diluted with EtOAc (15 mL) and neutralized with 1N HCl (4 mL) to pH 4. Buffer (5 mL, pH=5) was added and the mixture extracted with EtOAc. The organic layer was washed with brine and dried (MgSO₄). The crude product (S)-2-(5-bromo-2,6-dimethyl-4-(8-azaspiro[4.5]decan-8-yl)pyridin-3-yl)-2-(tert-butoxy)acetic acid 190 mg was carried forward. ¹H NMR (500 MHz, DMSO) δ 5.86 (s, 1H), 3.87 (t, J 10.3 Hz, 1H), 3.23 (t, J=11.7 Hz, 1H), 3.10 (d, J=11.7 Hz, 1H), 2.61 (d, J=11.7 Hz, 1H), 2.41 (s, 3H), 1.91 (s, 3H), 1.68-1.48 (series of m, 9H), 1.42-1.37 (m, 3H), 1.13 (s, 9H). UPLC (M+H)=455.2.

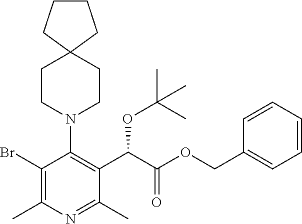 [SMILES: Cc1nc(C)c([C@H](OC(C)(C)C)C(=O)OCc2ccccc2)c(N2CCC3(CCCC3)CC2)c1Br]

### (S)-Benzyl 2-(5-bromo-2,6-dimethyl-4-(8-azaspiro[4.5]decan-8-yl)pyridin-3-yl)-2-(tert-butoxy)acetate

Benzylbromide (0.023 mL, 0.189 mmol) was added to a stirred suspension of (S)-2-(5-bromo-2,6-dimethyl-4-(8-azaspiro[4.5]decan-8-yl)pyridin-3-yl)-2-(tert-butoxy)acetic acid (78 mg, 0.172 mmol) and cesium carbonate (56.1 mg, 0.172 mmol) in anhydrous acetonitrile (1 mL) and DMF (0.5 mL). The mixture was stirred for 12 h at rt, filtered, and the filtrate was charged (DCM) to a 40 g ISCO silica gel cartridge and gradient elution (0-25% EtOAc/hexanes) using an Isolera chromatography station gave (S)-benzyl 2-(5-bromo-2,6-dimethyl-4-(8-azaspiro[4.5]decan-8-yl)pyridin-3-yl)-2-(tert-butoxy)acetate 80 mg (86%). ¹H NMR (500 MHz, CDCl3) δ 7.34-7.30 (m, 3H), 7.26-7.23 (m, 2H), 6.24 (br. s, 1H), 5.21, 5.07 (AB, J_{AB}=12.3 Hz, 2H), 3.92 (dt, J=12.1, 2.3 Hz, 1H), 3.33 (dt, J=11.8, 2.3 Hz, 1H), 2.78 (d, J=11.5 Hz, 1H), 2.63 (s, 3H), 2.62 (br. s, 1H), 2.52 (s, 3H), 1.68-1.38 (series of m, 12H), 1.21 (s, 9H). UPLC (M+H)=545.5.

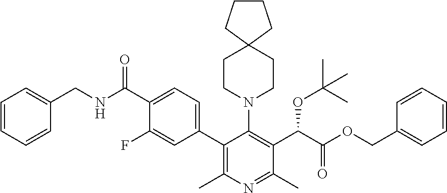 [SMILES: Cc1nc(C)c([C@H](OC(C)(C)C)C(=O)OCc2ccccc2)c(N2CCC3(CCCC3)CC2)c1-c1ccc(C(=O)NCc2ccccc2)c(F)c1]

### (S)-Benzyl 2-(5-(4-(benzylcarbamoyl)-3-fluorophenyl)-2,6-dimethyl-4-(8-azaspiro[4.5]decan-8-yl)pyridin-3-yl)-2-(tert-butoxy)acetate

The Pd(Ph₃P)₄ (34 mg, 0.029 mmol) was added to an argon purged and degassed solution of (S)-benzyl 2-(5-bromo-2,6-dimethyl-4-(8-azaspiro[4.5]decan-8-yl)pyridin-3-yl)-2-(tert-butoxy)acetate (80 mg, 0.147 mmol), (4-(benzylcarbamoyl)-3-fluorophenyl)boronic acid (44 mg, 0.162 mmol), and sodium carbonate (78 mg, 0.74 mmol) in dioxane (1 mL) and water (0.25 mL) and stirred in a screw-capped pressure vessel for 16 h at 85° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-benzyl 2-(5-(4-(benzylcarbamoyl)-3-fluorophenyl)-2,6-dimethyl-4-(8-azaspiro[4.5]decan-8-yl)pyridin-3-yl)-2-(tert-butoxy)acetate 62 mg (61%). ¹H NMR (500 MHz, DMSO) δ 7.90-7.85 (m, 1H), 7.41-7.40 (m, 2H), 7.37-7.32 (m, 7H), 7.29-7.26 (m, 2H), 7.07-7.01 (m, 1H), 6.01 (br. s, 1H), 5.26, 5.20 (AB, J_{AB}=12.0 Hz, 2H), 4.64 (s, 2H), 3.37-3.32 (m, 4H), 2.49 (s, 3H), 2.17/2.16 (s, 3H), 1.54-1.52 (m, 4H), 1.25 (br. s, 8H), 1.21 (s, 9H). UPLC (M+H)=692.5.

### Example 24

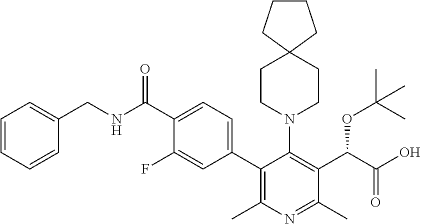 [SMILES: Cc1nc(C)c([C@H](OC(C)(C)C)C(=O)O)c(N2CCC3(CCCC3)CC2)c1-c1ccc(C(=O)NCc2ccccc2)c(F)c1]

### (S)-2-(5-(4-(Benzylcarbamoyl)-3-fluorophenyl)-2,6-dimethyl-4-(8-azaspiro[4.5]decan-8-yl)pyridin-3-yl)-2-(tert-butoxy)acetic acid

The solution of (S)-benzyl 2-(5-(4-(benzylcarbamoyl)-3-fluorophenyl)-2,6-dimethyl-4-(8-azaspiro[4.5]decan-8-yl)pyridin-3-yl)-2-(tert-butoxy)acetate (48 mg, 0.069 mmol) in MeOH (0.5 mL) was added to a suspension of Pearlman's Catalyst (10 mg, 0.071 mmol) in dry MeOH (1.5 mL) at rt. The flask was evacuated and charged with hydrogen (ballon) and stirred for 1 h, filtered, and concentrated. The crude product was purified by prep HPLC to obtain (S)-2-(5-(4-(benzylcarbamoyl)-3-fluorophenyl)-2,6-dimethyl-4-(8-azaspiro[4.5]decan-8-yl)pyridin-3-yl)-2-(tert-butoxy)acetic acid 27 mg (65%). ¹H NMR (500 MHz, DMSO) δ 8.98-8.94 (m, 1H), 7.78-7.73 (m, 1H), 7.42-7.27 (m, 6H), 7.14-7.05 (m, 1H), 5.86 (s, 1H), 4.51 (d, J=5.8 Hz, 2H), 3.27 (br. s, 2H), 2.76-2.71 (m, 1H), 2.46 (s, 3H), 2.38-2.31 (m, 1H), 2.08/2.07 (s, 3H), 1.85 (br. s, 1H), 1.60-1.23 (series of m, 10H), 1.19-1.10 (m, 9H), 1.02-0.95 (m, 1H). UPLC (M+H)=602.4.

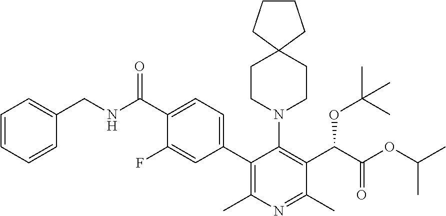 [SMILES: Cc1nc(C)c([C@H](OC(C)(C)C)C(=O)OC(C)C)c(N2CCC3(CCCC3)CC2)c1-c1ccc(C(=O)NCc2ccccc2)c(F)c1]

### (S)-Isopropyl 2-(5-(4-(benzylcarbamoyl)-3-fluorophenyl)-2,6-dimethyl-4-(8-azaspiro[4.5]decan-8-yl)pyridin-3-yl)-2-(tert-butoxy)acetate

The Pd(Ph₃P)₄ (70 mg, 0.061 mmol) was added to an argon purged and degassed solution of (S)-isopropyl 2-(5-bromo-2,6-dimethyl-4-(8-azaspiro[4.5]decan-8-yl)pyridin-3-yl)-2-(tert-butoxy)acetate (150 mg, 0.30 mmol), (4-(benzylcarbamoyl)-3-fluorophenyl)boronic acid (91 mg, 0.33 mmol), and sodium carbonate (160 mg, 0.74 mmol) in dioxane (2 mL) and water (0.4 mL) and stirred in a screw-capped pressure vessel for 16 h at 85° C. The reaction was allowed to cool, diluted with EtOAc, and the organic layer was washed with brine and dried (MgSO₄). The crude product was charged (DCM) to a 40 g ISCO silica gel cartridge and gradient elution (0-20% EtOAc/hexanes) using an Isolera chromatography station gave (S)-isopropyl 2-(5-(4-(benzylcarbamoyl)-3-fluorophenyl)-2,6-dimethyl-4-(8-azaspiro[4.5]decan-8-yl)pyridin-3-yl)-2-(tert-butoxy)acetate 110 mg (57%). ¹H NMR (500 MHz, DMSO) δ 8.98-8.94 (m, 1H), 7.79-7.73 (m, 1H), 7.412 (d, J=11 Hz, 0.6H), 7.37-7.34 (m, 4H), 7.30-7.26 (m, 1.5H), 7.14 (d, J=11 Hz, 0.4H), 7.07 (d, J=8.4 Hz, 0.5H), 5.99 (br. s, 1H), 4.99-4.94 (m, 1H), 4.52 (d, J=6.2 Hz, 2H), 3.35 (br. s, 1H), 3.32-3.17 (m, 1H), 2.77-2.72 (m, 1H), 2.46 (s, 3H), 2.39-2.34 (m, 1H), 2.09/2.08 (s, 3H), 1.91-1.85 (m, 1H), 1.56-1.26 (series of m, 9H), 1.20-1.18 (m, 3H), 1.16-1.14 (m, 13H), 1.01-0.96 (m, 1H). UPLC (M+H)=644.5.

### Example 25

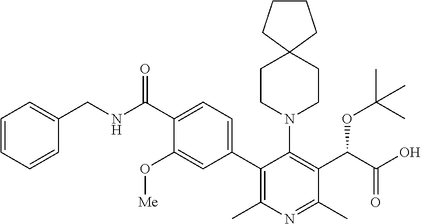 [SMILES: COc1cc(-c2c(C)nc(C)c([C@H](OC(C)(C)C)C(=O)O)c2N2CCC3(CCCC3)CC2)ccc1C(=O)NCc1ccccc1]

### (S)-2-(5-(4-(Benzylcarbamoyl)-3-methoxyphenyl)-2,6-dimethyl-4-(8-azaspiro[4.5]decan-8-yl)pyridin-3-yl)-2-(tert-butoxy)acetic acid

Potassium hydroxide (10.98 mg, 0.196 mmol) was added to a solution of (S)-isopropyl 2-(5-(4-(benzylcarbamoyl)-3-fluorophenyl)-2,6-dimethyl-4-(8-azaspiro[4.5]decan-8-yl)pyridin-3-yl)-2-(tert-butoxy)acetate (97 mg, 0.098 mmol) in MeOH (2 mL) and the solution was refluxed for 6. The reaction mixture was cooled, diluted with EtOAc, neutralized (1M HCl soln) to pH 4, and the organic layer was washed with brine, and dried (MgSO4). The crude product was purified by prep HPLC to obtain (S)-2-(5-(4-(benzylcarbamoyl)-3-methoxyphenyl)-2,6-dimethyl-4-(8-azaspiro[4.5]decan-8-yl)pyridin-3-yl)-2-(tert-butoxy)acetic acid 45 mg (100%). ¹H NMR (500 MHz, DMSO) δ UPLC (M+H)=614.4.

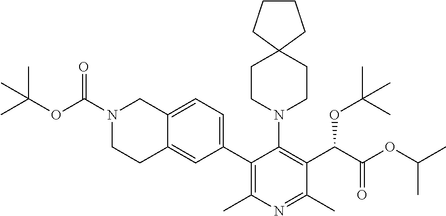 [SMILES: Cc1nc(C)c([C@H](OC(C)(C)C)C(=O)OC(C)C)c(N2CCC3(CCCC3)CC2)c1-c1ccc2c(c1)CCN(C(=O)OC(C)(C)C)C2]

### (S)-tert-Butyl 6-(5-(1-(tert-butoxy)-2-isopropoxy-2-oxoethyl)-2,6-dimethyl-4-(8-azaspiro[4.5]decan-8-yl)pyridin-3-yl)-3,4-dihydroisoquinoline-2(1H)-carboxylate

The Pd(Ph₃P)₄ (70 mg, 0.061 mmol) was added to an argon purged and degassed solution of (S)-isopropyl 2-(5-bromo-2,6-dimethyl-4-(8-azaspiro[4.5]decan-8-yl)pyridin-3-yl)-2-(tert-butoxy)acetate (300 mg, 0.61 mmol), tert-butyl 6-(4,4,5,5-tetramethyl-1,3,2-dioxaborolan-2-yl)-3,4-dihydroiso quinoline-2(1H)-carboxylate (239 mg, 0.67 mmol), and sodium carbonate (321 mg, 3.03 mmol) in DME (4 mL) and water (1.0 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-tert-butyl 6-(5-(1-(tert-butoxy)-2-isopropoxy-2-oxoethyl)-2,6-dimethyl-4-(8-azaspiro[4.5]decan-8-yl)pyridin-3-yl)-3,4-dihydroisoquinoline-2(1H)-carboxylate 340 mg (87%). ¹H NMR (500 MHz, DMSO) δ 7.27-7.24 (m, 1H), 7.13-7.11 (m, 1H), 6.93 (br. s, 1H), 6.00 (s, 1H), 4.98-4.91 (m, 1H), 4.66-4.49 (m, 2H), 3.64-3.57 (m, 2H), 3.21-3.17 (m, 1H), 2.82-2.68 (m, 3H), 2.43 (s, 3H), 2.32-2.30/2.19-2.17 (m, 1H), 2.10/2.06 (s, 3H), 1.77-1.69 (m, 1H), 1.54-1.25 (series of m, 11H), 1.43 (s, 9H), 1.18-1.13 (m, 6H), 1.12 (s, 9H), 0.92-0.87 (m, 1H). UPLC (M+H)=648.5

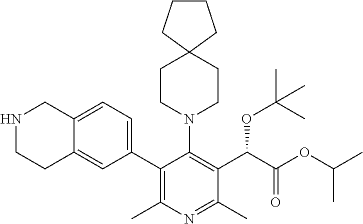 [SMILES: Cc1nc(C)c([C@H](OC(C)(C)C)C(=O)OC(C)C)c(N2CCC3(CCCC3)CC2)c1-c1ccc2c(c1)CCNC2]

### (S)-Isopropyl 2-(tert-butoxy)-2-(2,6-dimethyl-4-(8-azaspiro[4 5]decan-8-yl)-5-(1,2,3,4-tetrahydroisoquinolin-6-yl)pyridin-3-yl)acetate: The (S)-tert-butyl 6-(5-(1-(tert-butoxy)-2-isopropoxy-2-oxoethyl)-2,6-dimethyl-4-(8-azaspiro[4.5]decan-8-yl)pyridin-3-yl)-3,4-dihydroisoquinoline-2(1H)-carboxylate (340 mg, 0.525 mmol) was dissolved in cold (0° C.) 4N HCl in dioxane (2 mL) and the solution was stirred at rt for 2 h and concentrated to remove solvent. There was obtained (S)-isopropyl 2-(tert-butoxy)-2-(2,6-dimethyl-4-(8-azaspiro[4.5]decan-8-yl)-5-(1,2,3,4-tetrahydroisoquinolin-6-yl)pyridin-3-yl)acetate.2HCl 287 mg (100%). ¹H NMR (500 MHz, DMSO) δ 7.14-7.10 (m, 1H), 7.04-7.02 (m, 1H), 6.86-6.84 (m, 1H), 6.00 (s, 1H), 4.97-4.93 (m, 1H), 3.96 (s, 2H), 3.22-2.95 (m, 3H), 2.80-2.71 (m, 3H), 2.43 (s, 3H), 2.30/2.19 (d, J=12 Hz, 1H), 2.10/2.07 (s, 3H), 1.93/1.79 (t, J=12 Hz, 1H), 1.54-1.41 (m, 5H), 1.38-1.24 (m, 4H), 1.19-1.17 (m, 3H), 1.15-1.12 (s, 12H), 0.97 (br. s, 1H). UPLC (M+H)=548.5.

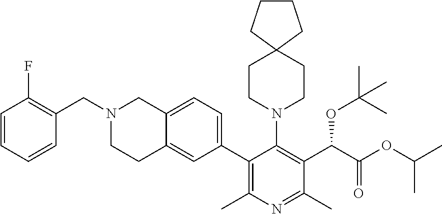 [SMILES: Cc1nc(C)c([C@H](OC(C)(C)C)C(=O)OC(C)C)c(N2CCC3(CCCC3)CC2)c1-c1ccc2c(c1)CCN(Cc1ccccc1F)C2]

### (S)-Isopropyl 2-(tert-butoxy)-2-(5-(2-(2-fluorobenzyl)-1,2,3,4-tetrahydroisoquinolin-6-yl)-2,6-dimethyl-4-(8-azaspiro[4.5]decan-8-yl)pyridin-3-yl)acetate

A suspension of (S)-isopropyl 2-(tert-butoxy)-2-(2,6-dimethyl-4-(8-azaspiro[4.5]decan-8-yl)-5-(1,2,3,4-tetrahydroisoquinolin-6-yl)pyridin-3-yl)acetate.2 HCl (100 mg, 0.161 mmol), sodium acetate (26.4 mg, 0.322 mmol), and 2-fluorobenzaldehyde (0.034 mL, 0.322 mmol) was stirred for 2 h in anhydrous DMF (2 mL). Sodium cyanoborohydride (50.6 mg, 0.806 mmol) was added in one portion and stirring was continued at rt for 16 h. The reaction mixture was diluted with EtOAc, washed with sat'd NaHCO₃ soln, brine, dried (MgSO₄). The crude product was subjected to prep HPLC to obtain (S)-isopropyl 2-(tert-butoxy)-2-(5-(2-(2-fluorobenzyl)-1,2,3,4-tetrahydroisoquinolin-6-yl)-2,6-dimethyl-4-(8-azaspiro[4.5]decan-8-yl)pyridin-3-yl)acetate 35.4 mg (33.5%). ¹H NMR (500 MHz, CDCl3) δ 7.62 (br. s, 1H), 7.54-7.46 (m, 1H), 7.32-7.12 (m, 4H), 6.93-6.88 (m, 1H), 5.58 (s, 1H), 5.15-5.10 (m, 1H), 5.45 (s, 2H), (protons adj nitrogen not evident), 2.75 (m, 3H), 2.68 (br. s, 2H), 2.31 (s, 3H), 1.56 (br. s, 4H), 1.42 (br, s, 3H), 1.30-1.27 (m, 11H), 1.17 (s, 9H). UPLC (M+H)=656.5.

### Example 26

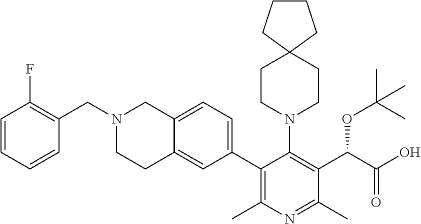 [SMILES: Cc1nc(C)c([C@H](OC(C)(C)C)C(=O)O)c(N2CCC3(CCCC3)CC2)c1-c1ccc2c(c1)CCN(Cc1ccccc1F)C2]

### (S)-2-(tert-Butoxy)-2-(5-(2-(2-fluorobenzyl)-1,2,3,4-tetrahydroisoquinolin-6-yl)-2,6-dimethyl-4-(8-azaspiro[4.5]decan-8-yl)pyridin-3-yl)acetic acid

Potassium hydroxide (100 mg, 1.78 mmol) was added to a solution of (S)-isopropyl 2-(tert-butoxy)-2-(5-(2-(2-fluorobenzyl)-1,2,3,4-tetrahydroisoquinolin-6-yl)-2,6-dimethyl-4-(8-azaspiro[4.5]decan-8-yl)pyridin-3-yl)acetate (35 mg, 0.053 mmol) in EtOH (1 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(tert-butoxy)-2-(5-(2-(2-fluorobenzyl)-1,2,3,4-tetrahydroisoquinolin-6-yl)-2,6-dimethyl-4-(8-azaspiro[4.5]decan-8-yl)pyridin-3-yl)acetic acid 15.5 mg (45%). ¹H NMR (500 MHz, DMSO) δ 7.51-7.49 (m, 1H), 7.35 (br. s, 1H), 7.23-7.20 (m, 2H), 7.13-7.10 (m, 1H), 7.05-7.03 (m, 1H), 6.85-6.83 (m, 1H), 5.87/5.85 (s, 1H), 3.76-3.63 (m, 3H), 3.39 (br. s, 1H), 2.85-2.74 (m, 4H), 2.55 (s, 2H), 2.43 (s, 3H), 2.29-2.26/2.17-2.15 (m, 1H), 2.10/2.06 (s, 3H), 1.57-1.23 (series of m, 10H), 1.12 (s, 9H), 0.96 (br. s, 1H). UPLC (M+H)=614.4.

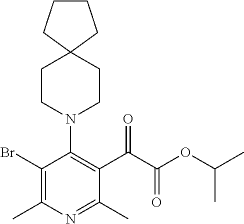 [SMILES: Cc1nc(C)c(C(=O)C(=O)OC(C)C)c(N2CCC3(CCCC3)CC2)c1Br]

### Isopropyl 2-(5-bromo-2,6-dimethyl-4-(7-azaspiro[3.5]nonan-7-yl)pyridin-3-yl)-2-oxoacetate

To a solution of 7-azaspiro[3.5]nonane (0.30 g, 2.396 mmol) and DIEA (1.3 mL, 7.19 mmol) in anhydrous CH₃CN (10 mL) was added isopropyl 2-(5-bromo-4-chloro-2,6-dimethylpyridin-3-yl)-2-oxoacetate (800 mg, 2.4 mmol) at rt. The resulting mixture was placed in a pre-heated oil bath (80° C.) and stirred for 18 h; cooled, and concentrated. The reaction mixture was diluted with ether, washed with water, brine, dried (MgSO₄), and charged (DCM) to a 80 g ISCO silica gel cartridge and gradient elution (0-20% EtOAc/hexanes) using an Isolera chromatography station to give isopropyl 2-(5-bromo-2,6-dimethyl-4-(7-azaspiro[3.5]nonan-7-yl)pyridin-3-yl)-2-oxoacetate 683 mg (67%). ¹H NMR (500 MHz, DMSO) δ 5.08-5.03 (m, 1H), 2.97 (br. s, 4H), 2.59 (s, 3H), 2.29 (s, 3H), 1.87-1.83 (m, 2H), 1.75-1.72 (m, 4H), 1.52 (br. s, 4H), 1.27 (d, J=6.2 Hz, 6H). UPLC (M+H)=425.3.

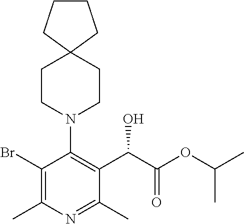 [SMILES: Cc1nc(C)c([C@H](O)C(=O)OC(C)C)c(N2CCC3(CCCC3)CC2)c1Br]

### (S)-Isopropyl 2-(5-bromo-2,6-dimethyl-4-(7-azaspiro[3.5]nonan-7-yl)pyridin-3-yl)-2-hydroxyacetate

The 0.63 mL of benzo[d][1,3,2]dioxaborole (702 mg, 2.93 mmol) was added to a nitrogen purged solution of isopropyl 2-(5-bromo-2,6-dimethyl-4-(7-azaspiro[3.5]nonan-7-yl)pyridin-3-yl)-2-oxoacetate (620 mg, 1.47 mmol) and 1M (R)-1-methyl-3,3-diphenylhexahydropyrrolo[1,2-c][1,3,2]oxazaborole (0.59 mL, 0.59 mmol) in toluene (15 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine, and dried (MgSO₄). The crude product was charged (DCM) to a 80 g ISCO silica gel cartridge and gradient elution (0-30% EtOAc/hexanes) using an Isolera chromatography station gave (S)-isopropyl 2-(5-bromo-2,6-dimethyl-4-(7-azaspiro[3.5]nonan-7-yl)pyridin-3-yl)-2-hydroxyacetate 620 mg (98%). ¹H NMR (500 MHz, DMSO) δ 5.85 (s, 1H), 4.97-4.92 (m, 1H), 3.50 (t, J=10.6 Hz, 1H), 3.30 (t, J=10.6 Hz, 1H), 2.75 (d, J=11.7 Hz, 1H), 2.66 (d, J=11.0 Hz, 1H), 2.51 (s, 3H), 2.37 (s, 3H), 1.89-1.82 (m, 4H), 1.75-1.55 (m, 6H), 1.14 (d, J=6.2 Hz, 3H), 1.07 (d, J=6.2 Hz, 3H). UPLC (M+H)=427.3.

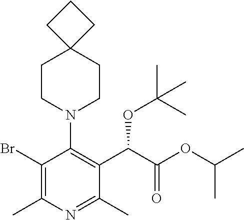 [SMILES: Cc1nc(C)c([C@H](OC(C)(C)C)C(=O)OC(C)C)c(N2CCC3(CCC3)CC2)c1Br]

### (S)-Isopropyl 2-(5-bromo-2,6-dimethyl-4-(7-azaspiro[3.5]nonan-7-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(7-azaspiro[3.5]nonan-7-yl)pyridin-3-yl)-2-hydroxyacetate (620 mg, 1.46 mmol) and 0.14 mL of 70% HClO₄ in DCM (10 mL) for 20 min. The reaction mixture was allowed to warm to rt and stirred for 48 h in a pressure sealed vessel. The reaction mixture was diluted with DCM, washed with 1M Na₂CO₃ soln, and dried over MgSO₄. The crude product was charged (DCM) to a 80 g ISCO silica gel cartridge and gradient elution (0-20% EtOAc/hexanes) using an Isolera chromatography station gave (S)-isopropyl 2-(5-bromo-2,6-dimethyl-4-(7-azaspiro[3.5]nonan-7-yl)pyridin-3-yl)-2-(tert-butoxy)acetate 440 mg (63%). ¹H NMR (500 MHz, DMSO) δ 6.16 (s, 1H), 4.93-4.88 (m, 1H), 3.77 (br. s, 1H), 3.23 (t, J=11.7 Hz, 1H), 2.81 (d, J=9.5 Hz, 1H), 2.57 (br. s, 1H), 2.51 (s, 3H), 2.41 (s, 3H), 1.88-1.70 (m, 8H), 1.59-1.52 (m, 2H), 1.15 (d, J=6.2 Hz, 3H), 1.12 (s, 9H), 1.06 (d, J=6.2 Hz, 3H). UPLC (M+H)=483.4.

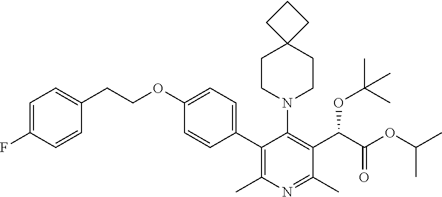 [SMILES: Cc1nc(C)c([C@H](OC(C)(C)C)C(=O)OC(C)C)c(N2CCC3(CCC3)CC2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(7-azaspiro[3.5]nonan-7-yl)pyridin-3-yl)acetate

The tretrakis (132 mg, 0.114 mmol) was added to an argon purged and degassed solution (S)-isopropyl 2-(5-bromo-2,6-dimethyl-4-(7-azaspiro[3.5]nonan-7-yl)pyridin-3-yl)-2-(tert-butoxy)acetate (275 mg, 0.571 mmol), (4-(4-fluorophenethoxy)phenyl)boronic acid (163 mg, 0.63 mmol), and potassium phosphate tribasic (909 mg, 4.3 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (5-50% EtOAc/hexanes) using an Isolera chromatography station gave (S)-isopropyl 2-(tert-butoxy)-2-(5-(4-(4-fluorophenethoxy)phenyl)-2,6-dimethyl-4-(7-azaspiro[3.5]nonan-7-yl)pyridin-3-yl)acetate 276 mg (78%). ¹H NMR (500 MHz, DMSO) δ 7.38-7.36 (m, 2H), 7.18 (d, J 8.1 Hz, 1H), 7.13 (t, J 8.8 Hz, 2H), 7.01-6.98 (m, 3H), 6.00 (s, 1H), 4.96-4.92 (m, 1H), 4.26-4.18 (m, 2H), 3.43-3.36 (m, 2H), 3.12-3.09 (m, 1H), 3.05 (t, J=6.6 Hz, 2H), 2.65-2.61 (m, 1H), 2.43 (s, 3H), 2.24-2.22 (m, 1H), 2.04 (s, 3H), 1.81-1.71 (m, 3H), 1.63-1.41 (m, 6H), 1.33-1.24 (m, 2H), 1.18 (d, J=6.2 Hz, 3H), 1.14-1.11 (m, 12H). UPLC (M+H)=617.6.

### Example 27

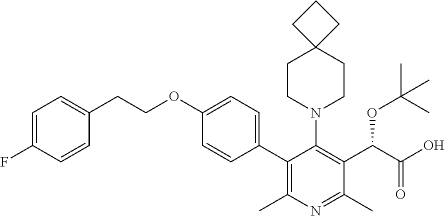 [SMILES: Cc1nc(C)c([C@H](OC(C)(C)C)C(=O)O)c(N2CCC3(CCC3)CC2)c1-c1ccc(OCCc2ccc(F)cc2)cc1]

### (S)-2-(tert-Butoxy)-2-(5-(4-(4-fluorophenethoxy)phenyl)-2,6-dimethyl-4-(7-azaspiro[3.5]nonan-7-yl)pyridin-3-yl)acetic acid

The potassium hydroxide (223 mg, 3.97 mmol) was added to a solution of (S)-isopropyl 2-(tert-butoxy)-2-(5-(4-(4-fluorophenethoxy)phenyl)-2,6-dimethyl-4-(7-azaspiro[3.5]nonan-7-yl)pyridin-3-yl)acetate (245 mg, 0.397 mmol) in ethanol (4 mL) and stirred for 4 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy) phenyl)-2,6-dimethyl-4-(7-azaspiro[3.5]nonan-7-yl)pyridin-3-yl)acetic acid 47 mg (20%). ¹H NMR (500 MHz, DMSO) δ 7.39-7.36 (m, 2H), 7.19 (d, J=8.1 Hz, 1H), 7.13 (t, J=8.8 Hz, 2H), 7.00-6.96 (m, 3H), 5.88 (s, 1H), 4.27-4.18 (m, 2H), 3.38 (br. s, 2H), 3.22 (br. s, 1H), 3.05 (t, J=6.2 Hz, 2H), 2.63 (br. s, 1H), 2.43 (s, 3H), 2.22 (br. s, 1H), 2.04 (s, 3H), 1.76-1.72 (m, 2H), 1.63-1.25 (m, 7H), 1.12 (s, 9H). UPLC (M+H)=475.5.

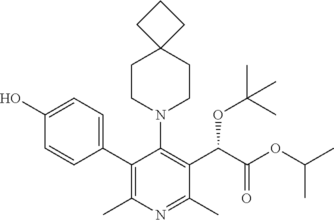 [SMILES: Cc1nc(C)c([C@H](OC(C)(C)C)C(=O)OC(C)C)c(N2CCC3(CCC3)CC2)c1-c1ccc(O)cc1]

### (S)-Isopropyl 2-(tert-butoxy)-2-(5-(4-hydroxyphenyl)-2,6-dimethyl-4-(7-azaspiro[3.5]nonan-7-yl)pyridin-3-yl)acetate

The Pd(Ph₃P)₄ (144 mg, 0.125 mmol) was added to an argon purged and degassed solution of (S)-isopropyl 2-(5-bromo-2,6-dimethyl-4-(7-azaspiro[3.5]nonan-7-yl)pyridin-3-yl)-2-(tert-butoxy)acetate (300 mg, 0.623 mmol), (4-((tert-butyldimethylsilyl)oxy)phenyl)boronic acid (173 mg, 0.69 mmol), and potassium phosphate tribasic (992 mg, 4.67 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO4). UPLC (M+H)=609.5. The crude silyl ether was taken up in dry THF (4 mL) and treated with TBAF (0.685 ml, 0.685 mmol) at 0° C. The mixture was allowed to warm and stirred at rt for 2 h before it was diluted with EtOAc and water. The organic layer was separated, washed with brine, dried over MgSO₄. The crude product was charged (DCM) to a 40 g ISCO silica gel cartridge and gradient elution (0-50% EtOAc/hexanes) using an Isolera chromatography station gave (S)-isopropyl 2-(tert-butoxy)-2-(5-(4-hydroxyphenyl)-2,6-dimethyl-4-(7-azaspiro[3.5]nonan-7-yl)pyridin-3-yl)acetate 234 mg (76%). ¹H NMR (500 MHz, DMSO) δ 7.06 (d, J=8.4 Hz, 1H), 6.87-6.81 (m, 3H), 6.01 (s, 1H), 4.97-4.92 (m, 1H), 3.09 (d, J=10.6 Hz, 1H), 2.65 (t, J=11.0 Hz, 1H), 2.42 (s, 3H), 2.23 (d, J=12.5 Hz, 1H), 2.06 (s, 3H), 1.85 (t, J=11.7 Hz, 1H), 1.77-1.29 (series of m, 12H), 1.19 (d, J=6.2 Hz, 3H), 1.14-1.12 (m, 12H). UPLC (M+H)=495.3.

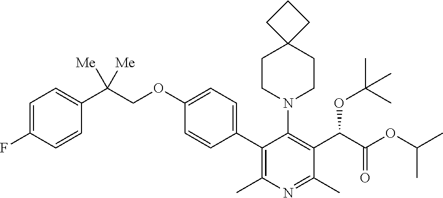 [SMILES: Cc1nc(C)c([C@H](OC(C)(C)C)C(=O)OC(C)C)c(N2CCC3(CCC3)CC2)c1-c1ccc(OCC(C)(C)c2ccc(F)cc2)cc1]

### (S)-Isopropyl 2-(tert-butoxy)-2-(5-(4-(2-(4-fluorophenyl)-2-methylpropoxy)phenyl)-2,6-dimethyl-4-(7-azaspiro[3.5]nonan-7-yl)pyridin-3-yl)acetate

To a stirred solution of (S)-isopropyl 2-(tert-butoxy)-2-(5-(4-hydroxyphenyl)-2,6-dimethyl-4-(7-azaspiro[3.5]nonan-7-yl)pyridin-3-yl)acetate (100 mg, 0.20 mmol), 2-(4-fluorophenyl)-2-methylpropan-1-ol (170 mg, 1.01 mmol) and triphenylphosphine (266 mg, 1.01 mmol) in dry THF (2 mL) was added DIAD (0.197 mL, 1.03 mmol) at rt and the solution was heated at 70° C. for 16 h in a screw capped vessel. An additional equivalent of reagents was added at rt and the reaction mixture was heated at 70° C. for 16 h, cooled, diluted with EtOAc, washed with brine, and dried (MgSO₄). The crude product was charged (DCM) to a 40 g ISCO silica gel cartridge and gradient elution (0-50% EtOAc/hexanes) using an Isolera chromatography station gave (S)-isopropyl 2-(tert-butoxy)-2-(5-(4-(2-(4-fluorophenyl)-2-methylpropoxy)phenyl)-2,6-dimethyl-4-(7-azaspiro[3.5]nonan-7-yl)pyridin-3-yl)acetate 41 mg (31%). UPLC (M+H)=645.5.

### Example 28

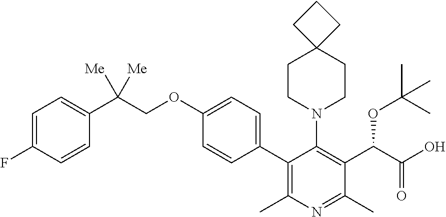 [SMILES: Cc1nc(C)c([C@H](OC(C)(C)C)C(=O)O)c(N2CCC3(CCC3)CC2)c1-c1ccc(OCC(C)(C)c2ccc(F)cc2)cc1]

Potassium hydroxide (114 mg, 2.06 mmol) was added to a solution of (S)-isopropyl 2-(tert-butoxy)-2-(5-(4-(2-(4-fluorophenyl)-2-methylpropoxy)phenyl)-2,6-dimethyl-4-(7-azaspiro[3.5]nonan-7-yl)pyridin-3-yl)acetate (41 mg, 0.064 mmol) in absolute EtOH (2 mL) and stirred for 6 h at 90° C. The mixture was diluted with EtOAc (15 mL) and neutralized with 1N HCl (4 mL) to pH 4. Buffer (5 mL, pH=5) was added and the mixture extracted with EtOAc. The organic layer was washed with brine and dried (MgSO₄). The crude product was purified by prep HPLC to obtain (S)-2-(tert-butoxy)-2-(5-(4-(2-(4-fluorophenyl)-2-methylpropoxy)phenyl)-2,6-dimethyl-4-(7-azaspiro[3.5]nonan-7-yl)pyridin-3-yl)acetic acid 17.7 mg (44%). ¹H NMR (500 MHz, DMSO) δ 7.53-7.50 (m, 2H), 7.17-7.11 (m, 3H), 6.98-6.95 (m, 3H), 5.81 (s, 1H), 4.08, 4.02 (AB, J_{AB}=9.2 Hz, 2H), 3.32-3.28 (m, 2H), 2.60 (br. s, 1H), 2.42 (s, 3H), 2.21-2.19 (m, 1H), 2.03 (s, 3H), 1.76-1.23 (series of m, 10H), 1.41 (s, 6H), 1.11 (s, 9H). UPLC (M+H)=603.4.

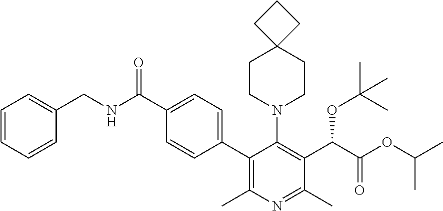 [SMILES: Cc1nc(C)c([C@H](OC(C)(C)C)C(=O)OC(C)C)c(N2CCC3(CCC3)CC2)c1-c1ccc(C(=O)NCc2ccccc2)cc1]

### (S)-Isopropyl 2-(5-(4-(benzylcarbamoyl)phenyl)-2,6-dimethyl-4-(7-azaspiro[3.5]nonan-7-yl)pyridin-3-yl)-2-(tert-butoxy)acetate

The tretrakis (132 mg, 0.114 mmol) was added to an argon purged and degassed solution (S)-isopropyl 2-(5-bromo-2,6-dimethyl-4-(7-azaspiro[3.5]nonan-7-yl)pyridin-3-yl)-2-(tert-butoxy)acetate (275 mg, 0.571 mmol), (4-(benzylcarbamoyl)phenyl)boronic acid (160 mg, 0.63 mmol), and potassium phosphate tribasic (909 mg, 4.3 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (5-50% EtOAc/hexanes) using an Isolera chromatography station gave (S)-isopropyl 2-(5-(4-(benzylcarbamoyl)phenyl)-2,6-dimethyl-4-(7-azaspiro[3.5]nonan-7-yl)pyridin-3-yl)-2-(tert-butoxy)acetate 292 mg (84%). ¹H NMR (500 MHz, DMSO) δ 9.17-9.14 (m, 1H), 7.99 (d, J=8.1 Hz, 2H), 7.45 (d, J=7.7 Hz, 1H), 7.34-7.31 (m, 4H), 7.26-7.23 (m, 1H), 7.21 (d, J=7.7 Hz, 1H), 5.99 (s, 1H), 4.98-4.94 (m, 1H), 4.52 (d, J=5.9 Hz, 2H), 3.44-3.42 (m, 1H), 3.15 (d, J=10.6 Hz, 1H), 2.64 (t, J=11 Hz, 1H), 2.46 (s, 3H), 2.27 (d, J=11.4 Hz, 1H), 2.04 (s, 3H), 1.72-1.22 (m, 10H), 1.19 (d, J=6.2 Hz, 3H), 1.15 (d, J=6.2 Hz, 3H), 1.12 (s, 9H). UPLC (M+H)=612.6.

### Example 29

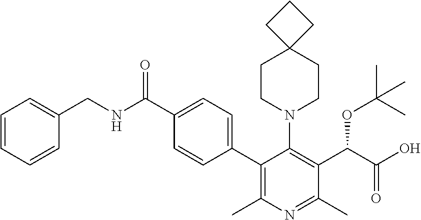 [SMILES: Cc1nc(C)c([C@H](OC(C)(C)C)C(=O)O)c(N2CCC3(CCC3)CC2)c1-c1ccc(C(=O)NCc2ccccc2)cc1]

### (S)-2-(5-(4-(Benzylcarbamoyl)phenyl)-2,6-dimethyl-4-(7-azaspiro[3.5]nonan-7-yl)pyridin-3-yl)-2-(tert-butoxy)acetic acid

The potassium hydroxide (243 mg, 4.33 mmol) was added to a solution of (S)-isopropyl 2-(5-(4-(benzylcarbamoyl)phenyl)-2,6-dimethyl-4-(7-azaspiro[3.5]nonan-7-yl)pyridin-3-yl)-2-(tert-butoxy) acetate (245 mg, 0.397 mmol) in ethanol (4 mL) and stirred for 4 h at 90° C. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(7-azaspiro [3.5]nonan-7-yl)pyridin-3-yl)-2-(tert-butoxy)acetic acid 41 mg (17%). ¹H NMR (500 MHz, DMSO) δ 9.17-9.15 (m, 1H), 7.98 (d, J=7.9 Hz, 2H), 7.46 (d, J=7.6 Hz, 1H), 7.34-7.32 (m, 4H), 7.26-7.24 (m, 1H), 7.19 (d, J=7.6 Hz, 1H), 5.82 (s, 1H), 4.52 (d, J=5.5 Hz, 2H), 3.36-3.34 (m, 2H), 2.6 (br. S, 1H), 2.46 (s, 3H), 2.26-2.24 (m, 1H), 2.04 (s, 3H), 1.72-1.47 (m, 8H), 1.39-1.21 (m, 2H), 1.12 (s, 9H). UPLC (M+H)=570.5.

### Example 30

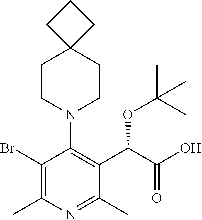 [SMILES: Cc1nc(C)c([C@H](OC(C)(C)C)C(=O)O)c(N2CCC3(CCC3)CC2)c1Br]

### (S)-2-(5-Bromo-2,6-dimethyl-4-(7-azaspiro[3.5]nonan-7-yl)pyridin-3-yl)-2-(tert-butoxy)acetic acid

Potassium hydroxide (583 mg, 10.40 mmol) was added to a solution of (S)-isopropyl 2-(5-bromo-2,6-dimethyl-4-(7-azaspiro[3.5]nonan-7-yl)pyridin-3-yl)-2-(tert-butoxy)acetate (500 mg, 1.04 mmol) in absolute EtOH (10 mL) and stirred for 6 h at 90° C. and then at rt for 10 h. The mixture was diluted with EtOAc (15 mL) and neutralized with 1N HCl (4 mL) to pH 4. Buffer (5 mL, pH=5) was added and the mixture extracted with EtOAc. The organic layer was washed with brine and dried (MgSO₄). The crude product (S)-2-(5-bromo-2,6-dimethyl-4-(7-azaspiro[3.5]nonan-7-yl)pyridin-3-yl)-2-(tert-butoxy)acetic acid was carried forward (assume theoretical yield). UPLC (M+H)=441.2.

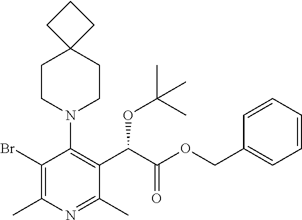 [SMILES: Cc1nc(C)c([C@H](OC(C)(C)C)C(=O)OCc2ccccc2)c(N2CCC3(CCC3)CC2)c1Br]

### (S)-Benzyl 2-(5-bromo-2,6-dimethyl-4-(7-azaspiro[3.5]nonan-7-yl)pyridin-3-yl)-2-(tert-butoxy)acetate

Benzylbromide (0.14 mL, 1.14 mmol) was added to a stirred suspension (S)-2-(5-bromo-2,6-dimethyl-4-(7-azaspiro[3.5]nonan-7-yl)pyridin-3-yl)-2-(tert-butoxy)acetic acid (456 mg, 1.04 mmol) and cesium carbonate (338 mg, 1.04 mmol) in anhydrous acetonitrile (10 mL). The mixture was stirred for 12 h at rt, filtered, and the filtrate was charged (DCM) to a 40 g ISCO silica gel cartridge and gradient elution (0-25% EtOAc/hexanes) using an Isolera chromatography station gave (S)-benzyl 2-(5-bromo-2,6-dimethyl-4-(7-azaspiro[3.5]nonan-7-yl)pyridin-3-yl)-2-(tert-butoxy)acetate 450 mg (82%). ¹H NMR (500 MHz, DMSO) δ 7.37-7.33 (m, 3H), 7.27-7.25 (m, 2H), 6.06 (br. s, 1H), 5.19, 5.05 (AB, J_{AB}=12.5 Hz, 2H), 3.71 (t, J=8.8 Hz, 1H), 3.35 (br. s, 1H), 3.18-3.10 (m, 1H), 2.72-2.69 (m, 1H), 2.51 (s, 3H), 2.40 (s, 3H), 1.88-1.66 (series of m, 7H), 1.53-1.48 (m, 3H), 1.13 (s, 9H). UPLC (M+H)=531.3.

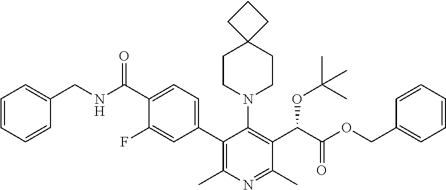 [SMILES: Cc1nc(C)c([C@H](OC(C)(C)C)C(=O)OCc2ccccc2)c(N2CCC3(CCC3)CC2)c1-c1ccc(C(=O)NCc2ccccc2)c(F)c1]

### (S)-Benzyl 2-(5-(4-(benzylcarbamoyl)-3-fluorophenyl)-2,6-dimethyl-4-(7-azaspiro[3.5]nonan-7-yl)pyridin-3-yl)-2-(tert-butoxy)acetate

The Pd(Ph₃P)₄ (103 mg, 0.09 mmol) was added to an argon purged and degassed solution of (S)-benzyl 2-(5-bromo-2,6-dimethyl-4-(7-azaspiro[3.5]nonan-7-yl)pyridin-3-yl)-2-(tert-butoxy)acetate (237 mg, 0.45 mmol), (4-(benzylcarbamoyl)-3-fluorophenyl)boronic acid (134 mg, 0.49 mmol), and sodium carbonate (237 mg, 2.24 mmol) in dioxane (3 mL) and water (0.75 mL) and stirred in a screw-capped pressure vessel for 16 h at 85° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-benzyl 2-(5-(4-(benzylcarbamoyl)-3-fluorophenyl)-2,6-dimethyl-4-(7-azaspiro[3.5]nonan-7-yl)pyridin-3-yl)-2-(tert-butoxy)acetate 220 mg (73%). UPLC (M+H)=678.4.

### Example 31

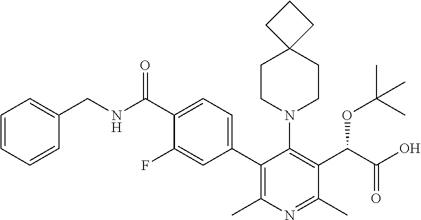 [SMILES: Cc1nc(C)c([C@H](OC(C)(C)C)C(=O)O)c(N2CCC3(CCC3)CC2)c1-c1ccc(C(=O)NCc2ccccc2)c(F)c1]

### (S)-2-(5-(4-(Benzylcarbamoyl)-3-fluorophenyl)-2,6-dimethyl-4-(7-azaspiro[3.5]nonan-7-yl)pyridin-3-yl)-2-(tert-butoxy)acetic acid

The a solution of (S)-benzyl 2-(5-(4-(benzylcarbamoyl)-3-fluorophenyl)-2,6-dimethyl-4-(7-azaspiro[3.5]nonan-7-yl)pyridin-3-yl)-2-(tert-butoxy)acetate (80 mg, 0.12 mmol) in MeOH (0.5 mL) was added to a suspension of Pearlman's Catalyst (16 mg, 0.12 mmol) in dry MeOH (1.5 mL) at rt. The flask was evacuated and charged with hydrogen (ballon) and stirred for 2 h, filtered, and concentrated. The crude product was purified by prep HPLC to obtain (S)-2-(5-(4-(benzylcarbamoyl)-3-fluorophenyl)-2,6-dimethyl-4-(7-azaspiro[3.5]nonan-7-yl)pyridin-3-yl)-2-(tert-butoxy)acetic acid 41.6 mg (60%). ¹H NMR (500 MHz, DMSO) δ 8.95 (s, 1H), 7.75-7.71 (m, 1H), 7.42-7.34 (m, 4.5H), 7.29-7.26 (m, 1.5H), 7.09/7.02 (d, J=10.3 Hz, 1H), 5.82 (s, 1H), 4.51 (d, J=5.9 Hz, 2H), 3.36-3.31 (m, 2H), 2.63 (br. s, 1H), 2.45 (s, 3H), 2.34-2.26 (m, 1H), 2.08/2.07 (s, 3H), 1.78-1.72 (m, 2H), 1.67-1.31 (series of m, 8H), 1.13 (m, 9H). UPLC (M+H)=588.4.

### Example 32

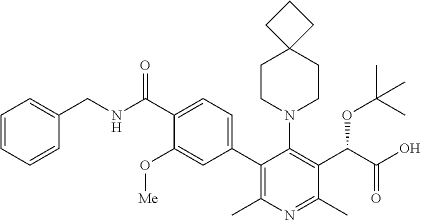 [SMILES: COc1cc(-c2c(C)nc(C)c([C@H](OC(C)(C)C)C(=O)O)c2N2CCC3(CCC3)CC2)ccc1C(=O)NCc1ccccc1]

### (S)-2-(5-(4-(Benzylcarbamoyl)-3-methoxyphenyl)-2,6-dimethyl-4-(7-azaspiro[3.5]nonan-7-yl)pyridin-3-yl)-2-(tert-butoxy)acetic acid

Potassium hydroxide (64 mg, 1.14 mmol) was added to a solution of (S)-benzyl 2-(5-(4-(benzylcarbamoyl)-3-fluorophenyl)-2,6-dimethyl-4-(7-azaspiro[3.5]nonan-7-yl)pyridin-3-yl)-2-(tert-butoxy)acetate (74 mg, 0.114 mmol) in MeOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(5-(4-(benzylcarbamoyl)-3-methoxyphenyl)-2,6-dimethyl-4-(7-azaspiro[3.5]nonan-7-yl)pyridin-3-yl)-2-(tert-butoxy)acetic acid 38 mg (55%). UPLC (M+H)=600.4.

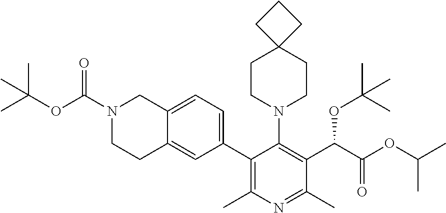 [SMILES: Cc1nc(C)c([C@H](OC(C)(C)C)C(=O)OC(C)C)c(N2CCC3(CCC3)CC2)c1-c1ccc2c(c1)CCN(C(=O)OC(C)(C)C)C2]

### (S)-tert-Butyl 6-(5-(1-(tert-butoxy)-2-isopropoxy-2-oxoethyl)-2,6-dimethyl-4-(7-azaspiro[3.5]nonan-7-yl)pyridin-3-yl)-3,4-dihydroisoquinoline-2(1H)-carboxylate

The Pd(Ph₃P)₄ (72 mg, 0.062 mmol) was added to an argon purged and degassed solution of (S)-isopropyl 2-(5-bromo-2,6-dimethyl-4-(7-azaspiro[3.5]nonan-7-yl)pyridin-3-yl)-2-(tert-butoxy)acetate (300 mg, 0.62 mmol), tert-butyl 6-(4,4,5,5-tetramethyl-1,3,2-dioxaborolan-2-yl)-3,4-dihydroisoquinoline-2(1H)-carboxylate (246 mg, 0.62 mmol), and sodium carbonate (330 mg, 3.12 mmol) in dioxane (4 mL) and water (1 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-tert-butyl 6-(5-(1-(tert-butoxy)-2-isopropoxy-2-oxoethyl)-2,6-dimethyl-4-(7-azaspiro[3.5]nonan-7-yl)pyridin-3-yl)-3,4-dihydroisoquinoline-2(1H)-carboxylate 280 mg (71%). UPLC (M+H)=634.4.

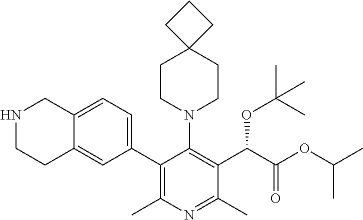 [SMILES: Cc1nc(C)c([C@H](OC(C)(C)C)C(=O)OC(C)C)c(N2CCC3(CCC3)CC2)c1-c1ccc2c(c1)CCNC2]

### (S)-Isopropyl 2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetate

The (S)-tert-butyl 6-(5-(1-(tert-butoxy)-2-isopropoxy-2-oxoethyl)-2,6-dimethyl-4-(7-azaspiro[3.5]nonan-7-yl)pyridin-3-yl)-3,4-dihydroisoquinoline-2(1H)-carboxylate (280 mg, 0.442 mmol) was dissolved in cold (0° C.) 4N HCl in dioxane (3 mL) and the solution was stirred at rt for 2 h and concentrated to remove solvent. There was obtained (S)-isopropyl 2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetate, 2 HCl 268 mg (99%). ¹H NMR (500 MHz, DMSO) δ 7.10-7.07 (m, 1H), 7.03-6.99 (m, 1H), 6.82-6.79 (m, 1H), 5.98 (s, 1H), 4.98-4.94 (m, 1H), 3.92 (s, 2H), 3.15-2.92 (m, 3H), 2.74-2.61 (m, 3H), 2.43 (s, 3H), 2.27/2.14 (d, J=12 Hz, 1H), 2.10/2.06 (s, 3H), 1.77-1.30 (m, 11H), 1.20-1.18 (m, 3H), 1.16-1.11 (s, 12H). UPLC (M+H)=534.4.

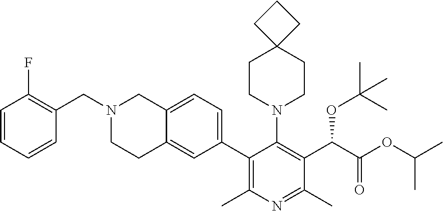 [SMILES: Cc1nc(C)c([C@H](OC(C)(C)C)C(=O)OC(C)C)c(N2CCC3(CCC3)CC2)c1-c1ccc2c(c1)CCN(Cc1ccccc1F)C2]

### (S)-Isopropyl 2-(tert-butoxy)-2-(5-(2-(2-fluorobenzyl)-1,2,3,4-tetrahydroisoquinolin-6-yl)-2,6-dimethyl-4-(7-azaspiro[3.5]nonan-7-yl)pyridin-3-yl)acetate

A suspension of (S)-isopropyl 2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetate, 2 HCl (75 mg, 0.124 mmol), sodium acetate (20.3 mg, 0.25 mmol), and 2-fluorobenzaldehyde (0.026 mL, 0.25 mmol) was stirred for 2 h in anhydrous DMF (2 mL). Sodium cyanoborohydride (38.8 mg, 0.62 mmol) was added in one portion and stirring was continued at rt for 16 h. The reaction mixture was diluted with EtOAc, washed with sat'd NaHCO₃ soln, brine, dried (MgSO₄). The crude product was subjected to prep HPLC to obtain (S)-isopropyl 2-(tert-butoxy)-2-(5-(2-(2-fluorobenzyl)-1,2,3,4-tetrahydroisoquinolin-6-yl)-2,6-dimethyl-4-(7-azaspiro[3.5]nonan-7-yl)pyridin-3-yl)acetate 27 mg (34%). UPLC (M+H)=642.5.

### Example 33

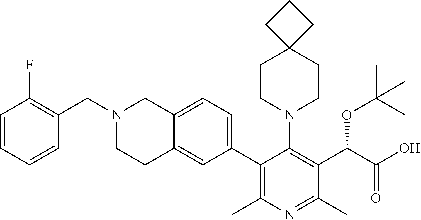 [SMILES: Cc1nc(C)c([C@H](OC(C)(C)C)C(=O)O)c(N2CCC3(CCC3)CC2)c1-c1ccc2c(c1)CCN(Cc1ccccc1F)C2]

### (S)-2-(tert-Butoxy)-2-(5-(2-(2-fluorobenzyl)-1,2,3,4-tetrahydroisoquinolin-6-yl)-2,6-dimethyl-4-(7-azaspiro[3.5]nonan-7-yl)pyridin-3-yl)acetic acid

Potassium hydroxide (69 mg, 1.24 mmol) was added to a solution of (S)-isopropyl 2-(tert-butoxy)-2-(5-(2-(2-fluorobenzyl)-1,2,3,4-tetrahydroisoquinolin-6-yl)-2,6-dimethyl-4-(7-azaspiro[3.5]nonan-7-yl)pyridin-3-yl)acetate (27 mg, 0.042 mmol) in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(tert-butoxy)-2-(5-(2-(2-fluorobenzyl)-1,2,3,4-tetrahydro-isoquinolin-6-yl)-2,6-dimethyl-4-(7-azaspiro[3.5]nonan-7-yl)pyridin-3-yl)acetic acid 12.6 mg (16.5%). ¹H NMR (500 MHz, DMSO) δ 7.49 (t, J=5.9 Hz, 1H), 7.35 (br. s, 1H), 7.22-7.19 (m, 2H), 7.10 (t, J 8.4 Hz, 1H), 7.04-7.03 (m, 1H), 6.81-6.80 (m, 1H), 5.85/5.84 (s, 1H), 3.74-3.73 (m, 2H), 3.66-3.65 (m, 2H), 3.32-3.21 (m, 1H), 2.85-2.65 (m, 5H), 2.43 (s, 3H), 2.25-2.22/2.12-2.10 (m, 1H), 2.09/2.05 (s, 3H), 1.76-1.24 (series of m, 11H), 1.11 (s, 9H). UPLC (M+H)=600.4.

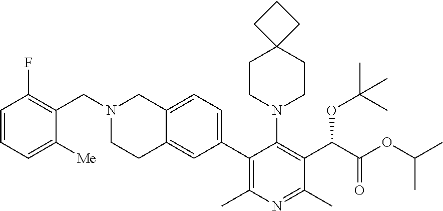 [SMILES: Cc1cccc(F)c1CN1CCc2cc(-c3c(C)nc(C)c([C@H](OC(C)(C)C)C(=O)OC(C)C)c3N3CCC4(CCC4)CC3)ccc2C1]

### (S)-Isopropyl 2-(tert-butoxy)-2-(5-(2-(2-fluoro-6-methylbenzyl)-1,2,3,4-tetrahydroisoquinolin-6-yl)-2,6-dimethyl-4-(7-azaspiro[3.5]nonan-7-yl)pyridin-3-yl)acetate

A suspension of (S)-isopropyl 2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetate, 2 HCl (75 mg, 0.124 mmol), sodium acetate (20.3 mg, 0.25 mmol), and 2-fluoro-4-methylbenzaldehyde (0.03 mL, 0.25 mmol) was stirred for 2 h in anhydrous DMF (2 mL). Sodium cyanoborohydride (38.8 mg, 0.62 mmol) was added in one portion and stirring was continued at rt for 16 h. The reaction mixture was diluted with EtOAc, washed with sat'd NaHCO₃ soln, brine, dried (MgSO₄). The crude product was subjected to prep HPLC to obtain (S)-isopropyl 2-(tert-butoxy)-2-(5-(2-(2-fluoro-6-methylbenzyl)-1,2,3,4-tetrahydroisoquinolin-6-yl)-2,6-dimethyl-4-(7-azaspiro[3.5]nonan-7-yl)pyridin-3-yl)acetate 34 mg (42%). UPLC (M+H)=656.4.

### Example 34

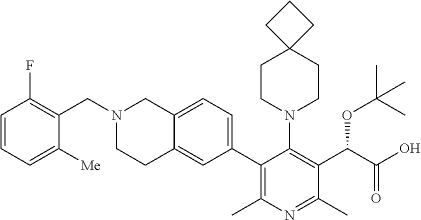 [SMILES: Cc1cccc(F)c1CN1CCc2cc(-c3c(C)nc(C)c([C@H](OC(C)(C)C)C(=O)O)c3N3CCC4(CCC4)CC3)ccc2C1]

### (S)-2-(tert-Butoxy)-2-(5-(2-(2-fluoro-6-methylbenzyl)-1,2,3,4-tetrahydroisoquinolin-6-yl)-2,6-dimethyl-4-(7-azaspiro[3.5]nonan-7-yl)pyridin-3-yl)acetic acid

Potassium hydroxide (69 mg, 1.24 mmol) was added to a solution of (S)-isopropyl 2-(tert-butoxy)-2-(5-(2-(2-fluoro-6-methylbenzyl)-1,2,3,4-tetrahydroisoquinolin-6-yl)-2,6-dimethyl-4-(7-azaspiro[3.5]nonan-7-yl)pyridin-3-yl)acetate (34 mg, 0.052 mmol) in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(tert-butoxy)-2-(5-(2-(2-fluoro-6-methyl benzyl)-1,2,3,4-tetrahydroisoquinolin-6-yl)-2,6-dimethyl-4-(7-azaspiro[3.5]nonan-7-yl)pyridin-3-yl)acetic acid 17 mg (22%). ¹H NMR (500 MHz, DMSO) δ 7.26-7.22 (m, 1H), 7.11-7.01 (m, 4H), 6.81-6.79 (m, 1H), 5.85/5.83 (s, 1H), 3.68 (s, 2H), 3.63 (s, 2H), 3.31-3.18 (m, 1H), 2.81-2.61 (m, 5H), 2.43 (s, 3H), 2.41 (s, 3H), 2.11/2.05 (s, 3H), 1.75-1.73 (m, 2H), 1.64-1.24 (series of m, 10H), 1.11 (s, 9H). UPLC (M+H)=614.4.

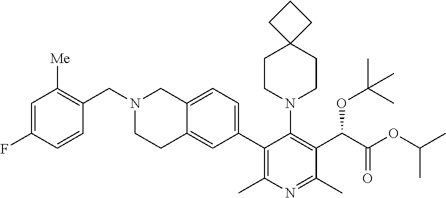 [SMILES: Cc1cc(F)ccc1CN1CCc2cc(-c3c(C)nc(C)c([C@H](OC(C)(C)C)C(=O)OC(C)C)c3N3CCC4(CCC4)CC3)ccc2C1]

### (S)-Isopropyl 2-(tert-butoxy)-2-(5-(2-(4-fluoro-2-methylbenzyl)-1,2,3,4-tetrahydroisoquinolin-6-yl)-2,6-dimethyl-4-(7-azaspiro[3.5]nonan-7-yl)pyridin-3-yl)acetate

A suspension of (S)-isopropyl 2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetate, 2 HCl (60 mg, 0.10 mmol), sodium acetate (16.2 mg, 0.20 mmol), and 4-fluoro-2-methylbenzaldehyde (0.024 mL, 0.20 mmol) was stirred for 2 h in anhydrous DMF (2 mL). Sodium cyanoborohydride (31 mg, 0.50 mmol) was added in one portion and stirring was continued at rt for 16 h. The reaction mixture was diluted with EtOAc, washed with sat'd NaHCO₃ soln, brine, dried (MgSO₄). The crude product was subjected to prep HPLC to obtain (S)-isopropyl 2-(tert-butoxy)-2-(5-(2-(4-fluoro-2-methylbenzyl)-1,2,3,4-tetrahydroisoquinolin-6-yl)-2,6-dimethyl-4-(7-azaspiro[3.5]nonan-7-yl)pyridin-3-yl)acetate 22.5 mg (35%). UPLC (M+H)=656.5.

### Example 35

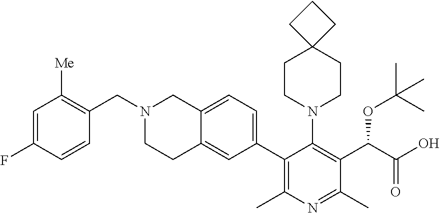 [SMILES: Cc1cc(F)ccc1CN1CCc2cc(-c3c(C)nc(C)c([C@H](OC(C)(C)C)C(=O)O)c3N3CCC4(CCC4)CC3)ccc2C1]

### (S)-2-(tert-Butoxy)-2-(5-(2-(4-fluoro-2-methylbenzyl)-1,2,3,4-tetrahydroisoquinolin-6-yl)-2,6-dimethyl-4-(7-azaspiro[3.5]nonan-7-yl)pyridin-3-yl)acetic acid

Potassium hydroxide (56 mg, 0.99 mmol) was added to a solution of (S)-isopropyl 2-(tert-butoxy)-2-(5-(2-(4-fluoro-2-methylbenzyl)-1,2,3,4-tetrahydroisoquinolin-6-yl)-2,6-dimethyl-4-(7-azaspiro[3.5]nonan-7-yl)pyridin-3-yl)acetate (22.5 mg, 0.034 mmol) in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(tert-butoxy)-2-(5-(2-(4-fluoro-2-methylbenzyl)-1,2,3,4-tetrahydroisoquinolin-6-yl)-2,6-dimethyl-4-(7-azaspiro[3.5]nonan-7-yl)pyridin-3-yl)acetic acid 17.5 mg (29%). ¹H NMR (500 MHz, DMSO) δ 7.34-7.31 (m, 1H), 7.10-7.02 (m, 3H), 7.00-6.98 (m, 1H), 6.80 (s, 1H), 5.85/5.83 (s, 1H), 3.62-3.61 (m, 4H), 3.32-3.20 (m, 1H), 2.84-2.79 (m, 2H), 2.72-2.62 (m, 3H), 2.43 (s, 3H), 2.37/2.35 (s, 3H), 2.41 (s, 3H), 2.11/2.06 (s, 3H), 1.76-1.73 (m, 2H), 1.65-1.24 (series of m, 10H), 1.11 (s, 9H). UPLC (M+H)=614.5.

### Example 36

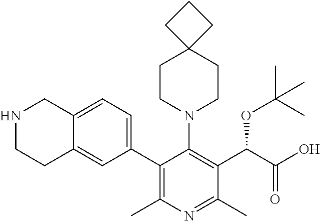 [SMILES: Cc1nc(C)c([C@H](OC(C)(C)C)C(=O)O)c(N2CCC3(CCC3)CC2)c1-c1ccc2c(c1)CCNC2]

### (S)-2-(tert-Butoxy)-2-(2,6-dimethyl-4-(7-azaspiro[3.5]nonan-7-yl)-5-(1,2,3,4-tetrahydroisoquinolin-6-yl)pyridin-3-yl)acetic acid

Potassium hydroxide (74 mg, 1.32 mmol) was added to a solution of (S)-isopropyl 2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetate, 2 HCl (80 mg, 0.132 mmol) in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). NOTE: Compound water soluble, therefore the aqueous layer was partially concentrated and the slurry was suction filtered. The filtrate was taken up in MeOH and combined with organic layer above. The crude product was subjected to prep HPLC to obtain (S)-2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetic acid 33 mg (47%). ¹H NMR (500 MHz, MeOD) δ 7.47-7.43 (m, 1H), 7.40-7.38 (m, 1H), 7.09-7.07 (m, 1H), 5.70/5.69 (s, 1H), 4.55-4.46 (m, 2H), 3.64-3.56 (m, 2H), 3.34-3.32 (m, 4H), 3.26-3.17 (m, 2H), 2.77 (s, 3H), 2.35/2.34 (s, 3H), 1.91-1.83 (m, 2H), 1.75-1.60 (series of m, 8H), 1.23 (s, 9H). UPLC (M+H)=492.3.

### Example 37

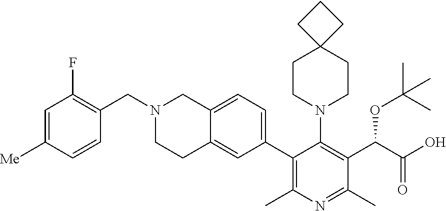 [SMILES: Cc1ccc(CN2CCc3cc(-c4c(C)nc(C)c([C@H](OC(C)(C)C)C(=O)O)c4N4CCC5(CCC5)CC4)ccc3C2)c(F)c1]

### (S)-2-(tert-Butoxy)-2-(5-(2-(2-fluoro-4-methylbenzyl)-1,2,3,4-tetrahydroisoquinolin-6-yl)-2,6-dimethyl-4-(7-azaspiro[3.5]nonan-7-yl)pyridin-3-yl)acetic acid

A suspension of (S)-2-(tert-butoxy)-2-(2,6-dimethyl-4-(7-azaspiro[3.5]nonan-7-yl)-5-(1,2,3,4-tetrahydroisoquinolin-6-yl)pyridin-3-yl)acetic acid (34 mg, 0.07 mmol), acetic acid (0.004 mL, 0.20 mmol), and 2-fluoro-4-methylbenzaldehyde (0.014 mL, 0.14 mmol) was stirred for 2 h in anhydrous DMF (2 mL). Sodium cyanoborohydride (22 mg, 0.35 mmol) was added in one portion and stirring was continued at rt for 16 h. The reaction mixture was diluted with EtOAc, washed with sat'd NaHCO₃ soln, brine, dried (MgSO₄). The crude product was purified by prep HPLC to obtain (S)-2-(tert-butoxy)-2-(5-(2-(2-fluoro-4-methylbenzyl)-1,2,3,4-tetrahydroisoquinolin-6-yl)-2,6-dimethyl-4-(7-azaspiro[3.5]nonan-7-yl)pyridin-3-yl)acetic acid 7.2 mg (17%). ¹H NMR (500 MHz, DMSO) δ 7.38-7.33 (m, 1H), 7.09 (t, J=7.7 Hz, 1H), 7.04-7.02 (m, 3H), 6.80 (br. s, 1H), 5.78/5.76 (s, 1H), 3.69-3.68 (m, 2H), 3.64-3.62 (m, 2H), 2.85-2.79 (m, 2H), 2.72-2.62 (m, 4H), 2.55 (s, 2H), 2.42 (s, 3H), 2.32 (s, 3H), 2.09/2.04 (s, 3H), 1.76-1.73 (m, 2H), 1.65-1.29 (series of m, 8H), 1.11 (s, 9H). UPLC (M+H)=614.4.

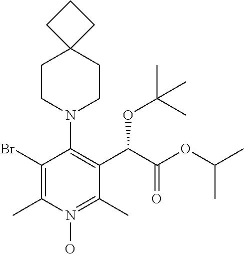

### (S)-3-Bromo-5-(1-(tert-butoxy)-2-isopropoxy-2-oxoethyl)-2,6-dimethyl-4-(7-azaspiro[3.5]nonan-7-yl)pyridine 1-oxide

To a stirred solution of (S)-isopropyl 2-(5-bromo-2,6-dimethyl-4-(7-azaspiro[3.5]nonan-7-yl)pyridin-3-yl)-2-(tert-butoxy)acetate (2.80 g, 5.82 mmol) in dry DCM (60 mL) was added m-CPBA (1.95 g, 8.72 mmol) at rt. The reaction mixture was stirred 2 h, diluted with DCM (60 mL), washed with sat'd NaHCO₃ soln, and dried (MgSO₄). There was obtained (S)-3-bromo-5-(1-(tert-butoxy)-2-isopropoxy-2-oxoethyl)-2,6-dimethyl-4-(7-azaspiro[3.5]nonan-7-yl)pyridine 1-oxide 3 g (100%) which was carried forward without purification. ¹H NMR (500 MHz, DMSO) δ 6.24 (s, 1H), 4.95-4.90 (m, 1H), 3.78-3.73 (m, 1H), 3.35-3.25 (m, 1H), 2.81 (d, J=10.3 Hz, 1H), 2.63 (d, J=9.3 Hz, 1H), 2.58 (s, 3H), 2.36 (s, 3H), 1.89-1.74 (m, 8H), 1.59-1.54 (m, 2H), 1.17-1.15 (m, 12H), 1.08 (d, J=6.2 Hz, 3H). UPLC (M+H)=499.2.

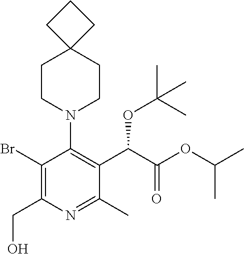 [SMILES: Cc1nc(CO)c(Br)c(N2CCC3(CCC3)CC2)c1[C@H](OC(C)(C)C)C(=O)OC(C)C]

### (S)-Isopropyl 2-(5-bromo-6-(hydroxymethyl)-2-methyl-4-(7-azaspiro[3.5]nonan-7-yl)pyridin-3-yl)-2-(tert-butoxy)acetate

To a stirred solution of (S)-3-bromo-5-(1-(tert-butoxy)-2-isopropoxy-2-oxoethyl)-2,6-dimethyl-4-(7-azaspiro[3.5]nonan-7-yl)pyridine 1-oxide (2.90 g, 5.82 mmol) in anhydrous DCM (30 mL) was added TFAA (1.64 mL, 11.64 mmol) at rt, and the reaction mixture was heated at reflux for 2.5 h. The solution was cooled and MeOH (10 mL) and Et₃N (1 mL) were added and the solution was stirred 30 min, and concentrated. The crude product was charged (DCM) to a 220 g ISCO silica gel cartridge and gradient elution (0-20% EtOAc/hexanes) using an Isolera chromatography station gave two fractions, a regioisomer 392 mg (14%) and the desired (S)-isopropyl 2-(5-bromo-6-(hydroxymethyl)-2-methyl-4-(7-azaspiro[3.5]nonan-7-yl)pyridin-3-yl)-2-(tert-butoxy)acetate 1.8 g (63%). ¹H NMR (500 MHz, DMSO) δ 6.18 (s, 1H), 4.95-4.90 (m, 1H), 4.56 (d, J=5.5 Hz, 2H), 3.81 (br. s, 1H), 3.25 (t, J=11 Hz, 1H), 2.87 (d, J=10 Hz, 1H), 2.62 (br. s, 1H), 2.48 (s, 3H), 1.90-1.72 (series of m, 8H), 1.62-1.52 (m, 2H), 1.17 (d, J=6.2 Hz, 3H), 1.14 (s, 9H), 1.08 (d, J=6.2 Hz, 3H). UPLC (M+H)=499.2.

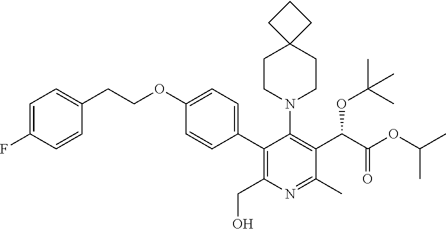 [SMILES: Cc1nc(CO)c(-c2ccc(OCCc3ccc(F)cc3)cc2)c(N2CCC3(CCC3)CC2)c1[C@H](OC(C)(C)C)C(=O)OC(C)C]

### (S)-Isopropyl 2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetate

The Pd(Ph₃P)₄ (418 mg, 0.362 mmol) was added to an argon purged and degassed solution of (S)-isopropyl 2-(5-bromo-6-(hydroxymethyl)-2-methyl-4-(7-azaspiro[3.5]nonan-7-yl)pyridin-3-yl)-2-(tert-butoxy)acetate (1.8 g, 3.62 mmol), (4-(4-fluorophenethoxy)phenyl)boronic acid (1.04 g, 3.98 mmol), sodium carbonate (1.9 g, 18.1 mmol) in dioxane (30 mL) and water (7.5 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 120 g ISCO silica gel cartridge and gradient elution (0-50% EtOAc/hexanes) using an Isolera chromatography station gave (S)-isopropyl 2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetate 1.8 g (79%). ¹H NMR (500 MHz, DMSO) δ 7.40-7.37 (m, 2H), 7.24 (d, J 8.4 Hz, 1H), 7.14 (t, J=8.1 Hz, 2H), 7.00 (s, 3H), 6.01 (s, 1H), 4.99-4.94 (m, 1H), 4.26-4.21 (m, 2H), 4.16, 3.99 (dd, J=13, 4.9 Hz, 2H), 3.29 (s, 1H), 3.14 (d, J=11 Hz, 1H), 3.06 (t, J=6.7 Hz, 2H), 2.67 (t, J=11 Hz, 1H), 2.50 (s, 3H), 2.25 (d, J=9.6 Hz, 1H), 1.84-1.73 (m, 2H), 1.66-1.43 (m, 6H), 1.34-1.25 (m, 2H), 1.20 (d, J=6.1 Hz, 3H), 1.15 (d, J=6.3 Hz, 3H), 1.13 (s, 9H). UPLC (M+H)=633.4.

### Example 38

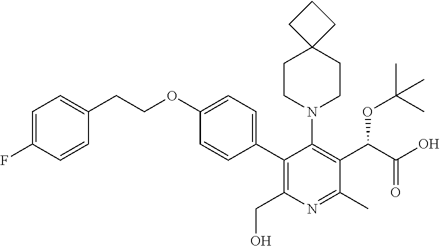 [SMILES: Cc1nc(CO)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-6-(hydroxymethyl)-2-methyl-4-(7-azaspiro[3.5]nonan-7-yl)pyridin-3-yl)acetic acid

Potassium hydroxide (35.5 mg, 0.63 mmol) was added to a solution of (S)-isopropyl 2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetate (40 mg, 0.063 mmol) in abs EtOH (2 mL) temperature was raised to 80° C. for 6 h. The reaction mixture was cooled, diluted with EtOAc, and neutralized with 1N HCl (pH=5). The organic layer was washed with brine and dried (MgSO₄), and the crude product was purified by prep HPLC to obtain (S)-2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetic acid 7.7 mg (20.6%). UPLC (M+H)=591.3.

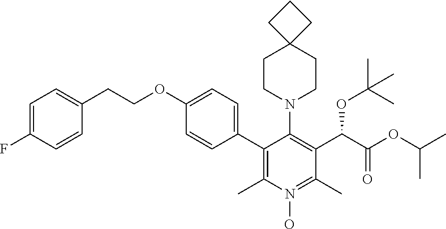

### (S)-Isopropyl 2-(tert-butoxy)-2-(5-(4-(4-fluorophenethoxy)phenyl)-2,6-dimethyl-4-(7-azaspiro[3.5]nonan-7-yl)pyridin-3-yl)acetate N-oxide

The Pd(Ph₃P)₄ (0.265 g, 0.229 mmol) was added to a nitrogen purged and degassed solution of (S)-3-bromo-5-(1-(tert-butoxy)-2-isopropoxy-2-oxoethyl)-2,6-dimethyl-4-(7-azaspiro[3.5]nonan-7-yl)pyridine 1-oxide (1.14 g, 2.292 mmol), (4-(4-fluorophenethoxy)phenyl)boronic acid (0.656 g, 2.52 mmol), and sodium carbonate (1.214 g, 11.46 mmol) in dioxane (18 mL) and water (4 mL) stirred for 18 h at 90° C. The reaction mixture was diluted with EtOAc and the organic layer was washed with water, brine and dried (MgSO₄). The crude product was charged (DCM) to a 80 g ISCO silica gel cartridge and gradient elution (5-100% MeOH/EtOAc) using an Isolera chromatography station and gave (S)-isopropyl 2-(tert-butoxy)-2-(5-(4-(4-fluorophenethoxy)phenyl)-2,6-dimethyl-4-(7-azaspiro[3.5]nonan-7-yl)pyridin-3-yl)acetate N-oxide 804 mg (55%). UPLC (M+H)=633.4.

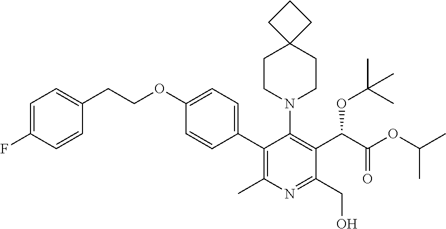 [SMILES: Cc1nc(CO)c([C@H](OC(C)(C)C)C(=O)OC(C)C)c(N2CCC3(CCC3)CC2)c1-c1ccc(OCCc2ccc(F)cc2)cc1]

### (S)-Isopropyl 2-(tert-butoxy)-2-(5-(4-(4-fluorophenethoxy)phenyl)-2-(hydroxymethyl)-6-methyl-4-(7-azaspiro[3.5]nonan-7-yl)pyridin-3-yl)acetate

The TFAA (0.134 mL, 0.948 mmol) was added to a solution of (S)-isopropyl 2-(tert-butoxy)-2-(5-(4-(4-fluorophenethoxy)phenyl)-2,6-dimethyl-4-(7-azaspiro[3.5]nonan-7-yl)pyridin-3-yl)acetate N-oxide (300 mg, 0.474 mmol) in DCM (3 mL) and stirred for 2.5 h at 65° C. MeOH (5 mL) and Et3N (1 mL) were added and stirring was continued for 30 min. The reaction mixture was diluted with DCM and washed with water, and dry (Na₂SO₄). The crude product was charged (DCM) to a 40 g ISCO silica gel cartridge and gradient elution (5-100% MeOH/EtOAc) using an Isolera chromatography station and gave (S)-isopropyl 2-(tert-butoxy)-2-(5-(4-(4-fluorophenethoxy)phenyl)-2-(hydroxymethyl)-6-methyl-4-(7-azaspiro[3.5]nonan-7-yl)pyridin-3-yl)acetate 90 mg (30%) as the minor product; ¹H NMR (500 MHz, DMSO) δ 7.40-7.37 (m, 2H), 7.23 (d, J=7.7 Hz, 1H), 7.14 (t, J=8.8H, 2H), 7.04-7.01 (m, 3H), 5.96 (br. s, 1H), 4.96-4.91 (m, 1H), 4.71-4.4.67 (m, 1H), 4.55-4.50 (m, 1H), 4.27-4.21 (m, 2H), 3.35-3.34 (m, 2H), 3.06 (t, J=6.6 Hz, 2H), 2.62 (br. s, 1H), 2.28 (br. s, 1H), 2.13 (s, 3H), 1.86-1.27 (series of m, 10H), 1.20 (d, J=6.2 Hz, 3H), 1.14 (d, J=6.6 Hz, 3H), 1.12 (s, 9H). UPLC (M+H)=633.4. The major product was identical with (S)-isopropyl 2-(tert-butoxy)-2-(5-(4-(4-fluorophenethoxy)phenyl)-6-(hydroxy methyl)-2-methyl-4-(7-azaspiro[3.5]nonan-7-yl)pyridin-3-yl)acetate 160 mg (53%) as reported above.

### Example 39

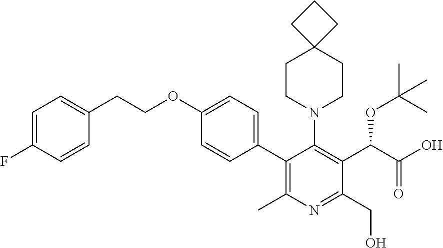 [SMILES: Cc1nc(CO)c([C@H](OC(C)(C)C)C(=O)O)c(N2CCC3(CCC3)CC2)c1-c1ccc(OCCc2ccc(F)cc2)cc1]

### (S)-2-(tert-Butoxy)-2-(5-(4-(4-fluorophenethoxy)phenyl)-2-(hydroxymethyl)-6-methyl-4-(7-azaspiro[3.5]nonan-7-yl)pyridin-3-yl)acetic acid

The potassium hydroxide (22.17 mg, 0.395 mmol) was added to a solution of (S)-isopropyl 2-(tert-butoxy)-2-(5-(4-(4-fluorophenethoxy)phenyl)-2-(hydroxymethyl)-6-methyl-4-(7-azaspiro[3.5]nonan-7-yl)pyridin-3-yl)acetate (25 mg, 0.040 mmol) in ethanol (1.5 mL) and stirred for 3 h at 90° C. The reaction mixture was cooled, diluted with EtOAc, and neutralized with 1N HCl (pH=5). The organic layer was washed with brine and dried (MgSO₄), and the crude product was purified by prep HPLC to obtain (S)-2-(tert-butoxy)-2-(5-(4-(4-fluorophenethoxy)phenyl)-2-(hydroxymethyl)-6-methyl-4-(7-azaspiro[3.5]nonan-7-yl)pyridin-3-yl)acetic acid 19.4 mg (83%). ¹H NMR (500 MHz, DMSO) δ 7.39-7.36 (m, 2H), 7.23 (d, J 8.1 Hz, 1H), 7.14 (t, J=8.8H, 2H), 7.03-7.00 (m, 3H), 5.85 (s, 1H), 4.68, 4.50 (AB, J_{AB}=14.3 Hz, 2H), 4.26-4.21 (m, 2H), 3.38 (br. s, 2H), 3.20 (br. s, 1H), 3.06 (t, J=6.6 Hz, 2H), 2.61 (br. s, 1H), 2.13 (s, 3H), 1.80-1.27 (series of m, 10H), 1.12 (s, 9H). UPLC (M+H)=591.3.

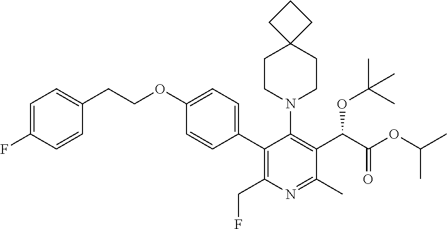 [SMILES: Cc1nc(CF)c(-c2ccc(OCCc3ccc(F)cc3)cc2)c(N2CCC3(CCC3)CC2)c1[C@H](OC(C)(C)C)C(=O)OC(C)C]

### Isopropyl (S)-2-(tert-butoxy)-2-(6-(fluoromethyl)-5-(4-(4-fluorophenethoxy)phenyl)-2-methyl-4-(7-azaspiro[3.5]nonan-7-yl)pyridin-3-yl)acetate

Deoxofluor (0.015 mL, 0.08 mmol) was added to a stirred solution of (S)-isopropyl 2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetate (25 mg, 0.04 mmol) in dry DCM (0.35 mL) at rt and the solution was stirred at rt for 16 h. The reaction mixture was diluted with DCM, washed with sat. NaHCO₃ soln, brine, and dried (MgSO₄), filtered and concentrated to give product which was used in the next step without purification. UPLC (M+H)=635.4.

### Example 40

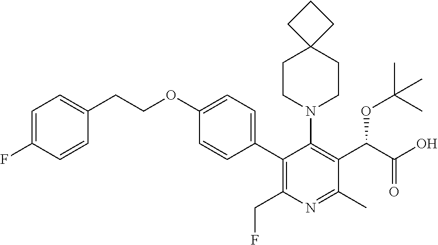 [SMILES: Cc1nc(CF)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(6-(fluoromethyl)-5-(4-(4-fluorophenethoxy)phenyl)-2-methyl-4-(7-azaspiro[3.5]nonan-7-yl)pyridin-3-yl)acetic acid

KOH (22.2 mg, 0.39 mmol) was added to the EtOH solution above crude product and the temperature was raised to 80° C. for 6 h. The reaction mixture was cooled, diluted with EtOAc, and neutralized with 1N HCl (pH=5). The organic layer was washed with brine and dried (MgSO₄), and the crude product was purified by prep HPLC to obtain (S)-2-(tert-butoxy)-2-(6-(fluoromethyl)-5-(4-(4-fluorophenethoxy)phenyl)-2-methyl-4-(7-azaspiro[3.5]nonan-7-yl)pyridin-3-yl)acetic acid 18 mg (73%). ¹H NMR (500 MHz, DMSO) δ 7.39-7.36 (m, 2H), 7.23 (d, J=9.1 Hz, 1H), 7.14 (t, J=8.8H, 2H), 7.05-6.99 (m, 3H), 5.87 (s, 1H), 5.11-4.89 (m, 2H), 4.27-4.21 (m, 2H), 3.42 (br. s, 3H), 3.06 (t, J=6.2 Hz, 2H), 2.95-2.90 (m, 1H), 2.51 (s, 3H), 1.76-1.24 (series of m, 10H), 1.12 (s, 9H). UPLC (M+H)=593.3.

### Example 41

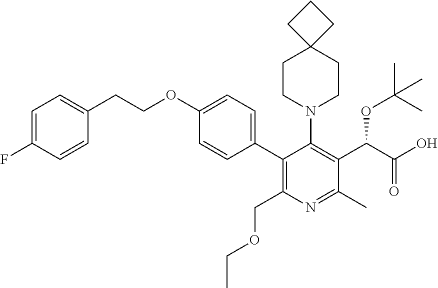 [SMILES: CCOCc1nc(C)c([C@H](OC(C)(C)C)C(=O)O)c(N2CCC3(CCC3)CC2)c1-c1ccc(OCCc2ccc(F)cc2)cc1]

### (S)-2-(tert-Butoxy)-2-(6-(ethoxymethyl)-5-(4-(4-fluorophenethoxy)phenyl)-2-methyl-4-(7-azaspiro[3.5]nonan-7-yl)pyridin-3-yl)acetic acid

The (S)-2-(tert-butoxy)-2-(6-(ethoxymethyl)-5-(4-(4-fluorophenethoxy)phenyl)-2-methyl-4-(7-azaspiro[3.5]nonan-7-yl)pyridin-3-yl)acetic acid 3.7 mg (13.6% pure) was obtained from the above reaction as a by-product. ¹H NMR (500 MHz, MeOD) δ 7.37-7.32 (m, 3H), 7.11-7.02 (m, 5H), 5.71 (s, 1H), 4.39, 4.26 (AB, J_{AB}13.5 Hz, 2H), 4.31-4.27 (m, 2H), 3.48 (q, J=6.9 Hz, 2H), 3.33 (br. s, 4H), 3.12 (t, J=6.6 Hz, 2H), 1.89-1.84 (m, 2H), 1.75-1.50 (series of m, 8H), 1.22 (s, 9H), 1.17 (t, J=6.9 Hz, 3H). UPLC (M+H)=619.3.

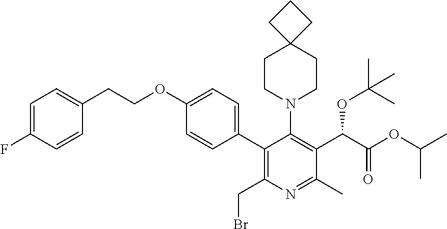 [SMILES: Cc1nc(CBr)c(-c2ccc(OCCc3ccc(F)cc3)cc2)c(N2CCC3(CCC3)CC2)c1[C@H](OC(C)(C)C)C(=O)OC(C)C]

### (S)-Isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate

To a solution of (S)-isopropyl 2-(tert-butoxy)-2-(5-(4-(4-fluorophenethoxy)phenyl)-6-(hydroxymethyl)-2-methyl-4-(7-azaspiro[3.5]nonan-7-yl)pyridin-3-yl)acetate (300 mg, 0.47 mmol) in DCM (5 mL) was added carbon tetrabromide (173 mg, 0.52 mmol) followed by triphenylphosphine (137 mg, 0.521 mmol) and the solution was stirred at room temp for 16 h. The reaction mixture was diluted with DCM, washed with water, brine, and dried over (MgSO₄). The crude product was charged (DCM) to a 40 g ISCO silica gel cartridge and gradient elution (0-50% EtOAc/hexanes) using an Isolera chromatography station gave (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate 245 mg (74%). ¹H NMR (500 MHz, CDCl3) δ 7.36 (dd, J=8.3, 2.0 Hz, 1H), 7.31-7.23 (m, 3H), 7.08-6.97 (m, 3H), 6.94 (dd, J=8.5, 2.8 Hz, 1H), 6.06 (s, 1H), 5.14-5.08 (m, 1H), 4.33 (d, J=9.3 Hz, 1H), 4.24 (dt, J=7.0, 2.8 Hz, 2H), 4.17 (d, J=9.2 Hz, 1H), 3.21 (br. s, 1H), 3.14 (t, J=6.8 Hz, 2H), 3.05 (t, J=7.0 Hz, 1H), 2.69 (br. s, 1H), 2.63 (s, 3H), 2.36-2.00 (m, 1H), 1.87-1.39 (series of m, 10H), 1.27 (d, J=6.3 Hz, 3H), 1.25 (d, J=6.3 Hz, 3H), 1.20 (s, 9H). UPLC (M+H)=697.3.

### Example 42

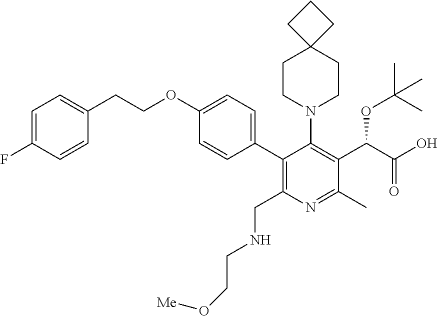 [SMILES: COCCNCc1nc(C)c([C@H](OC(C)(C)C)C(=O)O)c(N2CCC3(CCC3)CC2)c1-c1ccc(OCCc2ccc(F)cc2)cc1]

### (S)-2-(tert-Butoxy)-2-(5-(4-(4-fluorophenethoxy)phenyl)-6-(((2-methoxyethyl)amino)methyl)-2-methyl-4-(7-azaspiro[3.5]nonan-7-yl)pyridin-3-yl)acetic acid

2-Methoxyethanamine (0.027 mL, 0.302 mmol) was added to a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (35 mg, 0.05 mmol) in abs. EtOH (1 mL) and the solution was stirred for 2 h at rt. UPLC (M+H)=690.5. KOH (28.2 mg, 0.50 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-6-(((2-methoxyethyl)amino)methyl)-2-methyl-4-(7-azaspiro[3.5]nonan-7-yl)pyridin-3-yl)acetic acid 20 mg (61%). ¹H NMR (500 MHz, DMSO) δ 7.39-7.36 (m, 2H), 7.22-7.20 (m, 1H), 7.14 (t, J=8.8H, 2H), 7.01-6.99 (m, 3H), 5.80 (s, 1H), 4.26-4.19 (m, 2H), 3.51, 3.33 (AB, J_{AB}14 Hz, 2H), 3.44 (br. s, 2H), 3.37-3.33 (m, 2H), 3.17 (s, 3H), 3.05 (t, J=6.6 Hz, 2H), 2.66-2.61 (m, 3H), 2.48 (s, 3H), 2.23-2.21 (br. s, 1H), 1.76-1.24 (series of m, 10H), 1.11 (s, 9H). UPLC (M+H)=648.4.

### Example 43

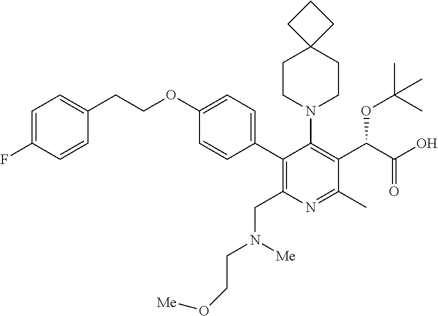 [SMILES: COCCN(C)Cc1nc(C)c([C@H](OC(C)(C)C)C(=O)O)c(N2CCC3(CCC3)CC2)c1-c1ccc(OCCc2ccc(F)cc2)cc1]

### (S)-2-(tert-Butoxy)-2-(5-(4-(4-fluorophenethoxy)phenyl)-6-(((2-methoxyethyl)(methyl)amino)methyl)-2-methyl-4-(7-azaspiro[3.5]nonan-7-yl)pyridin-3-yl)acetic acid

2-Methoxy-N-methylethanamine (0.033 mL, 0.302 mmol) was added to a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (35 mg, 0.05 mmol) in abs. EtOH (1 mL) and the solution was stirred for 2 h at rt. UPLC (M+H)=704.5. KOH (28.2 mg, 0.50 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-6-(((2-methoxyethyl)(methyl)amino)methyl)-2-methyl-4-(7-azaspiro[3.5]nonan-7-yl)pyridin-3-yl)acetic acid 25 mg (72%). ¹H NMR (500 MHz, DMSO) δ 7.38-7.36 (m, 2H), 7.32 (d, J=7.7 Hz, 1H), 7.13 (t, J=8.8H, 2H), 6.99-6.96 (m, 3H), 5.88 (s, 1H), 4.26-4.19 (m, 2H), 3.38 (br. s, 3H), 3.25-3.20 (m, 3H), 3.14 (t, J=5.9 Hz, 2H), 3.06 (s, 3H), 3.05 (t, J=6.6 Hz, 2H), 2.64 (br. s, 1H), 2.47 (s, 3H), 2.37 (br. s, 1H), 2.21 (br. s, 1H), 2.09 (s, 3H), 1.76-1.24 (series of m, 10H), 1.11 (s, 9H). UPLC (M+H)=662.4.

### Example 44

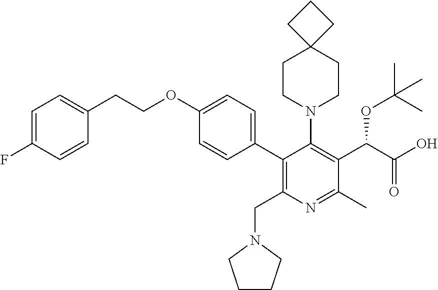 [SMILES: Cc1nc(CN2CCCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-(pyrrolidin-1-ylmethyl)-4-(7-azaspiro[3.5]nonan-7-yl)pyridin-3-yl)acetic acid

Pyrrolidine (0.025 mL, 0.30 mmol) was added to a solution of of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (35 mg, 0.05 mmol) in abs. EtOH (1 mL) and the solution was stirred for 2 h at rt. UPLC (M+H)=686.5. KOH (28.2 mg, 0.50 mmol) was added to the solution above and the temperature was raised to 80° C. for 6 h. The reaction mixture was cooled, diluted with EtOAc, and neutralized with 1N HCl (pH=5). The organic layer was washed with brine and dried (MgSO₄), and the crude product was purified by prep HPLC to obtain (S)-2-(tert-butoxy)-2-(5-(4-(4-fluorophenethoxy)phenyl)-2-methyl-6-(pyrrolidin-1-ylmethyl)-4-(7-azaspiro[3.5]nonan-7-yl)pyridin-3-yl)acetic acid 27 mg (83%). ¹H NMR (500 MHz, DMSO) δ 7.39-7.36 (m, 2H), 7.29 (d, J=7.7 Hz, 1H), 7.14 (t, J=8.8H, 2H), 6.99-6.96 (m, 3H), 5.81 (s, 1H), 4.26-4.18 (m, 2H), 3.52-3.36 (br. s, 7H), 3.27 (d, J=12.8 Hz, 1H), 3.05 (t, J=6.2 Hz, 2H), 2.63 (br. s, 1H), 2.47 (s, 3H), 2.20 (br. s, 1H), 1.76-1.24 (series of m, 14H), 1.11 (s, 9H). UPLC (M+H)=644.5.

### Example 45

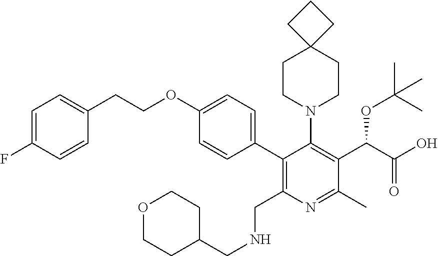 [SMILES: Cc1nc(CNCC2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-4-(7-azaspiro[3.5]nonan-7-yl)-6-((((tetrahydro-2H-pyran-4-yl)methyl)amino) methyl)pyridin-3-yl)acetic acid

(Tetrahydro-2H-pyran-4-yl)methanamine (0.045 mL, 0.431 mmol) was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (50 mg, 0.072 mmol) in abs. EtOH (1 mL) and the solution was stirred for 2 h at rt. UPLC (M+H)=730.5. KOH (40.16 mg, 0.72 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-4-(7-azaspiro[3.5]nonan-7-yl)-6-((((tetrahydro-2H-pyran-4-yl)methyl)amino)methyl)pyridin-3-yl)acetic acid 27 mg (53%). ¹H NMR (500 MHz, DMSO) δ 7.39-7.36 (m, 2H), 7.22 (d, J=7.3 Hz, 1H), 7.13 (t, J=8.8H, 2H), 7.01-6.97 (m, 3H), 5.76 (s, 1H), 4.26-4.21 (m, 2H), 3.79-3.75 (m, 2H), 3.47, 3.21 (AB, J_{AB}14.3 Hz, 2H), 3.44 (br. s, 1H), 3.23 (br. s, 1H), 3.21-3.16 (m, 2H), 3.05 (t, J=6.6 Hz, 2H), 2.61 (br. s, 1H), 2.48 (s, 3H), 2.37 (d, J=6.2 Hz, 2H), 2.21 (br. s, 1H), 1.76-1.27 (series of m, 13H), 1.10 (s, 9H). 1.08-1.03 (m, 2H). UPLC (M+H)=688.4.

### Example 46

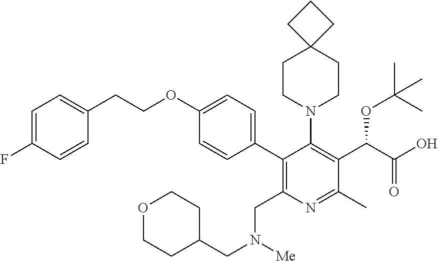 [SMILES: Cc1nc(CN(C)CC2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-((methyl((tetrahydro-2H-pyran-4-yl)methyl)amino)methyl)-4-(7-azaspiro[3.5]nonan-7-yl)pyridin-3-yl)acetic acid

N-Methyl-1-(tetrahydro-2H-pyran-4-yl)methanamine (0.051 mL, 0.431 mmol) was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (50 mg, 0.072 mmol) in abs. EtOH (1 mL) and the solution was stirred for 2 h at rt. UPLC (M+H)=744.5. KOH (40.3 mg, 0.72 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-6-((methyl((tetrahydro-2H-pyran-4-yl)methyl)amino)methyl)-4-(7-azaspiro[3.5]nonan-7-yl)pyridin-3-yl)acetic acid 43 mg (86%). ¹H NMR (500 MHz, DMSO) δ 7.38-7.35 (m, 2H), 7.33 (d, J=7.3 Hz, 1H), 7.13 (t, J=8.8H, 2H), 6.97-6.93 (m, 3H), 5.86 (s, 1H), 4.27-4.19 (m, 2H), 3.68 (t, J=12.4 Hz, 2H), 3.29-3.26 (m, 2H), 3.18-3.03 (m, 7H), 2.63 (br. s, 1H), 2.45 (s, 3H), 2.20-2.21 (m, 2H), 2.00 (s, 3H), 1.75-1.25 (series of m, 13H), 1.10 (s, 9H), 0.91-0.87 (m, 2H). UPLC (M+H)=702.4.

### Example 47

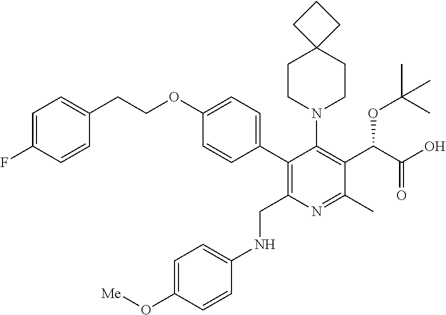 [SMILES: COc1ccc(NCc2nc(C)c([C@H](OC(C)(C)C)C(=O)O)c(N3CCC4(CCC4)CC3)c2-c2ccc(OCCc3ccc(F)cc3)cc2)cc1]

### (S)-2-(tert-Butoxy)-2-(5-(4-(4-fluorophenethoxy)phenyl)-6-(((4-methoxyphenyl)-amino)methyl)-2-methyl-4-(7-azaspiro[3.5]nonan-7-yl)pyridin-3-yl)acetic acid

4-Methoxyaniline (53.1 mg, 0.431 mmol) was added to a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (50 mg, 0.072 mmol) in abs. EtOH (1 mL) and the solution was stirred for 3 h at 55° C. UPLC (M+H)=738.5. KOH (40.3 mg, 0.72 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-6-(((4-methoxyphenyl)amino)methyl)-2-methyl-4-(7-azaspiro[3.5]nonan-7-yl)pyridin-3-yl)acetic acid 14 mg (26%). ¹H NMR (500 MHz, DMSO) δ 7.38-7.38 (m, 2H), 7.29 (d, J=9.2 Hz, 1H), 7.13 (t, J=8.3 Hz, 2H), 7.04-6.99 (m, 3H), 6.65 (d, J=8.8 Hz, 2H), 6.40 (d, J=8.8 Hz, 2H), 5.88 (s, 1H), 4.26-4.20 (m, 2H), 3.83, 3.37 (AB, J_{AB}=14.3 Hz, 2H), 3.59 (s, 3H), 3.52 (br. s, 1H), 3.25 (br. s, 1H), 3.04 (t, J=6.6 Hz, 2H), 2.67-2.62 (m, 1H), 2.55 (s, 3H), 2.24 (d, J=10.6 Hz, 1H), 1.77-1.25 (series of m, 10H), 1.12 (s, 9H). UPLC (M+H)=696.4.

### Example 48

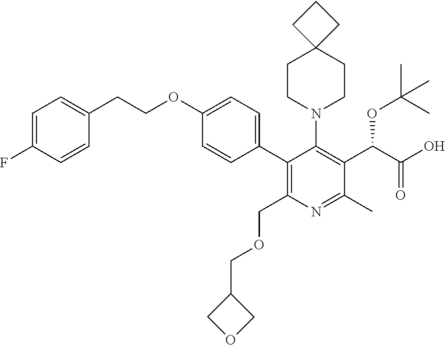 [SMILES: Cc1nc(COCC2COC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-((oxetan-3-ylmethoxy)methyl)-4-(7-azaspiro[3.5]nonan-7-yl)pyridin-3-yl)acetic acid

Sodium hydride (8.62 mg, 0.22 mmol) was added to a solution of oxetan-3-ylmethanol (0.017 mL, 0.22 mmol) in dry dioxane (0.3 mL) at rt. After 15 min there was added a solution of(S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in dry dioxane (0.3 mL) and the solution was stirred for 2 h at rt. UPLC (M+H)=703.4. KOH (20.16 mg, 0.359 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-6-((oxetan-3-ylmethoxy)methyl)-4-(7-azaspiro[3.5]nonan-7-yl)pyridin-3-yl)acetic acid 14 mg (59%). ¹H NMR (500 MHz, DMSO) δ 7.39-7.36 (m, 2H), 7.22 (d, J=8.8 Hz, 1H), 7.14 (t, J=8.8H, 2H), 7.00-6.98 (m, 3H), 5.81 (s, 1H), 4.53 (t, J=6.2 Hz, 2H), 4.26-4.13 (m, 5H), 4.00 (d, J=9.5 Hz, 1H), 3.40 (br. s, 3H), 3.05 (t, J=6.6 Hz, 2H), 3.03-2.97 (m, 1H), 2.63 (br. s, 1H), 2.46 (s, 3H), 2.22 (br. s, 1H), 1.78-1.25 (series of m, 11H), 1.11 (s, 9H). UPLC (M+H)=661.5.

### Example 49

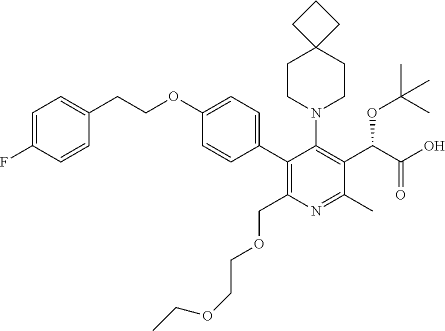 [SMILES: CCOCCOCc1nc(C)c([C@H](OC(C)(C)C)C(=O)O)c(N2CCC3(CCC3)CC2)c1-c1ccc(OCCc2ccc(F)cc2)cc1]

### (S)-2-(tert-Butoxy)-2-(6-((2-ethoxyethoxy)methyl)-5-(4-(4-fluoro-phenethoxy)phenyl)-2-methyl-4-(7-azaspiro[3.5]nonan-7-yl)pyridin-3-yl)acetic acid

Sodium hydride (8.62 mg, 0.22 mmol) was added to a solution of 2-ethoxyethanol (0.021 mL, 0.22 mmol) in dry dioxane (0.3 mL) at rt. After 15 min there was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in dry dioxane (0.3 mL) and the solution was stirred for 2 h at rt. UPLC (M+H)=705.4. KOH (20.16 mg, 0.359 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(6-((2-ethoxyethoxy)methyl)-5-(4-(4-fluoro-phenethoxy)phenyl)-2-methyl-4-(7-azaspiro[3.5]nonan-7-yl)pyridin-3-yl)acetic acid 19 mg (79%). ¹H NMR (500 MHz, DMSO) δ 7.39-7.36 (m, 2H), 7.27 (d, J=8.1 Hz, 1H), 7.13 (t, J=8.8H, 2H), 6.99-6.96 (m, 3H), 5.80 (s, 1H), 4.27-4.18 (m, 2H), 4.12, 3.98 (AB, J_{AB}=9.9 Hz, 2H), 3.41 (br. s, 2H), 3.35-3.27 (m, 6H), 3.05 (t, J=6.6 Hz, 2H), 2.64 (br. s, 1H), 2.47 (s, 3H), 2.19 (br. s, 1H), 1.77-1.24 (series of m, 10H), 1.11 (s, 9H), 1.05 (t, J=7.0 Hz, 3H). UPLC (M+H)=663.4.

### Example 50

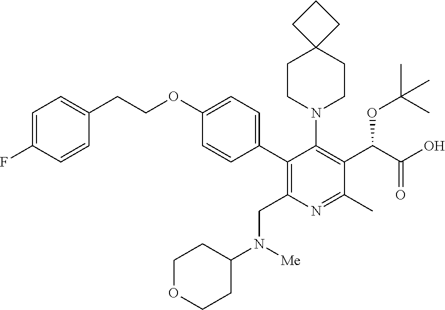 [SMILES: Cc1nc(CN(C)C2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-((methyl(tetrahydro-2H-pyran-4-yl)amino)methyl)-4-(7-azaspiro[3.5]nonan-7-yl)pyridin-3-yl)acetic acid

N-Methyltetrahydro-2H-pyran-4-amine (12.4 mg, 0.11 mmol) was added to a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) and Hunig's base (0.02 mL, 0.11 mmol) in abs. EtOH (0.5 mL) and the solution was stirred for 4 h at rt. UPLC (M+H)=730.5. KOH (20.16 mg, 0.36 mmol) was added to the solution above and the temperature was raised to 80° C. for 16 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-6-((methyl(tetrahydro-2H-pyran-4-yl)amino)methyl)-4-(7-azaspiro[3.5]nonan-7-yl)pyridin-3-yl)acetic acid 8.3 mg (33%). ¹H NMR (500 MHz, DMSO) δ 7.39-7.36 (m, 2H), 7.31 (d, J 8.4 Hz, 1H), 7.13 (t, J=9.1 Hz, 2H), 7.06-7.04 (m, 3H), 5.86 (s, 1H), 4.27-4.19 (m, 2H), 3.92-3.87 (m, 2H), 3.80 (d, J=14.8 Hz, 1H), 3.41 (br. s, 3H), 3.24 (t, J=9.9 Hz, 2H), 3.21-3.16 (m, 2H), 3.06 (t, J=6.2 Hz, 2H), 2.72/2.58 (s, 3H), 2.55 (s, 3H), 2.26 (br. s, 1H), 1.84-1.47 (series of m, 14H), 1.13 (s, 9H). UPLC (M+H)=688.5.

### Example 51

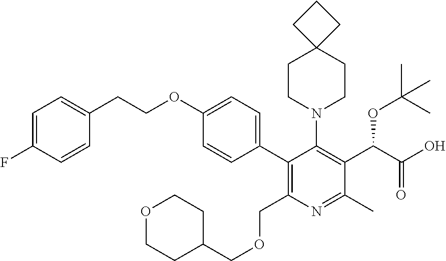 [SMILES: Cc1nc(COCC2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-4-(7-azaspiro[3.5]nonan-7-yl)-6-(((tetrahydro-2H-pyran-4-yl)methoxy)methyl)pyridin-3-yl)acetic acid

Sodium hydride (8.62 mg, 0.22 mmol) was added to a solution of (tetrahydro-2H-pyran-4-yl)methanol (0.025 mL, 0.22 mmol) in dry dioxane (0.3 mL) at rt. After 15 min there was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in dry dioxane (0.3 mL) and the solution was stirred for 16 h at rt. UPLC (M+H)=731.5. KOH (20.16 mg, 0.359 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-4-(7-azaspiro[3.5]nonan-7-yl)-6-(((tetrahydro-2H-pyran-4-yl)methoxy)methyl)pyridin-3-yl)acetic acid 18 mg (79%). ¹H NMR (500 MHz, DMSO) δ 7.39-7.36 (m, 2H), 7.23 (d, J=8.8 Hz, 1H), 7.14 (t, J=8.8H, 2H), 7.00-6.98 (m, 3H), 5.89 (s, 1H), 4.27-4.18 (m, 2H), 4.12, 3.95 (AB, J_{AB}=9.5 Hz, 2H), 3.76 (dd, J=10.6, 2.9 Hz, 2H), 3.39-3.36 (m, 4H), 3.25-3.17 (m, 4H), 3.05 (t, J=6.6 Hz, 2H), 3.01 (d, J=6.2 Hz, 2H), 2.68-2.63 (m, 1H), 2.47 (s, 3H), 2.23-2.19 (m, 1H), 1.79-1.24 (series of m, 13H), 1.12 (s, 9H), 1.07-1.00 (m, 2H). UPLC (M+H)=689.5.

### Example 52

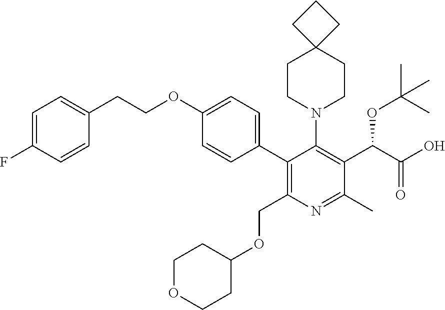 [SMILES: Cc1nc(COC2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-4-(7-azaspiro[3.5]nonan-7-yl)-6-(((tetrahydro-2H-pyran-4-yl)oxy)methyl)pyridin-3-yl)acetic acid

Sodium hydride (8.62 mg, 0.22 mmol) was added to a solution of tetrahydro-2H-pyran-4-ol (0.020 mL, 0.22 mmol) in dry dioxane (0.3 mL) at rt. After 15 min there was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in dry dioxane (0.3 mL) and the solution was stirred for 2 h at rt. UPLC (M+H)=717.6. KOH (20.16 mg, 0.359 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-4-(7-azaspiro[3.5]nonan-7-yl)-6-(((tetrahydro-2H-pyran-4-yl)oxy)methyl)pyridin-3-yl)acetic acid 12 mg (51%). ¹H NMR (500 MHz, DMSO) δ 7.38-7.36 (m, 2H), 7.26 (d, J=8.4 Hz, 1H), 7.13 (t, J=8.8H, 2H), 7.01-6.99 (m, 3H), 5.83 (s, 1H), 4.28-4.20 (m, 3H), 4.20, 4.00 (AB, J_{AB}=9.2 Hz, 2H), 3.70-3.66 (m, 1H), 3.64-3.60 (m, 1H), 3.23-3.15 (m, 2H), 3.05 (t, J=6.6 Hz, 2H), 2.64 (br. s, 1H), 2.47 (s, 3H), 2.23-2.21 (m, 1H), 1.76-1.15 (series of m, 14H), 1.12 (s, 9H). UPLC (M+H)=675.5.

### Example 53

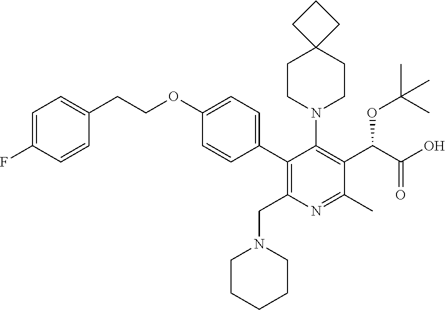 [SMILES: Cc1nc(CN2CCCCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-(piperidin-1-ylmethyl)-4-(7-azaspiro[3.5]nonan-7-yl)pyridin-3-yl)acetic acid

Piperidine (0.04 mL, 0.36 mmol) was added to a solution of of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in abs. EtOH (0.5 mL) and the solution was stirred for 2 h at rt. UPLC (M+H)=700.5. KOH (20.2 mg, 0.36 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-6-(piperidin-1-ylmethyl)-4-(7-azaspiro[3.5]nonan-7-yl)pyridin-3-yl)acetic acid 22 mg (94%). ¹H NMR (500 MHz, DMSO) δ 7.38-7.35 (m, 2H), 7.32 (d, J=9.2 Hz, 1H), 7.13 (t, J=9.2H, 2H), 6.97-6.96 (m, 3H), 5.87 (s, 1H), 4.26-4.18 (m, 2H), 3.27 (br. s, 1H), 3.11 (s, 1H), 3.04 (t, J=6.6 Hz, 2H), 2.90 (s, 1H), 2.74 (s, 1H), 2.64 (br. s, 1H), 2.46 (s, 3H), 2.25-2.15 (m, 5H), 1.75-1.24 (series of m, 16H), 1.12 (s, 9H). UPLC (M+H)=658.4.

### Example 54

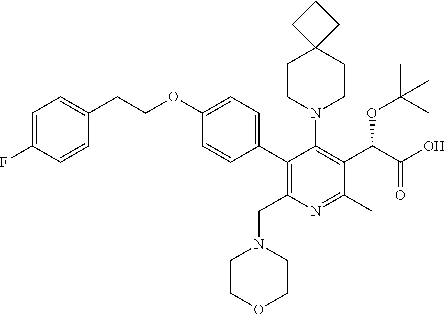 [SMILES: Cc1nc(CN2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-(morpholinomethyl)-4-(7-azaspiro[3.5]nonan-7-yl)pyridin-3-yl)acetic acid

Morpholine (0.03 mL, 0.36 mmol) was added to a solution of of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in abs. EtOH (0.5 mL) and the solution was stirred for 2 h at rt. UPLC (M+H)=702.5. KOH (20.2 mg, 0.36 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-6-(morpholinomethyl)-4-(7-azaspiro[3.5]nonan-7-yl)pyridin-3-yl)acetic acid 17 mg (72%). ¹H NMR (500 MHz, DMSO) δ 7.38-7.35 (m, 2H), 7.33 (d, J=8.4 Hz, 1H), 7.13 (t, J=9.2H, 2H), 6.98-6.97 (m, 3H), 5.89 (s, 1H), 4.26-4.18 (m, 2H), (not all protons evident), 3.14 (br. s, 1H), 3.23 (br. s, 1H), 3.11 (t, J 11.4 Hz, 1H), 3.04 (t, J=6.6 Hz, 2H), 2.90 (s, 1H), 2.74 (s, 1H), 2.64 (br. s, 1H), 2.46 (s, 3H), 2.25-2.16 (m, 5H), 1.78-1.24 (series of m, 10H), 1.12 (s, 9H). UPLC (M+H)=660.4.

### Example 55

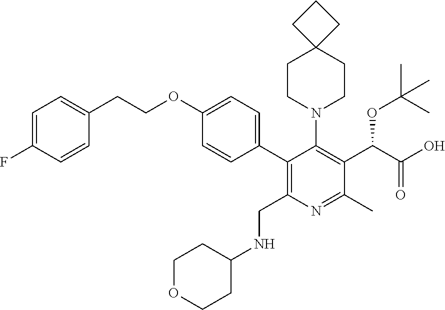 [SMILES: Cc1nc(CNC2CCOCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-4-(7-azaspiro[3.5]nonan-7-yl)-6-(((tetrahydro-2H-pyran-4-yl)amino)methyl)pyridin-3-yl)acetic acid

Tetrahydro-2H-pyran-4-amine (10.90 mg, 0.11 mmol) was added to a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in abs. EtOH (0.5 mL) and the solution was stirred for 48 h at 50° C. UPLC (M+H)=716.5. KOH (20.16 mg, 0.359 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)-phenyl)-2-methyl-4-(7-azaspiro[3.5]nonan-7-yl)-6-(((tetrahydro-2H-pyran-4-yl)amino)methyl)pyridin-3-yl)acetic acid 6.3 mg (26%). ¹H NMR (500 MHz, DMSO) δ 7.39-7.36 (m, 2H), 7.24 (d, J=7.7 Hz, 1H), 7.13 (t, J=8.8 Hz, 2H), 7.01-7.00 (m, 3H), 5.81 (s, 1H), 4.26-4.20 (m, 2H), 3.75-3.72 (m, 2H), 3.22-3.18 (m, 2H), 3.05 (t, J=6.6 Hz, 2H), 2.65-2.56 (m, 2H), (protons adj nitrogen not evident), 2.49 (s, 3H), 2.25-2.20 (m, 1H), 1.76-1.16 (series of m, 14H), 1.12 (s, 9H). UPLC (M+H)=674.5.

### Example 56

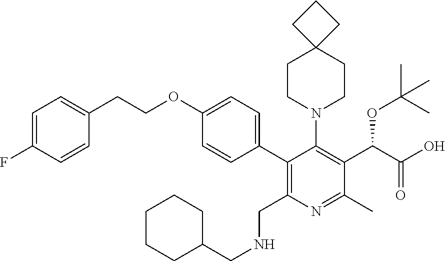 [SMILES: Cc1nc(CNCC2CCCCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(6-(((cyclohexylmethyl)amino)methyl)-5-(4-(4-fluorophenethoxy)phenyl)-2-methyl-4-(7-azaspiro[3.5]nonan-7-yl)pyridin-3-yl)acetic acid

Cyclohexylmethanamine (0.028 mL, 0.22 mmol) was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in abs. EtOH (1 mL) and the solution was stirred for 2 h at rt. UPLC (M+H)=728.5. KOH (20.16 mg, 0.36 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(6-(((cyclohexylmethyl)amino)methyl)-5-(4-(4-fluorophen-ethoxy)phenyl)-2-methyl-4-(7-azaspiro[3.5]nonan-7-yl)pyridin-3-yl)acetic acid 23.5 mg (95%). ¹H NMR (500 MHz, DMSO) δ 7.39-7.36 (m, 2H), 7.22 (d, J=7.3 Hz, 1H), 7.14 (t, J=8.8 Hz, 2H), 7.01-6.99 (m, 3H), 5.81 (s, 1H), 4.27-4.21 (m, 2H), 3.06 (t, J=6.6 Hz, 2H), (protons adj nitrogen not evident), 2.66-2.59 (m, 1H), 2.49 (s, 3H), 2.31 (d, J=6.6 Hz, 2H), 2.23 (br. s, 1H), 1.76-1.25 (series of m, 16H), 1.12-1.09 (m, 12H), 0.84-0.78 (m, 2H). UPLC (M+H)=686.5.

### Example 57

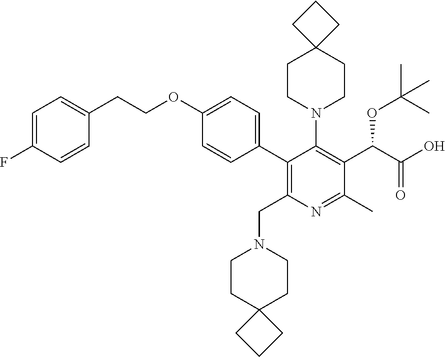 [SMILES: Cc1nc(CN2CCC3(CCC3)CC2)c(-c2ccc(OCCc3ccc(F)cc3)cc2)c(N2CCC3(CCC3)CC2)c1[C@H](OC(C)(C)C)C(=O)O]

### (S)-2-(6-(7-Azaspiro[3.5]nonan-7-ylmethyl)-5-(4-(4-fluorophenethoxy)phenyl)-2-methyl-4-(7-azaspiro[3.5]nonan-7-yl)pyridin-3-yl)-2-(tert-butoxy)acetic acid

7-Azaspiro[3.5]nonane (27.0 mg, 0.216 mmol) was added to a solution of of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in abs. EtOH (0.5 mL) and the solution was stirred for 2 h at rt. UPLC (M+H)=740.5. KOH (20.2 mg, 0.36 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(6-(7-azaspiro[3.5]nonan-7-ylmethyl)-5-(4-(4-fluorophenethoxy)phenyl)-2-methyl-4-(7-azaspiro[3.5]nonan-7-yl)pyridin-3-yl)-2-(tert-butoxy)acetic acid 13 mg (52%). ¹H NMR (500 MHz, DMSO) δ 7.39-7.36 (m, 2H), 7.32 (d, J 8.8 Hz, 1H), 7.14 (t, J=8.8 Hz, 2H), 6.97-6.95 (m, 3H), 5.84 (s, 1H), 4.26-4.19 (m, 2H), 3.05 (t, J=6.6 Hz, 2H), (protons adj nitrogen not evident), 2.65-2.62 (m, 1H), 2.45 (s, 3H), 2.23-2.20 (m, 1H), 2.13-2.04 (m, 4H), 1.80-1.24 (series of m, 20H), 1.11 (s, 9H). UPLC (M+H)=698.4.

### Example 58

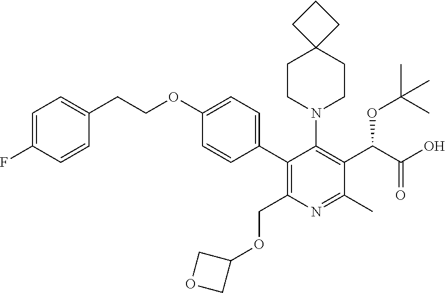 [SMILES: Cc1nc(COC2COC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(5-(4-(4-fluorophenethoxy)phenyl)-2-methyl-6-((oxetan-3-yloxy)methyl)-4-(7-azaspiro[3.5]nonan-7-yl)pyridin-3-yl)acetic acid

Sodium hydride (8.62 mg, 0.22 mmol) was added to a solution of oxetan-3-ol (16 mg, 0.22 mmol) in dry dioxane (0.3 mL) at rt. After 15 min there was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in dry dioxane (0.3 mL) and the solution was stirred for 1 h at rt, acetonitrile (0.3 mL) was added, and the temperature was raised to 50° C. for 1.5 h. UPLC (M+H)=689.4. KOH (20.16 mg, 0.359 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(5-(4-(4-fluorophenethoxy)phenyl)-2-methyl-6-((oxetan-3-yloxy)methyl)-4-(7-azaspiro[3.5]nonan-7-yl)pyridin-3-yl)acetic acid 9.6 mg (40%). UPLC (M+H)=647.3.

### Example 59

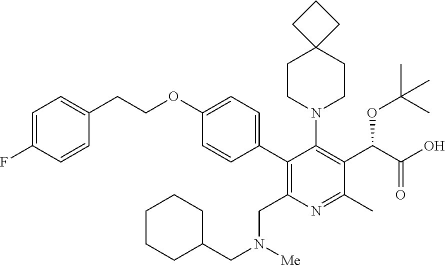 [SMILES: Cc1nc(CN(C)CC2CCCCC2)c(-c2ccc(OCCc3ccc(F)cc3)cc2)c(N2CCC3(CCC3)CC2)c1[C@H](OC(C)(C)C)C(=O)O]

### (S)-2-(tert-Butoxy)-2-(6-(((cyclohexylmethyl)(methyl)amino) methyl)-5-(4-(4-fluorophenethoxy)phenyl)-2-methyl-4-(7-azaspiro[3.5]nonan-7-yl)pyridin-3-yl)acetic acid

1-Cyclohexyl-N-methylmethanamine (0.033 mL, 0.22 mmol) was added a solution of (S)-isopropyl 2-(6-(bromomethyl)-5-(4-(4-fluorophenethoxy)phenyl)-2-methyl-4-(7-azaspiro[3.5]nonan-7-yl)pyridin-3-yl)-2-(tert-butoxy)acetate (25 mg, 0.036 mmol) in abs. EtOH (1 mL) and the solution was stirred for 2 h at rt. UPLC (M+H)=742.5. KOH (20.16 mg, 0.36 mmol) was added to the solution above and the temperature was raised to 80° C. for 6 h. TFA (0.028 mL, 0.359 mmol) was added and the reaction mixture was concentrated. The crude product was purified by prep HPLC to obtain (S)-2-(tert-butoxy)-2-(6-(((cyclohexylmethyl)(methyl)amino) methyl)-5-(4-(4-fluorophenethoxy)phenyl)-2-methyl-4-(7-azaspiro[3.5]nonan-7-yl)pyridin-3-yl)acetic acid 20.6 mg (77%). ¹H NMR (500 MHz, DMSO) δ 7.38-7.36 (m, 3H), 7.13 (t, J=8.8 Hz, 2H), 6.98-6.92 (m, 3H), 5.82 (s, 1H), 4.27-4.19 (m, 2H), (proton adj to nitrogen not evident), 3.13-3.04 (m, 3H), 2.64-2.60 (m, 1H), 2.45 (s, 3H), 2.19 (br. s, 1H), 2.12-2.08 (m, 1H), 1.98 (s, 3H), 1.76-1.14 (series of m, 17H), 1.10 (m, 9H), 1.05-1.00 (m, 2H), 0.68-0.54 (m, 2H). UPLC (M+H)=686.5.

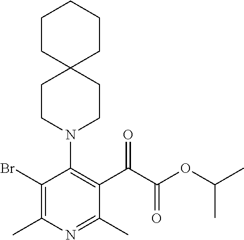 [SMILES: Cc1nc(C)c(C(=O)C(=O)OC(C)C)c(N2CCC3(CCCCC3)CC2)c1Br]

### Isopropyl 2-(5-bromo-2,6-dimethyl-4-(3-azaspiro[5.5]undecan-3-yl)pyridin-3-yl)-2-oxoacetate

To a solution 3-azaspiro[5.5]undecane (1 g, 6.52 mmol) and DIEA (3.4 mL, 19.6 mmol) in anhydrous CH₃CN (25 mL) was added isopropyl 2-(5-bromo-4-chloro-2,6-dimethylpyridin-3-yl)-2-oxoacetate (2.2 g, 6.52 mmol) at rt. The resulting mixture was placed in a pre-heated oil bath (80° C.) and stirred for 18 h; cooled, and concentrated. The crude product was charged (DCM) to a 80 g ISCO silica gel cartridge and gradient elution (5-35% EtOAc/hexanes) using an Isolera chromatography station to give isopropyl 2-(5-bromo-2,6-dimethyl-4-(3-azaspiro[5.5]undecan-3-yl)pyridin-3-yl)-2-oxoacetate 1.42 g (48%). ¹H NMR (500 MHz, CDCl3) δ 5.07-5.02 (m, 1H), 3.39-3.34 (m, 2H), 3.18 (s, 2H), 2.61 (s, 3H), 2.29 (s, 3H), 1.40-1.35 (m, 14H), 1.29 (d, J=6.2 Hz, 6H). UPLC (M+H)=453.3.

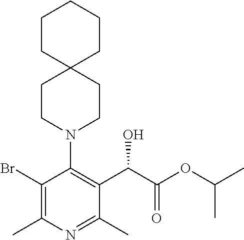 [SMILES: Cc1nc(C)c([C@H](O)C(=O)OC(C)C)c(N2CCC3(CCCCC3)CC2)c1Br]

### (S)-Isopropyl 2-(5-bromo-2,6-dimethyl-4-(3-azaspiro[5.5]undecan-3-yl)pyridin-3-yl)-2-hydroxyacetate

The 1.1 mL of benzo[d][1,3,2]dioxaborole (697 mg, 5.66 mmol) was added to a nitrogen purged solution of isopropyl 2-(5-bromo-2,6-dimethyl-4-(3-azaspiro[5.5]undecan-3-yl)pyridin-3-yl)-2-oxoacetate (1.42 g, 3.15 mmol) and 0.94 mL of (R)-1-methyl-3,3-diphenylhexahydropyrrolo[1,2-c][1,3,2]oxazaborole (262 mg, 0.94 mmol) in toluene (40 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine and dried (MgSO₄). The crude product was charged (DCM) to a 40 g ISCO silica gel cartridge and gradient elution (5-75% EtOAc/hexanes) using an Isolera chromatography station gave (S)-isopropyl 2-(5-bromo-2,6-dimethyl-4-(3-azaspiro[5.5]undecan-3-yl)pyridin-3-yl)-2-hydroxyacetate 1.4 g (98%). ¹H NMR (500 MHz, DMSO) δ 5.89 (s, 1H), 4.97-4.92 (m, 1H), 3.69 (t, J=11.4 Hz, 1H), 3.49 (t, J=11 Hz, 1H), 2.71 (d, J=11 Hz, 1H), 2.61 (d, J=11.7 Hz, 1H), 2.53 (s, 3H), 2.37 (s, 3H), 1.60-1.53 (m, 4H), 1.44-1.42 (m, 8H), 1.28 (br. s, 2H), 1.15 (d, J=6.3 Hz, 3H), 1.07 (d, J=6.2 Hz, 3H). UPLC (M+H)=455.4.

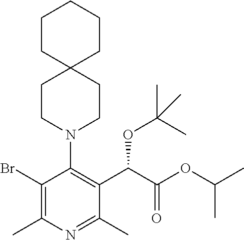 [SMILES: Cc1nc(C)c([C@H](OC(C)(C)C)C(=O)OC(C)C)c(N2CCC3(CCCCC3)CC2)c1Br]

### (S)-Isopropyl 2-(5-bromo-2,6-dimethyl-4-(3-azaspiro[5.5]undecan-3-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(3-azaspiro[5.5]undecan-3-yl)pyridin-3-yl)-2-hydroxyacetate (1.4 g, 3.09 mmol) and 0.6 mL of 70% HClO₄ in DCM (25 mL) for 20 min. The reaction mixture was allowed to warm to rt and stirred for 18 h in a pressure sealed vessel. The reaction mixture was diluted with DCM, washed with 1M Na2CO3 soln, and dried over MgSO₄. The crude product was charged (DCM) to a 80 g ISCO silica gel cartridge and gradient elution (5-35% EtOAc/hexanes) using an Isolera chromatography station gave (S)-isopropyl 2-(5-bromo-2,6-dimethyl-4-(3-azaspiro[5.5]undecan-3-yl)pyridin-3-yl)-2-(tert-butoxy)acetate. ¹H NMR (500 MHz, DMSO) δ 6.18 (s, 1H), 4.93-4.88 (m, 1H), 3.97 (br. s, 1H), 3.39-3.36 (m, 2H), 2.78 (br. s, 1H), 2.52 (s, 3H), 2.41 (s, 3H), 1.72 (br. s, 1H), 1.62-1.55 (m, 3H), 1.45-1.37 (m, 8H), 1.29-1.27 (m, 2H), 1.15-1.14 (m, 12H), 1.07 (d, J=6.2 Hz, 3H). UPLC (M+H)=510.5.

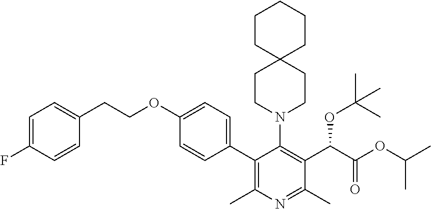 [SMILES: Cc1nc(C)c([C@H](OC(C)(C)C)C(=O)OC(C)C)c(N2CCC3(CCCCC3)CC2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(3-azaspiro[5.5]undecan-3-yl)pyridin-3-yl)acetate

The tretrakis (34 mg, 0.029 mmol) was added to a nitrogen purged and degassed solution (S)-isopropyl 2-(5-bromo-2,6-dimethyl-4-(3-azaspiro[5.5]undecan-3-yl)pyridin-3-yl)-2-(tert-butoxy)acetate (150 mg, 0.294 mmol), (4-(4-fluorophenethoxy)phenyl)boronic acid (84 mg, 0.324 mmol), and sodium carbonate (187 mg, 1.8 mmol) in dioxane (4.5 mL) and water (0.9 mL) and stirred in a screw-capped pressure vessel for 4 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(3-azaspiro[5.5]undecan-3-yl)pyridin-3-yl)acetate 110 mg (59%). ¹H NMR (500 MHz, DMSO) δ 7.38-7.35 (m, 2H), 7.20 (d, J=7.7 Hz, 1H), 7.13 (t, J=8.4 Hz, 2H), 7.03-7.01 (m, 3H), 5.98 (s, 1H), 4.97-4.93 (m, 1H), 4.25-4.21 (m, 2H), 3.41-3.39 (m, 4H), 3.04 (t, J=6.2 Hz, 2H), 2.43 (s, 3H), 2.16 (br. s, 1H), 2.06 (s, 3H), 1.98 (br. s, 1H), 1.49 (br. s, 1H), 1.32-1.23 (m, 7H), 1.18 (d, J=5.9 Hz, 3H), 1.15-1.12 (s, 16H). UPLC (M+H)=645.7.

### Example 60

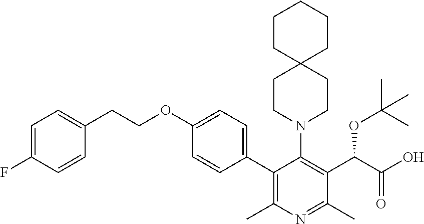 [SMILES: Cc1nc(C)c([C@H](OC(C)(C)C)C(=O)O)c(N2CCC3(CCCCC3)CC2)c1-c1ccc(OCCc2ccc(F)cc2)cc1]

### (S)-2-(tert-Butoxy)-2-(5-(4-(4-fluorophenethoxy)phenyl)-2,6-dimethyl-4-(3-azaspiro[5.5]undecan-3-yl)pyridin-3-yl)acetic acid

The potassium hydroxide (47.8 mg, 0.87 mmol) was added to a solution of (S)-isopropyl 2-(tert-butoxy)-2-(5-(4-(4-fluorophenethoxy)phenyl)-2,6-dimethyl-4-(3-azaspiro[5.5]undecan-3-yl)pyridin-3-yl)acetate (56 mg, 0.087 mmol) in ethanol (2 mL) and stirred for 4 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)-phenyl)-2,6-dimethyl-4-(3-azaspiro[5.5]undecan-3-yl)pyridin-3-yl)acetic acid 33.4 mg (64%). ¹H NMR (500 MHz, DMSO) δ 7.38-7.35 (m, 2H), 7.20 (d, J=8.4 Hz, 1H), 7.14 (t, J=8.8 Hz, 2H), 7.03-7.00 (m, 3H), 5.82 (s, 1H), 4.25-4.21 (m, 2H), 3.28-3.25 (m, 2H), 3.04 (t, J=6.6 Hz, 2H), 2.83-2.78 (m, 2H), 2.43 (s, 3H), 2.14-2.12 (m, 1H), 2.05 (s, 3H), 1.96-1.91 (m, 1H), 1.37-1.30 (m, 4H), 1.26-1.14 (m, 6H), 1.12 (s, 9H), 0.99-0.97 (m, 2H). UPLC (M+H)=603.6.

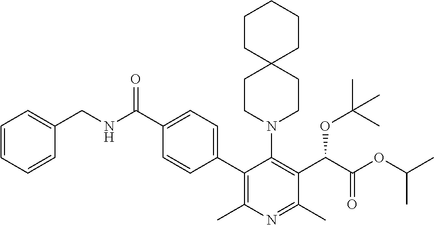 [SMILES: Cc1nc(C)c([C@H](OC(C)(C)C)C(=O)OC(C)C)c(N2CCC3(CCCCC3)CC2)c1-c1ccc(C(=O)NCc2ccccc2)cc1]

### (S)-Isopropyl 2-(5-(4-(benzylcarbamoyl)phenyl)-2,6-dimethyl-4-(3-azaspiro[5.5]undecan-3-yl)pyridin-3-yl)-2-(tert-butoxy)acetate

The Pd(Ph3P)4 (34 mg, 0.029 mmol) was added to a nitrogen purged and degassed solution (S)-isopropyl 2-(5-bromo-2,6-dimethyl-4-(3-azaspiro[5.5]undecan-3-yl)pyridin-3-yl)-2-(tert-butoxy)acetate (150 mg, 0.294 mmol), (4-(benzylcarbamoyl)phenyl)boronic acid (83 mg, 0.324 mmol), and sodium carbanate (187 mg, 1.8 mmol) in dioxane (4.5 mL) and water (0.9 mL) and stirred in a screw-capped pressure vessel for 4 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)-phenyl)-2,6-dimethyl-4-(3-azaspiro[5.5]undecan-3-yl)pyridin-3-yl)-2-(tert-butoxy)acetate 71 mg (38%). ¹H NMR (500 MHz, DMSO) δ 9.17-9.15 (m, 1H), 8.03-7.98 (m, 2H), 7.46 (d, J=7.7 Hz, 1H), 7.34-7.33 (m, 4H), 7.26-7.25 (m, 2H), 5.96 (s, 1H), 4.98-4.95 (m, 1H), 4.52 (d, J=5.9 Hz, 2H), 3.45 (br. s, 2H), 3.12-3.10 (s, 1H), 2.86-2.81 (m, 1H), 2.45 (s, 3H), 2.21-2.18 (m, 1H), 2.06 (s, 3H), 1.50 (m, 1H), 1.35-1.22 (m, 6H), 1.20 (d, J=6.2 Hz, 3H), 1.16-1.13 (m, 17H), 0.95 (br. s, 1H). UPLC (M+H)=640.7.

### Example 61

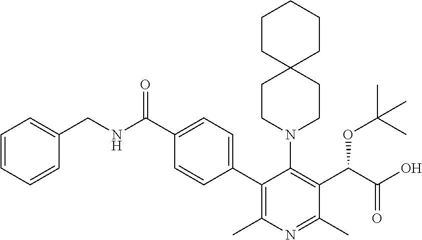 [SMILES: Cc1nc(C)c([C@H](OC(C)(C)C)C(=O)O)c(N2CCC3(CCCCC3)CC2)c1-c1ccc(C(=O)NCc2ccccc2)cc1]

### (S)-2-(5-(4-(Benzylcarbamoyl)phenyl)-2,6-dimethyl-4-(3-azaspiro[5.5]undecan-3-yl)pyridin-3-yl)-2-(tert-butoxy)acetic acid

The potassium hydroxide (53.5 mg, 0.95 mmol) was added to a solution of (S)-isopropyl 2-(5-(4-(benzylcarbamoyl)phenyl)-2,6-dimethyl-4-(3-azaspiro[5.5]undecan-3-yl)pyridin-3-yl)-2-(tert-butoxy)acetate (61 mg, 0.095 mmol) in ethanol (2 mL) and stirred for 4 h at 95° C. The reaction mixture was cooled, neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(3-azaspiro[5.5]undecan-3-yl)pyridin-3-yl)-2-(tert-butoxy)acetic acid 35.9 mg (63%). ¹H NMR (500 MHz, DMSO) δ 9.17-9.15 (m, 1H), 8.02-7.98 (m, 2H), 7.46 (d, J=7.3 Hz, 1H), 7.34-7.33 (m, 4H), 7.26-7.23 (m, 2H), 5.82 (s, 1H), 4.52 (d, J=6.2 Hz, 2H), 3.49 (br. s, 2H), 3.30-3.27 (m, 1H), 2.84-2.79 (m, 1H), 2.45 (s, 3H), 2.18-2.16 (m, 1H), 2.05 (s, 3H), 1.87-1.83 (m, 1H), 1.37-1.15 (m, 11H), 1.13 (s, 9H), 0.93 (br. s, 1H). UPLC (M+H)=598.6.

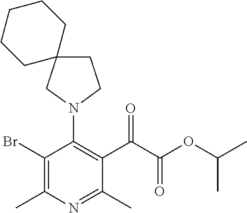 [SMILES: Cc1nc(C)c(C(=O)C(=O)OC(C)C)c(N2CCC3(CCCCC3)C2)c1Br]

### Isopropyl 2-(5-bromo-2,6-dimethyl-4-(2-azaspiro[4.5]decan-2-yl)pyridin-3-yl)-2-oxoacetate

To a solution of 2-azaspiro[4.5]decane (988 mg, 7.1 mmol) and DIEA (3.7 mL, 21.3 mmol) in anhydrous CH₃CN (35 mL) was added isopropyl 2-(5-bromo-4-chloro-2,6-dimethylpyridin-3-yl)-2-oxoacetate (2.3 g, 7.1 mmol) at rt. The resulting mixture was placed in a pre-heated oil bath (80° C.) and stirred for 18 h; cooled, and concentrated. The crude product was charged (DCM) to a 80 g ISCO silica gel cartridge and gradient elution (5-35% EtOAc/hexanes) using an Isolera chromatography station to give isopropyl 2-(5-bromo-2,6-dimethyl-4-(2-azaspiro[4.5]decan-2-yl)pyridin-3-yl)-2-oxoacetate 1.5 g (48%). ¹H NMR (500 MHz, CDCl3) δ 5.05-5.00 (m, 1H), 3.35-3.27 (m, 2H), 2.93 (s, 2H), 2.61 (s, 3H), 2.31 (s, 3H), 1.75 (t, J=6.6 Hz, 2H), 1.47-1.34 (m, 10H), 1.28 (d, J=6.2 Hz, 6H). UPLC (M+H)=439.3.

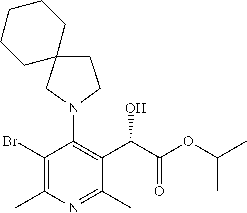 [SMILES: Cc1nc(C)c([C@H](O)C(=O)OC(C)C)c(N2CCC3(CCCCC3)C2)c1Br]

### (S)-Isopropyl 2-(5-bromo-2,6-dimethyl-4-(2-azaspiro[4.5]decan-2-yl)pyridin-3-yl)-2-hydroxyacetate

The 2 mL of benzo[d][1,3,2]dioxaborole (740 mg, 6.17 mmol) was added to a nitrogen purged solution of isopropyl 2-(5-bromo-2,6-dimethyl-4-(2-azaspiro[4.5]decan-2-yl)pyridin-3-yl)-2-oxoacetate (1.5 g, 3.43 mmol) and 1M (R)-1-methyl-3,3-diphenylhexahydropyrrolo[1,2-c][1,3,2]oxazaborole (1.0 mL, 1.0 mmol) in toluene (50 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine, and dried (MgSO₄). The crude product was charged (DCM) to a 120 g ISCO silica gel cartridge and gradient elution (5-30% EtOAc/hexanes) using an Isolera chromatography station gave (S)-isopropyl 2-(5-bromo-2,6-dimethyl-4-(2-azaspiro[4.5]decan-2-yl)pyridin-3-yl)-2-hydroxyacetate 1.2 g (80%) as a mixture of diastereomers. ¹H NMR (500 MHz, DMSO) δ 5.98/5.67 (d, J=4.7 Hz, 1H), 4.96-4.91 (m, 1H), 3.43-3.42 (m, 1H), 3.25-3.20 (m, 1H), 3.13 (d, J 8.1 Hz, 1H), 2.99 (d, J=7.7 Hz, 1H), 2.53 (s, 3H), 2.36 (s, 3H), 1.85-1.78 (m, 2H), 1.61-1.36 (m, 10H), 1.15 (d, J=6.2 Hz, 3H), 1.07 (d, J=6.2 Hz, 3H). UPLC (M+H)=441.3.

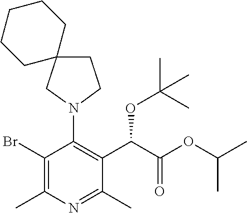 [SMILES: Cc1nc(C)c([C@H](OC(C)(C)C)C(=O)OC(C)C)c(N2CCC3(CCCCC3)C2)c1Br]

### (S)-Isopropyl 2-(5-bromo-2,6-dimethyl-4-(2-azaspiro[4.5]decan-2-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(2-azaspiro[4.5]decan-2-yl)pyridin-3-yl)-2-hydroxyacetate (1.18 g, 2.7 mmol) and 0.5 mL of 70% HClO₄ in DCM (25 mL) for 20 min. The reaction mixture was allowed to warm to rt and stirred for 18 h in a pressure sealed vessel. The reaction mixture was diluted with DCM, washed with 1M Na₂CO₃ soln, and dried over MgSO₄. The crude product was charged (DCM) to a 80 g ISCO silica gel cartridge and gradient elution (5-35% EtOAc/hexanes) using an Isolera chromatography station gave (S)-isopropyl 2-(5-bromo-2,6-dimethyl-4-(2-azaspiro[4.5]decan-2-yl)pyridin-3-yl)-2-(tert-butoxy)acetate 1.12 g (84%) as a mixture of diastereomers. ¹H NMR (500 MHz, DMSO) δ 5.96 (s, 1H), 4.93-4.88 (m, 1H), 3.58 (br. s, 1H), 3.32 (d, J=8.1 Hz, 1H), 3.18-3.13 (m, 1H), 2.91 (d, J=8.1 Hz, 1H), 2.53 (s, 3H), 2.41 (s, 3H), 1.91-1.86 (m, 1H), 1.82-1.79 (m, 1H), 1.67-1.65 (m, 1H), 1.55-1.31 (m, 9H), 1.16 (d, J=6.2 Hz, 3H), 1.14 (s, 9H), 1.06 (d, J=6.2 Hz, 3H). UPLC (M+H)=497.4.

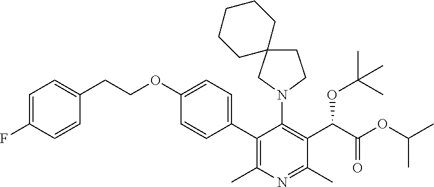 [SMILES: Cc1nc(C)c([C@H](OC(C)(C)C)C(=O)OC(C)C)c(N2CCC3(CCCCC3)C2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(2-azaspiro[4.5]decan-2-yl)pyridin-3-yl)acetate

The tretrakis (35 mg, 0.03 mmol) was added to an argon purged and degassed solution (S)-isopropyl 2-(5-bromo-2,6-dimethyl-4-(2-azaspiro[4.5]decan-2-yl)pyridin-3-yl)-2-(tert-butoxy)acetate (150 mg, 0.30 mmol), (4-(4-fluorophenethoxy)phenyl)boronic acid (87 mg, 0.33 mmol), and sodium carbonate (193 mg, 1.82 mmol) in dioxane (4.5 mL) and water (0.9 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(2-azaspiro[4.5]decan-2-yl)pyridin-3-yl)acetate 110 mg (58%) as a mixture of diastereomers. ¹H NMR (500 MHz, DMSO) δ 7.39-7.36 (m, 2H), 7.20 (d, J=8.8 Hz, 1H), 7.13 (t, J=8.8 Hz, 2H), 7.03-6.97 (m, 3H), 5.78 (s, 1H), 4.97-4.92 (m, 1H), 4.22-4.21 (m, 2H), 3.04 (t, J=6.6 Hz, 2H), 2.91 (d, J=9.1 Hz, 1H), 2.79-2.74 (m, 2H), 2.59 (d, J=8.4 Hz, 1H), 2.40 (s, 3H), 2.05 (s, 3H), 1.40-1.21 (m, 12H), 1.19 (d, J=6.2 Hz, 3H), 1.14 (d, J=6.2 Hz, 3H), 1.10 (s, 9H). UPLC (M+H)=631.6.

### Example 62

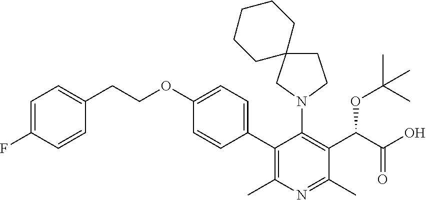 [SMILES: Cc1nc(C)c([C@H](OC(C)(C)C)C(=O)O)c(N2CCC3(CCCCC3)C2)c1-c1ccc(OCCc2ccc(F)cc2)cc1]

### (S)-2-(tert-Butoxy)-2-(5-(4-(4-fluorophenethoxy)phenyl)-2,6-dimethyl-4-(2-azaspiro[4.5]decan-2-yl)pyridin-3-yl)acetic acid

The potassium hydroxide (89 mg, 1.59 mmol) was added to a solution of (S)-isopropyl 2-(tert-butoxy)-2-(5-(4-(4-fluorophenethoxy)phenyl)-2,6-dimethyl-4-(2-azaspiro[4.5]decan-2-yl)pyridin-3-yl)acetate (100 mg, 0.159 mmol) in ethanol (1.5 mL) and stirred for 3.5 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)-phenyl)-2,6-dimethyl-4-(2-azaspiro[4.5]decan-2-yl)pyridin-3-yl)acetic acid 48 mg (50%) as a mixture of diastereomers. ¹H NMR (500 MHz, DMSO) δ 7.39-7.36 (m, 2H), 7.19 (d, J=8.8 Hz, 1H), 7.13 (t, J=8.8 Hz, 2H), 7.02-7.00 (m, 3H), 5.73 (s, 1H), 4.23-4.20 (m, 2H), 3.04 (t, J=6.6 Hz, 2H), 2.8-2.88 (m, 1H), 2.85-2.81 (m, 1H), 2.77-2.74 (m, 1H), 2.56 (d, J=9.2 Hz, 1H), 2.42 (s, 3H), 2.05 (s, 3H), 1.38-1.17 (m, 12H), 1.10 (s, 9H). UPLC (M+H)=589.6.

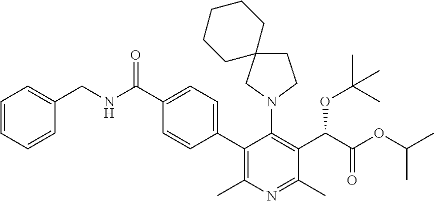 [SMILES: Cc1nc(C)c([C@H](OC(C)(C)C)C(=O)OC(C)C)c(N2CCC3(CCCCC3)C2)c1-c1ccc(C(=O)NCc2ccccc2)cc1]

### (S)-Isopropyl 2-(5-(4-(benzylcarbamoyl)phenyl)-2,6-dimethyl-4-(2-azaspiro[4.5]decan-2-yl)pyridin-3-yl)-2-(tert-butoxy)acetate

The tretrakis (35 mg, 0.03 mmol) was added to an argon purged and degassed solution (S)-isopropyl 2-(5-bromo-2,6-dimethyl-4-(2-azaspiro[4.5]decan-2-yl)pyridin-3-yl)-2-(tert-butoxy)acetate (150 mg, 0.30 mmol), (4-(benzylcarbamoyl)phenyl)boronic acid (85 mg, 0.33 mmol), and sodium carbonate (193 mg, 1.82 mmol) in dioxane (4.5 mL) and water (0.9 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-2,6-dimethyl-4-(2-azaspiro[4.5]decan-2-yl)pyridin-3-yl)-2-(tert-butoxy)acetate 105 mg (55%) as a mixture of diastereomers. ¹H NMR (500 MHz, DMSO) δ 9.16-9.13 (m, 1H), 8.01-7.98 (m, 2H), 7.47 (d, J=7.7 Hz, 1H), 7.35-7.34 (m, 4H), 7.26-7.22 (m, 2H), 5.77 (s, 1H), 4.99-4.94 (m, 1H), 4.52 (d, J=5.9 Hz, 2H), 2.90-2.88 (m. 1H), 2.80-2.76 (m, 2H), 2.63 (d, J=8.8 Hz, 1H), 2.43 (s, 3H), 2.07 (s, 3H), 1.40-1.23 (m, 12H), 1.21 (d, J=5.9 Hz, 3H), 1.15 (d, J=6.2 Hz, 3H), 1.12 (s, 9H). UPLC (M+H)=626.6.

### Example 63

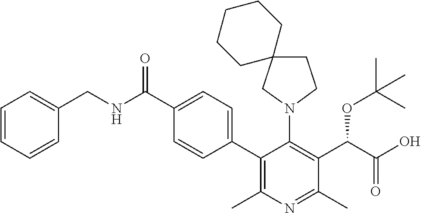 [SMILES: Cc1nc(C)c([C@H](OC(C)(C)C)C(=O)O)c(N2CCC3(CCCCC3)C2)c1-c1ccc(C(=O)NCc2ccccc2)cc1]

### (S)-2-(5-(4-(Benzylcarbamoyl)phenyl)-2,6-dimethyl-4-(2-azaspiro[4.5]decan-2-yl)pyridin-3-yl)-2-(tert-butoxy)acetic acid

The potassium hydroxide (81 mg, 1.44 mmol) was added to a solution of (S)-isopropyl 2-(5-(4-(benzylcarbamoyl)phenyl)-2,6-dimethyl-4-(2-azaspiro[4.5]decan-2-yl)pyridin-3-yl)-2-(tert-butoxy)acetate (90 mg, 0.144 mmol) in ethanol (2 mL) and stirred for 3 h at 90° C. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(2-azaspiro[4.5]decan-2-yl)pyridin-3-yl)-2-(tert-butoxy)acetic acid 54 mg (64%) as a mixture of diastereomers. ¹H NMR (500 MHz, DMSO) δ 9.16-9.13 (m, 1H), 8.00-7.98 (m, 2H), 7.45 (d, J=7.7 Hz, 1H), 7.34-7.33 (m, 4H), 7.26-7.23 (m, 2H), 5.70 (s, 1H), 4.52 (d, J=6.2 Hz, 2H), 2.87-2.79 (m, 3H), 2.59 (d, J=8.8 Hz, 1H), 2.45 (s, 3H), 2.05 (s, 3H), 1.37-1.14 (m, 12H), 1.11 (s, 9H). UPLC (M+H)=584.6.

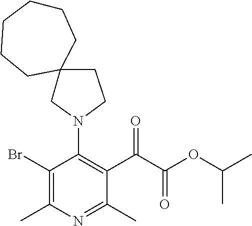 [SMILES: Cc1nc(C)c(C(=O)C(=O)OC(C)C)c(N2CCC3(CCCCCC3)C2)c1Br]

### Isopropyl 2-(5-bromo-2,6-dimethyl-4-(2-azaspiro[4.6]undecan-2-yl)pyridin-3-yl)-2-oxoacetate

To a solution 2-azaspiro[4.6]undecane (1 g, 6.52 mmol) and DIEA (3.4 mL, 19.6 mmol) in anhydrous CH₃CN (35 mL) was added isopropyl 2-(5-bromo-4-chloro-2,6-dimethylpyridin-3-yl)-2-oxoacetate (2.2 g, 6.52 mmol) at rt. The resulting mixture was placed in a pre-heated oil bath (80° C.) and stirred for 18 h; cooled, and concentrated. The crude product was charged (DCM) to a 80 g ISCO silica gel cartridge and gradient elution (5-35% EtOAc/hexanes) using an Isolera chromatography station to isopropyl 2-(5-bromo-2,6-dimethyl-4-(2-azaspiro[4.6]undecan-2-yl)pyridin-3-yl)-2-oxoacetate 2.1 g (71%). ¹H NMR (500 MHz, CDCl3) δ 5.05-5.00 (m, 1H), 3.28 (t, J=7.0 Hz, 2H), 2.88 (s, 2H), 2.61 (s, 3H), 2.30 (s, 3H), 1.76 (t, J=7.0 Hz, 2H), 1.63-1.41 (m, 12H), 1.28 (d, J=6.2 Hz, 6H). UPLC (M+H)=453.4.

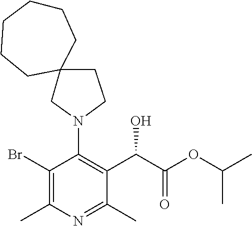 [SMILES: Cc1nc(C)c([C@H](O)C(=O)OC(C)C)c(N2CCC3(CCCCCC3)C2)c1Br]

### (S)-Isopropyl 2-(5-bromo-2,6-dimethyl-4-(2-azaspiro[4.6]undecan-2-yl)pyridin-3-yl)-2-hydroxyacetate

The 1.6 mL of benzo[d][1,3,2]dioxaborole (1.0 g, 8.37 mmol) was added to a nitrogen purged solution of isopropyl 2-(5-bromo-2,6-dimethyl-4-(2-azaspiro[4.6]undecan-2-yl)pyridin-3-yl)-2-oxoacetate (2.1 g, 4.65 mmol) and 1M (R)-1-methyl-3,3-diphenylhexahydropyrrolo[1,2-c][1,3,2]oxazaborole (1.4 mL, 1.4 mmol) in toluene (65 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine, and dried (MgSO₄). The crude product was charged (DCM) to a 80 g ISCO silica gel cartridge and gradient elution (5-45% EtOAc/hexanes) using an Isolera chromatography station gave (S)-isopropyl 2-(5-bromo-2,6-dimethyl-4-(2-azaspiro[4.6]undecan-2-yl)pyridin-3-yl)-2-hydroxyacetate 1.2 g (57%) as a mixture of diastereomers. ¹H NMR (500 MHz, DMSO) δ 5.98/5.72 (d, J=4.4 Hz, 1H), 4.96-4.91 (m, 1H), 3.38 (br. s, 1H), 3.25-3.21 (m, 1H), 3.07 (d, J=7.3 Hz, 1H), 3.01 (d, J=7.3 Hz, 1H), 2.53 (s, 3H), 2.35 (s, 3H), 1.84-1.61 (m, 6H), 1.53-1.45 (m, 8H), 1.15 (d, J=6.2 Hz, 3H), 1.07 (d, J=5.9 Hz, 3H). UPLC (M+H)=455.3.

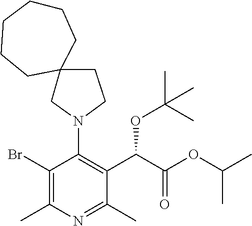 [SMILES: Cc1nc(C)c([C@H](OC(C)(C)C)C(=O)OC(C)C)c(N2CCC3(CCCCCC3)C2)c1Br]

### (S)-Isopropyl 2-(5-bromo-2,6-dimethyl-4-(2-azaspiro[4.6]undecan-2-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(2-azaspiro[4.6]undecan-2-yl)pyridin-3-yl)-2-hydroxyacetate (1.2 g, 2.65 mmol) and 0.5 mL of 70% HClO₄ in DCM (25 mL) for 20 min. The reaction mixture was allowed to warm to rt and stirred for 18 h in a pressure sealed vessel. The reaction mixture was diluted with DCM, washed with 1M Na₂CO₃ soln, and dried over MgSO₄. The crude product was charged (DCM) to a 80 g ISCO silica gel cartridge and gradient elution (5-35% EtOAc/hexanes) using an Isolera chromatography station gave (S)-isopropyl 2-(5-bromo-2,6-dimethyl-4-(2-azaspiro[4.6]undecan-2-yl)pyridin-3-yl)-2-(tert-butoxy)acetate 703 mg (52%) as a mixture of diastereomers. ¹H NMR (500 MHz, DMSO) δ 6.01 (s, 1H), 4.92-4.87 (m, 1H), 3.55 (br. s, 1H), 3.31 (d, J=7.7 Hz, 1H), 3.18-3.15 (m, 1H), 2.91 (d, J=7.7 Hz, 1H), 2.53 (s, 3H), 2.41 (s, 3H), 1.92-1.81 (m, 3H), 1.70-1.64 (m, 3H), 1.55-1.40 (m, 8H), 1.16 (d, J=6.6 Hz, 3H), 1.14 (s, 9H), 1.05 (d, J=6.2 Hz, 3H). UPLC (M+H)=511.4.

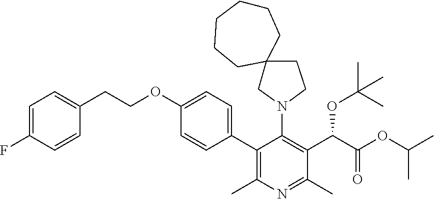 [SMILES: Cc1nc(C)c([C@H](OC(C)(C)C)C(=O)OC(C)C)c(N2CCC3(CCCCCC3)C2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(2-azaspiro[4.6]undecan-2-yl)pyridin-3-yl)acetate

The tretrakis (22.7 mg, 0.02 mmol) was added to a nitrogen purged and degassed solution (S)-isopropyl 2-(5-bromo-2,6-dimethyl-4-(2-azaspiro[4.6]undecan-2-yl)pyridin-3-yl)-2-(tert-butoxy)acetate (100 mg, 0.20 mmol), (4-(4-fluorophenethoxy)phenyl)boronic acid (56 mg, 0.22 mmol), and sodium carbonate (125 mg, 1.20 mmol) in dioxane (3 mL) and water (0.6 mL) and stirred in a screw-capped pressure vessel for 4 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(2-azaspiro[4.6]undecan-2-yl)pyridin-3-yl)acetate 55 mg (43%) as a mixture of diastereomers. ¹H NMR (500 MHz, DMSO) δ 7.39-7.36 (m, 2H), 7.20 (d, J=8.4 Hz, 1H), 7.13 (t, J=8.8 Hz, 2H), 7.04-7.01 (m, 3H), 5.72 (s, 1H), 4.98-4.93 (m, 1H), 4.22 (t, J=6.6 Hz, 2H), 3.05 (t, J=6.2 Hz, 2H), 2.86-2.81 (m, 3H), 2.64 (br. s, 1H), 2.44 (s, 3H), 2.08 (s, 3H), 1.47-1.23 (m, 14H), 1.19 (d, J=6.2 Hz, 3H), 1.15 (d, J=6.2 Hz, 3H), 1.11 (s, 9H). UPLC (M+H)=645.7.

### Example 64

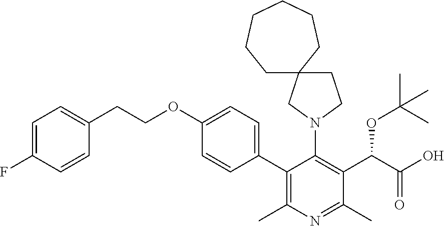 [SMILES: Cc1nc(C)c([C@H](OC(C)(C)C)C(=O)O)c(N2CCC3(CCCCCC3)C2)c1-c1ccc(OCCc2ccc(F)cc2)cc1]

### (S)-2-(tert-Butoxy)-2-(5-(4-(4-fluorophenethoxy)phenyl)-2,6-dimethyl-4-(2-azaspiro[4.6]undecan-2-yl)pyridin-3-yl)acetic acid

The potassium hydroxide (43.5 mg, 0.78 mmol) was added to a solution of (S)-isopropyl 2-(tert-butoxy)-2-(5-(4-(4-fluorophenethoxy)phenyl)-2,6-dimethyl-4-(2-azaspiro[4.6]undecan-2-yl)pyridin-3-yl)acetate (50 mg, 0.078 mmol) in ethanol (2 mL) and stirred for 3.5 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)-phenyl)-2,6-dimethyl-4-(2-azaspiro[4.6]undecan-2-yl)pyridin-3-yl)acetic acid 35.5 mg (76%) as a mixture of diastereomers. ¹H NMR (500 MHz, DMSO) δ 7.38-7.36 (m, 2H), 7.19 (d, J 8.4 Hz, 1H), 7.13 (t, J 8.8 Hz, 2H), 7.02-6.99 (m, 3H), 5.74 (s, 1H), 4.22 (d, J=6.6 Hz, 2H), 3.04 (t, J=6.6 Hz, 2H), 2.89-2.83 (m, 2H), 2.80-2.76 (m, 1H), 2.55 (d, J=8.8 Hz, 1H), 2.42 (s, 3H), 2.05 (s, 3H), 1.46-1.21 (m, 14H), 1.10 (s, 9H). UPLC (M+H)=603.6.

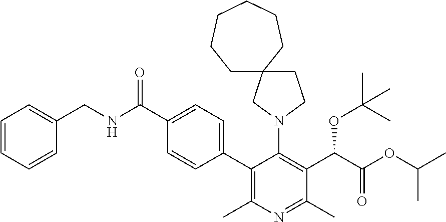 [SMILES: Cc1nc(C)c([C@H](OC(C)(C)C)C(=O)OC(C)C)c(N2CCC3(CCCCCC3)C2)c1-c1ccc(C(=O)NCc2ccccc2)cc1]

### (S)-Isopropyl 2-(5-(4-(benzylcarbamoyl)phenyl)-2,6-dimethyl-4-(2-azaspiro[4.6]undecan-2-yl)pyridin-3-yl)-2-(tert-butoxy)acetate

The tretrakis (27.2 mg, 0.024 mmol) was added to an argon purged and degassed solution (S)-isopropyl 2-(5-bromo-2,6-dimethyl-4-(2-azaspiro[4.6]undecan-2-yl)pyridin-3-yl)-2-(tert-butoxy)acetate (120 mg, 0.24 mmol), (4-(benzylcarbamoyl)phenyl)boronic acid (66 mg, 0.26 mmol), and sodium carbonate (150 mg, 1.41 mmol) in dioxane (4 mL) and water (0.7 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-2,6-dimethyl-4-(2-azaspiro[4.6]undecan-2-yl)pyridin-3-yl)-2-(tert-butoxy)acetate 88 mg (58%) as a mixture of diastereomers. ¹H NMR (500 MHz, DMSO) δ 9.15-9.14 (m, 1H), 7.99 (t, J=6.6 Hz, 2H), 7.46 (d, J=7.7 Hz, 1H), 7.34-7.33 (m, 4H), 7.28-7.21 (m, 2H), 5.78 (s, 1H), 4.98-4.93 (m, 1H), 4.52 (d, J=5.9 Hz, 2H), 2.84-2.78 (m, 3H), 2.60 (d, J=8.4 Hz, 1H), 2.43 (s, 3H), 2.06 (s, 3H), 1.44-1.22 (m, 14H), 1.20 (d, J=6.2 Hz, 3H), 1.15 (d, J=6.2 Hz, 3H), 1.12 (s, 9H). UPLC (M+H)=640.7.

### Example 65

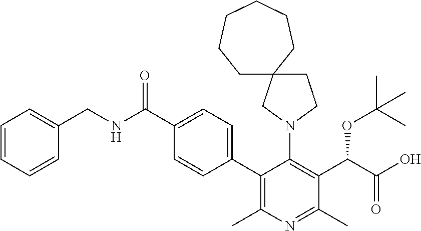 [SMILES: Cc1nc(C)c([C@H](OC(C)(C)C)C(=O)O)c(N2CCC3(CCCCCC3)C2)c1-c1ccc(C(=O)NCc2ccccc2)cc1]

### (S)-2-(5-(4-(Benzylcarbamoyl)phenyl)-2,6-dimethyl-4-(2-azaspiro[4.6]undecan-2-yl)pyridin-3-yl)-2-(tert-butoxy) acetic acid

The potassium hydroxide (65.4 mg, 1.20 mmol) was added to a solution of (S)-isopropyl 2-(5-(4-(benzylcarbamoyl)phenyl)-2,6-dimethyl-4-(2-azaspiro[4.6]undecan-2-yl)pyridin-3-yl)-2-(tert-butoxy)acetate (74.6 mg, 0.12 mmol) in ethanol (2 mL) and stirred for 3.5 h at 90° C. The reaction mixture was neutralized with 1N HCl soln, extracted with EtOAc, and the organic layer was washed with brine, and dried (MgSO₄). The crude material was purified by prep HPLC to obtain (S)-2-(5-(4-(benzylcarbamoyl)phenyl)-2,6-dimethyl-4-(2-azaspiro[4.6]undecan-2-yl)pyridin-3-yl)-2-(tert-butoxy) acetic acid 42 mg (61%) as a mixture of diastereomers. ¹H NMR (500 MHz, DMSO) δ 9.16-9.13 (m, 1H), 7.99 (t, J=6.2 Hz, 2H), 7.45 (d, J=7.7 Hz, 1H), 7.34-7.33 (m, 4H), 7.27-7.23 (m, 2H), 5.73 (s, 1H), 4.52 (d, J=5.9 Hz, 2H), 2.87-2.79 (m, 3H), 2.58 (d, J=8.4 Hz, 1H), 2.45 (s, 3H), 2.05 (s, 3H), 1.42-1.21 (m, 14H), 1.11 (s, 9H). UPLC (M+H)=598.6.

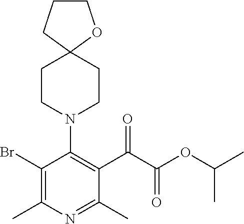 [SMILES: Cc1nc(C)c(C(=O)C(=O)OC(C)C)c(N2CCC3(CCCO3)CC2)c1Br]

### Isopropyl 2-(5-bromo-2,6-dimethyl-4-(1-oxa-8-azaspiro[4.5]decan-8-yl)pyridin-3-yl)-2-oxoacetate

To a solution 1-oxa-8-azaspiro[4.5]decane, HCl (150 mg, 0.84 mmol) and DIEA (0.44 mL, 2.53 mmol) in anhydrous CH₃CN (10 mL) was added isopropyl 2-(5-bromo-4-chloro-2,6-dimethylpyridin-3-yl)-2-oxoacetate (282 mg, 0.84 mmol) at rt. The resulting mixture was placed in a pre-heated oil bath (80° C.) and stirred for 18 h; cooled, and concentrated. The crude product was charged (DCM) to a 40 g ISCO silica gel cartridge and gradient elution (5-35% EtOAc/hexanes) using an Isolera chromatography station to give isopropyl 2-(5-bromo-2,6-dimethyl-4-(1-oxa-8-azaspiro[4.5]decan-8-yl)pyridin-3-yl)-2-oxoacetate 176 mg (47%). ¹H NMR (500 MHz, CDCl3) δ 5.09-5.04 (m, 1H), 3.73 (t, J=7.0 Hz, 2H), 3.36-3.35 (m, 4H), 2.61 (s, 3H), 2.30 (s, 3H), 1.89-1.84 (m, 2H), 1.66 (t, J=8.1 Hz, 2H), 1.56-1.51 (m, 4H), 1.30 (d, J=5.9 Hz, 6H). UPLC (M+H)=441.2.

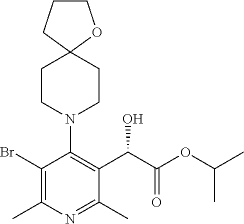 [SMILES: Cc1nc(C)c([C@H](O)C(=O)OC(C)C)c(N2CCC3(CCCO3)CC2)c1Br]

### (S)-Isopropyl 2-(5-bromo-2,6-dimethyl-4-(1-oxa-8-azaspiro[4.5]decan-8-yl)pyridin-3-yl)-2-hydroxyacetate

The 0.3 mL of benzo[d][1,3,2]dioxaborole (84 mg, 0.70 mmol) was added to a nitrogen purged solution of isopropyl 2-(5-bromo-2,6-dimethyl-4-(1-oxa-8-azaspiro[4.5]decan-8-yl)pyridin-3-yl)-2-oxoacetate (170 mg, 0.39 mmol) and 1M (R)-1-methyl-3,3-diphenylhexahydropyrrolo[1,2-c][1,3,2]oxazaborole (0.12 mL, 0.12 mmol) in toluene (15 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine, and dried (MgSO₄). The crude product was charged (DCM) to a 24 g ISCO silica gel cartridge and gradient elution (5-35% EtOAc/hexanes) using an Isolera chromatography station gave (S)-isopropyl 2-(5-bromo-2,6-dimethyl-4-(1-oxa-8-azaspiro[4.5]decan-8-yl)pyridin-3-yl)-2-hydroxyacetate 120 mg (71%) as a mixture of diastereomers. ¹H NMR (500 MHz, DMSO) δ 5.88/5.80 (s, 1H), 4.96-4.93 (m, 1H), 3.80/3.88 (t, J=11.7 Hz, 1H), 3.77-3.72 (m, 2H), 3.47/3.29 (t, J=11.7 Hz, 1H), 2.96/2.89 (d, J=9.2 Hz, 1H), 2.73/2.61 (d, J=8.8 Hz, 1H), 2.52 (s, 3H), 2.37 (s, 3H), 1.91-1.84 (m, 2H), 1.78-1.65 (m, 4H), 1.59-1.53 (m, 2H), 1.15 (d, J=6.2 Hz, 3H), 1.07 (d, J=5.1 Hz, 3H). UPLC (M+H)=443.2.

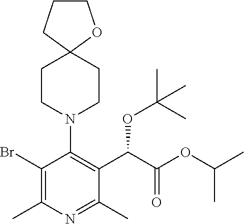 [SMILES: Cc1nc(C)c([C@H](OC(C)(C)C)C(=O)OC(C)C)c(N2CCC3(CCCO3)CC2)c1Br]

### (S)-Isopropyl 2-(5-bromo-2,6-dimethyl-4-(1-oxa-8-azaspiro[4.5]decan-8-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(1-oxa-8-azaspiro[4.5]decan-8-yl)pyridin-3-yl)-2-hydroxyacetate (110 mg, 0.25 mmol) and 0.07 mL of 70% HClO₄ in DCM (5 mL) for 20 min. The reaction mixture was allowed to warm to rt and stirred for 18 h in a pressure sealed vessel. The reaction mixture was diluted with DCM, washed with 1M Na₂CO₃ soln, and dried over MgSO₄. The crude product was charged (DCM) to a 24 g ISCO silica gel cartridge and gradient elution (5-55% EtOAc/hexanes) using an Isolera chromatography station gave (S)-isopropyl 2-(5-bromo-2,6-dimethyl-4-(1-oxa-8-azaspiro[4.5]decan-8-yl)pyridin-3-yl)-2-(tert-butoxy)acetate 98 mg (79%) as a mixture of diastereomers. ¹H NMR (500 MHz, DMSO) δ 6.20 (br. s, 1H), 4.93-4.89 (m, 1H), 4.08/3.77 (br. s, 1H), 3.78-3.72 (m, 2H), 3.50/3.27 (t, J=10.6 Hz, 1H), 3.01/2.80 (br. s, 1H), 2.80/2.87 (br. s, 1H), 2.52 (s, 3H), 2.42 (s, 3H), 1.93-1.85 (m, 2H), 1.82-1.57 (m, 6H), 1.15-1.14 (m, 12H), 1.07 (d, J=5.9 Hz, 3H). UPLC (M+H)=499.3.

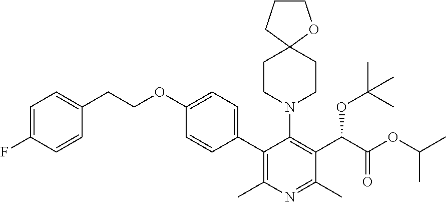 [SMILES: Cc1nc(C)c([C@H](OC(C)(C)C)C(=O)OC(C)C)c(N2CCC3(CCCO3)CC2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(1-oxa-8-azaspiro[4.5]decan-8-yl)pyridin-3-yl)acetate

The tretrakis (20.9 mg, 0.018 mmol) was added to nitrogen purged and degassed solution (S)-isopropyl 2-(5-bromo-2,6-dimethyl-4-(1-oxa-8-azaspiro[4.5]decan-8-yl)pyridin-3-yl)-2-(tert-butoxy)acetate (90 mg, 0.18 mmol), (4-(4-fluorophenethoxy)phenyl)boronic acid (56 mg, 0.22 mmol), and sodium carbonate (115 mg, 1.10 mmol) in dioxane (3 mL) and water (0.6 mL) and stirred in a screw-capped pressure vessel for 4 at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(1-oxa-8-azaspiro[4.5]decan-8-yl)pyridin-3-yl)acetate 91 mg (79%) as a mixture of diastereomers. ¹H NMR (500 MHz, DMSO) δ 7.40-7.37 (m, 2H), 7.22-7.13 (m, 3H), 7.02-6.98 (m, 3H), 5.89 (s, 1H), 4.97-4.93 (m, 1H), 4.24 (t, J=7.0 Hz, 2H), 3.64-3.54 (m, 2H), 3.39-3.55 (m, 1H), 3.29-3.26/3.12-3.09 (m, 1H), 3.06 (t, J=6.2 Hz, 2H), 2.90/2.65 (t, J=12.0 Hz, 1H), 2.44/2.43 (s, 3H), 2.29-2.26/2.24-2.10 (m, 1H), 2.05/2.02 (s, 3H), 1.81-1.23 (m, 8H), 1.19 (d, J=6.2 Hz, 3H), 1.14-1.12 (m, 12H). UPLC (M+H)=633.5.

### Example 66

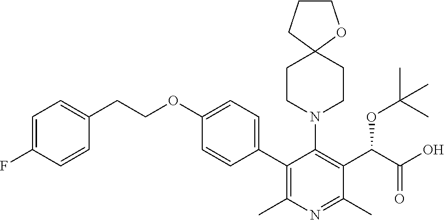 [SMILES: Cc1nc(C)c([C@H](OC(C)(C)C)C(=O)O)c(N2CCC3(CCCO3)CC2)c1-c1ccc(OCCc2ccc(F)cc2)cc1]

### (S)-2-(tert-Butoxy)-2-(5-(4-(4-fluorophenethoxy)phenyl)-2,6-dimethyl-4-(1-oxa-8-azaspiro[4.5]decan-8-yl)pyridin-3-yl)acetic acid

The potassium hydroxide (72 mg, 1.30 mmol) was added to a solution of (S)-isopropyl 2-(tert-butoxy)-2-(5-(4-(4-fluorophenethoxy)phenyl)-2,6-dimethyl-4-(1-oxa-8-azaspiro[4.5]decan-8-yl)pyridin-3-yl)acetate (81 mg, 0.13 mmol) in ethanol (3 mL) and stirred for 3 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)-phenyl)-2,6-dimethyl-4-(1-oxa-8-azaspiro[4.5]decan-8-yl)pyridin-3-yl)acetic acid 43 mg (57%) as a mixture of diastereomers. ¹H NMR (500 MHz, DMSO) δ 7.40-7.37 (m, 2H), 7.19-7.12 (m, 3H), 7.01-6.97 (m, 3H), 5.82 (s, 1H), 4.23 (d, J=6.3 Hz, 2H), 3.45 (br. s, 1H), 3.27 (br. s, 1H), 3.06-3.05 (m, 2H), 2.87/2.64 (t, J=10.6 Hz, 1H), 2.44 (s, 3H), 2.27-2.25/2.12-2.08 (m, 1H), 2.05/2.01 (s, 3H), 1.78-1.22 (m, 8H), 1.13 (s, 9H). UPLC (M+H)=591.4.

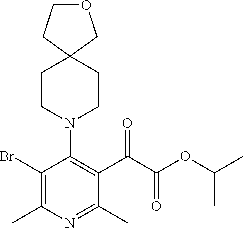 [SMILES: Cc1nc(C)c(C(=O)C(=O)OC(C)C)c(N2CCC3(CCOC3)CC2)c1Br]

### Isopropyl 2-(5-bromo-2,6-dimethyl-4-(2-oxa-8-azaspiro[4.5]decan-8-yl)pyridin-3-yl)-2-oxoacetate

To a solution 2-oxa-8-azaspiro[4.5]decane, HCl (1 g, 5.63 mmol) and DIEA (2.9 mL, 16.9 mmol) in anhydrous CH₃CN (35 mL) was added isopropyl 2-(5-bromo-4-chloro-2,6-dimethylpyridin-3-yl)-2-oxoacetate (1.9 g, 5.63 mmol) at rt. The resulting mixture was placed in a pre-heated oil bath (80° C.) and stirred for 18 h; cooled, and concentrated. The crude product was charged (DCM) to a 80 g ISCO silica gel cartridge and gradient elution (5-35% EtOAc/hexanes) using an Isolera chromatography station to give isopropyl 2-(5-bromo-2,6-dimethyl-4-(2-oxa-8-azaspiro[4.5]decan-8-yl)pyridin-3-yl)-2-oxoacetate 990 mg (40%). ¹H NMR (500 MHz, CDCl3) δ 5.08-5.05 (m, 1H), 3.75 (t, J=7.3 Hz, 2H), 3.37 (s, 2H), 3.36-3.34 (m, 4H), 2.61 (s, 3H), 2.30 (s, 3H), 1.73-1.71 (m, 2H), 1.50 (br. s, 4H), 1.29 (d, J=6.2 Hz, 6H). UPLC (M+H)=441.1.

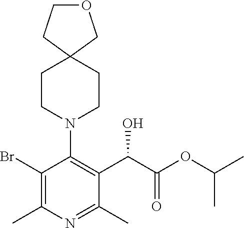 [SMILES: Cc1nc(C)c([C@H](O)C(=O)OC(C)C)c(N2CCC3(CCOC3)CC2)c1Br]

### (S)-Isopropyl 2-(5-bromo-2,6-dimethyl-4-(2-oxa-8-azaspiro[4.5]decan-8-yl)pyridin-3-yl)-2-hydroxyacetate

The 1.7 mL of benzo[d][1,3,2]dioxaborole (481 mg, 4.02 mmol) was added to a nitrogen purged solution of isopropyl 2-(5-bromo-2,6-dimethyl-4-(2-oxa-8-azaspiro[4.5]decan-8-yl)pyridin-3-yl)-2-oxoacetate (980 mg, 2.23 mmol) and 1M (R)-1-methyl-3,3-diphenylhexahydropyrrolo[1,2-c][1,3,2]oxazaborole (0.7 mL, 0.67 mmol) in toluene (20 mL) at −60° C. and allowed to warm to −15° C. before being placed in the freezer 18 h. The reaction was quenched with 1M Na₂CO₃, diluted with EtOAc, and stirred for 30 min. The organic layer was washed with sat'd Na₂CO₃ soln, brine, and dried (MgSO₄). The crude product was charged (DCM) to a 40 g ISCO silica gel cartridge and gradient elution (5-55% EtOAc/hexanes) using an Isolera chromatography station gave (S)-isopropyl 2-(5-bromo-2,6-dimethyl-4-(2-oxa-8-azaspiro[4.5]decan-8-yl)pyridin-3-yl)-2-hydroxyacetate 900 mg (91%) as a mixture of diastereomers. ¹H NMR (500 MHz, DMSO) δ 5.93-5.88 (m, 1H), 4.95-4.93 (m, 1H), 7.79 (t, J=7.3 Hz, 1H), 3.74 (t, J=7.0 Hz, 1H), 3.67-3.64 (m, 1H), 3.53-3.45 (m, 1H), 3.42 (s, 2H), 2.85 (br. s, 1H), 2.75 (br. s, 1H), 2.53 (s, 3H), 2.38 (s, 3H), 1.81 (t, J=6.6 Hz, 1H), 1.72-1.47 (m, 5H), 1.15 (d, J=4.8 Hz, 3H), 1.08 (d, J=5.9 Hz, 3H). UPLC (M+H)=443.1.

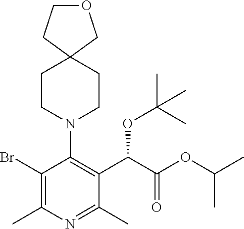 [SMILES: Cc1nc(C)c([C@H](OC(C)(C)C)C(=O)OC(C)C)c(N2CCC3(CCOC3)CC2)c1Br]

### (S)-Isopropyl 2-(5-bromo-2,6-dimethyl-4-(2-oxa-8-azaspiro[4.5]decan-8-yl)pyridin-3-yl)-2-(tert-butoxy)acetate

The isobutylene gas was bubbled into a nitrogen purged, cooled (0° C.) solution of (S)-isopropyl 2-(5-bromo-2,6-dimethyl-4-(2-oxa-8-azaspiro[4.5]decan-8-yl)pyridin-3-yl)-2-hydroxyacetate (860 mg, 1.95 mmol) and 0.3 mL of 70% HClO₄ in DCM (15 mL) for 20 min. The reaction mixture was allowed to warm to rt and stirred for 18 h in a pressure sealed vessel. The reaction mixture was diluted with DCM, washed with 1M Na₂CO₃ soln, and dried over MgSO₄. The crude product was charged (DCM) to a 40 g ISCO silica gel cartridge and gradient elution (5-35% EtOAc/hexanes) using an Isolera chromatography station gave (S)-isopropyl 2-(5-bromo-2,6-dimethyl-4-(2-oxa-8-azaspiro[4.5]decan-8-yl)pyridin-3-yl)-2-(tert-butoxy)acetate 271 mg (28%) as a mixture of diastereomers and recovered starting material. ¹H NMR (500 MHz, DMSO) δ 6.19 (br. s, 1H), 4.93-4.89 (m, 1H), 3.81 (t, J=7.0 Hz, 1H), 3.74 (t, J=7.3 Hz, 1H), 3.66-3.62 (m, 1H), 3.45-3.41 (m, 1H), 3.38 (s, 2H), 2.92 (br. s, 1H), 2.68 (br. s, 1H), 2.53 (s, 3H), 2.43 (s, 3H), 1.85 (t, J=6.6 Hz, 1H), 1.74-1.50 (m, 5H), 1.15-1.14 (m, 12H), 1.07 (d, J=5.9 Hz, 3H). UPLC (M+H)=499.2.

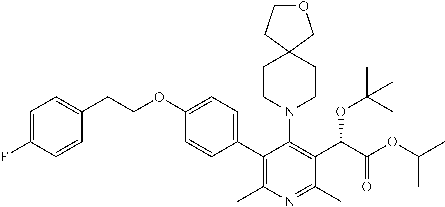 [SMILES: Cc1nc(C)c([C@H](OC(C)(C)C)C(=O)OC(C)C)c(N2CCC3(CCOC3)CC2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(2-oxa-8-azaspiro[4.5]decan-8-yl)pyridin-3-yl)acetate

The tretrakis (59.2 mg, 0.051 mmol) was added to nitrogen purged and degassed solution (S)-isopropyl 2-(5-bromo-2,6-dimethyl-4-(2-oxa-8-azaspiro[4.5]decan-8-yl)pyridin-3-yl)-2-(tert-butoxy)acetate (170 mg, 0.18 mmol), (4-(4-fluorophenethoxy)phenyl)boronic acid (98 mg, 0.38 mmol), and sodium carbonate (217 mg, 2.0 mmol) in dioxane (4.5 mL) and water (0.9 mL) and stirred in a screw-capped pressure vessel for 4 at 90° C. The reaction was allowed to cool, diluted with EtOAc, and the organic layer was washed with brine and dried (MgSO₄). The crude product was charged (DCM) to a 24 g ISCO silica gel cartridge and gradient elution (5-65% EtOAc/hexanes) using an Isolera chromatography station gave (S)-isopropyl 2-(tert-butoxy)-2-(5-(4-(4-fluorophenethoxy)phenyl)-2,6-dimethyl-4-(2-oxa-8-azaspiro[4.5]decan-8-yl)pyridin-3-yl)acetate 107 mg (49.5%) as a mixture of diastereomers. ¹H NMR (500 MHz, DMSO) δ 7.38 (br. s, 2H), 7.22-7.19 (m, 1H), 7.14 (t, J=9.2 Hz, 2H), 7.04 (br. s, 3H), 6.03 (s, 1H), 4.96-4.94 (m, 1H), 4.27-4.21 (m, 2H), 3.64-3.60 (m, 2H), 3.36-3.17 (s, 2H), 3.07-3.04 (m, 2H), 2.78-2.73/2.66-2.60 (m, 1H), 2.45 (s, 3H), 2.38-2.32 (m, 1H), 2.06 (s, 3H), 1.92/1.78 (t, J=12.5 Hz, 1H), 1.70-1.30 (m, 5H), 1.19 (d, J=5.9 Hz, 3H), 1.14-1.13 (m, 12H). UPLC (M+H)=633.4.

### Example 67

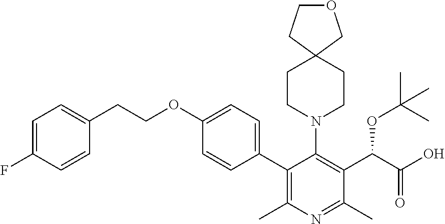 [SMILES: Cc1nc(C)c([C@H](OC(C)(C)C)C(=O)O)c(N2CCC3(CCOC3)CC2)c1-c1ccc(OCCc2ccc(F)cc2)cc1]

### (S)-2-(tert-Butoxy)-2-(5-(4-(4-fluorophenethoxy)phenyl)-2,6-dimethyl-4-(2-oxa-8-azaspiro[4.5]decan-8-yl)pyridin-3-yl)acetic acid

The potassium hydroxide (56 mg, 1.0 mmol) was added to a solution of (S)-isopropyl 2-(tert-butoxy)-2-(5-(4-(4-fluorophenethoxy)phenyl)-2,6-dimethyl-4-(2-oxa-8-azaspiro[4.5]decan-8-yl)pyridin-3-yl)acetate (63 mg, 0.10 mmol) in ethanol (2 mL) and stirred for 3 h at 90° C. The reaction mixture was neutralized with 1N HCl soln, extracted with EtOAc, and the organic layer was washed with brine, and dried (MgSO₄). The crude material was purified by prep HPLC to obtain (S)-2-(tert-butoxy)-2-(5-(4-(4-fluorophenethoxy)-phenyl)-2,6-dimethyl-4-(2-oxa-8-azaspiro[4.5]decan-8-yl)pyridin-3-yl)acetic acid 17 mg (29%) as a mixture of diastereomers. ¹H NMR (500 MHz, DMSO) δ 7.38 (br. s, 2H), 7.22-7.19 (m, 1H), 7.14 (t, J=8.8 Hz, 2H), 7.03 (br. s, 3H), 5.86/5.84 (s, 1H), 4.26-4.11 (m, 2H), 3.61 (br. s, 2H), 3.38-3.32 (m, 3H), 3.26-3.23 (m, 1H), 3.07-3.05 (m, 2H), 2.76-2.71/2.65-2.60 (m, 1H), 2.45 (s, 3H), 2.35-2.30 (m, 1H), 2.05 (s, 3H), 1.76-1.20 (m, 6H), 1.13 (s, 9H). UPLC (M+H)=591.4.

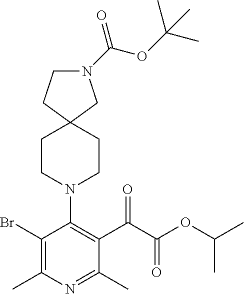 [SMILES: Cc1nc(C)c(C(=O)C(=O)OC(C)C)c(N2CCC3(CCN(C(=O)OC(C)(C)C)C3)CC2)c1Br]

### tert-Butyl 8-(3-bromo-5-(2-isopropoxy-2-oxoacetyl)-2,6-dimethylpyridin-4-yl)-2,8-diazaspiro[4.5]decane-2-carboxylate

To a solution of tert-butyl 2,8-diazaspiro[4.5]decane-2-carboxylate (500 mg, 2.1 mmol) and DIEA (1.1 mL, 6.2 mmol) in anhydrous CH₃CN (20 mL) was added isopropyl 2-(5-bromo-4-chloro-2,6-dimethylpyridin-3-yl)-2-oxoacetate (696 mg, 2.1 mmol) at rt. The resulting mixture was placed in a pre-heated oil bath (80° C.) and stirred for 18 h before being cooled, concentrated, and charged (DCM) to a 40 g ISCO silica gel cartridge and gradient elution (0-50% EtOAc/hexanes) using an Isolera chromatography station to give tert-butyl 8-(3-bromo-5-(2-isopropoxy-2-oxoacetyl)-2,6-dimethylpyridin-4-yl)-2,8-diazaspiro[4.5]decane-2-carboxylate 1.07 g (96%). ¹H NMR (500 MHz, CDCl3) δ 5.10-5.05 (m, 1H), 3.47 (br. s, 4H), 3.30 (br. s, 2H), 3.11 (br. s, 2H), 2.62 (s, 3H), 2.30 (s, 3H), 1.72 (br. s, 2H), 1.47 (br. s, 4H), 1.40 (s, 9H), 1.30 (d, J=5.9 Hz, 6H). UPLC (M+H)=540.3.

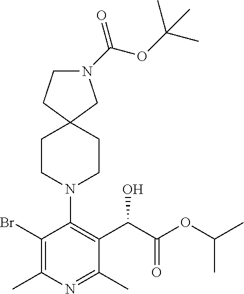 [SMILES: Cc1nc(C)c([C@H](O)C(=O)OC(C)C)c(N2CCC3(CCN(C(=O)OC(C)(C)C)C3)CC2)c1Br]

### (S)-tert-Butyl 8-(3-bromo-5-(1-hydroxy-2-isopropoxy-2-oxoethyl)-2,6-dimethylpyridin-4-yl)-2,8-diazaspiro[4.5]decane-2-carboxylate

The benzo[d][1,3,2]dioxaborole (0.41 mL, 1.89 mmol; 50% soln in toluene) was added to a nitrogen purged solution of tert-butyl 8-(3-bromo-5-(2-isopropoxy-2-oxoacetyl)-2,6-dimethylpyridin-4-yl)-2,8-diazaspiro[4.5]decane-2-carboxylate (1.02 g, 1.89 mmol) and 0.56 mL of 1M (R)-1-methyl-3,3-diphenylhexahydropyrrolo[1,2-c][1,3,2]oxazaborole (0.56 mmol) in toluene (20 mL) cooled to −50° C. The reaction was allowed to slowly warm to −15° C. and placed in the freezer for 18 h before being quenched with 1M Na2CO3 (3 mL) and stirred for 20 min. The organic layer was diluted with EtOAc and washed with brine and dried (MgSO₄). The crude product was charged (DCM) to a 80 g ISCO silica gel cartridge and gradient elution (5-50% EtOAc/hexanes) using an Isolera chromatography station gave (S)-tert-butyl 8-(3-bromo-5-(1-hydroxy-2-isopropoxy-2-oxoethyl)-2,6-dimethylpyridin-4-yl)-2,8-diazaspiro[4.5]decane-2-carboxylate 1.01 g (99%) as a mixture of diastereomers. UPLC (M+H)=542.2.

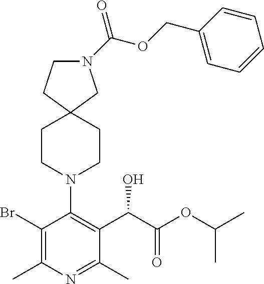 [SMILES: Cc1nc(C)c([C@H](O)C(=O)OC(C)C)c(N2CCC3(CCN(C(=O)OCc4ccccc4)C3)CC2)c1Br]

### (S)-Benzyl 8-(3-bromo-5-(1-hydroxy-2-isopropoxy-2-oxoethyl)-2,6-dimethylpyridin-4-yl)-2,8-diazaspiro[4.5]decane-2-carboxylate

A cold soln of 4M HCl in dioxane (3 mL, 12.00 mmol) was added to (S)-tert-butyl 8-(3-bromo-5-(1-hydroxy-2-isopropoxy-2-oxoethyl)-2,6-dimethylpyridin-4-yl)-2,8-diazaspiro[4.5]decane-2-carboxylate (0.45 g, 0.833 mmol) in dry Dioxane (10 mL). The mixture was stirred for 1 h at rt and concentrate. The residue was taken up in DCM and triethylamine (0.464 mL, 3.33 mmol) was added followed by CBZ—Cl (0.119 mL, 0.833 mmol) at 0° C. under nitrogen. The reaction mixture was allowed to warm up to rt, stirred for 2 h, diluted with EtOAc, washed with water, brine, and dried over (MgSO4). The crude product was charged (DCM) to a 40 g ISCO silica gel cartridge and gradient elution (0-75% EtOAc/hexanes) using an Isolera chromatography station gave (S)-benzyl 8-(3-bromo-5-(1-hydroxy-2-isopropoxy-2-oxoethyl)-2,6-dimethylpyridin-4-yl)-2,8-diazaspiro[4.5]decane-2-carboxylate 410 mg (86%). UPLC (M+H)=576.2.

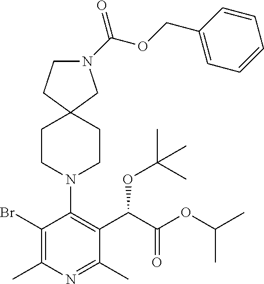 [SMILES: Cc1nc(C)c([C@H](OC(C)(C)C)C(=O)OC(C)C)c(N2CCC3(CCN(C(=O)OCc4ccccc4)C3)CC2)c1Br]

### (S)-Benzyl 8-(3-bromo-5-(1-(tert-butoxy)-2-isopropoxy-2-oxoethyl)-2,6-dimethylpyridin-4-yl)-2,8-diazaspiro[4.5]decane-2-carboxylate

The isobutylene gas was bubbled into a nitrogen purged, cooled (0° C.) solution of (S)-benzyl 8-(3-bromo-5-(1-hydroxy-2-isopropoxy-2-oxoethyl)-2,6-dimethylpyridin-4-yl)-2,8-diazaspiro[4.5]decane-2-carboxylate (400 mg, 0.70 mmol) and 0.07 mL of 70% HClO₄ in DCM (6 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 40 g ISCO silica gel cartridge and gradient elution (5-35% EtOAc/hexanes) using an Isolera chromatography station gave (S)-benzyl 8-(3-bromo-5-(1-(tert-butoxy)-2-isopropopoxy-2-oxoethyl)-2,6-dimethylpyridin-4-yl)-2,8-diazaspiro[4.5]decane-2-carboxylate 350 mg (80%): ¹H NMR (500 MHz, DMSO) δ 7.38-7.36 (m, 4H), 7.34-7.31 (m, 1H), 6.19 (br. s, 1H), 5.11-5.07 (m, 2H), 4.9-4.89 (m, 1H), 3.83 (br. s, 2H), 3.49-3.33 (m, 3H), 3.20-3.17 (m, 1H), 2.92 (br. s, 1H), 2.71-2.63 (m, 1H), 2.53 (s, 3H), 2.43 (s, 3H), 1.94-1.88 (m, 1H), 1.77-1.53 (m, 5H), 1.15-1.14 (m, 12H), 1.07 (d, J=5.9 Hz, 3H). UPLC (M+H)=632.3.

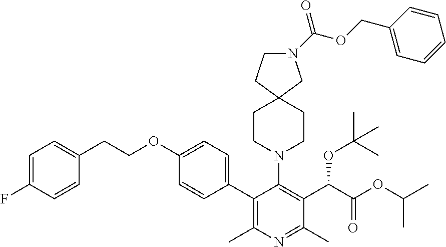 [SMILES: Cc1nc(C)c([C@H](OC(C)(C)C)C(=O)OC(C)C)c(N2CCC3(CCN(C(=O)OCc4ccccc4)C3)CC2)c1-c1ccc(OCCc2ccc(F)cc2)cc1]

### (S)-Benzyl 8-(3-(1-(tert-butoxy)-2-isopropoxy-2-oxoethyl)-5-(4-(4-fluorophenethoxy)phenyl)-2,6-dimethylpyridin-4-yl)-2,8-diazaspiro [4.5]decane-2-carboxylate

The Pd(Ph₃P)₄ (37.6 mg, 0.033 mmol) was added to an argon purged and degassed solution of (S)-benzyl 8-(3-bromo-5-(1-(tert-butoxy)-2-isopropoxy-2-oxoethyl)-2,6-dimethylpyridin-4-yl)-2,8-diazaspiro[4.5]decane-2-carboxylate (205 mg, 0.325 mmol), (4-(4-fluorophenethoxy)phenyl)boronic acid (93 mg, 0.358 mmol), and sodium carbonate (172 mg, 1.63 mmol) in dioxane (2.5 mL) and water (0.63 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-70% EtOAc/hexanes) using an Isolera chromatography station gave (S)-benzyl 8-(3-(1-(tert-butoxy)-2-isopropoxy-2-oxoethyl)-5-(4-(4-fluorophenethoxy)phenyl)-2,6-dimethylpyridin-4-yl)-2,8-diazaspiro [4.5]decane-2-carboxylate 240 mg (97%). ¹H NMR (500 MHz, DMSO) δ 7.42-7.26 (m, 7H), 7.22-7.19 (m, 1H), 7.16-7.08 (m, 2H), 7.05-6.98 (m, 3H), 6.01 (s, 1H), 5.08-5.00 (m, 2H), 4.97-4.91 (m, 1H), 4.30-4.22 (m, 2H), 3.31-3.20 (m, 2H), 3.10-2.91 (m, 4H), 2.82-2.67 (m, 2H), 2.44 (s, 3H), 2.36-2.31 (m, 1H), 2.06 (s, 3H), 1.80 (t, J=11.7 Hz, 1H), 1.63-1.24 (series of m, 6H), 1.19 (d, J=6.2 Hz, 3H), 1.14-1.13 (m, 12H). UPLC (M+H)=766.4.

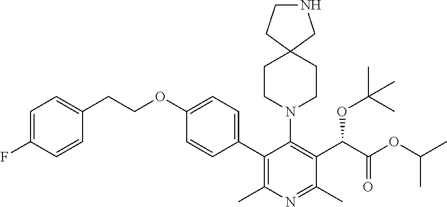 [SMILES: Cc1nc(C)c([C@H](OC(C)(C)C)C(=O)OC(C)C)c(N2CCC3(CCNC3)CC2)c1-c1ccc(OCCc2ccc(F)cc2)cc1]

### (S)-Isopropyl 2-(tert-butoxy)-2-(5-(4-(4-fluorophenethoxy)phenyl)-2,6-dimethyl-4-(2,8-diazaspiro[4.5]decan-8-yl)pyridin-3-yl)acetate

To a solution of (S)-benzyl 8-(3-(1-(tert-butoxy)-2-isopropoxy-2-oxoethyl)-5-(4-(4-fluorophenethoxy) phenyl)-2,6-dimethylpyridin-4-yl)-2,8-diazaspiro[4.5]decane-2-carboxylate (100 mg, 0.131 mmol) in MeOH (0.5 mL) was added to a suspension of Pearlman's Catalyst (18 mg, 0.171 mmol) in dry MeOH (2.5 mL) at rt. The flask was evacuated and charged with hydrogen (ballon) and stirred for 4 h. Additional catalyst (25 mg) was added and the solution was placed under pressure (50 psi) on Parr shaker for 5 h. The reaction mixture was filtered, concentrated, and there was obtained (S)-isopropyl 2-(tert-butoxy)-2-(5-(4-(4-fluorophenethoxy)phenyl)-2,6-dimethyl-4-(2,8-diazaspiro[4.5]decan-8-yl)pyridin-3-yl)acetate (yield not determined). UPLC (M+H)=632.5.

### Example 68

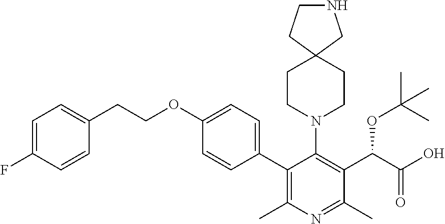 [SMILES: Cc1nc(C)c([C@H](OC(C)(C)C)C(=O)O)c(N2CCC3(CCNC3)CC2)c1-c1ccc(OCCc2ccc(F)cc2)cc1]

### (S)-2-(tert-Butoxy)-2-(5-(4-(4-fluorophen ethoxy)phenyl)-2,6-dimethyl-4-(2,8-diazaspiro[4.5]decan-8-yl)pyridin-3-yl)acetic acid

Potassium hydroxide (73.2 mg, 1.31 mmol) was added to a solution of (S)-isopropyl 2-(tert-butoxy)-2-(5-(4-(4-fluorophenethoxy)phenyl)-2,6-dimethyl-4-(2,8-diazaspiro[4.5]decan-8-yl)pyridin-3-yl)acetate in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep to obtain (S)-2-(tert-butoxy)-2-(5-(4-(4-fluorophen ethoxy)phenyl)-2,6-dimethyl-4-(2,8-diazaspiro[4.5]decan-8-yl)pyridin-3-yl)acetic acid as a mixture of diastereomers 30 mg (37% for two steps). ¹H NMR (500 MHz, DMSO) δ 7.39-7.36 (m, 2H), 7.20-7.13 (m, 3H), 7.08-6.93 (m, 3H), 5.63/5.53 (br. s, 1H), 4.24-4.20 (m, 2H), 3.81 (s, 1H), 3.38-3.36 (m, 4H), 3.05 (t, J=6.2 Hz, 2H), 2.98 (br. s, 1H), 2.65 (s, 1H), 2.42 (s, 3H), 2.29 (br. s, 1H), 2.03 (s, 3H), 1.64-1.31 (series of m, 6H), 1.10/1.08 (s, 9H). UPLC (M+H)=590.4.

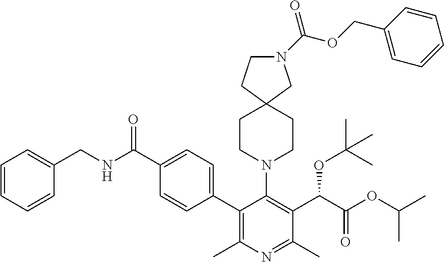 [SMILES: Cc1nc(C)c([C@H](OC(C)(C)C)C(=O)OC(C)C)c(N2CCC3(CCN(C(=O)OCc4ccccc4)C3)CC2)c1-c1ccc(C(=O)NCc2ccccc2)cc1]

### (S)-Benzyl 8-(3-(4-(benzylcarbamoyl)phenyl)-5-(1-(tert-butoxy)-2-isopropoxy-2-oxoethyl)-2,6-dimethylpyridin-4-yl)-2,8-diazaspiro[4.5]decane-2-carboxylate

The Pd(Ph₃P)₄ (19.2 mg, 0.017 mmol) was added to an argon purged and degassed solution of (S)-benzyl 8-(3-bromo-5-(1-(tert-butoxy)-2-isopropoxy-2-oxoethyl)-2,6-dimethylpyridin-4-yl)-2,8-diazaspiro[4.5]decane-2-carboxylate (105 mg, 0.167 mmol), (4-(benzylcarbamoyl)phenyl)boronic acid (47 mg, 0.18 mmol), and sodium carbonate (88 mg, 0.833 mmol) in dioxane (1.5 mL) and water (0.4 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-benzyl 8-(3-(4-(benzylcarbamoyl)phenyl)-5-(1-(tert-butoxy)-2-isopropoxy-2-oxoethyl)-2,6-dimethylpyridin-4-yl)-2,8-diazaspiro[4.5]decane-2-carboxylate 120 mg (95%). ¹H NMR (500 MHz, DMSO) δ 9.17-9.12 (m, 1H), 8.05-7.99 (m, 2H), 7.50-7.47 (m, 1H), 7.37-7.24 (m, 11H), 6.00 (s, 1H), 5.02 (s, 2H), 4.99-4.93 (m, 1H), 4.54-4.51 (m, 2H), 3.35 (d, J=4.4 Hz, 2H), 3.30-3.20 (m, 2H), 3.11-2.90 (m, 2H), 2.81-2.65 (m, 2H), 2.47 (s, 3H), 2.37 (br. s, 1H), 2.05 (s, 3H), 1.87-1.78 (m, 1H), 1.65-1.22 (series of m, 6H), 1.20-1.15 (m, 15H). UPLC (M+H)=761.5.

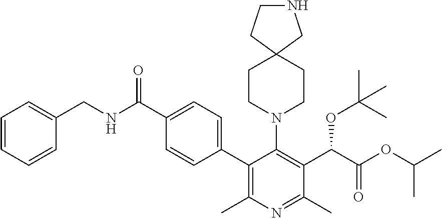 [SMILES: Cc1nc(C)c([C@H](OC(C)(C)C)C(=O)OC(C)C)c(N2CCC3(CCNC3)CC2)c1-c1ccc(C(=O)NCc2ccccc2)cc1]

### (S)-Isopropyl 2-(5-(4-(benzylcarbamoyl)phenyl)-2,6-dimethyl-4-(2,8-diazaspiro[4.5]decan-8-yl)pyridin-3-yl)-2-(tert-butoxy)acetate

The a solution of (S)-benzyl 8-(3-(4-(benzylcarbamoyl)phenyl)-5-(1-(tert-butoxy)-2-isopropoxy-2-oxoethyl)-2,6-dimethylpyridin-4-yl)-2,8-diazaspiro[4.5]decane-2-carboxylate (100 mg, 0.131 mmol) in MeOH (0.5 mL) was added to a suspension of Pearlman's Catalyst (18 mg, 0.171 mmol) in dry MeOH (2.5 mL) at rt. The flask was evacuated and charged with hydrogen (ballon) and stirred for 4 h, an additional catalyst (25 mg) was added. The reaction mixture was stirred 16 h, filtered, concentrated, and there was obtained (S)-isopropyl 2-(5-(4-(benzylcarbamoyl)-phenyl)-2,6-dimethyl-4-(2,8-diazaspiro[4.5]decan-8-yl)pyridin-3-yl)-2-(tert-butoxy)acetate 71 mg (87%). UPLC (M+H)=627.4.

### Example 69

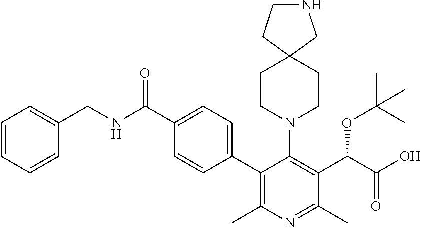 [SMILES: Cc1nc(C)c([C@H](OC(C)(C)C)C(=O)O)c(N2CCC3(CCNC3)CC2)c1-c1ccc(C(=O)NCc2ccccc2)cc1]

### (S)-2-(5-(4-(Benzylcarbamoyl) phenyl)-2,6-dimethyl-4-(2,8-diazaspiro[4.5]decan-8-yl)pyridin-3-yl)-2-(tert-butoxy)acetic acid

Potassium hydroxide (73.4 mg, 1.31 mmol) was added to a solution of (S)-isopropyl 2-(5-(4-(benzyl carbamoyl)phenyl)-2,6-dimethyl-4-(2,8-diazaspiro[4.5]decan-8-yl)pyridin-3-yl)-2-(tert-butoxy)acetate (71 mg, 0.113 mmol) in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(5-(4-(benzylcarbamoyl) phenyl)-2,6-dimethyl-4-(2,8-diazaspiro[4.5]decan-8-yl)pyridin-3-yl)-2-(tert-butoxy)acetic acid as a mixture of diastereomers 32.5 mg (41%). ¹H NMR (500 MHz, DMSO) δ 9.16-9.14 (m, 1H), 8.05-7.94 (m, 2H), 7.46-7.43 (m, 1H), 7.34-7.32 (m, 4H), 7.27-7.20 (m, 1H), 7.15-7.13 (m, 1H), 5.53/5.45 (s, 1H), 4.53-4.40 (m, 2H), 3.82 (br. s, 1H), 3.38 (br. s, 4H), 2.99-2.93 (m, 1H), 2.61 (br. s, 1H), 2.43 (s, 3H), 2.33-2.29 (m, 1H), 2.04/2.03 (s, 3H), 1.62-1.30 (series of m, 6H), 1.09/1.07 (s, 9H). UPLC (M+H)=585.4.

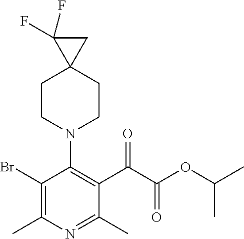 [SMILES: Cc1nc(C)c(C(=O)C(=O)OC(C)C)c(N2CCC3(CC2)CC3(F)F)c1Br]

### Isopropyl 2-(5-bromo-4-(1,1-difluoro-6-azaspiro[2.5]octan-6-yl)-2,6-dimethylpyridin-3-yl)-2-oxoacetate

To a solution of 1,1-difluoro-6-azaspiro[2.5]octane, HCl (500 mg, 2.72 mmol) and DIEA (1.1 g, 8.2 mmol) in anhydrous CH₃CN (20 mL) was added isopropyl 2-(5-bromo-4-chloro-2,6-dimethylpyridin-3-yl)-2-oxoacetate (911 mg, 2.2 mmol) at rt. The resulting mixture was placed in a pre-heated oil bath (80° C.) and stirred for 18 h before being cooled, concentrated, and charged (DCM) to a 40 g ISCO silica gel cartridge and gradient elution (0-25% EtOAc/hexanes) using an Isolera chromatography station to give isopropyl 2-(5-bromo-4-(1,1-difluoro-6-azaspiro[2.5]octan-6-yl)-2,6-dimethylpyridin-3-yl)-2-oxoacetate 427 mg (35%). ¹H NMR (500 MHz, DMSO) δ 5.13-5.08 (m, 1H), 3.34-3.33 (m, 2H), (protons adj to nitrogen not evident), 2.62 (s, 3H), 2.32 (s, 3H), 1.65-1.50 (m, 4H), 1.35 (t, J=7.7 Hz, 2H), 1.30 (d, J=6.2 Hz, 6H). UPLC (M+H)=447.1.

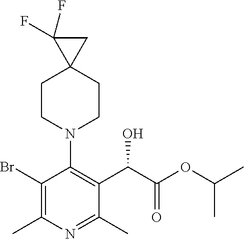 [SMILES: Cc1nc(C)c([C@H](O)C(=O)OC(C)C)c(N2CCC3(CC2)CC3(F)F)c1Br]

### (S)-Isopropyl 2-(5-bromo-4-(1,1-difluoro-6-azaspiro[2.5]octan-6-yl)-2,6-dimethylpyridin-3-yl)-2-hydroxyacetate

The benzo[d][1,3,2]dioxaborole (0.73 mL, 1.74 mmol; 50% soln in toluene) was added to a nitrogen purged solution of isopropyl 2-(5-bromo-4-(1,1-difluoro-6-azaspiro[2.5]octan-6-yl)-2,6-dimethylpyridin-3-yl)-2-oxoacetate (388 mg, 0.87 mmol) and 0.3.5 mL of 1M (R)-1-methyl-3,3-diphenylhexahydropyrrolo[1,2-c][1,3,2]oxazaborole (0.35 mmol) in toluene (20 mL) cooled to −50° C. The reaction was allowed to slowly warm to −15° C. and placed in the freezer for 18 h before being quenched with 1M Na₂CO₃ (3 mL) and stirred for 20 min. The organic layer was diluted with EtOAc and washed with brine and dried (MgSO₄). The crude product was charged (DCM) to a 40 g ISCO silica gel cartridge and gradient elution (0-50% EtOAc/hexanes) using an Isolera chromatography station gave (S)-isopropyl 2-(5-bromo-4-(1,1-difluoro-6-azaspiro[2.5]octan-6-yl)-2,6-dimethylpyridin-3-yl)-2-hydroxyacetate 3.45 mg (89%). UPLC (M+H)=449.2.

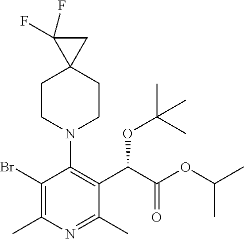 [SMILES: Cc1nc(C)c([C@H](OC(C)(C)C)C(=O)OC(C)C)c(N2CCC3(CC2)CC3(F)F)c1Br]

### (S)-Isopropyl 2-(5-bromo-4-(1,1-difluoro-6-azaspiro[2.5]octan-6-yl)-2,6-dimethylpyridin-3-yl)-2-(tert-butoxy)acetate

The isobutylene gas was bubbled into a nitrogen purged, cooled (0° C.) solution of (S)-isopropyl 2-(5-bromo-4-(1,1-difluoro-6-azaspiro[2.5]octan-6-yl)-2,6-dimethylpyridin-3-yl)-2-hydroxyacetate (320 mg, 0.72 mmol) and 0.07 mL of 70% HClO₄ in DCM (6 mL) for 20 min. The reaction mixture was allowed to warm to rt and stirred for 18 h in a pressure sealed vessel, diluted with DCM, washed with 1M Na₂CO₃ soln, and dried over MgSO₄. The crude product was charged (DCM) to a 40 g ISCO silica gel cartridge and gradient elution (0-35% EtOAc/hexanes) using an Isolera chromatography station gave (S)-isopropyl 2-(5-bromo-4-(1,1-difluoro-6-azaspiro[2.5]octan-6-yl)-2,6-dimethylpyridin-3-yl)-2-(tert-butoxy)acetate 344 mg (95%). UPLC (M+H)=503.2.

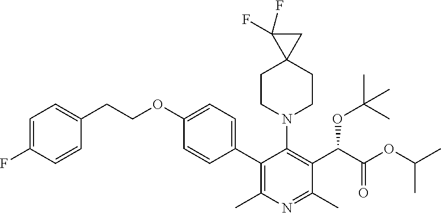 [SMILES: Cc1nc(C)c([C@H](OC(C)(C)C)C(=O)OC(C)C)c(N2CCC3(CC2)CC3(F)F)c1-c1ccc(OCCc2ccc(F)cc2)cc1]

### Isopropyl 2-(tert-butoxy)-2-(4-(1,1-difluoro-6-azaspiro[2.5]octan-6-yl)-5-(4-(4-fluorophenethoxy)phenyl)-2,6-dimethylpyridin-3-yl)acetate

The Pd(Ph₃P)₄ (25.2 mg, 0.022 mmol) was added to an argon purged and degassed solution of (S)-isopropyl 2-(5-bromo-4-(1,1-difluoro-6-azaspiro[2.5]octan-6-yl)-2,6-dimethylpyridin-3-yl)-2-(tert-butoxy)acetate (110 mg, 0.22 mmol), (4-(4-fluorophenethoxy)phenyl)boronic acid (63 mg, 0.24 mmol), and sodium carbonate (116 mg, 1.10 mmol) in dioxane (2 mL) and water (0.5 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-isopropyl 2-(tert-butoxy)-2-(4-(1,1-difluoro-6-azaspiro[2.5]octan-6-yl)-5-(4-(4-fluorophenethoxy)phenyl)-2,6-dimethylpyridin-3-yl)acetate (yield not determined). ¹H NMR (500 MHz, DMSO) δ 7.39-7.37 (m, 2H), 7.22-7.21 (m, 1H), 7.14 (t, J=8.8 Hz, 2H), 7.02-7.00 (m, 3H), 6.00 (br. s, 1H), 4.98-4.93 (m, 1H), 4.26-4.22 (m, 2H), 3.39-3.38 (m, 3H), 3.06 (t, J=6.6 Hz, 2H), 2.57 (br. s, 1H), 2.47 (s, 3H), 2.05 (s, 3H), 1.74 (br. s, 4H), 1.19 (d, J=6.2 Hz, 3H), 1.15-1.13 (m, 14H). UPLC (M+H)=639.3.

### Example 70

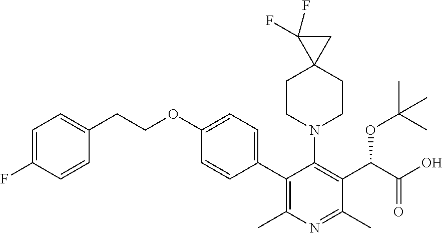 [SMILES: Cc1nc(C)c([C@H](OC(C)(C)C)C(=O)O)c(N2CCC3(CC2)CC3(F)F)c1-c1ccc(OCCc2ccc(F)cc2)cc1]

### (S)-2-(tert-Butoxy)-2-(4-(1,1-difluoro-6-azaspiro[2.5]octan-6-yl)-5-(4-(4-fluorophenethoxy)phenyl)-2,6-dimethylpyridin-3-yl)acetic acid

Potassium hydroxide (123 mg, 2.8 mmol) was added to a solution of (S)-isopropyl 2-(tert-butoxy)-2-(4-(1,1-difluoro-6-azaspiro[2.5]octan-6-yl)-5-(4-(4-fluorophenethoxy)phenyl)-2,6-dimethylpyridin-3-yl)acetate in EtOH (2 mL) and the solution was heated at reflux for 6 h. The reaction mixture was cooled, diluted with EtOAc, neutralized (1M HCl soln) to pH 4, and the organic layer was washed with brine, and dried (MgSO₄). The crude product was purified by prep HPLC to obtain (S)-2-(tert-butoxy)-2-(4-(1,1-difluoro-6-azaspiro[2.5]octan-6-yl)-5-(4-(4-fluorophenethoxy)phenyl)-2,6-dimethylpyridin-3-yl)acetic acid 61 mg (47% for two steps). ¹H NMR (500 MHz, DMSO) δ 7.40-7.36 (m, 2H), 7.22-7.18 (m, 1H), 7.16-7.12 (m, 2H), 7.04-6.99 (m, 3H), 5.81/5.76 (br. s, 1H), 4.26-4.22 (m, 2H), 3.45 (d, J=12 Hz, 1H), 3.06 (t, J=5.9 Hz, 2H), 2.68 (t, J=11 Hz, 1H), 2.60-2.57 (m, 1H), 2.48 (s, 3H), 2.04 (s, 3H), 2.00-1.95 (m, 1H), 1.89-1.70 (m, 3H), 1.31-1.28 (m, 1H), 1.16 (s, 9H), 0.99-0.96 (m, 1H), 0.92-0.89 (m, 1H). UPLC (M+H)=597.3.

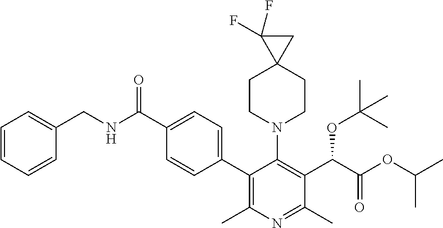 [SMILES: Cc1nc(C)c([C@H](OC(C)(C)C)C(=O)OC(C)C)c(N2CCC3(CC2)CC3(F)F)c1-c1ccc(C(=O)NCc2ccccc2)cc1]

### (S)-Isopropyl 2-(5-(4-(benzylcarbamoyl)phenyl)-4-(1,1-difluoro-6-azaspiro[2.5]octan-6-yl)-2,6-dimethylpyridin-3-yl)-2-(tert-butoxy)acetate

The Pd(Ph₃P)₄ (24.1 mg, 0.021 mmol) was added to an argon purged and degassed solution of (S)-isopropyl 2-(5-bromo-4-(1,1-difluoro-6-azaspiro[2.5]octan-6-yl)-2,6-dimethylpyridin-3-yl)-2-(tert-butoxy)acetate (105 mg, 0.21 mmol), (4-(benzylcarbamoyl)phenyl)boronic acid (59 mg, 0.23 mmol), and sodium carbonate (106 mg, 1.04 mmol) in dioxane (1.5 mL) and water (0.4 mL) and stirred in a screw-capped pressure vessel for 16 h at 90° C. The reaction was allowed to cool, diluted with EtOAc, and the organic layer was washed with brine and dried (MgSO₄). The crude product was charged (DCM) to a 40 g ISCO silica gel cartridge and gradient elution (0-50% EtOAc/hexanes) using an Isolera chromatography station gave (S)-isopropyl 2-(5-(4-(benzylcarbamoyl)phenyl)-4-(1,1-difluoro-6-azaspiro[2.5]octan-6-yl)-2,6-dimethylpyridin-3-yl)-2-(tert-butoxy)acetate 127 mg (96%). ¹H NMR (500 MHz, DMSO) δ 9.17 (br. s, 1H), 8.03-7.99 (m, 2H), 7.50-7.46 (m, 1H), 7.36-7.34 (m, 4H), 7.27-7.24 (m, 2H), 6.01/5.98 (br. s, 1H), 4.99-4.94 (m, 1H), 4.53-4.51 (m, 2H), 3.38-3.37 (m, 3H), 2.60 (br. s, 1H), 2.55 (s, 3H), 2.04 (s, 3H), 1.96-1.82 (m, 1H), 1.72-1.67 (m, 1H), 1.39-1.23 (m, 2H), 1.20-1.15 (m, 15), 1.09-1.00 (m, 1H), 0.98-0.93 (m, 1H). UPLC (M+H)=634.4.

### Example 71

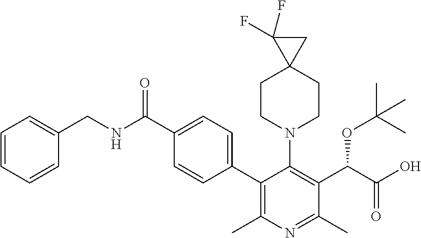 [SMILES: Cc1nc(C)c([C@H](OC(C)(C)C)C(=O)O)c(N2CCC3(CC2)CC3(F)F)c1-c1ccc(C(=O)NCc2ccccc2)cc1]

### (S)-2-(5-(4-(Benzylcarbamoyl)phenyl)-4-(1,1-difluoro-6-azaspiro[2.5]octan-6-yl)-2,6-dimethylpyridin-3-yl)-2-(tert-butoxy)acetic acid

Potassium hydroxide (117 mg, 2.1 mmol) was added to a solution of (S)-isopropyl 2-(5-(4-(benzylcarbamoyl)-phenyl)-4-(1,1-difluoro-6-azaspiro[2.5]octan-6-yl)-2,6-dimethylpyridin-3-yl)-2-(tert-butoxy)acetate (110 mg, 0.21 mmol) in EtOH (1.5 mL) and the solution was heated at 80° C. for 6 h. The reaction mixture was cooled, diluted with EtOAc, neutralized (1M HCl soln) to pH 5, and the organic layer was washed with brine, and dried (MgSO₄). The crude product was purified by prep HPLC to obtain (S)-2-(5-(4-(benzyl-carbamoyl)phenyl)-4-(1,1-difluoro-6-azaspiro[2.5]octan-6-yl)-2,6-dimethylpyridin-3-yl)-2-(tert-butoxy)acetic acid 74 mg (71%). ¹H NMR (500 MHz, DMSO) δ 9.18-9.15 (m, 1H), 8.03-7.98 (m, 2H), 7.48 (t, J=8.8 Hz, 1H), 7.36-7.34 (m, 4H), 7.27-7.22 (m, 2H), 5.79/5.74 (br. s, 1H), 4.52 (s, 2H), 3.55-3.52 (m, 1H), 3.40 (br. s, 3H), 2.55 (s, 3H), 2.03 (s, 3H), 2.01-1.95 (m, 1H), 1.85-1.65 (m, 2H), 1.33-1.28 (m, 1H), 1.17 (s, 9H), 1.07-1.03 (m, 1H), 0.99-0.91 (m, 1H). UPLC (M+H)=592.3.

### Biological Methods

Inhibition of HIV Replication:

A recombinant NL-RLuc proviral clone was constructed in which a section of the nef gene from NL4-3 was replaced with the Renilla Luciferase gene. This virus is fully infectious and can undergo multiple cycles of replication in cell culture. In addition, the luciferous reporter provides a simple and easy method for quantitating the extent of virus growth and consequently, the antiviral activity of test compounds. The plasmid pNLRLuc contains the proviral NL-Rluc DNA cloned into pUC 18 at the PvuII site. The NL-RLuc virus was prepared by transfection of 293T cells with the plasmid pNLRLuc. Transfections were performed using the LipofectAMINE PLUS kit from Invitrogen (Carlsbad, Calif.) according to the manufacturer and the virus generated was titered in MT-2 cells. For susceptibility analyses, the titrated virus was used to infect MT-2 cells in the presence of compound, and after 5 days of incubation, cells were processed and quantitated for virus growth by the amount of expressed luciferase. Assay media was RPMI 1640 supplemented with 10% heat inactivated fetal bovine serum (FBS), 100 units/ml penicillin G/100 units/ml streptomycin, 10 mM HEPES buffer pH 7.55 and 2 mM L-glutamine. The results from at least 2 experiments were used to calculate the EC₅₀ values. Luciferase was quantitated using the Dual Luciferase kit from Promega (Madison, Wis.). Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}](Johnson V A, Byington R T. Infectivity Assay. In Techniques in HIV Research. ed. Aldovini A, Walker B D. 71-76. New York: Stockton Press.1990). Results are shown in Table 1. Activity equal to A refers to a compound having an EC₅₀≤100 nM, while B and C denote compounds having an EC₅₀ between 100 nM and 1 uM (B) or >1 uM (C).

TABLE 1

| Example | Activity | EC₅₀ μM |
|---|---|---|
| 1 | B | 0.446 |
| 2 | A | 0.036 |
| 3 | A | |
| 4 | A | |
| 5 | A | |
| 6 | B | |
| 7 | B | 0.651 |
| 8 | A | |
| 9 | A | 0.083 |
| 10 | A | |
| 11 | A | |
| 12 | A | |
| 13 | A | |
| 14 | A | |
| 15 | A | |
| 16 | A | 0.003 |
| 17 | A | |
| 18 | B | 0.475 |
| 19 | B | |
| 20 | A | |
| 21 | A | |
| 22 | A | 0.026 |
| 23 | ND | ND |
| 24 | A | |
| 25 | A | |
| 26 | A | |
| 27 | A | |
| 28 | A | |
| 29 | A | 0.007 |
| 30 | ND | ND |
| 31 | A | |
| 32 | A | |
| 33 | A | |
| 34 | A | |
| 35 | A | 0.006 |
| 36 | ND | ND |
| 37 | A | |
| 38 | A | |
| 39 | A | |
| 40 | A | |
| 41 | A | |
| 42 | A | 0.002 |
| 43 | A | |
| 44 | A | |
| 45 | A | |
| 46 | A | |
| 47 | A | |
| 48 | A | |
| 49 | A | 0.006 |
| 50 | A | |
| 51 | A | |
| 52 | A | |
| 53 | A | |
| 54 | A | |
| 55 | A | 0.001 |
| 56 | A | |
| 57 | A | |
| 58 | A | |
| 59 | A | |
| 60 | A | |
| 61 | A | |
| 62 | A | 0.017 |
| 63 | A | |
| 64 | A | |
| 65 | A | |
| 66 | A | |
| 67 | A | |
| 68 | B | 0.731 |
| 69 | C | 9.656 |
| 70 | A | |
| 71 | A | 0.018 |

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1-10. (canceled)

11. A compound of Formula I 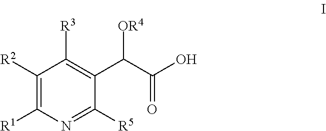
wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is is selected from tetrahydroisoquinolinyl or decahydroisoquinolinyl and is substituted with 0-3 substituents selected from halo, alkyl, and haloalkyl;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

12. (canceled)

13. A compound of Formula I 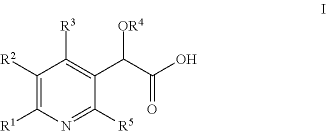
wherein:
R¹ is selected from H, alkyl, haloalkyl, hydroxyalkyl, alkoxyalkyl, (alkoxy)alkoxyalkyl, or (R⁶)alkyl;
R² is phenyl substituted with 1 R⁷ substituent and with 0-3 substituents selected from halo, alkyl, haloalkyl, alkoxy, and haloalkoxy;
or R² is selected from tetrahydroisoquinolinyl, ((Ar¹)alkyl)tetrahydroisoquinolinyl, or ((N-alkoxycarbonyl)tetrahydroisoquinolinyl
R³ is selected from azetidinyl, pyrrolidinyl, piperidinyl, or homopiperidinyl and contains a spirocyclic moiety wherein the spirocyclic moiety, including the carbon atom to which it is attached, forms C₃₋₇ cycloalkane, tetrahydrofuranyl, tetrahydropyranyl, pyrrolidinyl, N-alkylpyrrolidinyl, piperidinyl, N-alkylpiperidinyl, homopiperidinyl, or N-alkylpiperidinyl, and wherein the spirocyclic moiety is substituted with 0-3 halo or alkyl substituents;
R⁴ is selected from alkyl or haloalkyl;
R⁵ is selected from H, alkyl, haloalkyl, hydroxyalkyl, alkoxyalkyl, (alkoxy)alkoxyalkyl, or (R⁶)alkyl;
R⁶ is selected from (oxetanyl)oxy, ((oxetanyl)alkoxy)alkyl, (tetrahydropyranyloxy)alkyl, (tetrahydropyranyl)alkoxy)alkyl, or (R⁸)(R⁹)N;
R⁷ is selected from (Ar¹)alkoxy or ((Ar¹)alkyl)HNCO;
R⁸ is selected from hydrogen, alkyl, (cycloalkyl)alkyl, alkoxyalkyl, (tetrahydropyanyl)alkyl, tetrahydropyanyl, or alkoxyphenyl;
R⁹ is selected from hydrogen or alkyl;
or (R⁸)(R⁹)N taken together is selected from azetidinyl, pyrrolidinyl, piperidinyl, (spirocyclobutyl)piperidinyl, piperazinyl, or morpholinyl; and
Ar¹ is phenyl substituted with 0-3 substituents selected from halo, alkyl, haloalkyl, alkoxy, and haloalkoxy;
or a pharmaceutically acceptable salt thereof.

14-15. (canceled)

16. A pharmaceutical composition comprising a compound or salt of claim 11.

17. The composition of claim 16 further comprising at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

18. The composition of claim 17 wherein the other agent is dolutegravir.

19. A method for treating HIV infection comprising administering a compound of claim 11, or a pharmaceutically acceptable salt thereof, to a patient in need thereof.

20. The method of claim 19 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

21. The method of claim 20 wherein the other agent is dolutegravir.

22. (canceled)

23. A compound or salt of claim 11 wherein R² is phenyl substituted with 1 R⁷ substituent and with 0-3 substituents selected from halo, alkyl, haloalkyl, alkoxy, and haloalkoxy.

24. A compound or salt of claim 11 wherein R² is selected from tetrahydroisoquinolinyl, ((Ar¹)alkyl)tetrahydroisoquinolinyl, or ((N-alkoxycarbonyl)tetrahydroisoquinolinyl.

25. A compound or salt of claim 13 wherein R² is phenyl substituted with 1 R⁷ substituent and with 0-3 substituents selected from halo, alkyl, haloalkyl, alkoxy, and haloalkoxy.

26. A compound or of claim 13 wherein R² is selected from tetrahydroisoquinolinyl, ((Ar¹)alkyl)tetrahydroisoquinolinyl, or ((N-alkoxycarbonyl)tetrahydroisoquinolinyl.

27. A pharmaceutical composition comprising a compound or salt of claim 13.

28. The composition of claim 27 further comprising at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

29. The composition of claim 28 wherein the other agent is dolutegravir.

30. A method for treating HIV infection comprising administering a compound of claim 13, or a pharmaceutically acceptable salt thereof, to a patient in need thereof.

31. The method of claim 30 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

32. The method of claim 31 wherein the other agent is dolutegravir.
